SEQUENCE LISTING (6 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 1255 aa, Homo sapiens [sequence omitted]

SEQ ID NO: 3 - protein, 12 aa, synthetic construct
  REGION 1..12: note = Synthetic polypeptide
  MOD_RES 5: note = PHOSPHORYLATION
  MOD_RES 9: note = PHOSPHORYLATION
RAEDSGNESEGE

SEQ ID NO: 4 - protein, 22 aa, synthetic construct
  REGION 1..22: note = Synthetic polypeptide
  MOD_RES 15: note = PHOSPHORYLATION
  MOD_RES 19: note = PHOSPHORYLATION
  VARIANT 1..10: note = Amino acid is Gly, and some or all may be absent
XXXXXXXXXXRAEDSGNESEGE

SEQ ID NO: 5 - protein, 11 aa, synthetic construct
  REGION 1..11: note = Synthetic polypeptide
  MOD_RES 6: note = PHOSPHORYLATION
  MOD_RES 10: note = PHOSPHORYLATION
DRIIDSGLDSM

SEQ ID NO: 6 - protein, 21 aa, synthetic construct
  REGION 1..21: note = Synthetic polypeptide
  MOD_RES 16: note = PHOSPHORYLATION
  MOD_RES 20: note = PHOSPHORYLATION
  VARIANT 1..10: note = Amino acid is Gly, and some or all may be absent
XXXXXXXXXXDRIIDSGLDSM

(19) United States
(12) Patent Application Publication
(10) Pub. No.: US 2025/0388563 A1
(43) Pub. Date: Dec. 25, 2025

(54) HER3 LIGANDS AND USES THEREOF

(71) Applicant: The Regents of the University of California, Oakland, CA (US)

(72) Inventors: Natalia Jura, San Francisco, CA (US); Kevan M. Shokat, San Francisco, CA (US); Jose Carmona-Negron, San Francisco, CA (US); Ziyang Zhang, San Mateo, CA (US)

(21) Appl. No.: 18/730,685

(22) PCT Filed: Jan. 23, 2023
(86) PCT No.: PCT/US2023/061105

§ 371 Date: Jul. 19, 2024

Related U.S. Application Data
(60) Provisional application No. 63/301,915, filed on Jan. 21, 2022.

(51) Int. Cl. C07D 401/14; A61K 31/496; C07D 417/14

(52) U.S. Cl. CPC C07D 401/14; A61K 31/496; C07D 417/14

(57) ABSTRACT

Described herein, inter alia, are compositions of HERS ligands comprising degradation-increasing moiety, and compositions and methods for treating diseases associated with HERS activity, including cancer, in a patient in need of such treatment using the same. Further disclosed methods of treating a disease associated with EGFR activity, HER2 activity, HER4 activity, c-MET activity, RISK activity, MEK activity, MARK activity, RAF activity, BRAF activity, AKT activity, RAS activity, KRAS activity, or neuregulin activity in a patient in need of such treatment.

This application claims the benefit of priority to U.S. Provisional Application Ser. No. 63/301,915 filed Jan. 21, 2022. The disclosure of this application is incorporated herein by reference in its entirty and for all purposes.

### STATEMENT AS TO RIGHTS TO INVENTIONS MADE UNDER FEDERALLY SPONSORED RESEARCH AND DEVELOPMENT

This invention was made with government support under grant no. K12 GM081266 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 2025-03-10 Revised_Sequence_Listing 048536-726N01US.xml, created Mar. 10, 2025, 14,173 bytes, machine format IBM-PC, MS-Windows operating system, is hereby incorporated by reference.

### BACKGROUND

Pseudokinases are members of the large protein kinase family which do not exhibit substantial enzymatic activity. However, they do participate in signal transduction through orchestration of protein-protein interactions with other active enzymes. HER3 in particular binds to HER2 which is an active kinase.

There is evidence that HER3 inhibition is beneficial in treatment of head and neck, lung and prostate cancers. A spectrum of therapeutic anti-HER3 antibodies are at different stages in clinical trials, but they are inferior to small molecules due to difficulties with brain penetration, delivery, target, engagement, prosuction, storage, and cost. Thus, direct degradation of HER3 will represent a more significant strategy for inhibition. There is a need in the art for efficient and effective small molecule blockers of HER3 function. Disclosed herein are solutions to these and other problems in the art.

### BRIEF SUMMARY

Provided herein, inter alia, are ligands for HER3, and methods of using the same.

In an aspect is provided a compound having the formula:

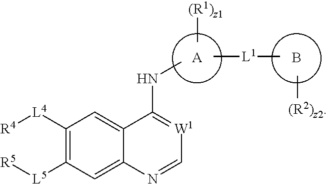

Ring A is aryl or heteroaryl. Ring B is aryl or heteroaryl. W¹ is N or C(R⁶). R¹ is independently a halogen, —CX¹₃, —CHX¹₂, —CH₂X¹, —CN, —SOₙ₁R⁷R⁸, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. R² is independently a halogen, —CX²₃, —CHX²₂, —CH₂X², —CN, —SOₙ₂R¹⁴, —SOᵥ₂NR¹¹R¹², —NHNH₂, —ONR¹¹R¹², —NHC═(O)NHNR¹¹R¹², —NHC═(O)NR¹¹R¹², —N(O)ₘ₂, —NR¹¹R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR¹¹OR¹³, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl. R⁴ is independently a hydrogen, halogen, —CX⁴₃, —CHX⁴₂, —CH₂X⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁴₃, —OCHX⁴₂, —OCH₂X⁴₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is a degradation-increasing moiety. R⁶ is a hydrogen, halogen, —CX⁶₃, —CHX⁶₂, —CH₂X⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁶₃, —OCHX⁶₂, —OCH₂X⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷, R⁸, R⁹, R¹⁰, R¹¹, R¹², R¹³, and R¹⁴ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R¹¹ and R¹² substituents bonded to the same nitrogen atom may optionally be joined to form a substituted or unsubstituted heterocycloalkyl or substituted or unsubstituted heteroaryl. L¹ is a bond, —O—, —NH—, —C(O)—, —C(O)NH—, —NHC(O)—, —C(O)O—, —OC(O)—, —NHC(O)NH—, —S—, substituted or unsubstituted alkylene or substituted or unsubstituted heteroalkylene. L⁴ is a bond or a divalent linker. L⁵ is a divalent linker. z1 and z2 are independently an integer from 0 to 7. m1, m2, v1, and v2 are independently 1 or 2. n1 and n2 are independently an integer from 0 to 4. X¹, X², X⁴, X⁶, and X^{A} are independently —Cl, —Br, —I, or —F.

In an aspect is provided a pharmaceutical composition including a compound described herein or a pharmaceutically acceptable salt thereof, and a pharmaceutically acceptable excipient.

In an aspect is provided a method of treating a disease associated with HER3 activity in a patient in need of such treatment, the method including administering a therapeutically effective amount of a compound described herein, or a pharmaceutically acceptable salt thereof.

In an aspect is provided a method of treating a disease associated with EGFR activity, HER2 activity, HER4 activity, c-MET activity, PI3K activity, MEK activity, MAPK activity, RAF activity, BRAF activity, AKT activity, RAS activity, KRAS activity, heregulin activity, or neuregulin activity in a patient in need of such treatment, the method including administering a therapeutically effective amount of a compound described herein, or a pharmaceutically acceptable salt thereof.

In an aspect is provided a method of treating cancer in a patient in need of such treatment, the method including administering a therapeutically effective amount of a compound described herein, or a pharmaceutically acceptable salt thereof.

In an aspect is provided a method of inhibiting HER3 activity, the method including contacting HER3 with an effective amount of a compound described herein, or a pharmaceutically acceptable salt thereof.

### BRIEF DESCRIPTION OF THE DRAWINGS

FIGS. 1A-ID. FIG. 1A depicts the structure of the HER3 pseudokinase domain with bosutinib. FIG. 1B depicts the structure of bosutinib. FIG. 1C depicts the structure of 179D. FIG. 1D depicts the structure of 050A.

FIG. 2A-2E. FIG. 2A depicts DSF-based measurement of binding of the botusinib, 179D and 050A compounds to the HER3 pseudokinase relative to the DMSO control. FIG. 2B shows comparative binding to the HER3 pseudokinase by 050A (1^{st} generation degraders) and 06-078. FIG. 2C shows comparative binding to the HER3 pseudokinase by 050A (1^{st} generation degraders) and 06-085. FIG. 2D shows comparative binding to the HER3 pseudokinase by 050A (1^{st} generation degraders) and 06-086. FIG. 2E shows the structures of 06-078, 06-085 and 06-086 comounds.

FIG. 3. FIG. 3 demonstrates the optimization of 179D and 50A warheads for the 2^{nd} generation HER3 gedraders (07-042, 02-012 and 01-13 warheads).

FIG. 4. FIG. 4 demonstrates difference in binding measured by DSF between 179D and 050A and 07-042, 02-012 and 01-13 warheads.

FIGS. 5A-5B. FIG. 5A depicts a DSF-based measurement of HER3 binding by compounds 02-016 and 02-004 compared to bosutinib alone. FIG. 5B depicts the structures of compounds 02-016 and 02-004.

FIGS. 6A-6B. FIG. 6A depicts a DSF-based measurement of HER3 binding by compounds 02-006 and 02-018 compared to bosutinib alone. FIG. 6B depicts the structures of compounds 02-006 and 02-018.

FIGS. 7A-7B. FIG. 7A depicts a DSF-based measurement of HER3 binding by compounds 02-017 and 02-005 compared to bosutinib alone. FIG. 7B depicts the structures of compounds 02-017 and 02-005.

FIGS. 8A-8B. FIG. 8A depicts a DSF-based measurement of HER3 binding by compounds 02-010, 02-001 and 02-013 compounds compared to bosutinib alone. FIG. 8B shows the structures of 02-010, 02-001 and 02-013 compounds.

FIGS. 9A-9B. FIG. 9A depicts a DSF-based measurement of HER3 binding by compounds 02-011, 02-002 and 02-014 compounds compared to bosutinib alone. FIG. 9B shows the structures of 02-011, 02-002 and 02-014 compounds.

FIGS. 10A-10B. FIG. 10A depicts a DSF-based measurement of HER3 binding by compounds 02-012, 02-003 and 02-015 compounds compared to bosutinib alone. FIG. 10B shows the structures of 02-012, 02-003 and 02-015 compounds.

FIGS. 11A-11B. FIG. 11A depicts a DSF-based measurement of HER3 binding by compounds 02-022, 02-07 and 02-025 compounds compared to bosutinib alone. FIG. 11B shows the structures of 02-022, 02-07 and 02-025 compounds.

FIGS. 12A-12B. FIG. 12A depicts a DSF-based measurement of HER3 binding by compounds 02-023, 02-08 and 02-026 compounds compared to bosutinib alone. FIG. 12B shows the structures of 02-023, 02-08 and 02-026 compounds.

FIGS. 13A-13B. FIG. 13A depicts a DSF-based measurement of HER3 binding by compounds 02-024, 02-09 and 02-027 compounds compared to bosutinib alone. FIG. 13B shows the structures of 02-024, 02-09 and 02-027 compounds.

FIGS. 14A-14B. FIG. 14A depicts a DSF-based measurement of HER3 binding by compounds 01-012, 01-08, 01-09 and 01-10 compared to bosutinib and 179D warheads. FIG. 14B shows the structures of 01-012, 01-08, 01-09 and 01-10 compounds.

FIGS. 15A-15D show Western blot analysis of the effect of treatment with bosutinib (4 hours), 179D- and 050A-based VHI(1) and VHL(2) degraders on the levels of endogenous HER3 and Src in MCF7 cells. FIG. 15A shows the results for bostinib-16-VHL1 (2-10), bostinib-19-VHL1 (02-01) and bostinib-22-VHL1 (02-13). FIG. 15B shows the results for 179D-16-VHL1 (2-11), 179D-19-VHL1 (02-02) and 179D-22-VHL1 (02-14). FIG. 15C shows the results for 050A-16-VHL1 (2-04), 050A-19-VHL1 (02-06) and 050A-22-VHL1 (02-05).

FIG. 15D shows the results for bostinib-10-VHL2 (2-07) and 179D-10-VHL2 (02-08).

FIGS. 16A-16D show Western blot analysis of the effect of treatment with 01-012, 01-013 and 01-010 compounds (24 hours) on the levels of endogenous HER3 in MCF7 cells. FIG. 16A shows the results for 01-012 and 01-013 compounds. FIG. 16B shows the results for 01-010 and Gefinitib-based PROTAC3. FIG. 16C shows the results for 02-01 and 02-01 in combinatiton with Gefinitib-based PROTAC3. FIG. 16D shows the results for 02-02 in combinatiton with Gefinitib-based PROTAC3 and 02-14 in combinatiton with Gefinitib-based PROTAC3.

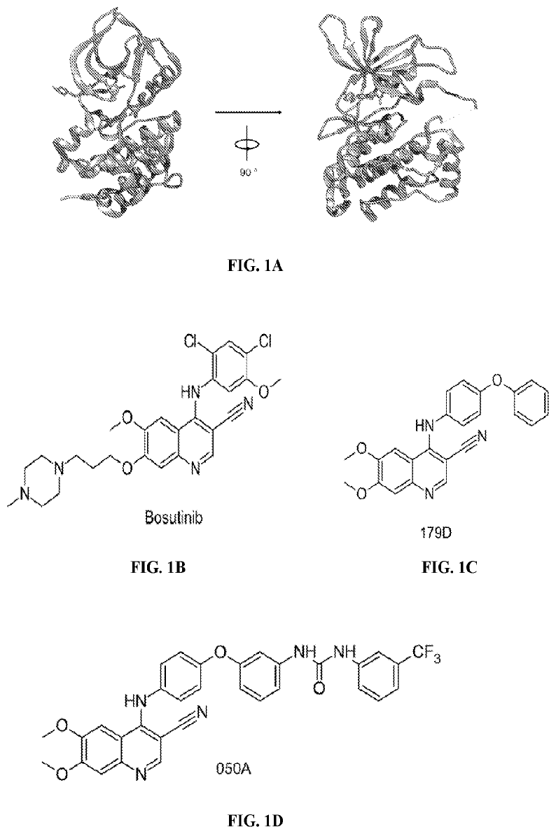

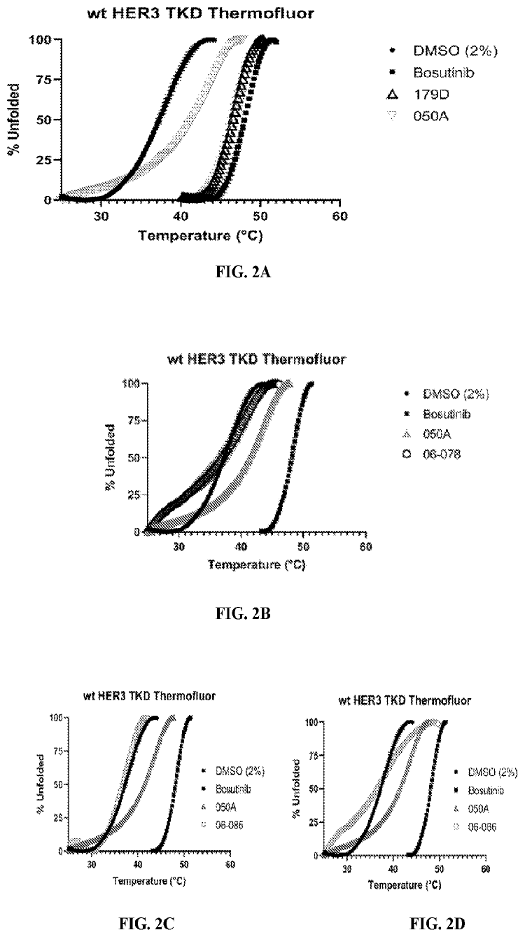

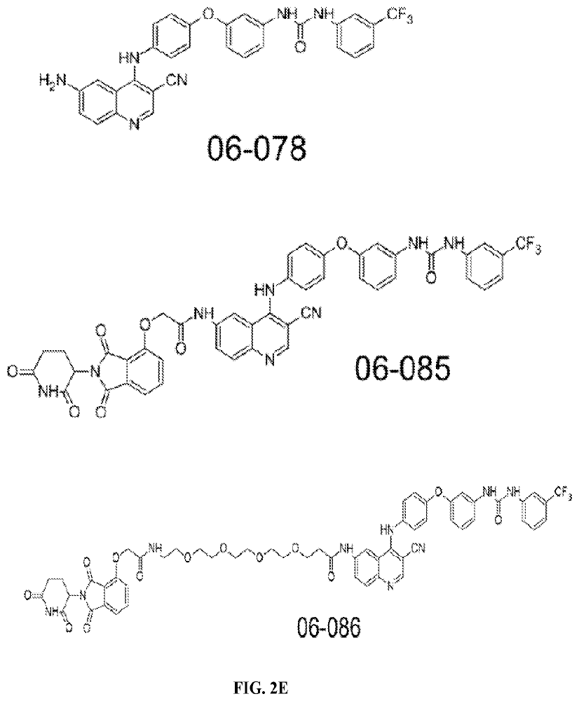

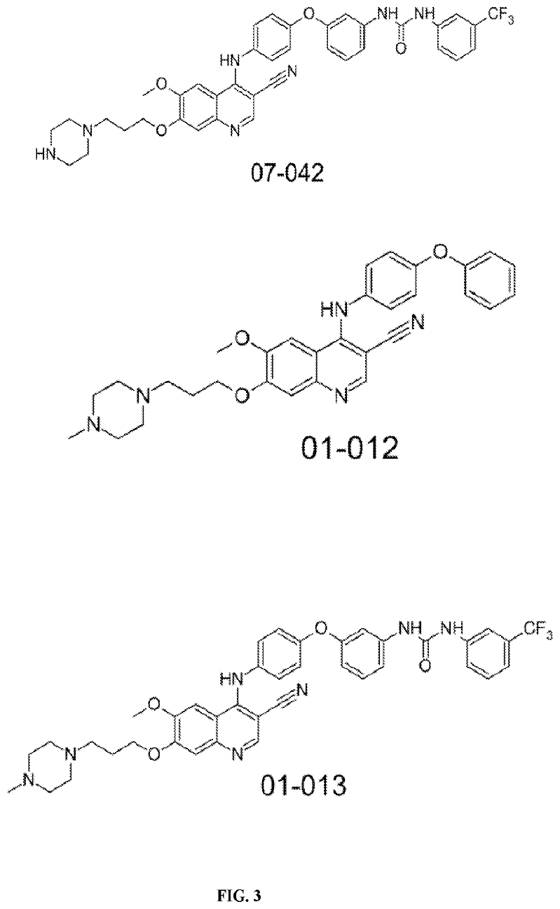

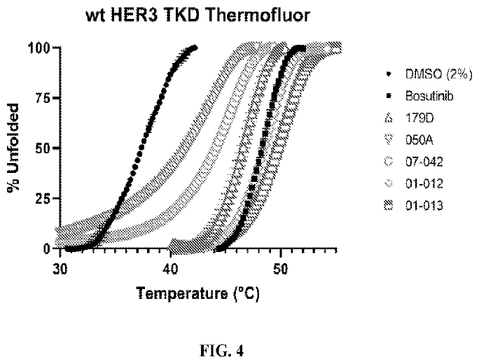

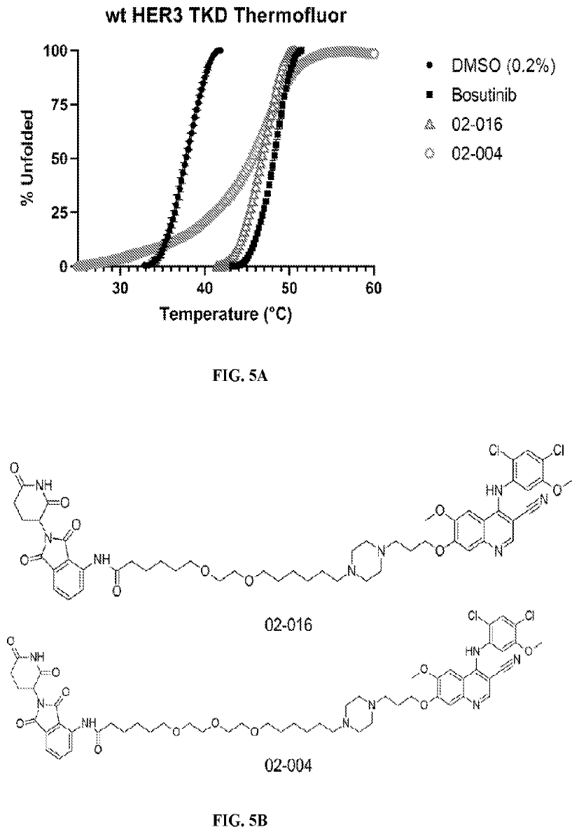

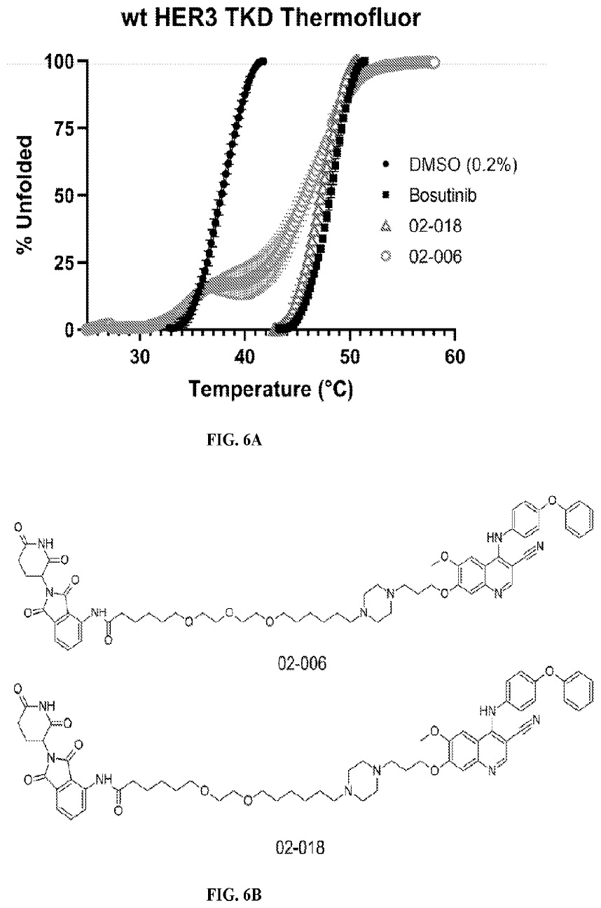

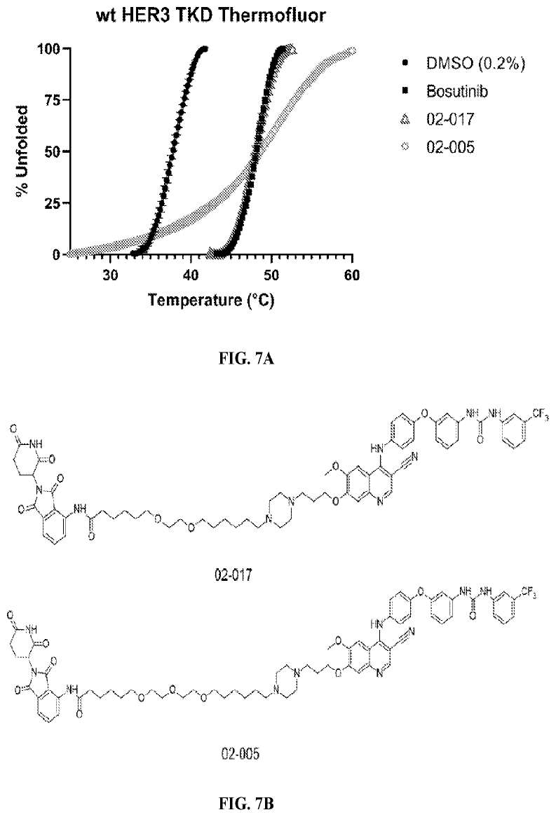

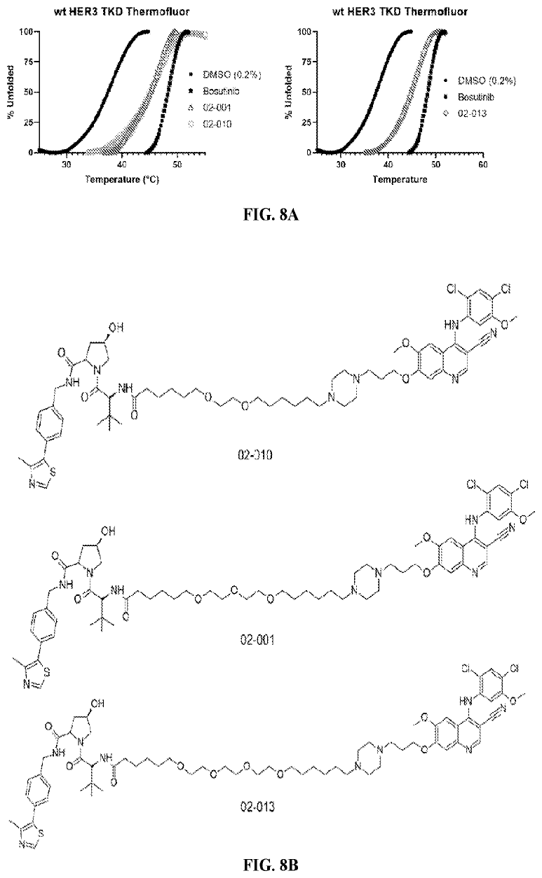

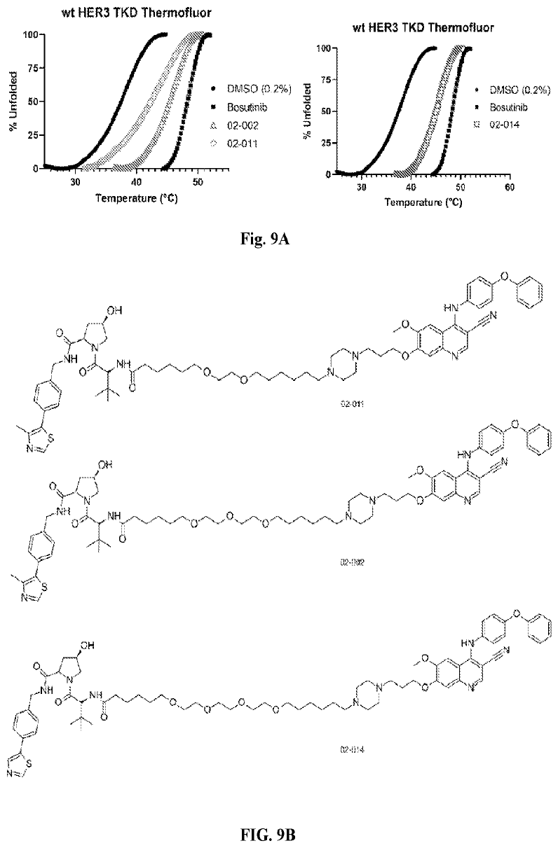

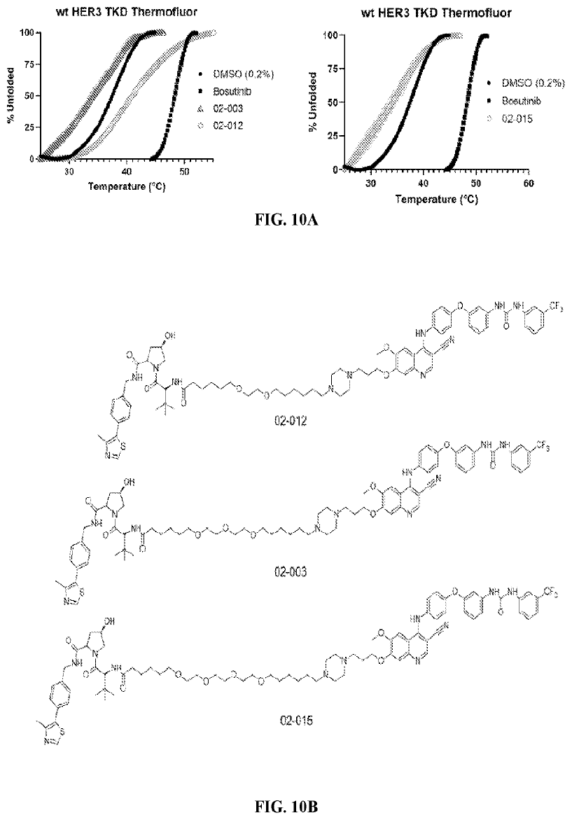

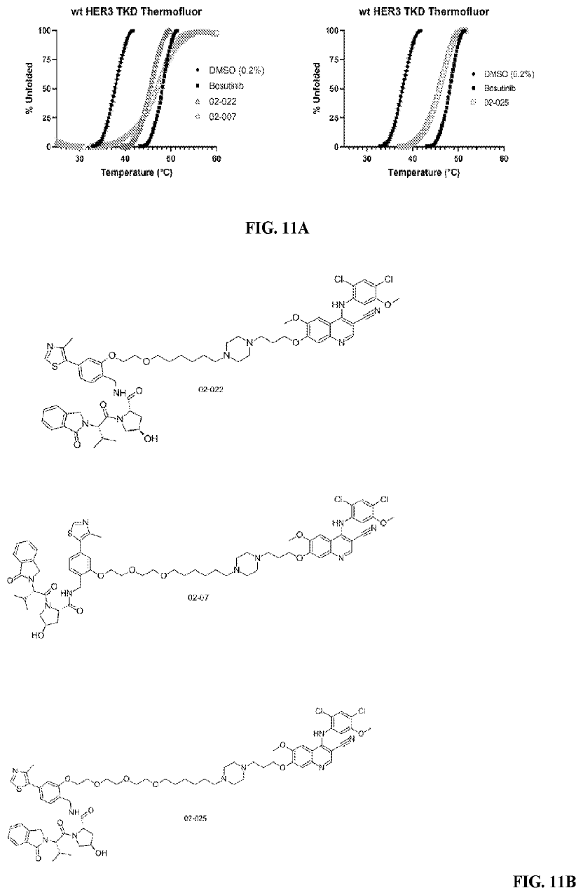

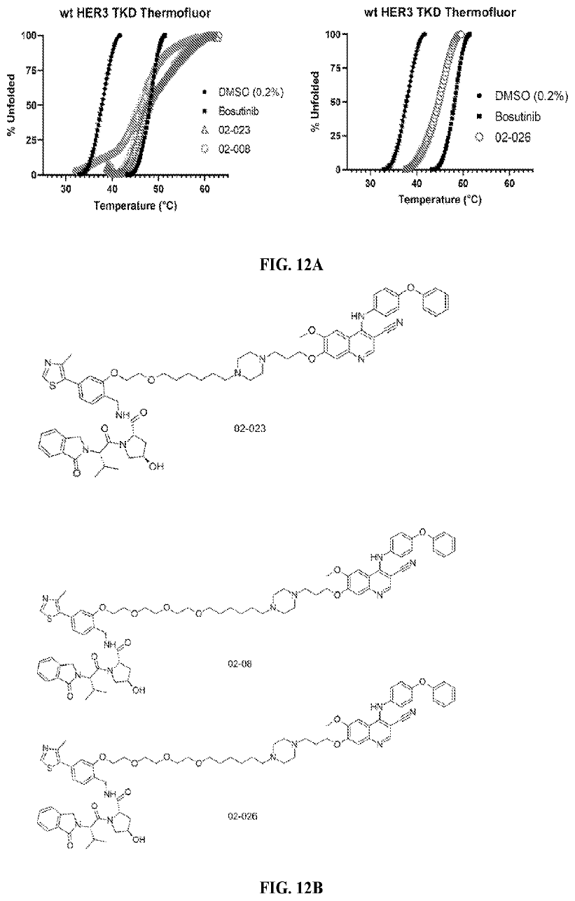

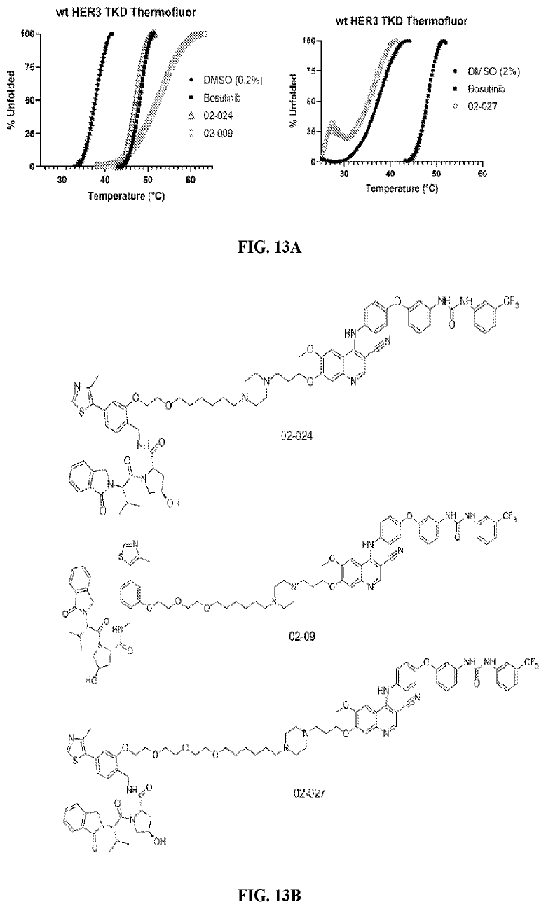

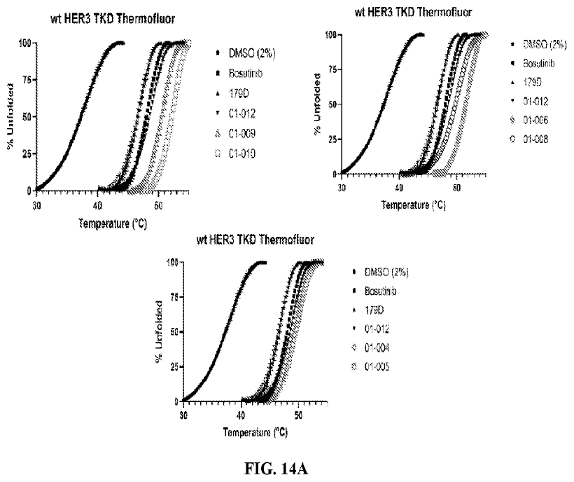

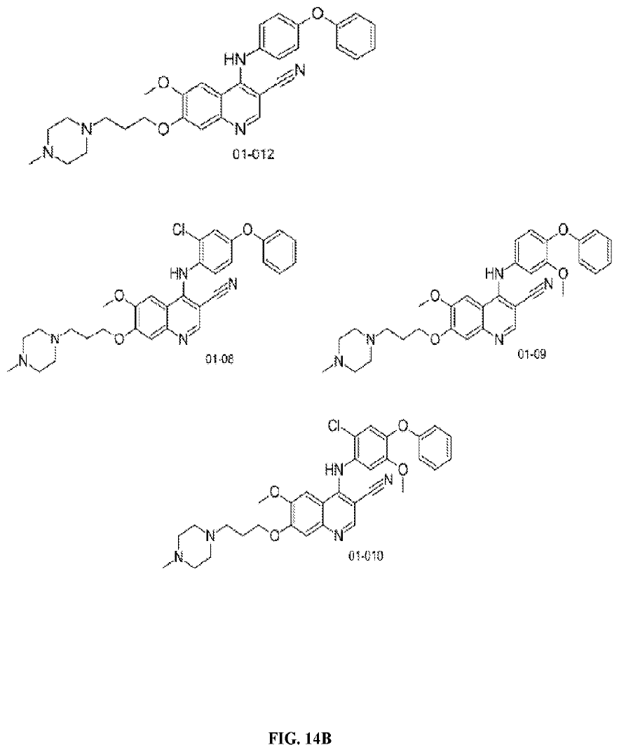

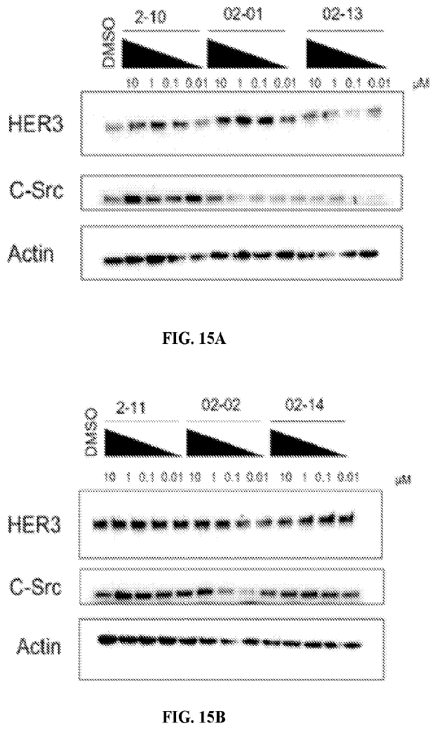

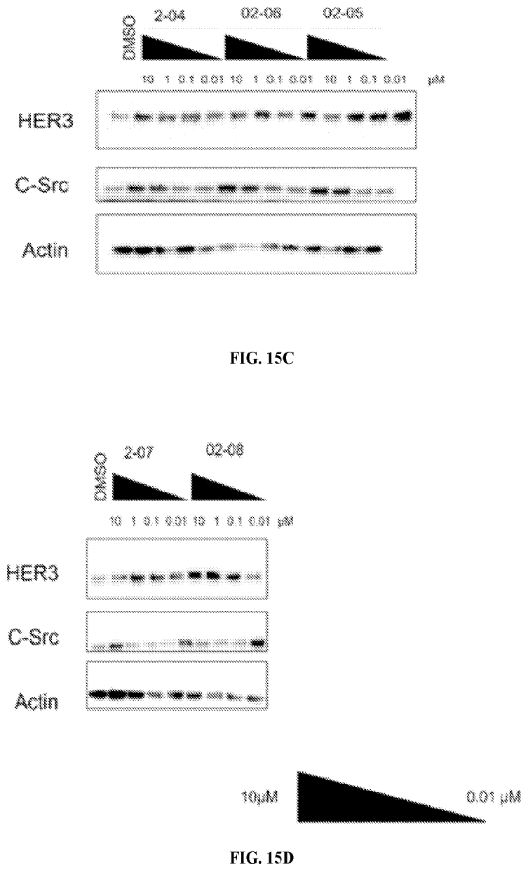

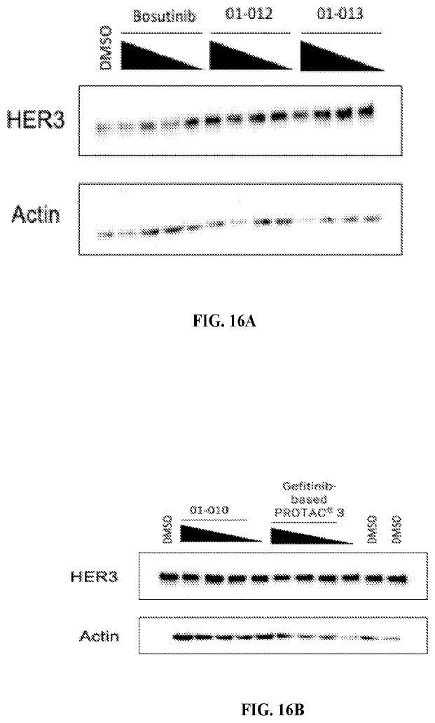

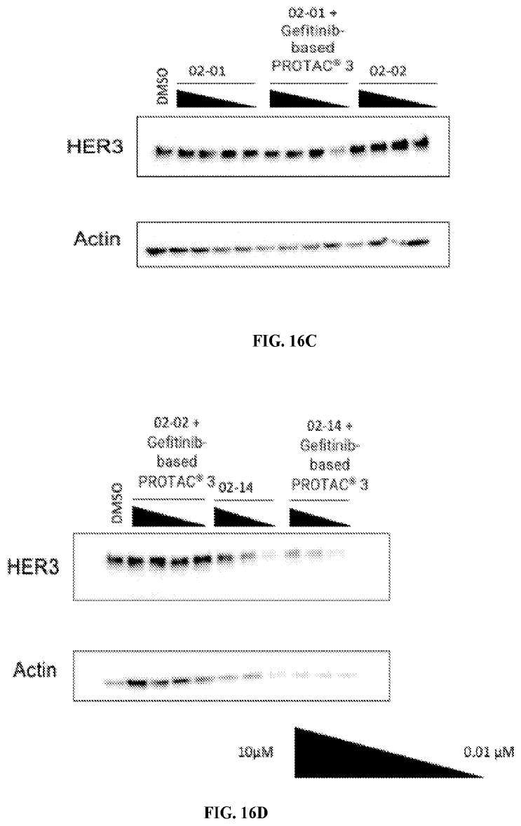

### DETAILED DESCRIPTION

### A. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di-and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds. An alkyl moiety may be fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched non-cyclic chain, or combinations thereof, including at least one carbon atom and at least one heteroatom (e.g., selected from the group consisting of O, N, P, Si, and S), and wherein the nitrogen and sulfur atoms may optionally be oxidized, and the nitrogen heteroatom may optionally be quaternized. The heteroatom(s) (e.g., O, N, P, S, and Si) may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH—₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P). In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R¹ may have a specified number of carbons (e.g., “C₁-C₄ alkylsulfonyl”).

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl) substituted with at least one substituent selected from:
   (i) oxo, halogen, —CF₃, —CN, —OH, —NH₂, —COOH, —CONH₂, —NO₂, —SH—SO₃H, —SO₄H, —SO₂NH₂, □NHNH₂, □ONH₂, □NHC═(O)NHNH₂, □NHC═(O) NH₂, —NHSO₂H, —NHC═(O)H, —NHC(O)—OH, —NHOH, —OCF₃, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), unsubstituted heteroaryl (e.g., 5 to 10 membered heteroaryl, 5 to 9 membered heteroaryl, or 5 to 6 membered heteroaryl), and
   (ii) alkyl (e.g., C₁-C₈alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl) substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), unsubstituted heteroaryl (e.g., 5 to 10 membered heteroaryl, 5 to 9 membered heteroaryl, or 5 to 6 membered heteroaryl), and
      (b) alkyl (e.g., C₁-C₈alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ^{13C}- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol [symbol] denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat diseases associated with HER3 activity. Certain methods described herein may treat diseases associated with HER3 activity (e.g., cancer) by inhibiting HER3 activity. For example, certain methods herein treat cancer. For example certain methods herein treat cancer by decreasing a symptom of cancer. Symptoms of cancer would be known or may be determined by a person of ordinary skill in the art. The term “treating” and conjugations thereof, include prevention of an injury, pathology, condition, or disease. In embodiments, treating does not include preventing.

An “effective amount” is an amount sufficient to accomplish a stated purpose (e.g. achieve the effect for which it is administered, treat a disease, reduce enzyme activity, increase enzyme activity, reduce protein function, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug or prodrug is an amount of a drug or prodrug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. cancer) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, a disease associated with HER3 activity may be treated with an agent (e.g. compound as described herein) effective for decreasing the level of HER3 activity.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein (e.g., HER3) or enzyme.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein.

The term “modulator” refers to a composition that increases or decreases the level of a target molecule or the function of a target molecule. In embodiments, a modulator is an anti-cancer agent. In embodiments, a modulator is a HER3 antagonist. In embodiments, a modulator is a HER3 agonist.

“Anti-cancer agent” or “anti-cancer drug” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent approved by the FDA or similar regulatory agency of a country other than the USA, for treating cancer. Examples of anti-cancer agents include, but are not limited to, anti-androgens (e.g., Casodex, Flutamide, MDV3100, or ARN-509), MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec.RTM.), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody,; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras famesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g. tomaymycin), carboplatin, CC-1065 and CC-1065 analogs including amino-CBIs, nitrogen mustards (such as chlorambucil and melphalan), dolastatin and dolastatin analogs (including auristatins: eg. monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g. mertansine), methotrexate, mitomycin C, taxoids, vinca alkaloids (such as vinblastine and vincristine), epothilones (e.g. epothilone B), camptothecin and its clinical analogs topotecan and irinotecan, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

“Patient” or “patient in need thereof” or “subject in need thereof” or “subject” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In some embodiments, a subject is human. In embodiments, a subject in need thereof is human.

“Disease” or “condition” refer to a state of being or health status of a patient or subject capable of being treated with a compound, pharmaceutical composition, or method provided herein. In some embodiments, the disease is a disease having the symptom of cell hyperproliferation. In some embodiments, the disease is a disease having the symptom of an aberrant level of HER3 activity. In some embodiments, the disease is a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma. In embodiments, the disease is brain cancer. In embodiments, the disease is neuroblastoma. In embodiments, the disease is glioblastoma.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include cancer of the prostate, thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples may include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat′l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. Substitution of one amino acid residue for another from a different class or group is referred to herein as a “non-conservative” substitution. In contrast, non-conservative amino acid substitutions tend to modify conformation and function of a protein.

### Example of Amino Acid Classification

| | |
|---|---|
| Small/Aliphatic residues: | Gly, Ala, Val, Leu, Ile |
| Cyclic Imino Acid: | Pro |
| Hydroxyl-containing Residues: | Ser, Thr |
| Acidic Residues: | Asp, Glu |
| Amide Residues: | Asn, Gln |
| Basic Residues: | Lys, Arg |
| Imidazole Residue: | His |
| Aromatic Residues: | Phe, Tyr, Trp |
| Sulfur-containing Residues: | Met, Cys |

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., BIOCHEMISTRY at pp. 13-15, 2nd ed. Lubert Stryer ed. (Stanford University); Henikoff et al., Proc. Nat′l Acad. Sci. USA (1992) 89:10915-10919; Lei et al., J. Biol. Chem. (1995) 270(20):11882-11886).

“Polypeptide,” “peptide,” and “protein” are used herein interchangeably and mean any peptide-linked chain of amino acids, regardless of length or post-translational modification. As noted below, the polypeptides described herein can be, e.g., wild-type proteins, biologically-active fragments of the wild-type proteins, or variants of the wild-type proteins or fragments. Variants, in accordance with the disclosure, can contain amino acid substitutions, deletions, or insertions. The substitutions can be conservative or non-conservative.

Following expression, the proteins can be isolated. The term “purified” or “isolated” as applied to any of the proteins described herein refers to a polypeptide that has been separated or purified from components (e.g., proteins or other naturally-occurring biological or organic molecules) which naturally accompany it, e.g., other proteins, lipids, and nucleic acid in a cell expressing the proteins. Typically, a polypeptide is purified when it constitutes at least 60 (e.g., at least 65, 70, 75, 80, 85, 90, 92, 95, 97, or 99) %, by weight, of the total protein in a sample.

An amino acid residue in a protein “corresponds” to a given residue when it occupies the same essential structural position within the protein as the given residue. For example, a selected residue in a selected protein corresponds to a particular amino acid in HER3 when the selected residue occupies the same essential spatial or other structural relationship as particular amino acid in HER3. In some embodiments, where a selected protein is aligned for maximum homology with the human HER3 protein, the position in the aligned selected protein aligning with a particular reside is said to correspond to that particular reside. Instead of a primary sequence alignment, a three dimensional structural alignment can also be used, e.g., where the structure of the selected protein is aligned for maximum correspondence with the human HER3 protein and the overall structures compared. In this case, an amino acid that occupies the same essential position as a particular reside in the structural model is said to correspond to the particular reside.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” is used in accordance with its plain and ordinary meaning and includes oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J Hosp. Pharm. 46:1576-1587, 1989). The compositions of the present invention can also be delivered as nanoparticles.

Pharmaceutical compositions provided by the present invention include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms (e.g. symptoms of cancer or aberrant HER3 activity). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer such as radiation or surgery.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “Receptor tyrosine-protein kinase erbB-3”, “human epidermal growth factor receptor 3”, “ERBB3”, or “HER3” refers to a pseudokinase (reduced activity or inactive kinase) that is a member of the epidermal growth factor receptor (EGFR/ERBB) family of receptor tyrosine kinases. The term “HER3” may refer to the nucleotide sequence or protein sequence of human HER3 (e.g., Entrez 2065, Uniprot P21860, RefSeq NM_001982, or RefSeq NP_001973). The term “HER3” includes both the wild-type form of the nucleotide sequences or proteins as well as any mutants thereof. In some embodiments, “HER3” is wild-type HER3 receptor. In some embodiments, “HER3” is one or more mutant forms. The term “HER3” XYZ refers to a nucleotide sequence or protein of a mutant HER3wherein the Y numbered amino acid of HER3 that normally has an X amino acid in the wildtype, instead has a Z amino acid in the mutant. In embodiments, an HER3 is the human HER3. In embodiments, the HER3 has the nucleotide sequence corresponding to reference number GI:317171925. In embodiments, the HER3 has the nucleotide sequence corresponding to RefSeq NM_001982.3. In embodiments, the HER3 has the protein sequence corresponding to reference number GI:54792100. In embodiments, the HER3 has the protein sequence corresponding to RefSeq NP_001973.2. In embodiments, the HER3 has the following amino acid sequence:

(SEQ ID NO: 1)
MRANDALQVLGLLFSLARGSEVGNSQAVCPGTLNGLSVTGDAENQYQTLYKLYERCEV
VMGNLEIVLTGHNADLSFLQWIREVTGYVLVAMNEFSTLPLPNLRVVRGTQVYDGKFAI
FVMLNYNTNSSHALRQLRLTQLTEILSGGVYIEKNDKLCHMDTIDWRDIVRDRDAEIVV
KDNGRSCPPCHEVCKGRCWGPGSEDCQTLTKTICAPQCNGHCFGPNPNQCCHDECAGG
CSGPQDTDCFACRHFNDSGACVPRCPQPLVYNKLTFQLEPNPHTKYQYGGVCVASCPH
NFVVDQTSCVRACPPDKMEVDKNGLKMCEPCGGLCPKACEGTGSGSRFQTVDSSNIDG
FVNCTKILGNLDFLITGLNGDPWHKIPALDPEKLNVFRTVREITGYLNIQSWPPHMHNFS
VFSNLTTIGGRSLYNRGFSLLIMKNLNVTSLGFRSLKEISAGRIYISANRQLCYHHSLNWT
KVLRGPTEERLDIKHNRPRRDCVAEGKVCDPLCSSGGCWGPGPGQCLSCRNYSRGGVC
VTHCNFLNGEPREFAHEAECFSCHPECQPMEGTATCNGSGSDTCAQCAHFRDGPHCVSS
CPHGVLGAKGPIYKYPDVQNECRPCHENCTQGCKGPELQDCLGQTLVLIGKTHLTMAL
TVIAGLVVIFMMLGGTFLYWRGRRIQNKRAMRRYLERGESIEPLDPSEKANKVLARIFK
ETELRKLKVLGSGVFGTVHKGVWIPEGESIKIPVCIKVIEDKSGRQSFQAVTDHMLAIGSL
DHAHIVRLLGLCPGSSLQLVTQYLPLGSLLDHVRQHRGALGPQLLLNWGVQIAKGMYY
LEEHGMVHRNLAARNVLLKSPSQVQVADFGVADLLPPDDKQLLYSEAKTPIKWMALES
IHFGKYTHQSDVWSYGVTVWELMTFGAEPYAGLRLAEVPDLLEKGERLAQPQICTIDV
YMVMVKCWMIDENIRPTFKELANEFTRMARDPPRYLVIKRESGPGIAPGPEPHGLTNKK
LEEVELEPELDLDLDLEAEEDNLATTTLGSALSLPVGTLNRPRGSQSLLSPSSGYMPMNQ
GNLGESCQESAVSGSSERCPRPVSLHPMPRGCLASESSEGHVTGSEAELQEKVSMCRSRS
RSRSPRPRGDSAYHSQRHSLLTPVTPLSPPGLEEEDVNGYVMPDTHLKGTPSSREGTLSS
VGLSSVLGTEEEDEDEEYEYMNRRRRHSPPHPPRPSSLEELGYEYMDVGSDLSASLGST
QSCPLHPVPIMPTAGTTPDEDYEYMNRQRDGGGPGGDYAAMGACPASEQGYEEMRAF
QGPGHQAPHVHYARLKTLRSLEATDSAFDNPDYWHSRLFPKANAQRT.

In embodiments, the HER3 is a mutant HER3. In embodiments, the mutant HER3 is associated with a disease that is not associated with wildtype HER3. In embodiments, the HER3 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above. In embodiments, the HER3 is a variant of the seqeuence above, including a shorter variant or mutated variant. In embodiments, the mutant HER3 is a splice variant. In embodiments, the mutant HER3 is a splice variant with aberrant activity relative to the widtype HER3. In embodiments, the mutant HER3 is a truncated splice variant with aberrant activity relative to the widtype HER3. In embodiments, the mutant HER3 is a splice variant lacking a portion of the wildtype HER3 with aberrant activity relative to the widtype HER3. In embodiments, the HER3 is described in Cancer Cell (2013) May 13 23, 603-617, which is herein incorporated in its entirety for all purposes.

The term “Receptor tyrosine-protein kinase erbB-2”, “human epidermal growth factor receptor 2”, “CD340”, “ERBB2”, “neu”, “HER2/neu”, or “HER2” refers to a member of the epidermal growth factor receptor (EGFR/ERBB) family of receptor tyrosine kinases. The term “HER2” may refer to the nucleotide sequence or protein sequence of human HER2 (e.g., Entrez 2064, Uniprot P04626, RefSeq NM_004448, or RefSeq NP_004439). The term “HER2” includes both the wild-type form of the nucleotide sequences or proteins as well as any mutants thereof. In some embodiments, “HER2” is wild-type HER3 receptor. In some embodiments, “HER2” is one or more mutant forms. The term “HER2” XYZ refers to a nucleotide sequence or protein of a mutant HER2 wherein the Y numbered amino acid of HER2 that normally has an X amino acid in the wildtype, instead has a Z amino acid in the mutant. In embodiments, an HER2 is the human HER2. In embodiments, the HER2 has the nucleotide sequence corresponding to reference number GI:584277099. In embodiments, the HER2 has the nucleotide sequence corresponding to RefSeq NM_004448.3. In embodiments, the HER2 has the protein sequence corresponding to reference number GI:54792096. In embodiments, the HER2 has the protein sequence corresponding to RefSeq NP_004439.2. In embodiments, the HER2 has the following amino acid sequence:

(SEQ ID NO: 2)
MELAALCRWGLLLALLPPGAASTQVCTGTDMKLRLPASPETHLDMLRHLYQGCQVVQ
GNLELTYLPTNASLSFLQDIQEVQGYVLIAHNQVRQVPLQRLRIVRGTQLFEDNYALAV
LDNGDPLNNTTPVTGASPGGLRELQLRSLTEILKGGVLIQRNPQLCYQDTILWKDIFHKN
NQLALTLIDTNRSRACHPCSPMCKGSRCWGESSEDCQSLTRTVCAGGCARCKGPLPTDC
CHEQCAAGCTGPKHSDCLACLHFNHSGICELHCPALVTYNTDTFESMPNPEGRYTFGAS
CVTACPYNYLSTDVGSCTLVCPLHNQEVTAEDGTQRCEKCSKPCARVCYGLGMEHLRE
VRAVTSANIQEFAGCKKIFGSLAFLPESFDGDPASNTAPLQPEQLQVFETLEEITGYLYIS
AWPDSLPDLSVFQNLQVIRGRILHNGAYSLTLQGLGISWLGLRSLRELGSGLALIHHNTH
LCFVHTVPWDQLFRNPHQALLHTANRPEDECVGEGLACHQLCARGHCWGPGPTQCVN
CSQFLRGQECVEECRVLQGLPREYVNARHCLPCHPECQPQNGSVTCFGPEADQCVACA
HYKDPPFCVARCPSGVKPDLSYMPIWKFPDEEGACQPCPINCTHSCVDLDDKGCPAEQR
ASPLTSIISAVVGILLVVVLGVVFGILIKRRQQKIRKYTMRRLLQETELVEPLTPSGAMPN
QAQMRILKETELRKVKVLGSGAFGTVYKGIWIPDGENVKIPVAIKVLRENTSPKANKEIL
DEAYVMAGVGSPYVSRLLGICLTSTVQLVTQLMPYGCLLDHVRENRGRLGSQDLLNWC
MQIAKGMSYLEDVRLVHRDLAARNVLVKSPNHVKITDFGLARLLDIDETEYHADGGKV
PIKWMALESILRRRFTHQSDVWSYGVTVWELMTFGAKPYDGIPAREIPDLLEKGERLPQ
PPICTIDVYMIMVKCWMIDSECRPRFRELVSEFSRMARDPQRFVVIQNEDLGPASPLDST
FYRSLLEDDDMGDLVDAEEYLVPQQGFFCPDPAPGAGGMVHHRHRSSSTRSGGGDLTL
GLEPSEEEAPRSPLAPSEGAGSDVFDGDLGMGAAKGLQSLPTHDPSPLQRYSEDPTVPL
PSETDGYVAPLTCSPQPEYVNQPDVRPQPPSPREGPLPAARPAGATLERPKTLSPGKNGV
VKDVFAFGGAVENPEYLTPQGGAAPQPHPPPAFSPAFDNLYYWDQDPPERGAPPSTFKG
TPTAENPEYLGLDVPV.

In embodiments, the HER2 is a mutant HER2. In embodiments, the mutant HER2 is associated with a disease that is not associated with wildtype HER2. In embodiments, the HER2 includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above. In embodiments, the HER2 is a variant of the seqeuence above, including a shorter variant or mutated variant. In embodiments, the mutant HER2 is a splice variant. In embodiments, the mutant HER2 is a splice variant with aberrant activity relative to the widtype HER2. In embodiments, the mutant HER2 is a truncated splice variant with aberrant activity relative to the widtype HER2. In embodiments, the mutant HER2 is a splice variant lacking a portion of the wildtype HER2 with aberrant activity relative to the widtype HER2.

The term “ligand” is used in accordance with its plain ordinary meaning and refers to a molecule (e.g., compound as described herein) capable of binding to another molecule (e.g., protein, receptor, enzyme, target, or cell). In embodiments, a ligand is a modulator, inhibitor, activator, agonist, or antagonist.

Provided herein are agents (e.g. compounds, drugs, therapeutic agents) that may be in a prodrug form. Prodrugs of the compounds described herein are those compounds that readily undergo chemical changes under select physiological conditions to provide the final agents (e.g. compounds, drugs, therapeutic agents). Additionally, prodrugs can be converted to agents (e.g. compounds, drugs, therapeutic agents) by chemical or biochemical methods in an ex vivo environment. Prodrugs described herein include compounds that readily undergo chemical changes under select physiological conditions to provide agents (e.g. compounds, drugs, prophylactic agents, therapeutic agents) to a biological system (e.g. in a subject, in a cancer cell, in the extracellular space near a cancer cell). For example, physiologically hydrolyzable esters or amides are esters or amides, respectively, that are hydrolyzed to the corresponding hydroxyl and carboxylic acid portions of the ester, or the corresponding amine and carboxylic acid portions of the amide, by a chemical or enzymatic reaction (e.g., esterase or amidase or protease) following administration to a subject.

“Analog” and “analogue” are used interchangeably and are used in accordance with their plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof or but differs in one or more components (e.g., different substituent(s), addition of substituent(s), removal of substituent(s)). Accordingly, an analog is a compound that is similar or comparable in function and appearance but not in structure or origin to a reference compound.

The term “derivative” is used in accordance with its plain ordinary meaning in Chemistry and refers to a compound that is derived (e.g., a product made from a reactant) from a similar compound by a chemical or physical process. “Derivative” is used in accordance with its plain ordinary meaning within Chemistry and Biology and refers to a compound (e.g., chemical) that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof; and the derivative is a compound that was derived from the reference compound through one or more chemical reaction(s) or the reference compound was derived from the derivative through one or more chemical reaction(s). Accordingly, a derivative is a compound that is similar or comparable in function and appearance but not in structure or origin to a reference compound. In embodiments of a derivative, an original substituent (e.g., substituent group) of a reference compound is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of a reference compound are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of a reference compound is replaced with a substituent (e.g., substituent group). In embodiments of a derivative, a plurality of hydrogen atoms of a reference compound are replaced with a plurality of substituents (e.g., substituent groups), wherein the substituents (e.g., substituent groups) are each optionally different.

The term “HER3 activity” is used in accordance with its plain ordinary meaning and refers to the function or activity of the HER3 protein. Examples of HER3 activity include dimerization (e.g., heterodimerization) or activation of the activity of a protein upon dimerization with HER3 (e.g., EGFR activity, HER2 activity, HER4 activity, or c-MET activity). In embodiments, HER3 activity is increasing or activating activity of a protein interacting with HER3 (e.g., PI3K activity, MEK activity, MAPK activity, RAF activity, BRAF activity, AKT activity, RAS activity, or KRAS activity). In embodiments HER3 activity is activation or increasing of activity of a signaling pathway by HER3 or activation of a component of a signaling pathway by HER3 (e.g., directly or through intervening components of the signaling pathway). In embodiments HER3 activity is activation of kinase activity of a protein that interacts (e.g., directly contacting HER3 or interactions with HER3 through intermediates) with HER3 (e.g., EGFR, HER2, HER4, c-MET, PI3K, MEK, MAPK, RAF, BRAF, AKT, RAS, or KRAS).

The term “degradation-increasing moiety” is used in accordance with its plain ordinary meaning and refers to a moiety capable of increasing the degradation of a protein or other biological molecule (e.g., including by binding to the protein or other biological molecule or by binding to the protein or other bioloigical molecule through a second moiety bonded to the degradation-increasing moiety (e.g., wherein the degradation-increasing moiety and second moiety are a compound described herein and the protein or other biological molecule bound to the compound described herein is a HER protein, for example HER3, and the HER protein (e.g., HER3) is degraded more than control (e.g., degradation in the absence of the compound including the degradation-increasing moiety))). In embodiments, a degradation-increasing moiety increases the interaction of a protein to be degraded with a cell's protein degradation components (e.g., ubiquitin ligase(s), proteasome, E3 ubiquitin ligase (e.g., cereblon, HECT, RING-finger, U-box, PHD-finger, APC, SCF complex (Skp1-Cullin-F-box protein complex), E3A, mdm2, EDD1, SOCS, LNXp80, CBX4, HACE1, CBLL1, HECTD1, HECTD2, HECTD3, HECW1, HECW2, HERC1, HERC2, HERC3, HERC4, HUWEl, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE4A, UBE4B, UBOX5, UBR5, WWP1, WWP2, VHL, or VHL-cullin-RING-ligase complex)). In embodiments, the degradation-increasing moiety binds (e.g., directly or through another moiety bonded to the degradation-increasing moiety (e.g., another portion of a compound that includes a degradation-increasing moiety)) to the protein to be degraded and a component of the cell's protein degradation effectors (e.g., E3 ubiquitin ligase). In embodiments, the degradation-increasing moiety is a thalidomide moiety or an analog, derivative, or prodrug thereof; phthalimide moiety or an analog, derivative, or prodrug thereof; adamantyl or an analog, derivative, or prodrug thereof; an IκBα phosphopeptide moiety or an analog, derivative, or prodrug thereof; nutlin moiety or an analog, derivative, or prodrug thereof; HIF-1α pentapeptide moiety or an analog, derivative, or prodrug thereof; or

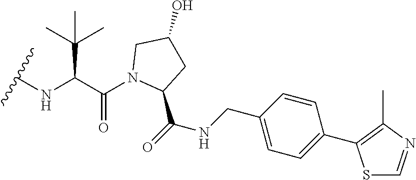 [SMILES: *N[C@H](C(=O)N1C[C@H](O)C[C@H]1C(=O)NCc1ccc(-c2scnc2C)cc1)C(C)(C)C]

or an analog, derivative, or prodrug thereof. In embodiments, the degradation-increasing moiety is described in Bioorg. Med. Chem. Lett (2008) 18 5904-5908; Cancer Cell (2011) August 16, 20, 158-172; Nat Chem Biol (2014) December vol 10, 1006-1012; Nat Chem Biol (2015) August vol 11 611-617; Oncogene (2008) 27, 7201-7211; P. Natl Acad Sci USA (2001) July 17, vol. 98(15) 8554-8559; Science (2015) 348, 1376-1381; U.S. Pat. Nos. 7,208,157; 7,041,298; US publ. no. 20150119435; US publ. no. 20040038358; or US publ. no. 20020068063, all of which are herein incorporated by reference in their entirely and for all purposes.

### B. Compounds

In an aspect is provided a compound having the formula:

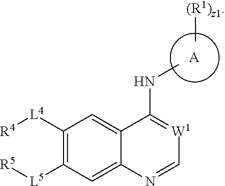

Ring A is aryl or heteroaryl. W¹ is N or C(R⁶). R¹ is independently a halogen, —CX¹₃, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. R⁴ is independently a hydrogen, halogen, —CX⁴₃, —CHX⁴₂, —CH₂X⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁴₃, —OCHX⁴₂, —OCH₂X⁴₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is a degradation-increasing moiety. R⁶ is independently a hydrogen, halogen, —CX⁶₃, —CHX⁶₂, —CH₂X⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁶₃, —OCHX⁶₂, —OCH₂X⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷, R⁸, R⁹, and R¹⁰ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl. L⁴ is a bond or a divalent linker. L⁵ is a divalent linker. z1 is independently an integer from 0 to 7. m1 and v1 are independently 1 or 2. n1 is independently an integer from 0 to 4. X¹, X⁴, X⁶, and X^{A} are independently —Cl, —Br, —I, or —F. In embodiments, R⁵ is a degradation-increasing moiety (e.g., as described herein, including in an embodiment). In embodiments, R⁵ is a degradation-increasing moiety selected from a thalidomide moiety or an analog, derivative, or prodrug thereof; phthalimide moiety or an analog, derivative, or prodrug thereof; adamantyl or an analog, derivative, or prodrug thereof; an IκBα phosphopeptide moiety or an analog, derivative, or prodrug thereof; nutlin moiety or an analog, derivative, or prodrug thereof; and HIF-1α pentapeptide moiety or an analog, derivative, or prodrug thereof; and

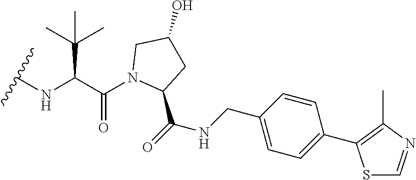 [SMILES: *N[C@H](C(=O)N1C[C@H](O)C[C@H]1C(=O)NCc1ccc(-c2scnc2C)cc1)C(C)(C)C]

or an analog, derivative, or prodrug thereof.

In an aspect is provided a compound having the formula:

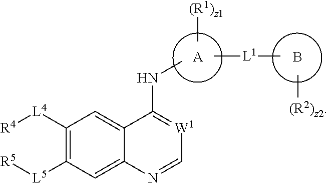

Ring A, Ring B, W¹, R¹, R⁴, R⁵, R⁶-R¹⁴, L⁴, L⁵, X¹, X⁴, X⁶, X^{A}, and z1 are as described herein.

Ring A is aryl or heteroaryl. Ring B is aryl or heteroaryl. W^{l} is N or C(R⁶). R¹ is independently a halogen, —CX¹³, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X′, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. R² is independently a halogen, —CX²₃, —CHX²₂, —CH₂X², —CN, —SOₙ₂R¹⁴, —SOᵥ₂NR¹¹R¹², —NHNH₂, —ONR¹¹R¹², —NHC═(O)NHNR¹¹R¹², —NHC═(O)NR¹¹R¹², N(O)ₘ₂, —NR¹¹R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR¹¹OR¹³, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl. R⁴ is independently a hydrogen, halogen, —CX⁴₃, —CHX⁴₂, —CH₂X⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁴₃, —OCHX⁴₂, —OCH₂X⁴₂, substituted or unsubstituted alkyl, substituted or unsubstituted heteroalkyl, substituted or unsubstituted cycloalkyl, substituted or unsubstituted. R⁵ is a degradation-increasing moiety. R⁶ is independently a hydrogen, halogen, —CX⁶₃, —CHX⁶₂, —CH₂X⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁶₃, —OCHX⁶₂, —OCH₂X⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷, R⁸, R⁹, R¹⁰, R¹¹, R², R¹, and R¹⁴ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R¹¹ and R¹² substituents bonded to the same nitrogen atom may optionally be joined to form a substituted or unsubstituted heterocycloalkyl or substituted or unsubstituted heteroaryl. L¹ is a bond, —O—, —NH—, —C(O)—, —C(O)NH—, —NHC(O)—, —C(O)O—, —OC(O)—, —NHC(O)NH—, —S—, substituted or unsubstituted alkylene or substituted or unsubstituted heteroalkylene. L⁴ is a bond or a divalent linker. L⁵ is a divalent linker. z1 is an integer from 0 to 7. z2 is an integer from 0 to 7. m1, and v1 are independently 1 or 2. m2 and v2 are independently 1 or 2. n1 is an integer from 0 to 4. n2 is an integer from 0 to 4. X¹, X², X⁴, X⁶, and X^{A} are independently —Cl, —Br, —I, or —F.

In embodiments, the compound has the formula:

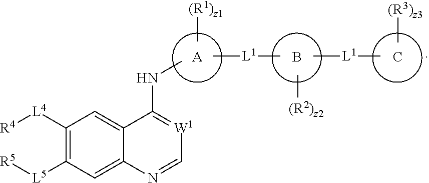

Ring A, Ring B, W¹, R′, R², R⁴, R⁵, L¹, L⁴, L⁵, X¹, X², X⁴, X⁶, X^{A}, z1, and z2 are as described herein.

In embodiments, Ring A is phenyl or 5 or 6 membered heteroaryl. In embodiments, Ring B is phenyl or 5 or 6 membered heteroaryl. Ring C is cycloalkyl, heterocycloalkyl, aryl, or heteroaryl. In embodiments, ring C is C₃-C₆ cycloalkyl, 3 to 6 membered heterocycloalkyl, phenyl, or 5 to 6 membered heteroaryl. In embodiments, R¹ is independently a halogen, —CX¹₃, —CHX¹₂, —CH₂X¹, —CN, —OH, —NH₂, —COOH, —CONH₂, —NO₂, —SH, substituted or unsubstituted C₁-C₄ alkyl, or 2 to 4 membered substituted or unsubstituted heteroalkyl. In embodiments, R² is independently a halogen, —CX²₃, —CHX²₂, —CH₂X², —CN, —OH, —NH₂, —COOH, —CONH₂, —NO₂, —SH, substituted or unsubstituted C₁-C₄ alkyl, or 2 to 4 membered substituted or unsubstituted heteroalkyl. R³ is independently a halogen, —CX³₃, —CHX³², —CH₂X³, —CN, —SOₙ₃R¹⁸, —SOᵥ₃NR¹⁵R¹⁶, —NHNH₂, —ONR¹⁵R¹⁶, —NHC═(O)NHNR¹⁵R¹⁶, —NHC═(O)NR¹⁵R¹⁶, —N(O)ₘ₃, —NR¹⁵R¹⁶, —C(O)R¹⁷, —C(O)—OR¹⁷, —C(O)NR¹⁵R¹⁶, —OR¹⁸, —NR¹⁵SO₂R¹⁷, —NR¹⁵C═(O)R¹⁷, —NR¹⁵C(O)—OR¹⁷, —NR¹⁵OR¹⁷, —OCX³₃, —OCHX³², —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl. In embodiments, R³ may also be oxo. In embodiments, R⁶ is independently halogen, —CN, —OH, —NH₂, —COOH, —CONH₂, —NO₂, —SH, substituted or unsubstituted C₁-C₄ alkyl, or 2 to 4 membered substituted or unsubstituted heteroalkyl. R¹⁵, R¹⁶, R¹⁷, and R¹⁸ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁵ and R¹⁶ substituents bonded to the same nitrogen atom may optionally be joined to form a substituted or unsubstituted heterocycloalkyl or substituted or unsubstituted heteroaryl. In embodiments, L¹ is —O—, substituted or unsubstituted C₁-C₃ alkylene or substituted or unsubstituted 2 to 3 membered heteroalkylene. L² is a bond, —O—, —NH—, —C(O)—, —C(O)NH—, —NHC(O)—, —C(O)O—, —OC(O)—, —NHC(O)NH—, —S—, substituted or unsubstituted C₁-C₃ alkylene or substituted or unsubstituted 2 to 3 membered heteroalkylene. In embodiments, L⁴ is a bond or a divalent linker. In embodiments, L⁵ is a divalent linker. In embodiments, z1 and z2 are independently an integer from 0 to 7. In embodiments, m1, m2, v1, and v2 are independently 1 or 2. In embodiments, n1 and n2 are independently an integer from 0 to 4. X³ is —Cl, —Br, —I, or —F. In embodiments, X¹, X², X⁴, X⁶, and X^{A} are independently —Cl, —Br, —I, or —F. z3 is independently an integer from 0 to 5. m3 is independently 1 or 2. v3 is independently 1 or 2. n3 is independently an integer from 0 to 4.

In embodiments, Ring A is phenyl. In embodiments, Ring A is a 5 to 6 membered heteroaryl. In embodiments, Ring A is a thienyl. In embodiments, Ring A is a 2-thienyl. In embodiments, Ring A is a 3-thienyl. In embodiments, Ring A is a pyridyl. In embodiments, Ring A is a 2-pyridyl. In embodiments, Ring A is a 3-pyridyl. In embodiments, Ring A is a 4-pyridyl. In embodiments, Ring A is a napththyl. In embodiments, Ring A is a 1-napththyl. In embodiments, Ring A is a 2-napththyl. In embodiments, Ring A is a quinolinyl. In embodiments, Ring A is a isoquinolinyl. In embodiments, Ring A is a 1-isoquinolinyl. In embodiments, Ring A is a 3-isoquinolinyl. In embodiments, Ring A is a 4-isoquinolinyl. In embodiments, Ring A is phenyl or 5 or 6 membered heteroaryl. In embodiments, the numbering of Ring A in this paragraph (e.g., 2-thienyl, 1-isoquinolinyl, etc.) refers the attachment point of the —NH— linker that connects Ring A to the fused ring moiety (i.e. the fused ring that contains W¹).

In embodiments,

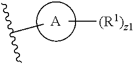

is

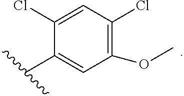 [SMILES: *c1cc(OC)c(Cl)cc1Cl]

In embodiments,

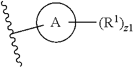

is

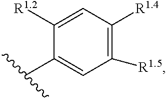

wherein R^{1.2} and R^{1.4} are halogen, and R^{1.5} is unsubstituted methoxy. In embodiments, z1 is 0. In embodiments, Ring A is aryl when z1 is non-zero. In embodiments, Ring A is a heteroaryl when z1 is non-zero. In embodiments, Ring A is a aryl (e.g., C₆-C₁₀ or phenyl) or 5 to 6 membered heteroaryl when z1 is non-zero. In embodiments, Ring A is aryl (e.g., C₆-C₁₀ or phenyl) when z1 is non-zero. In embodiments, Ring A is 5 to 6 membered heteroaryl when z1 is non-zero.

In embodiments, Ring B is phenyl. In embodiments, Ring B is a 5 to 6 membered heteroaryl. In embodiments, Ring B is a thienyl. In embodiments, Ring B is a 2-thienyl. In embodiments, Ring B is a 3-thienyl. In embodiments, Ring B is a pyridyl. In embodiments, Ring B is a 2-pyridyl. In embodiments, Ring B is a 3-pyridyl. In embodiments, Ring B is a 4-pyridyl. In embodiments, Ring B is a napththyl. In embodiments, Ring B is a 1-napththyl. In embodiments, Ring B is a 2-napththyl. In embodiments, Ring B is a quinolinyl. In embodiments, Ring B is a isoquinolinyl. In embodiments, Ring B is a 1-isoquinolinyl. In embodiments, Ring B is a 3-isoquinolinyl. In embodiments, Ring B is a 4-isoquinolinyl. In embodiments, Ring B is phenyl or 5 or 6 membered heteroaryl. In embodiments, the numbering of Ring B in this paragraph (e.g., 2-thienyl, 1-isoquinolinyl, etc.) refers the attachment point of the L¹ linker that connects Ring B to Ring A.

In embodiments, Ring B is

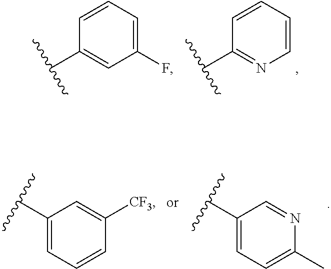

In embodiments, Ring B is

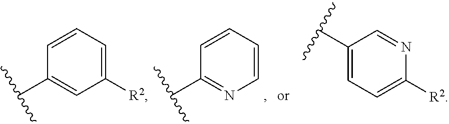

In embodiments, Ring B is

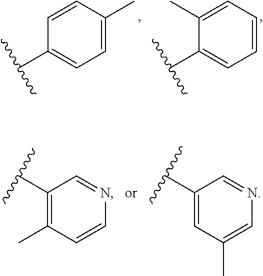

In embodiments, Ring B is

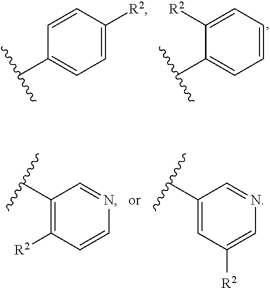

In embodiments, Ring B is a substituted aryl or substituted heteroaryl. In embodiments, Ring B is a substituted aryl. In embodiments, Ring B is a substituted heteroaryl. In embodiments, Ring B is a substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, Ring B is pyridyl when z2 is 1. In embodiments, Ring B is pyridyl when z2 is 2.

In embodiments,

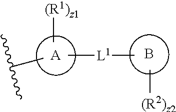

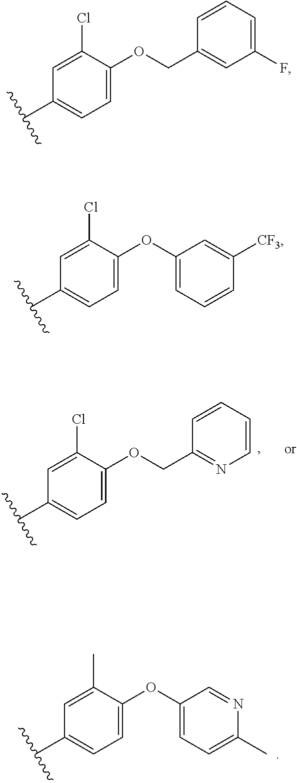 [SMILES: *c1ccc(OCc2cccc(F)c2)c(Cl)c1 ; *c1ccc(Oc2cccc(C(F)(F)F)c2)c(Cl)c1 ; *c1ccc(OCc2ccccn2)c(Cl)c1 ; *c1ccc(Oc2ccc(C)nc2)c(C)c1]

is

In embodiments,

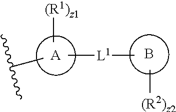

is

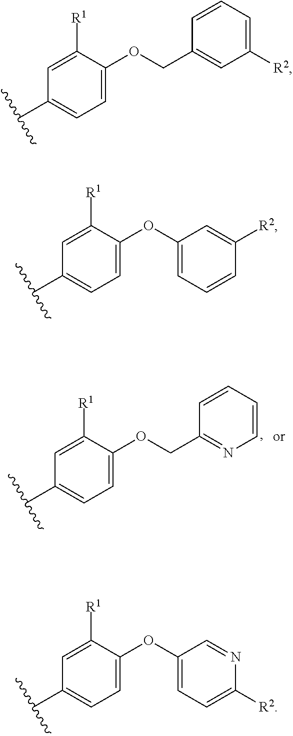

In embodiments,

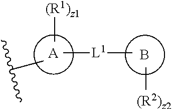

is

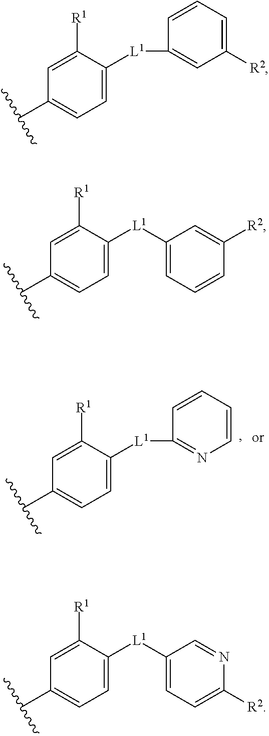

In embodiments,

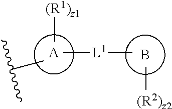

is

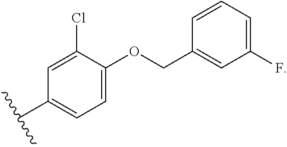 [SMILES: *c1ccc(OCc2cccc(F)c2)c(Cl)c1]

In embodiments,

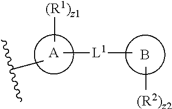

is

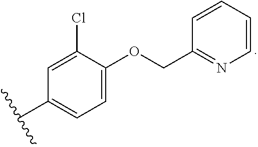 [SMILES: *c1ccc(OCc2ccccn2)c(Cl)c1]

In embodiments,

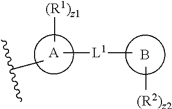

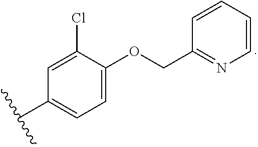 [SMILES: *c1ccc(OCc2ccccn2)c(Cl)c1]

In embodiments,

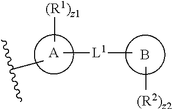

is

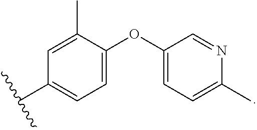 [SMILES: *c1ccc(Oc2ccc(C)nc2)c(C)c1]

In embodiments,

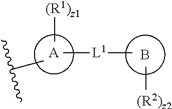

is

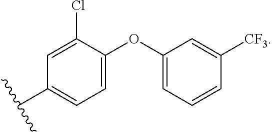 [SMILES: *c1ccc(Oc2cccc(C(F)(F)F)c2)c(Cl)c1]

In embodiments,

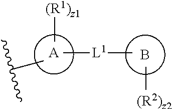

is

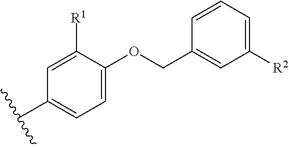

when W¹ is N. In embodiments,

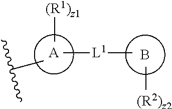

is

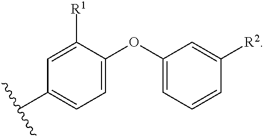

In embodiments,

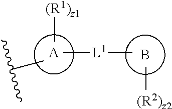

is

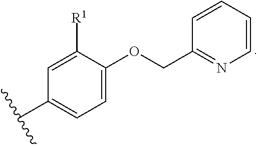

In embodiments,

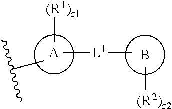

is

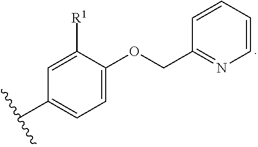

In embodiments,

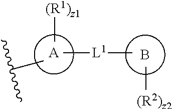

is

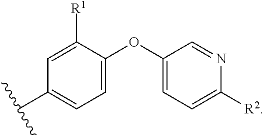

In embodiments,

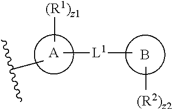

is

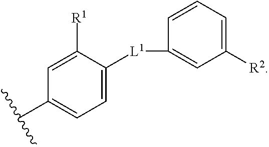

In embodiments,

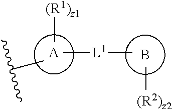

is

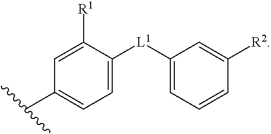

In embodiments,

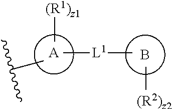

is

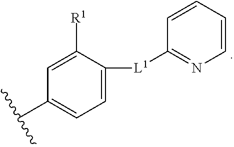

In embodiments,

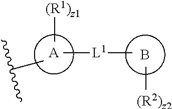

is

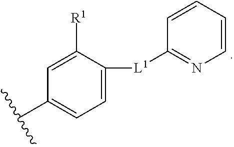

In embodiments,

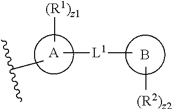

is

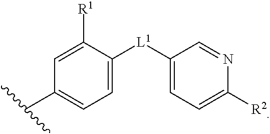

In embodiments, Ring C is cycloalkyl. In embodiments, Ring C is heterocycloalkyl. In embodiments, Ring C is aryl. In embodiments, Ring C is heteroaryl. In embodiments, Ring C is phenyl. In embodiments, Ring C is a 5 to 6 membered heteroaryl. In embodiments, Ring C is a thienyl. In embodiments, Ring C is a 2-thienyl. In embodiments, Ring C is a 3-thienyl. In embodiments, Ring C is a pyridyl. In embodiments, Ring C is a 2-pyridyl. In embodiments, Ring C is a 3-pyridyl. In embodiments, Ring C is a 4-pyridyl. In embodiments, Ring C is a C₃-C₆ cycloalkyl. In embodiments, Ring C is a C₃ cycloalkyl. In embodiments, Ring C is a C₄ cycloalkyl. In embodiments, Ring C is a C₅ cycloalkyl. In embodiments, Ring C is a C₆ cycloalkyl. In embodiments, Ring C is a 3 membered heterocycloalkyl. In embodiments, Ring C is a 4 membered heterocycloalkyl. In embodiments, Ring C is a 5 membered heterocycloalkyl. In embodiments, Ring C is a 6 membered heterocycloalkyl. In embodiments, the numbering of Ring C in this paragraph (e.g., 2-thienyl, 1-isoquinolinyl, etc.) refers the attachment point of the L² linker that connects Ring C to Ring B.

In embodiments,

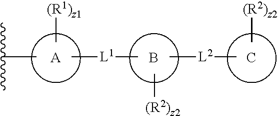

is

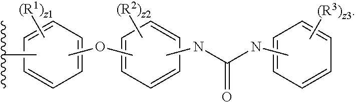

In embodiments,

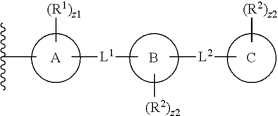

is

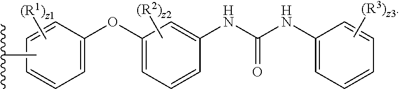

In embodiments,

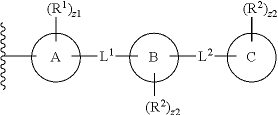

is

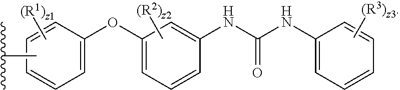

In embodiments,

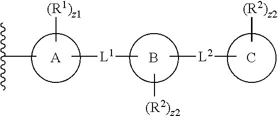

is

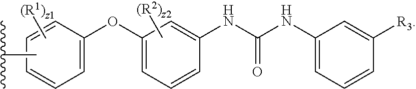

In embodiments,

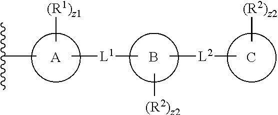

is

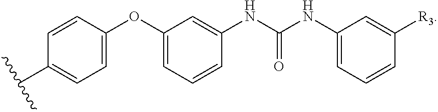

In embodiments,

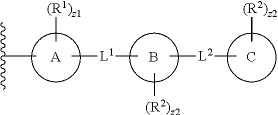

is

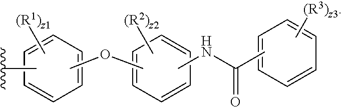

In embodiments,

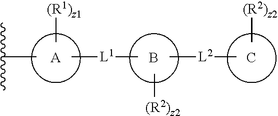

is

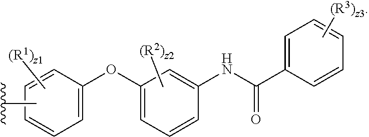

In embodiments,

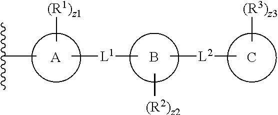

is

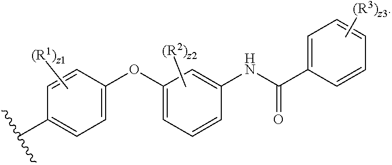

In embodiments,

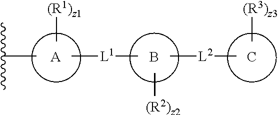

is

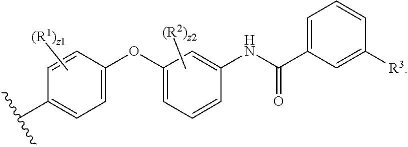

In embodiments,

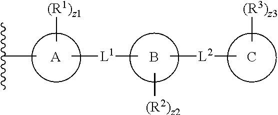

is

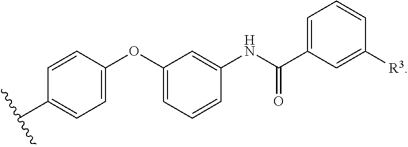

In embodiments, R¹ is independently a halogen, —CX¹₃, —CHX¹₂, —CH₂X¹, —CN, —OH, —NH₂, —COOH, —CONH₂, —NO₂, —SH, substituted or unsubstituted C₁-C₄ alkyl, or substituted or unsubstituted 2 to 4 membered heteroalkyl. In embodiments, R¹ is independently halogen, —CF₃, —CHF₂, —OCF₃, —OCHF₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R¹ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹ is independently halogen, —OH, —NH₂, —SH, unsubstituted C₁-C₄ alkyl, or unsubstituted 2 to 4 membered heteroalkyl. In embodiments, R¹ is independently halogen, —OH, unsubstituted methyl, or unsubstituted methoxy. In embodiments, R¹ is independently halogen. In embodiments, R¹ is independently —CF₃. In embodiments, R¹ is independently —CHF₂. In embodiments, R¹ is independently —CH₂F. In embodiments, R¹ is independently —OCF₃. In embodiments, R¹ is independently —OCHF₂. In embodiments, R¹ is independently —OCH₂F. In embodiments, R¹ is independently substituted or unsubstituted C₁-C₈ alkyl. In embodiments, R¹ is independently substituted or unsubstituted 2 to 8 membered heteroalkyl. In embodiments, R¹ is independently substituted or unsubstituted C₃-C₈ cycloalkyl. In embodiments, R¹ is independently substituted or unsubstituted 3 to 8 membered heterocycloalkyl. In embodiments, R¹ is independently substituted or unsubstituted C₆-C₁₀ aryl. In embodiments, R¹ is independently substituted or unsubstituted 5 to 10 membered heteroaryl. In embodiments, R¹ is independently —OH. In embodiments, R¹ is independently —NH₂. In embodiments, R¹ is independently —SH. In embodiments, R¹ is independently substituted or unsubstituted C₁-C₄ alkyl. In embodiments, R¹ is independently substituted or unsubstituted 2 to 4 membered heteroalkyl. In embodiments, R¹ is independently substituted or unsubstituted C₃-C₆ cycloalkyl. In embodiments, R¹ is independently substituted or unsubstituted 3 to 6 membered heterocycloalkyl. In embodiments, R¹ is independently substituted or unsubstituted phenyl. In embodiments, R¹ is independently substituted or unsubstituted 5 to 6 membered heteroaryl. In embodiments, R¹ is independently substituted C₁-C₈ alkyl. In embodiments, R¹ is independently substituted 2 to 8 membered heteroalkyl. In embodiments, R¹ is independently substituted C₃-C₈ cycloalkyl. In embodiments, R¹ is independently substituted 3 to 8 membered heterocycloalkyl. In embodiments, R¹ is independently substituted C₆-C₁₀ aryl. In embodiments, R¹ is independently substituted 5 to 10 membered heteroaryl. In embodiments, R¹ is independently substituted C₁-C₄ alkyl. In embodiments, R¹ is independently substituted 2 to 4 membered heteroalkyl. In embodiments, R¹ is independently substituted C₃-C₆ cycloalkyl. In embodiments, R¹ is independently substituted 3 to 6 membered heterocycloalkyl. In embodiments, R¹ is independently substituted phenyl. In embodiments, R¹ is independently substituted 5 to 6 membered heteroaryl. In embodiments, R¹ is independently unsubstituted C₁-C₈ alkyl. In embodiments, R¹ is independently unsubstituted 2 to 8 membered heteroalkyl. In embodiments, R¹ is independently unsubstituted C₃-C₈ cycloalkyl. In embodiments, R¹ is independently unsubstituted 3 to 8 membered heterocycloalkyl. In embodiments, R¹ is independently unsubstituted C₆-C₁₀ aryl. In embodiments, R¹ is independently unsubstituted 5 to 10 membered heteroaryl. In embodiments, R¹ is independently unsubstituted C₁-C₄ alkyl. In embodiments, R¹ is independently unsubstituted 2 to 4 membered heteroalkyl. In embodiments, R¹ is independently unsubstituted C₃-C₆ cycloalkyl. In embodiments, R¹ is independently unsubstituted 3 to 6 membered heterocycloalkyl. In embodiments, R¹ is independently unsubstituted phenyl. In embodiments, R¹ is independently unsubstituted 5 to 6 membered heteroaryl. In embodiments, R¹ is independently unsubstituted methyl. In embodiments, R¹ is independently unsubstituted ethyl. In embodiments, R¹ is independently unsubstituted isopropyl. In embodiments, R¹ is independently unsubstituted tert-butyl. In embodiments, R¹ is independently unsubstituted methoxy. In embodiments, R¹ is independently unsubstituted ethoxy. In embodiments, R¹ is independently —F. In embodiments, R¹ is independently —Cl. In embodiments, R¹ is independently —Br. In embodiments, R¹ is independently —I. In embodiments, R¹ is independently hydrogen.

In embodiments, z1 is 1. In embodiments, z1 is 0. In embodiments, z1 is 2. In embodiments, z1 is 3. In embodiments, z1 is 4. In embodiments, z1 is an integer from 0 to 4. In embodiments, z1 and z2 are 0. In embodiments, z1 is an integer from 0 to 1.

In embodiments, R² is independently a halogen, —CX²₃, —CHX²₂, —CH₂X², —CN, —OH, —NH₂, —COOH, —CONH₂, —NO₂, —SH, substituted or unsubstituted C₁-C₄ alkyl, or substituted or unsubstituted 2 to 4 membered heteroalkyl. In embodiments, R² is independently halogen, —CF₃, —CHF₂, —OCF₃, —OCHF₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R² is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R² is independently halogen, —OH, —NH₂, —SH, unsubstituted C₁-C₄ alkyl, or unsubstituted 2 to 4 membered heteroalkyl. In embodiments, R² is independently halogen, —OH, unsubstituted methyl, or unsubstituted methoxy. In embodiments, R² is independently halogen. In embodiments, R² is independently —CF₃. In embodiments, R² is independently —CHF₂. In embodiments, R² is independently —CH₂F. In embodiments, R² is independently —OCF₃. In embodiments, R² is independently —OCHF₂. In embodiments, R² is independently —OCH₂F. In embodiments, R² is independently substituted or unsubstituted C₁-C₈ alkyl. In embodiments, R² is independently substituted or unsubstituted 2 to 8 membered heteroalkyl. In embodiments, R² is independently substituted or unsubstituted C₃-C₈ cycloalkyl. In embodiments, R² is independently substituted or unsubstituted 3 to 8 membered heterocycloalkyl. In embodiments, R² is independently substituted or unsubstituted C₆-C₁₀ aryl. In embodiments, R² is independently substituted or unsubstituted 5 to 10 membered heteroaryl. In embodiments, R² is independently —OH. In embodiments, R² is independently —NH₂. In embodiments, R² is independently —SH. In embodiments, R² is independently substituted or unsubstituted C₁-C₄ alkyl. In embodiments, R² is independently substituted or unsubstituted 2 to 4 membered heteroalkyl. In embodiments, R² is independently substituted or unsubstituted C₃-C₆ cycloalkyl. In embodiments, R² is independently substituted or unsubstituted 3 to 6 membered heterocycloalkyl. In embodiments, R² is independently substituted or unsubstituted phenyl. In embodiments, R² is independently substituted or unsubstituted 5 to 6 membered heteroaryl. In embodiments, R² is independently substituted C₁-C₈ alkyl. In embodiments, R² is independently substituted 2 to 8 membered heteroalkyl. In embodiments, R² is independently substituted C₃-C₈ cycloalkyl. In embodiments, R² is independently substituted 3 to 8 membered heterocycloalkyl. In embodiments, R² is independently substituted C₆-C₁₀ aryl. In embodiments, R² is independently substituted 5 to 10 membered heteroaryl. In embodiments, R² is independently substituted C₁-C₄ alkyl. In embodiments, R² is independently substituted 2 to 4 membered heteroalkyl. In embodiments, R² is independently substituted C₃-C₆ cycloalkyl. In embodiments, R² is independently substituted 3 to 6 membered heterocycloalkyl. In embodiments, R² is independently substituted phenyl. In embodiments, R² is independently substituted 5 to 6 membered heteroaryl. In embodiments, R² is independently unsubstituted C₁-C₈ alkyl. In embodiments, R² is independently unsubstituted 2 to 8 membered heteroalkyl. In embodiments, R² is independently unsubstituted C₃-C₈ cycloalkyl. In embodiments, R² is independently unsubstituted 3 to 8 membered heterocycloalkyl. In embodiments, R² is independently unsubstituted C₆-C₁₀ aryl. In embodiments, R² is independently unsubstituted 5 to 10 membered heteroaryl. In embodiments, R² is independently unsubstituted C₁-C₄ alkyl. In embodiments, R² is independently unsubstituted 2 to 4 membered heteroalkyl. In embodiments, R² is independently unsubstituted C₃-C₆ cycloalkyl. In embodiments, R² is independently unsubstituted 3 to 6 membered heterocycloalkyl. In embodiments, R² is independently unsubstituted phenyl. In embodiments, R² is independently unsubstituted 5 to 6 membered heteroaryl. In embodiments, R² is independently unsubstituted methyl. In embodiments, R² is independently unsubstituted ethyl. In embodiments, R² is independently unsubstituted isopropyl. In embodiments, R² is independently unsubstituted tert-butyl. In embodiments, R² is independently unsubstituted methoxy. In embodiments, R² is independently unsubstituted ethoxy. In embodiments, R² is independently —F. In embodiments, R² is independently —Cl. In embodiments, R² is independently —Br. In embodiments, R² is independently —I. In embodiments, R² is independently hydrogen.

In embodiments, z2 is 1. In embodiments, z2 is 0. In embodiments, z2 is 2. In embodiments, z2 is 3. In embodiments, z2 is 4. In embodiments, z2 is 5. In embodiments, z2 is an integer from 0 to 4. In embodiments, z2 is an integer from 0 to 1.

In embodiments, R³ is independently a halogen, —CX³₃, —CHX³², —CH₂X³, —CN, —OH, —NH₂, —COOH, —CONH₂, —NO₂, —SH, substituted or unsubstituted C₁-C₄ alkyl, or substituted or unsubstituted 2 to 4 membered heteroalkyl. In embodiments, R³ is independently halogen, —CF₃, —CHF₂, —CH₂F, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R³ is independently halogen, —CF₃, —CHF₂, —OCF₃, —OCHF₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R³ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R³ is independently halogen, —OH, —NH₂, —SH, unsubstituted C₁-C₄ alkyl, or unsubstituted 2 to 4 membered heteroalkyl. In embodiments, R³ is independently halogen, —OH, unsubstituted methyl, or unsubstituted methoxy. In embodiments, R³ is independently halogen. In embodiments, R³ is independently —CF₃. In embodiments, R³ is independently —CHF₂. In embodiments, R³ is independently —CH₂F. In embodiments, R³ is independently —OCF₃. In embodiments, R³ is independently —OCHF₂. In embodiments, R³ is independently —OCH₂F. In embodiments, R³ is independently substituted or unsubstituted C₁-C₈ alkyl. In embodiments, R³ is independently substituted or unsubstituted 2 to 8 membered heteroalkyl. In embodiments, R³ is independently substituted or unsubstituted C₃-C₈ cycloalkyl. In embodiments, R³ is independently substituted or unsubstituted 3 to 8 membered heterocycloalkyl. In embodiments, R³ is independently substituted or unsubstituted C₆-C₁₀ aryl. In embodiments, R³ is independently substituted or unsubstituted 5 to 10 membered heteroaryl. In embodiments, R³ is independently —OH. In embodiments, R³ is independently —NH₂. In embodiments, R³ is independently —SH. In embodiments, R³ is independently substituted or unsubstituted C₁-C₄ alkyl. In embodiments, R³ is independently substituted or unsubstituted 2 to 4 membered heteroalkyl. In embodiments, R³ is independently substituted or unsubstituted C₃-C₆ cycloalkyl. In embodiments, R³ is independently substituted or unsubstituted 3 to 6 membered heterocycloalkyl. In embodiments, R³ is independently substituted or unsubstituted phenyl. In embodiments, R³ is independently substituted or unsubstituted 5 to 6 membered heteroaryl. In embodiments, R³ is independently substituted C₁-C₈ alkyl. In embodiments, R³ is independently substituted 2 to 8 membered heteroalkyl. In embodiments, R³ is independently substituted C₃-C₈ cycloalkyl. In embodiments, R³ is independently substituted 3 to 8 membered heterocycloalkyl. In embodiments, R³ is independently substituted C₆-C₁₀ aryl. In embodiments, R³ is independently substituted 5 to 10 membered heteroaryl. In embodiments, R³ is independently substituted C₁-C₄ alkyl. In embodiments, R³ is independently substituted 2 to 4 membered heteroalkyl. In embodiments, R³ is independently substituted C₃-C₆ cycloalkyl. In embodiments, R³ is independently substituted 3 to 6 membered heterocycloalkyl. In embodiments, R³ is independently substituted phenyl. In embodiments, R³ is independently substituted 5 to 6 membered heteroaryl. In embodiments, R³ is independently unsubstituted C₁-C₈ alkyl. In embodiments, R³ is independently unsubstituted 2 to 8 membered heteroalkyl. In embodiments, R³ is independently unsubstituted C₃-C₈ cycloalkyl. In embodiments, R³ is independently unsubstituted 3 to 8 membered heterocycloalkyl. In embodiments, R³ is independently unsubstituted C₆-C₁₀ aryl. In embodiments, R³ is independently unsubstituted 5 to 10 membered heteroaryl. In embodiments, R³ is independently unsubstituted C₁-C₄ alkyl. In embodiments, R³ is independently unsubstituted 2 to 4 membered heteroalkyl. In embodiments, R³ is independently unsubstituted C₃-C₆ cycloalkyl. In embodiments, R³ is independently unsubstituted 3 to 6 membered heterocycloalkyl. In embodiments, R³ is independently unsubstituted phenyl. In embodiments, R³ is independently unsubstituted 5 to 6 membered heteroaryl. In embodiments, R³ is independently unsubstituted methyl. In embodiments, R³ is independently unsubstituted ethyl. In embodiments, R³ is independently unsubstituted isopropyl. In embodiments, R³ is independently unsubstituted tert-butyl. In embodiments, R³ is independently unsubstituted methoxy. In embodiments, R³ is independently unsubstituted ethoxy. In embodiments, R³ is independently —F. In embodiments, R³ is independently —Cl. In embodiments, R³ is independently —Br. In embodiments, R³ is independently —I. In embodiments, R³ is independently hydrogen. In embodiments, R³ is —CF₃. In embodiments, R³ is a halogen.

In embodiments, z3 is 1. In embodiments, z3 is 0. In embodiments, z3 is 2. In embodiments, z3 is 3. In embodiments, z3 is 4. In embodiments, z3 is 5.

In embodiments, R⁴ is not a degradation-increasing moiety. In embodiments, -L⁴-R⁴ is not a degradation-increasing moiety. In embodiments, -L⁴-R⁴ does not include a degradation-increasing moiety. In embodiments, R⁴ is independently halogen, —CF₃, —CHF₂, —CH₂F, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁴ is independently halogen, —CF₃, —CHF₂, —OCF₃, —OCHF₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁴ is independently halogen, —OH, —NH₂, —SH, unsubstituted C₁-C₄ alkyl, or unsubstituted 2 to 4 membered heteroalkyl. In embodiments, R⁴ is independently halogen, —OH, unsubstituted methyl, or unsubstituted methoxy. In embodiments, R⁴ is independently halogen. In embodiments, R⁴ is independently —CF₃. In embodiments, R⁴ is independently —CHF₂. In embodiments, R⁴ is independently —CH₂F. In embodiments, R⁴ is independently —OCF₃. In embodiments, R⁴ is independently —OCHF₂. In embodiments, R⁴ is independently —OCH₂F. In embodiments, R⁴ is independently substituted or unsubstituted C₁-C₈ alkyl. In embodiments, R⁴ is independently substituted or unsubstituted 2 to 8 membered heteroalkyl. In embodiments, R⁴ is independently substituted or unsubstituted C₃-C₈ cycloalkyl. In embodiments, R⁴ is independently substituted or unsubstituted 3 to 8 membered heterocycloalkyl. In embodiments, R⁴ is independently substituted or unsubstituted C₆-C₁₀ aryl. In embodiments, R⁴ is independently substituted or unsubstituted 5 to 10 membered heteroaryl. In embodiments, R⁴ is independently —OH. In embodiments, R⁴ is independently —NH₂. In embodiments, R⁴ is independently —SH. In embodiments, R⁴ is independently substituted or unsubstituted C₁-C₄ alkyl. In embodiments, R⁴ is independently substituted or unsubstituted 2 to 4 membered heteroalkyl. In embodiments, R⁴ is independently substituted or unsubstituted C₃-C₆ cycloalkyl. In embodiments, R⁴ is independently substituted or unsubstituted 3 to 6 membered heterocycloalkyl. In embodiments, R⁴ is independently substituted or unsubstituted phenyl. In embodiments, R⁴ is independently substituted or unsubstituted 5 to 6 membered heteroaryl. In embodiments, R⁴ is independently substituted C₁-C₈ alkyl. In embodiments, R⁴ is independently substituted 2 to 8 membered heteroalkyl. In embodiments, R⁴ is independently substituted C₃-C₈ cycloalkyl. In embodiments, R⁴ is independently substituted 3 to 8 membered heterocycloalkyl. In embodiments, R⁴ is independently substituted C₆-C₁₀ aryl. In embodiments, R⁴ is independently substituted 5 to 10 membered heteroaryl. In embodiments, R⁴ is independently substituted C₁-C₄ alkyl. In embodiments, R⁴ is independently substituted 2 to 4 membered heteroalkyl. In embodiments, R⁴ is independently substituted C₃-C₆ cycloalkyl. In embodiments, R⁴ is independently substituted 3 to 6 membered heterocycloalkyl. In embodiments, R⁴ is independently substituted phenyl. In embodiments, R⁴ is independently substituted 5 to 6 membered heteroaryl. In embodiments, R⁴ is independently unsubstituted C₁-C₈ alkyl. In embodiments, R⁴ is independently unsubstituted 2 to 8 membered heteroalkyl. In embodiments, R⁴ is independently unsubstituted C₃-C₈ cycloalkyl. In embodiments, R⁴ is independently unsubstituted 3 to 8 membered heterocycloalkyl. In embodiments, R⁴ is independently unsubstituted C₆-C₁₀ aryl. In embodiments, R⁴ is independently unsubstituted 5 to 10 membered heteroaryl. In embodiments, R⁴ is independently unsubstituted C₁-C₄ alkyl. In embodiments, R⁴ is independently unsubstituted 2 to 4 membered heteroalkyl. In embodiments, R⁴ is independently unsubstituted C₃-C₆ cycloalkyl. In embodiments, R⁴ is independently unsubstituted 3 to 6 membered heterocycloalkyl. In embodiments, R⁴ is independently unsubstituted phenyl. In embodiments, R⁴ is independently unsubstituted 5 to 6 membered heteroaryl. In embodiments, R⁴ is —NH₂.

In embodiments, -L⁴-R⁴ is unsubstituted methoxy.

In embodiments, -L⁴-R⁴ is

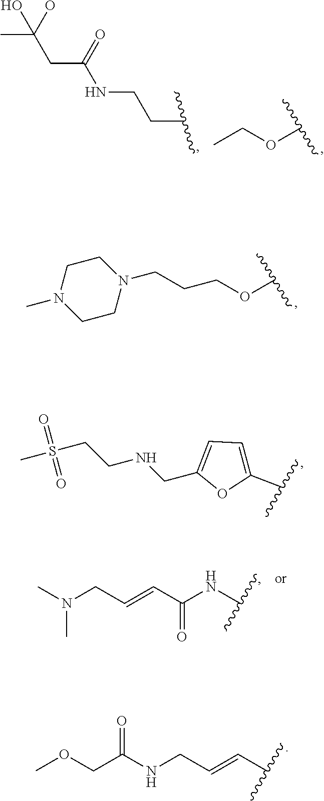

In embodiments, -L⁴-R⁴ is

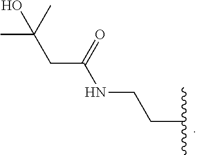 [SMILES: *CCNC(=O)CC(C)(C)O]

In embodiments, -L⁴-R⁴ is

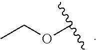 [SMILES: *OCC]

In embodiments, -L⁴-R⁴ is

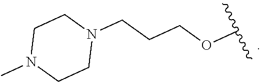 [SMILES: *OCCCN1CCN(C)CC1]

In embodiments, -L⁴-R⁴ is

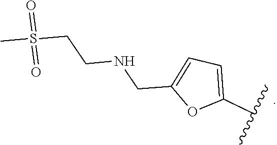 [SMILES: *c1ccc(CNCCS(C)(=O)=O)o1]

In embodiments, -L⁴-R⁴ is

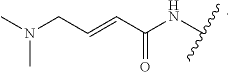 [SMILES: *NC(=O)/C=C/CN(C)C]

In embodiments, -L⁴-R⁴ is

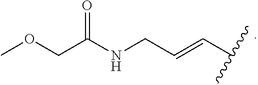 [SMILES: */C=C/CNC(=O)COC]

In embodiments, -L⁴-R⁴ is substituted or unsubstituted 5 to 10 membered heteroalkyl. In embodiments, -L⁴-R⁴ is substituted or unsubstituted 6 to 10 membered heteroalkyl. In embodiments, -L⁴-R⁴ is substituted or unsubstituted 7 to 10 membered heteroalkyl. In embodiments, -L⁴-R⁴ is substituted or unsubstituted 7 to 9 membered heteroalkyl. In embodiments, -L⁴-R⁴ is substituted or unsubstituted 5 membered heteroaryl. In embodiments, -L⁴-R⁴ is substituted or unsubstituted 5 to 6 membered heteroaryl. In embodiments, -L⁴-R⁴ is substituted or unsubstituted 4 to 6 membered heteroaryl. In embodiments, -L⁴-R⁴ is substituted or unsubstituted heteroaryl. In embodiments, -L⁴-R⁴ is substituted 5 membered heteroaryl. In embodiments, -L⁴-R⁴ is substituted 5 to 6 membered heteroaryl. In embodiments, -L⁴-R⁴ is substituted 4 to 6 membered heteroaryl. In embodiments, -L⁴-R⁴ is substituted heteroaryl. In embodiments, -L⁴-R⁴ is unsubstituted 5 membered heteroaryl. In embodiments, -L⁴-R⁴ is unsubstituted 5 to 6 membered heteroaryl. In embodiments, -L⁴-R⁴ is unsubstituted heteroaryl. In embodiments, -L⁴-R⁴ is unsubstituted 4 to 6 membered heteroaryl.

In embodiments, -L⁴-R⁴ is

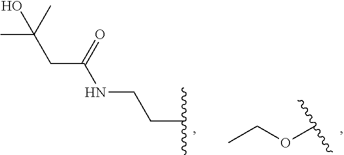

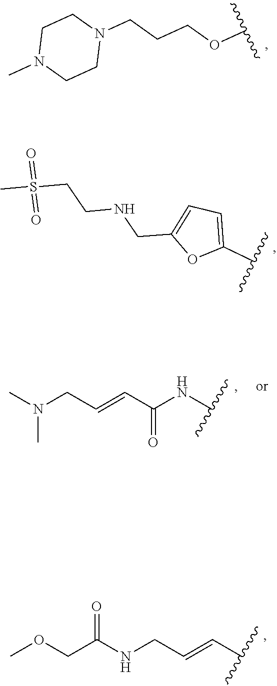

when R⁵ is a degradation-increasing moiety. In embodiments, -L⁴-R⁴ is

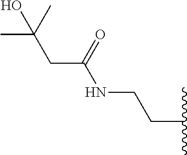 [SMILES: *CCNC(=O)CC(C)(C)O]

when R⁵ is a degradation-increasing moiety. In embodiments, -L⁴-R⁴ is

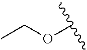 [SMILES: *OCC]

when R⁵ is a degradation-increasing moiety. In embodiments, -L⁴-R⁴ is

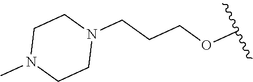 [SMILES: *OCCCN1CCN(C)CC1]

when R⁵ is a degradation-increasing moiety. In embodiments, -L⁴-R⁴ is

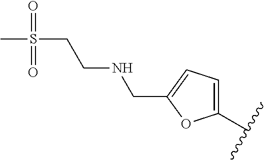 [SMILES: *c1ccc(CNCCS(C)(=O)=O)o1]

when R⁵ is a degradation-increasing moiety. In embodiments, -L⁴-R⁴ is

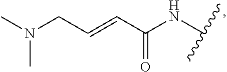 [SMILES: *NC(=O)/C=C/CN(C)C]

when R⁵ is a degradation-increasing moiety. In embodiments, -L⁴-R⁴ is

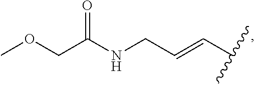 [SMILES: */C=C/CNC(=O)COC]

when R⁵ is a degradation-increasing moiety.

R⁵ is a degradation-increasing moiety.

In embodiments, R⁵ is substituted or unsubstituted alkylene or substituted or unsubstituted heteroalkylene. In embodiments, R⁵ is substituted or unsubstituted heteroalkylene. In embodiments, R⁵ is substituted or unsubstituted 2 to 26 membered heteroalkylene. In embodiments, R⁵ is substituted or unsubstituted 2 to 24 membered heteroalkylene. In embodiments, R⁵ is substituted or unsubstituted 2 to 22 membered heteroalkylene. In embodiments, R⁵ is substituted or unsubstituted 2 to 20 membered heteroalkylene. In embodiments, R⁵ is substituted or unsubstituted 2 to 18 membered heteroalkylene. In embodiments, R⁵ is substituted or unsubstituted 2 to 16 membered heteroalkylene. In embodiments, R⁵ is substituted or unsubstituted 2 to 14 membered heteroalkylene. In embodiments, R⁵ is substituted or unsubstituted 2 to 12 membered heteroalkylene. In embodiments, R⁵ is substituted or unsubstituted 2 to 10 membered heteroalkylene. In embodiments, R⁵ is substituted or unsubstituted 10 to 24 membered heteroalkylene. In embodiments, R⁵ is substituted or unsubstituted 10 to 22 membered heteroalkylene. In embodiments, R⁵ is substituted or unsubstituted 10 to 20 membered heteroalkylene. In embodiments, R⁵ is substituted or unsubstituted 10 to 18 membered heteroalkylene. In embodiments, R⁵ is substituted or unsubstituted 10 to 16 membered heteroalkylene. In embodiments, R⁵ is substituted or unsubstituted 10 to 14 membered heteroalkylene. In embodiments, R⁵ is substituted or unsubstituted 10 to 12 membered heteroalkylene. In embodiments, R⁵ is substituted or unsubstituted 15 to 24 membered heteroalkylene. In embodiments, R⁵ is substituted or unsubstituted 15 to 22 membered heteroalkylene. In embodiments, R⁵ is substituted or unsubstituted 15 to 20 membered heteroalkylene. In embodiments, R⁵ is substituted or unsubstituted 15 to 18 membered heteroalkylene. In embodiments, R⁵ is substituted or unsubstituted 15 to 16 membered heteroalkylene.

In embodiments, a substituted R⁵ (e.g., substituted alkylene and/or substituted heteroalkylene) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is substituted or unsubstituted 2 to 24 membered heteroalkylene having a

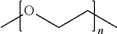

group. In embodiments, R⁵ is substituted or unsubstituted 2 to 22 membered heteroalkylene having a

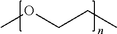

group. In embodiments, R⁵ is substituted or unsubstituted 2 to 20 membered heteroalkylene having a

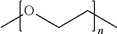

group. In embodiments, R⁵ is substituted or unsubstituted 2 to 18 membered heteroalkylene having a

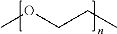

group. In embodiments, R⁵ is substituted or unsubstituted 2 to 16 membered heteroalkylene having a

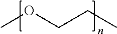

group. In embodiments, R⁵ is substituted or unsubstituted 2 to 14 membered heteroalkylene having a

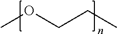

group. In embodiments, R⁵ is substituted or unsubstituted 2 to 12 membered heteroalkylene having a

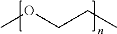

group. In embodiments, R⁵ is substituted or unsubstituted 2 to 10 membered heteroalkylene having a

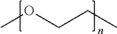

group. In embodiments, R⁵ is substituted or unsubstituted 10 to 24 membered heteroalkylene having a

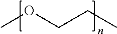

group. In embodiments, R⁵ is substituted or unsubstituted 10 to 22 membered heteroalkylene having a

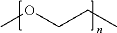

group. In embodiments, R⁵ is substituted or unsubstituted 10 to 20 membered heteroalkylene having a

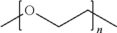

group. In embodiments, R⁵ is substituted or unsubstituted 10 to 18 membered heteroalkylene having a

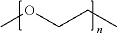

group. In embodiments, R⁵ is substituted or unsubstituted 10 to 16 membered heteroalkylene having a

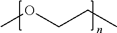

group. In embodiments, R⁵ is substituted or unsubstituted 10 to 14 membered heteroalkylene having a

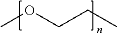

group. In embodiments, R⁵ is substituted or unsubstituted 10 to 14 membered heteroalkylene having a

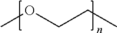

group. In embodiments, R⁵ is substituted or unsubstituted 15 to 24 membered heteroalkylene having a

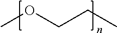

group. In embodiments, R⁵ is substituted or unsubstituted 15 to 22 membered heteroalkylene having a

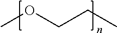

group. In embodiments, R⁵ is substituted or unsubstituted 15 to 20 membered heteroalkylene having a

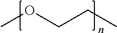

group. In embodiments, R⁵ is substituted or unsubstituted 15 to 28 membered heteroalkylene having a

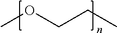

group. In embodiments, R⁵ is substituted or unsubstituted 15 to 16 membered heteroalkylene having a

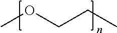

group.

In embodiments, n is 1 to 10. In embodiments, n is 1 to 9. In embodiments, n is 1 to 8. In embodiments, n is 1 to 7. In embodiments, n is 1 to 6. In embodiments, n is 1 to 5. In embodiments, n is 1 to 4. In embodiments, n is 1 to 3. In embodiments, n is 1 to 2. In embodiments, n is 2 to 10. In embodiments, n is 2 to 9. In embodiments, n is 2 to 8. In embodiments, n is 2 to 7. In embodiments, n is 2 to 6. In embodiments, n is 2 to 5. In embodiments, n is 2 to 4. In embodiments, n is 2 to 3. In embodiments, n is 3 to 10. In embodiments, n is 3 to 9. In embodiments, n is 3 to 8. In embodiments, n is 3 to 7. In embodiments, n is 3 to 6. In embodiments, n is 3 to 5. In embodiments, n is 3 to 4. In embodiments, n is 4 to 10. In embodiments, n is 4 to 9. In embodiments, n is 4 to 8. In embodiments, n is 4 to 7. In embodiments, n is 4 to 6. In embodiments, n is 4 to 5.

In embodiments, R⁵ is substituted or unsubstituted 2 to 22 membered heteroalkylene having a

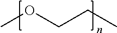

group, wherein n is 2 to 4. In embodiments, R⁵ is substituted or unsubstituted 10 to 22 membered heteroalkylene having a

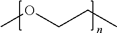

group, wherein n is 2 to 4. In embodiments, R⁵ is substituted or unsubstituted 15 to 22 membered heteroalkylene having a

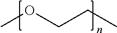

group, wherein n is 2 to 4.

In embodiments, the degradation-increasing moiety is thalidomide moiety or an analog, derivative, or prodrug thereof; phthalimide moiety or an analog, derivative, or prodrug thereof; adamantyl or an analog, derivative, or prodrug thereof; an IκBα phosphopeptide moiety or an analog, derivative, or prodrug thereof; nutlin moiety or an analog, derivative, or prodrug thereof; HIF-1α pentapeptide moiety or an analog, derivative, or prodrug thereof; or

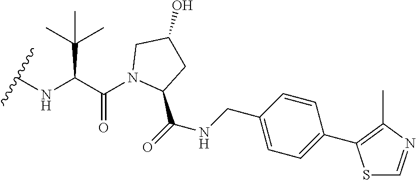 [SMILES: *N[C@H](C(=O)N1C[C@H](O)C[C@H]1C(=O)NCc1ccc(-c2scnc2C)cc1)C(C)(C)C]

or an analog, derivative, or prodrug thereof. In embodiments, the degradation-increasing moiety is thalidomide moiety or an analog, derivative, or prodrug thereof. In embodiments, R⁵ is

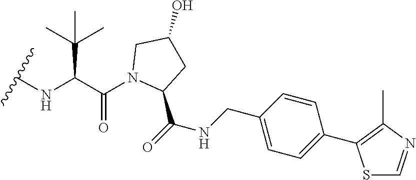 [SMILES: *N[C@H](C(=O)N1C[C@H](O)C[C@H]1C(=O)NCc1ccc(-c2scnc2C)cc1)C(C)(C)C]

or an analog, derivative, or prodrug thereof. In embodiments, R⁵ is

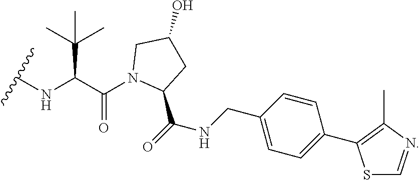 [SMILES: *N[C@H](C(=O)N1C[C@H](O)C[C@H]1C(=O)NCc1ccc(-c2scnc2C)cc1)C(C)(C)C]

In embodiments, R⁵ is a thalidomide moiety or an analog, derivative, or prodrug thereof. In embodiments, R⁵ is a thalidomide moiety. In embodiments, R⁵ is a lenalidomide moiety. In embodiments, R⁵ is a pomalidomide moiety. In embodiments, R⁵ is

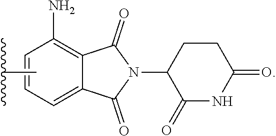

In embodiments, R⁵ is

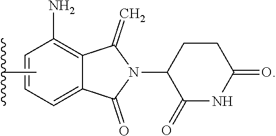

In embodiments, R⁵ is

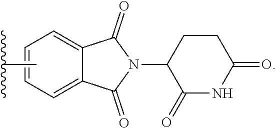

In embodiments, R⁵ is

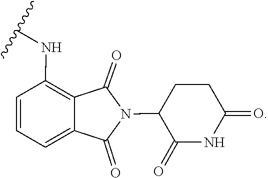 [SMILES: *Nc1cccc2c1C(=O)N(C1CCC(=O)NC1=O)C2=O]

In embodiments, R⁵ is

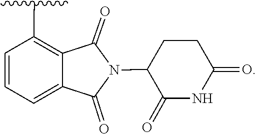 [SMILES: *c1cccc2c1C(=O)N(C1CCC(=O)NC1=O)C2=O]

In embodiments, R⁵ is a phthalimide moiety or an analog, derivative, or prodrug thereof. In embodiments, R⁵ is

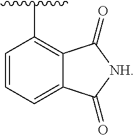 [SMILES: *c1cccc2c1C(=O)NC2=O]

In embodiments, R⁵ is

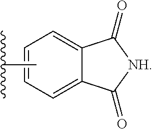

In embodiments, R⁵ is a phthalimide moiety. In embodiments, R⁵ includes a thalidomide moiety or an analog, derivative, or prodrug thereof. In embodiments, R⁵ includes a thalidomide moiety. In embodiments, R⁵ includes a lenalidomide moiety. In embodiments, R⁵ includes a pomalidomide moiety. In embodiments, R⁵ includes a phthalimide moiety or an analog, derivative, or prodrug thereof. In embodiments, R⁵ includes a phthalimide moiety.

In embodiments, R⁵ is adamantyl or an analog, derivative, or prodrug thereof. In embodiments, R⁵ is substituted adamantyl or an analog, derivative, or prodrug thereof. In embodiments, R⁵ is adamantyl. In embodiments, R⁴ is substituted adamantyl.

In embodiments, R⁵ is a phosphopeptide including a sequence of IκBα, or an analog, derivative, or prodrug thereof. In embodiments, R⁵ is a phosphopeptide consisting of a sequence of IκBα, or an analog, derivative, or prodrug thereof. In embodiments, R⁵ is—RAEDS*GNES*EGE (SEQ ID NO:3) or a derivative thereof (e.g., including one, two or three substitutions (e.g., conservative substitutions), wherein S* is a phosphoserine. In embodiments, R⁵ is—RAEDS*GNES*EGE (SEQ ID NO:3). In embodiments, R⁵ is -G₀₋₁₀RAEDS*GNES*EGE (SEQ ID NO:4) or a derivative thereof (e.g., including one, two or three substitutions (e.g., conservative substitutions), wherein S* is a phosphoserine. In embodiments, R⁵ is -G₀₋₁₀RAEDS*GNES*EGE (SEQ ID NO:4). In embodiments, R⁵ is -DRIIDS*GLDS*M (SEQ ID NO:5) or a derivative thereof (e.g., including one, two or three substitutions (e.g., conservative substitutions), wherein S* is a phosphoserine. In embodiments, R⁵ is -DRIIDS*GLDS*M (SEQ ID NO:5). In embodiments, R⁵ is -G₀₋₁₀DRIIDS*GLDS*M (SEQ ID NO:6) or a derivative thereof (e.g., including one, two or three substitutions (e.g., conservative substitutions), wherein S* is a phosphoserine. In embodiments, R⁵ is -G₀₋₁₀DRIIDS*GLDS*M (SEQ ID NO:6). “IκBα” is used in accordance with its well understood meaning and refers to a protein that inhibits NF-κB transcription factor. In embodiments, phosphopeptide including a sequence of IκBα is as described in P. Natl Acad Sci USA (2001) July 17, vol. 98(15) 8554-8559. In embodiments, R⁵ includes a phosphopeptide including a sequence of IκBα, or an analog, derivative, or prodrug thereof. In embodiments, R⁵ includes a phosphopeptide consisting of a sequence of IκBα, or an analog, derivative, or prodrug thereof. In embodiments, R⁵ includes -RAEDS*GNES*EGE (SEQ ID NO:3) or a derivative thereof (e.g., including one, two or three substitutions (e.g., conservative substitutions), wherein S* is a phosphoserine. In embodiments, R⁵ includes -RAEDS*GNES*EGE (SEQ ID NO:3). In embodiments, R⁵ is-G₀₁₀RAEDS*GNES*EGE (SEQ ID NO:4) or a derivative thereof (e.g., including one, two or three substitutions (e.g., conservative substitutions), wherein S* is a phosphoserine. In embodiments, R⁵ includes -G₀₋₁₀RAEDS*GNES*EGE (SEQ ID NO:4). In embodiments, R⁵ includes—DRIIDS*GLDS*M (SEQ ID NO:5) or a derivative thereof (e.g., including one, two or three substitutions (e.g., conservative substitutions), wherein S* is a phosphoserine. In embodiments, R⁵ includes -DRIIDS*GLDS*M (SEQ ID NO:5). In embodiments, R⁵ includes -G₀₋₁₀DRIIDS*GLDS*M (SEQ ID NO:6) or a derivative thereof (e.g., including one, two or three substitutions (e.g., conservative substitutions), wherein S* is a phosphoserine. In embodiments, R⁵ includes -G₀₋₁₀DRIIDS*GLDS*M (SEQ ID NO:6).

In embodiments, R⁵ is a nutlin moiety or an analog, derivative, or prodrug thereof. In embodiments, R⁵ is a nutlin-3 moiety or an analog, derivative, or prodrug thereof. In embodiments, R⁵ is a (±)-4-[4,5-Bis(4-chlorophenyl)-2-(2-isopropoxy-4-methoxy-phenyl)-4,5-dihydro-imidazole-1-carbonyl]-piperazin-2-one moiety. In embodiments, R⁵ is a (−)-4-[4,5-Bis(4-chlorophenyl)-2-(2-isopropoxy-4-methoxy-phenyl)-4,5-dihydro-imidazole-1-carbonyl]-piperazin-2-one moiety. In embodiments, R⁵ is a (±)-4-[4,5-Bis(4-chlorophenyl)-2-(2-isopropoxy-4-methoxy-phenyl)-4,5-dihydro-imidazole-1-carbonyl]-piperazin-2-one moiety. In embodiments, R⁵ is a nutlin moiety. In embodiments, R⁵ is a nutlin-3 moiety. “Nutlin” and “nutlin-3” are used in accordance with their well understood meaning and refer to cis-imidazoline analogs. In embodiments, nutlin or nutlin-3 is as described in Bioorg. Med. Chem. Lett (2008) 18 5904-5908. In embodiments, R⁵ includes a nutlin moiety or an analog, derivative, or prodrug thereof. In embodiments, R⁵ includes a nutlin-3 moiety or an analog, derivative, or prodrug thereof. In embodiments, R⁵ includes a (±)-4-[4,5-Bis(4-chlorophenyl)-2-(2-isopropoxy-4-methoxy-phenyl)-4,5-dihydro-imidazole-1-carbonyl]-piperazin-2-one moiety. In embodiments, R⁵ includes a (−)-4-[4,5-Bis(4-chlorophenyl)-2-(2-isopropoxy-4-methoxy-phenyl)-4,5-dihydro-imidazole-1-carbonyl]-piperazin-2-one moiety. In embodiments, R⁵ includes a (+)-4-[4,5-Bis(4-chlorophenyl)-2-(2-isopropoxy-4-methoxy-phenyl)-4,5-dihydro-imidazole-1-carbonyl]-piperazin-2-one moiety. In embodiments, R⁵ includes a nutlin moiety.

In embodiments, R⁵ is a hydroxyproline containing HIF-1α pentapeptide moiety or an analog, derivative, or prodrug thereof. In embodiments, R⁵ is -LAP*YI or a derivative thereof (e.g., including one, two or three substitutions (e.g., conservative substitutions), wherein P* is a hydroxyproline. In embodiments, R⁵ is -G₀₋₁₀LAP*YI or a derivative thereof (e.g., including one, two or three substitutions (e.g., conservative substitutions), wherein P* is a hydroxyproline. In embodiments, R⁵ is -LAP*YI. In embodiments, R⁵ is -G₀₋₁₀LAP*YI. “HIF-1α” is used in accordance with its well understood meaning and refers to the hypoxia-inducible factor 1-alpha transcription factor. In embodiments, HIF-1α is as described in Oncogene (2008) 27, 7201-7211. In embodiments, R⁵ includes a hydroxyproline containing HIF-1α pentapeptide moiety or an analog, derivative, or prodrug thereof. In embodiments, R⁵ includes -LAP*YI or a derivative thereof (e.g., including one, two or three substitutions (e.g., conservative substitutions), wherein P* is a hydroxyproline. In embodiments, R⁵ includes -G₀₋₁₀LAP*YI or a derivative thereof (e.g., including one, two or three substitutions (e.g., conservative substitutions), wherein P* is a hydroxyproline. In embodiments, R⁵ includes -LAP*YI. In embodiments, R⁴ includes -G₀₋₁₀LAP*YI.

In embodiments, R⁵ is a degradation-increasing moiety. In embodiments, the degradation-increasing moiety is a thalidomide moiety or a derivative, analog, or prodrug thereof. In embodiments, the degradation-increasing moiety is an analog thereof. In embodiments, the degradation-increasing moiety is a derivative thereof. In embodiments, the degradation-increasing moiety is a prodrug thereof (e.g., a physiologically hydrolyzable ester thereof or a physiologically hydrolyzable amide thereof). In embodiments, the degradation-increasing moiety is a thalidomide moiety. In embodiments of a derivative, an original substituent (e.g., substituent group) of a thalidomide moiety is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of a thalidomide moiety are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of a thalidomide moiety is replaced with a substituent (e.g., substituent group). In embodiments of a derivative, a plurality of hydrogen atoms of a thalidomide moiety are replaced with a plurality of substituents (e.g., substituent groups), wherein the substituents (e.g., substituent groups) are each optionally different.

In embodiments, R⁵ is a degradation-increasing moiety. In embodiments, the degradation-increasing moiety is a phthalimide moiety or a derivative, analog, or prodrug thereof. In embodiments, the degradation-increasing moiety is an analog thereof. In embodiments, the degradation-increasing moiety is a derivative thereof. In embodiments, the degradation-increasing moiety is a prodrug thereof (e.g., a physiologically hydrolyzable ester thereof or a physiologically hydrolyzable amide thereof). In embodiments, the degradation-increasing moiety is a phthalimide moiety. In embodiments of a derivative, an original substituent (e.g., substituent group) of a phthalimide moiety is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of a phthalimide moiety are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of a phthalimide moiety is replaced with a substituent (e.g., substituent group). In embodiments of a derivative, a plurality of hydrogen atoms of a phthalimide moiety are replaced with a plurality of substituents (e.g., substituent groups), wherein the substituents (e.g., substituent groups) are each optionally different.

In embodiments, R⁵ is a degradation-increasing moiety. In embodiments, the degradation-increasing moiety is adamantyl or a derivative, analog, or prodrug thereof. In embodiments, the degradation-increasing moiety is an analog thereof. In embodiments, the degradation-increasing moiety is a derivative thereof. In embodiments, the degradation-increasing moiety is a prodrug thereof (e.g., a physiologically hydrolyzable ester thereof or a physiologically hydrolyzable amide thereof). In embodiments, the degradation-increasing moiety is adamantyl. In embodiments of a derivative, an original substituent (e.g., substituent group) of adamantyl is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of adamantyl are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of adamantyl is replaced with a substituent (e.g., substituent group). In embodiments of a derivative, a plurality of hydrogen atoms of adamantyl are replaced with a plurality of substituents (e.g., substituent groups), wherein the substituents (e.g., substituent groups) are each optionally different.

In embodiments, R⁵ is a degradation-increasing moiety. In embodiments, the degradation-increasing moiety is an IκBα phosphopeptide moiety or a derivative, analog, or prodrug thereof. In embodiments, the degradation-increasing moiety is an analog thereof. In embodiments, the degradation-increasing moiety is a derivative thereof. In embodiments, the degradation-increasing moiety is a prodrug thereof (e.g., a physiologically hydrolyzable ester thereof or a physiologically hydrolyzable amide thereof). In embodiments, the degradation-increasing moiety is an IκBα phosphopeptide moiety. In embodiments of a derivative, an original substituent (e.g., substituent group) of an IκBα phosphopeptide moiety is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of an IκBα phosphopeptide moiety are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of an IκBα phosphopeptide moiety is replaced with a substituent (e.g., substituent group). In embodiments of a derivative, a plurality of hydrogen atoms of an IκBα phosphopeptide moiety are replaced with a plurality of substituents (e.g., substituent groups), wherein the substituents (e.g., substituent groups) are each optionally different.

In embodiments, R⁵ is a degradation-increasing moiety. In embodiments, the degradation-increasing moiety is a nutlin moiety or a derivative, analog, or prodrug thereof. In embodiments, the degradation-increasing moiety is an analog thereof. In embodiments, the degradation-increasing moiety is a derivative thereof. In embodiments, the degradation-increasing moiety is a prodrug thereof (e.g., a physiologically hydrolyzable ester thereof or a physiologically hydrolyzable amide thereof). In embodiments, the degradation-increasing moiety is a nutlin moiety. In embodiments of a derivative, an original substituent (e.g., substituent group) of a nutlin moiety is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of a nutlin moiety are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of a nutlin moiety is replaced with a substituent (e.g., substituent group). In embodiments of a derivative, a plurality of hydrogen atoms of a nutlin moiety are replaced with a plurality of substituents (e.g., substituent groups), wherein the substituents (e.g., substituent groups) are each optionally different.

In embodiments, R⁵ is a degradation-increasing moiety. In embodiments, the degradation-increasing moiety is a HIF-1α pentapeptide moiety or a derivative, analog, or prodrug thereof. In embodiments, the degradation-increasing moiety is an analog thereof. In embodiments, the degradation-increasing moiety is a derivative thereof. In embodiments, the degradation-increasing moiety is a prodrug thereof (e.g., a physiologically hydrolyzable ester thereof or a physiologically hydrolyzable amide thereof). In embodiments, the degradation-increasing moiety is a HIF-1α pentapeptide moiety. In embodiments of a derivative, an original substituent (e.g., substituent group) of a HIF-1α pentapeptide moiety is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of a HIF-1α pentapeptide moiety are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of a HIF-1α pentapeptide moiety is replaced with a substituent (e.g., substituent group). In embodiments of a derivative, a plurality of hydrogen atoms of a HIF-1α pentapeptide moiety are replaced with a plurality of substituents (e.g., substituent groups), wherein the substituents (e.g., substituent groups) are each optionally different.

In embodiments, R⁵ is a degradation-increasing moiety. In embodiments, the degradation-increasing moiety is

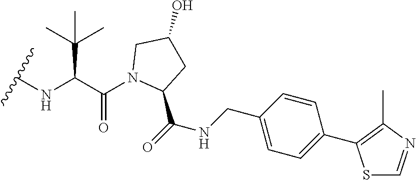 [SMILES: *N[C@H](C(=O)N1C[C@H](O)C[C@H]1C(=O)NCc1ccc(-c2scnc2C)cc1)C(C)(C)C]

or a derivative, analog, or prodrug thereof. In embodiments, the degradation-increasing moiety is an analog thereof. In embodiments, the degradation-increasing moiety is a derivative thereof. In embodiments, the degradation-increasing moiety is a prodrug thereof (e.g., a physiologically hydrolyzable ester thereof or a physiologically hydrolyzable amide thereof). In embodiments, the degradation-increasing moiety is

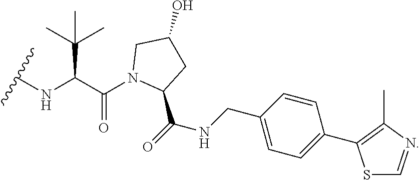 [SMILES: *N[C@H](C(=O)N1C[C@H](O)C[C@H]1C(=O)NCc1ccc(-c2scnc2C)cc1)C(C)(C)C]

In embodiments of a derivative, an original substituent (e.g., substituent group) of

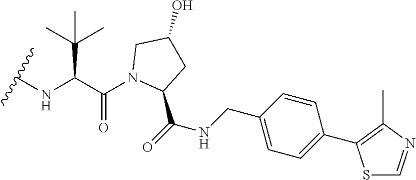 [SMILES: *N[C@H](C(=O)N1C[C@H](O)C[C@H]1C(=O)NCc1ccc(-c2scnc2C)cc1)C(C)(C)C]

is replaced with an alternative substituent (e.g., substituent group), wherein the alternative substituent (e.g., substituent group) is different from the original substituent (e.g., substituent group). In embodiments of a derivative, a plurality of original substituents (e.g., substituent groups) of

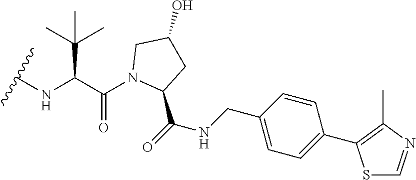 [SMILES: *N[C@H](C(=O)N1C[C@H](O)C[C@H]1C(=O)NCc1ccc(-c2scnc2C)cc1)C(C)(C)C]

are replaced with a plurality of alternative substituents (e.g., substituent groups), wherein the alternative substituents (e.g., substituent groups) are each optionally different and each alternative substituent (e.g., substituent group) is different from the original substituent (e.g., substituent group) it replaces. In embodiments of a derivative, a hydrogen atom of

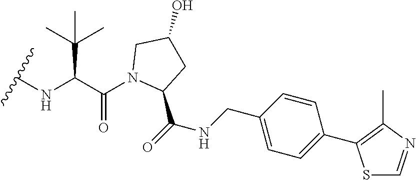 [SMILES: *N[C@H](C(=O)N1C[C@H](O)C[C@H]1C(=O)NCc1ccc(-c2scnc2C)cc1)C(C)(C)C]

is replaced with a substituent (e.g., substituent group).

In embodiments of a derivative, a plurality of hydrogen atoms of

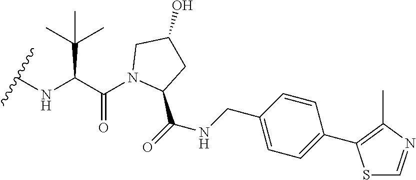 [SMILES: *N[C@H](C(=O)N1C[C@H](O)C[C@H]1C(=O)NCc1ccc(-c2scnc2C)cc1)C(C)(C)C]

are replaced with a plurality of substituents (e.g., substituent groups), wherein the substituents (e.g., substituent groups) are each optionally different.

In embodiment, -L⁵-R⁵ is substituted or unsubstituted 4 to 22 membered hereroalkyl. In embodiment, -L⁵-R⁵ is substituted or unsubstituted 4 to 20 membered hereroalkyl. In embodiment, -L⁵-R⁵ is substituted or unsubstituted 4 to 18 membered hereroalkyl. In embodiment, -L⁵-R⁵ is substituted or unsubstituted 4 to 16 membered hereroalkyl. In embodiment, -L⁵-R⁵ is substituted or unsubstituted 4 to 14 membered hereroalkyl. In embodiment, -L⁵-R⁵ is substituted or unsubstituted 4 to 12 membered hereroalkyl. In embodiment, -L⁵-R⁵ is substituted or unsubstituted 10 to 22 membered hereroalkyl. In embodiment, -L⁵-R⁵ is substituted or unsubstituted 10 to 20 membered hereroalkyl. In embodiment, -L⁵-R⁵ is substituted or unsubstituted 10 to 18 membered hereroalkyl. In embodiment, -L⁵-R⁵ is substituted or unsubstituted 10 to 16 membered hereroalkyl. In embodiment, -L⁵-R⁵ is substituted or unsubstituted 10 to 14 membered hereroalkyl. In embodiment, -L⁵-R⁵ is substituted or unsubstituted 15 to 22 membered hereroalkyl. In embodiment, -L⁵-R⁵ is substituted or unsubstituted 15 to 20 membered hereroalkyl. In embodiment, -L⁵-R⁵ is substituted or unsubstituted 15 to 18 membered hereroalkyl. In embodiment, -L⁵-R⁵ is substituted or unsubstituted 15 to 16 membered hereroalkyl. In embodiment, -L⁵-R⁵ is substituted or unsubstituted 5 to 10 membered hererocycloalkyl. In embodiment, -L⁵-R⁵ is substituted or unsubstituted 5 to 9 membered hererocycloalkyl. In embodiment, -L⁵-R⁵ is substituted or unsubstituted 5 to 8 membered hererocycloalkyl. In embodiment, -L⁵-R⁵ is substituted or unsubstituted 5 to 7 membered hererocycloalkyl. In embodiment, -L⁵-R⁵ is substituted or unsubstituted 5 to 6 membered hererocycloalkyl. In embodiment, -L⁵-R⁵ is substituted or unsubstituted 6 to 10 membered hererocycloalkyl. In embodiment, -L⁵-R⁵ is substituted or unsubstituted 6 to 9 membered hererocycloalkyl. In embodiment, -L⁵-R⁵ is substituted or unsubstituted 6 to 8 membered hererocycloalkyl. In embodiment, -L⁵-R⁵ is substituted or unsubstituted 6 to 7 membered hererocycloalkyl. In embodiment, -L⁵-R⁵ is substituted or unsubstituted 5 membered hererocycloalkyl. In embodiment, -L⁵-R⁵ is substituted or unsubstituted 6 membered hererocycloalkyl.

In embodiments, -L⁵-R⁵ is:

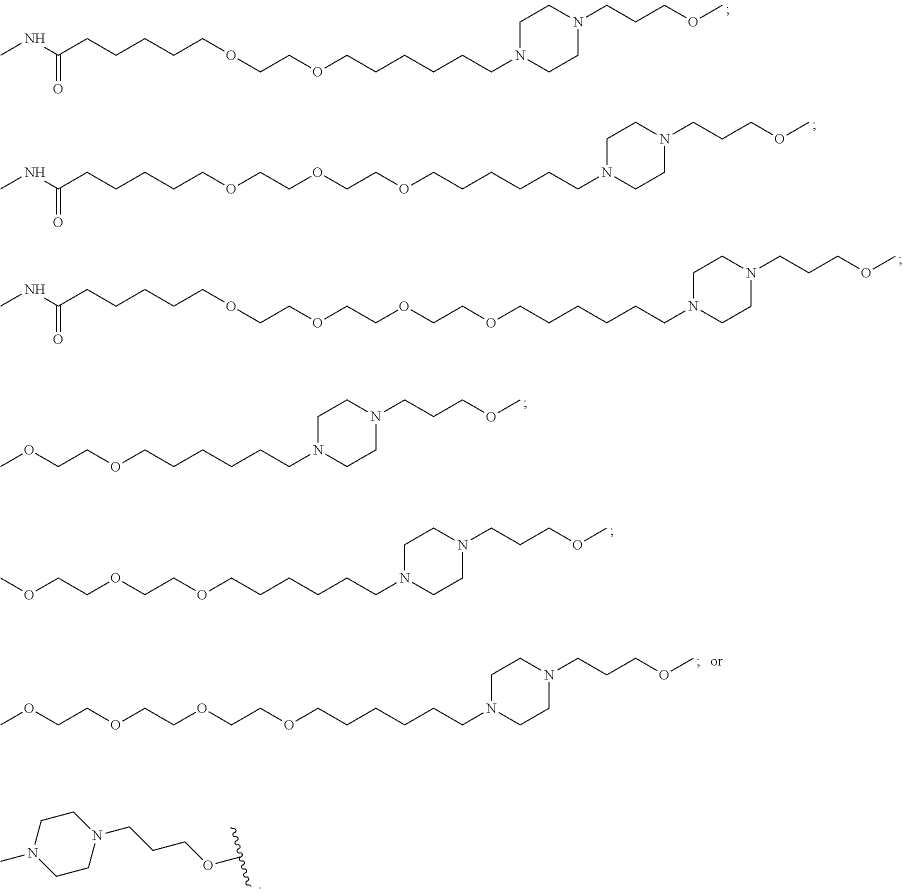

In embodiments, R⁶ is hydrogen, halogen, —CN, —OH, —NH₂, —COOH, —CONH₂, —NO₂, —SH, substituted or unsubstituted C₁-C₄ alkyl, or substituted or unsubstituted 2 to 4 membered heteroalkyl. In embodiments, R⁶ is —CN. In embodiments, R⁶ is hydrogen.

In embodiments, W¹ is N. In embodiments, W¹ is C(R⁶).

In embodiments, L¹ is abond, —O—, —NH—, —C(O)—, —C(O)NH—, —NHC(O)—, —C(O)O—, —OC(O)—, —NHC(O)NH—, —S—, substituted or unsubstituted C₁-C₃ alkylene or substituted or unsubstituted 2 to 3 membered heteroalkylene. In embodiments, L¹ is —O—, substituted or unsubstituted C₁-C₃ alkylene or substituted or unsubstituted 2 to 3 membered heteroalkylene. In embodiments, L¹ is —O—. In embodiments, L¹ is a bond. In embodiments, L¹ is —OCH₂—. In embodiments, L¹ is —CH₂O—.

In embodiments, L² is a bond, —O—, —NH—, —C(O)—, —C(O)NH—, —NHC(O)—, —C(O)O—, —OC(O)—, —NHC(O)NH—, —S—, substituted or unsubstituted C₁-C₃ alkylene or substituted or unsubstituted 2 to 3 membered heteroalkylene. In embodiments, L² is a bond. In embodiments, L² is —C(O)NH—. In embodiments, L² is —NHC(O)—. In embodiments, L² is —NHC(O)NH—. In embodiments, L² is —OCH₂—. In embodiments, L² is —CH₂O—.

In embodiments, the linker (e.g., linker that forms a divalent linker such as L⁴ and/or L⁵) is a polyethylene glycol linker. In embodiments, the linker (e.g., linker that forms a divalent linker such as L⁴ and/or L⁵) is hydrophilic. In embodiments, the linker (e.g., linker that forms a divalent linker such as L⁴ and/or L⁵) is hydrophobic. In embodiments, the linker (e.g., linker that forms a divalent linker such as L⁴ and/or L⁵) includes a disulfide bond. In embodiments, the linker (e.g., linker that forms a divalent linker such as L⁴ and/or L⁵) includes a hydrazone bond. In embodiments, the linker (e.g., linker that forms a divalent linker such as L⁴ and/or L⁵) includes an ester. In embodiments, the linker (e.g., linker that forms a divalent linker such as L⁴ and/or L⁵) includes a sulfonyl. In embodiments, the linker (e.g., linker that forms a divalent linker such as L⁴ and/or L⁵) includes a thioether. In embodiments, the linker (e.g., linker that forms a divalent linker such as L⁴ and/or L⁵) includes an ether. In embodiments, the linker (e.g., linker that forms a divalent linker such as L⁴ and/or L⁵) includes a phosphinate. In embodiments, the linker (e.g., linker that forms a divalent linker such as L⁴ and/or L⁵) includes an alkyloxime bond. In embodiments, the linker (e.g., linker that forms a divalent linker such as L⁴ and/or L⁵) includes one or more amino acids. In embodiments, the linker (e.g., linker that forms a divalent linker such as L⁴ and/or L⁵) consists of amino acids. In embodiments, the linker (e.g., linker that forms a divalent linker such as L⁴ and/or L⁵) includes amino acid derivatives. In embodiments, the linker (e.g., linker that forms a divalent linker such as L⁴ and/or L⁵) is a linker as described in Bioconjugate Techniques (Second Edition) by Greg T. Hermanson (2008), which is herein incorporated by referenced in its entirety for all purposes. In embodiments, the linker (e.g., linker that forms a divalent linker such as L⁴ and/or L⁵) is a linker as described in Flygare J A, Pillow T H, Aristoff P., Antibody-drug conjugates for the treatment of cancer. Chemical Biology and Drug Design. 2013 January;81(1):113-21, which is herein incorporated by referenced in its entirety for all purposes. In embodiments, the linker (e.g., linker that forms a divalent linker such as L⁴ and/or L⁵) is a linker as described in Drachman J G, Senter P D., Antibody-drug conjugates: the chemistry behind empowering antibodies to fight cancer. Hematology Am Soc Hematol Educ Program. 2013; 2013:306-10, which is herein incorporated by referenced in its entirety for all purposes. In embodiments, L⁴ and/or L⁵ are linkers resulting from bioconjugate chemistry as disclosed herein.

In embodiments, L⁴ is L^{4A}-L^{4B}-L^{4C} and L^{4A} L^{4B}, and L^{4C} are each independently a bond, —O—, —NH—, —C(O)—, —C(O)NH—, —NHC(O)—, —C(O)O—, —OC(O)—, —NHC(O)NH—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁴ is a bond, —NH—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁴ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁴ is L^{4A}-L^{4B}-L^{4C}. In embodiments, L^{4A} is a bond, —NH—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{4B} is a bond, —NH—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{4C} is a bond, —NH—, —S—, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{4A} is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{4B} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{4C} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L^{4A} is substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L^{4A} is substituted or unsubstituted 3 to 8 membered heteroalkylene. In embodiments, L^{4A} is —CH₂CH₂OCH₂—. In embodiments, L^{4A} is unsubstituted 3 to 8 membered heteroalkylene. In embodiments, L^{4A} is unsubstituted 3 to 6 membered heteroalkylene. In embodiments, L^{4A} is unsubstituted 3 to 5 membered heteroalkylene. In embodiments, L^{4A} is a bond.

In embodiments, L^{4B} is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L^{4B} is a substituted or unsubstituted 5 to 10 membered heteroarylene. In embodiments, L^{4B} is a bond. In embodiments, L^{4B} is a substituted or unsubstituted 5 to 6 membered heteroarylene. In embodiments, L^{4B} is an unsubstituted 5 to 6 membered heteroarylene. In embodiments, L^{4B} is unsubstituted divalent triazole. In embodiments, L^{4B} is unsubstituted divalent 1H-1,2,3-triazole. In embodiments, L^{4B} is unsubstituted divalent 2H-1,2,3-triazole. In embodiments, L^{4B} is unsubstituted divalent furan.

In embodiments, L^{4C} is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L^{4C} is a substituted or unsubstituted 2 to 12 membered heteroalkylene. In embodiments, L^{4C} is a substituted or unsubstituted 2 to 32 membered heteroalkylene. In embodiments, L^{4C} is a bond.

In embodiments, L^{4C} is a divalent oligomer of ethylene oxide. In embodiments, L^{4C} is a divalent polyethylene glycol. In embodiments, L^{4C} is a divalent oligomer of ethylene oxide having 2 to 30 linear atoms (carbon and oxygen) between the two termini connecting to the remainder of the compound. In embodiments, L^{4C} is —(CH₂CH₂O)ₓCH₂CH₂— and x is an integer from 1 to 16. In embodiments, x is an integer from 2 to 15. In embodiments, x is an integer from 3 to 14. In embodiments, x is an integer from 4 to 12. In embodiments, x is an integer from 5 to 10. In embodiments, x is an integer from 5 to 8. In embodiments, x is an integer from 6 to 7.

In embodiments, L⁵ is L^{5A}-L^{5B}-L^{5C}-L^{5D}-L^{5E}. In embodiments, L^{5A} L^{5B} L^{5C}, L^{5D} and L^{5E} are each independently a bond, —O—, —NH—,a —C(O)—, —C(O)NH—, —NHC(O)—, —C(O)O—, —OC(O)—, —NHC(O)NH—, —S—, substituted or unsubstituted alkylene, substituted or unsubstituted heteroalkylene, substituted or unsubstituted, substituted or unsubstituted cycloalkylene, substituted or unsubstituted heterocycloalkylene, substituted or unsubstituted arylene or substituted or unsubstituted heteroarylene. In embodiments, L^{5A} L^{5B} L^{5C}, L^{5D}, and L^{5E} are each independently a bond, —O—, —NH—, —C(O)—, —C(O)NH—, —NHC(O)—, —C(O)O—, —OC(O)—, —NHC(O)NH—, —S—, substituted or unsubstituted C₁-C₃ alkylene, substituted or unsubstituted 2 to 4 membered heteroalkylene, substituted or unsubstituted C₃-C₆ cycloalkylene, substituted or unsubstituted 3 to 6 membered heterocycloalkylene, substituted or unsubstituted C₆ arylene, or substituted or unsubstituted 5 to 6 membered heteroarylene. In embodiments, at least one of L^{5A} L^{5B} L^{5C}, L^{5D}, and L^{5E} is substituted or unsubstituted piperazinylene. In embodiments, L^{5A} is substituted or unsubstituted piperazinylene. In embodiments, L^{5B} is substituted or unsubstituted piperazinylene. In embodiments, L^{5C} is substituted or unsubstituted piperazinylene. In embodiments, L^{5D} is substituted or unsubstituted piperazinylene. In embodiments, L^{5E} is substituted or unsubstituted piperazinylene.

In embodiments, L^{5A} L^{5B} L^{5C}, and L^{5E} are each independently a bond, O, substituted or unsubstituted C₁-C₃ alkylene or substituted or unsubstituted 2 to 4 membered heteroalkylene. In embodiments, L^{5A} is a bond, O, substituted or unsubstituted C₁-C₃ alkylene or substituted or unsubstituted 2 to 4 membered heteroalkylene. In embodiments, L^{5B} is a bond, O, substituted or unsubstituted C₁-C₃ alkylene or substituted or unsubstituted 2 to 4 membered heteroalkylene. In embodiments, L^{5C} is a bond, O, substituted or unsubstituted C₁-C₃ alkylene or substituted or unsubstituted 2 to 4 membered heteroalkylene. In embodiments, L^{5E} is a bond, O, substituted or unsubstituted C₁-C₃ alkylene or substituted or unsubstituted 2 to 4 membered heteroalkylene.

In embodiments, L^{5A} is a bond. In embodiments, L^{5A} is a bond or O. In embodiments, L^{5A} is a bond, O, or substituted or unsubstituted C₁-C₃ alkylene. In embodiments, L^{5A} is a bond, O, or substituted or unsubstituted 2 to 4 membered heteroalkylene.

In embodiments, L^{5B} is a bond. In embodiments, L^{5B} is a bond or O. In embodiments, L^{5B} is a bond, O, or substituted or unsubstituted C₁-C₃ alkylene. In embodiments, L^{5B} is a bond, O, or substituted or unsubstituted 2 to 4 membered heteroalkylene.

In embodiments, L^{5C} is a bond. In embodiments, L^{5C} is a bond or O. In embodiments, L^{5C} is a bond, O, or substituted or unsubstituted C₁-C₃ alkylene. In embodiments, L^{5C} is a bond, O, or substituted or unsubstituted 2 to 4 membered heteroalkylene.

In embodiments, L^{5E} is a bond. In embodiments, L^{5E} is a bond or O. In embodiments, L^{5E} is a bond, O, or substituted or unsubstituted C₁-C₃ alkylene. In embodiments, L^{5E} is a bond, O, or substituted or unsubstituted 2 to 4 membered heteroalkylene. In embodiments, L^{5E} is a bond or substituted or unsubstituted 2 to 4 membered heteroalkylene.

In embodiments, L^{5A} is a bond, L^{5B} is O, and L^{5C} is substituted or unsubstituted C₁-C₃ alkylene.

Conjugates described herein may be synthesized using bioconjugate or conjugate chemistry. Conjugate chemistry includes coupling two molecules together to form an adduct. Conjugation may be a covalent modification. Currently favored classes of conjugate chemistry reactions available with reactive known reactive groups are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the bioconjugation reaction is a click chemistry reaction (Angewandte Chemie International Edition 40 (11): 2004-2021). In embodiments, the bioconjugation reaction is a Huisgen cyclization of azides. In embodiments, the bioconjugation reaction is a copper catalyzed Huisgen cyclization of azides.

Useful reactive functional groups used for conjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.

The reactive functional groups can be chosen such that they do not participate in, or interfere with, the chemical stability of the conjugate described herein. Alternatively, a reactive functional group can be protected from participating in the crosslinking reaction by the presence of a protecting group.

In embodiments, X¹ is independently —Cl. In embodiments, X¹ is independently —Br. In embodiments, X¹ is independently —I. In embodiments, X¹ is independently —F. In embodiments, X² is independently —Cl. In embodiments, X² is independently —Br. In embodiments, X² is independently —I. In embodiments, X² is independently —F. In embodiments, X³ is independently —Cl. In embodiments, X³ is independently —Br. In embodiments, X³ is independently —I. In embodiments, X³ is independently —F. In embodiments, X⁴ is independently —Cl. In embodiments, X⁴ is independently —Br. In embodiments, X⁴ is independently —I. In embodiments, X⁴ is independently —F. In embodiments, X⁶ is independently —Cl. In embodiments, X⁶ is independently —Br. In embodiments, X⁶ is independently —I. In embodiments, X⁶ is independently —F. In embodiments, X^{A} is independently —Cl. In embodiments, X^{A} is independently —Br. In embodiments, X^{A} is independently —I. In embodiments, X^{A} is independently —F.

In embodiments, the compound has the formula:

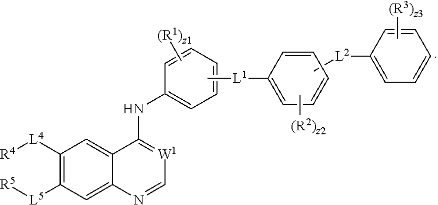

W¹, R¹, R², R³, R⁴, R⁵, L¹, L², L⁴, L⁵, z1, z2, and z3 are as described herein.

In embodiments, the compound has the formula:

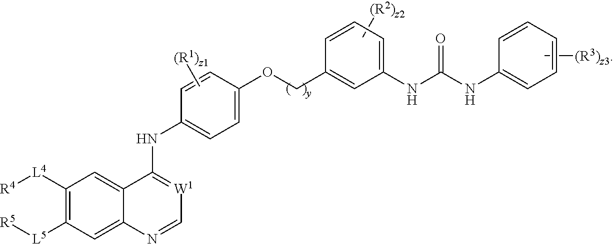

W¹, R¹, R², R³, R⁴, R⁵, L⁴, L⁵, z1, z2, and z3 are as described herein. Y is 0 or 1. In embodiments, Y is 0. In embodiments, Y is 1.

In embodiments, the compound has the formula:

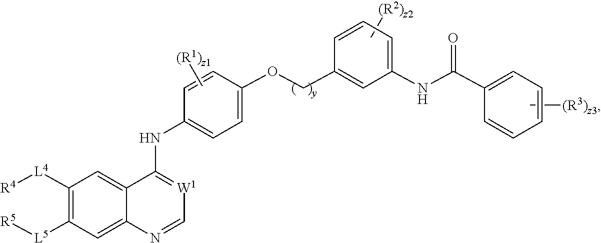

wherein W¹, Y, R¹, R², R³, R⁴, R⁵, L⁴, L⁵, z1, z2, and z3 are as described herein.

In embodiments, R¹ is independently halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{1A}-substituted or unsubstituted alkyl (e.g., C₁-C₈, C₁-C₆, C₁-C₄, or C₁-C₂), R^{1A}-substituted or unsubstituted heteroalkyl (e.g., 2 to 8 membered, 2 to 6 membered, 4 to 6 membered, or 2 to 3 membered), R^{1A}-substituted or unsubstituted cycloalkyl (e.g., C₃-C₈, C₃-C₆, C₄-C₆, or C₅-C₆), R^{1A}-substituted or unsubstituted heterocycloalkyl (e.g., 3 to 6 membered, 4 to 6 membered, 4 to 5 membered, or 5 to 6 membered), R^{1A} substituted or unsubstituted aryl (e.g., C₆-C₁₀ or phenyl), or R^{1A}-substituted or unsubstituted heteroaryl (e.g., 5 to 10 membered, 5 to 9 membered, or 5 to 6 membered). X¹ is independently —F, —Cl, —Br, or —I. In embodiments, R¹ is independently hydrogen. In embodiments, R¹ is independently methyl. In embodiments, R¹ is independently ethyl. In embodiments, R¹ is independently hydrogen, oxo, halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₁-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, two adjacent R¹ substituents may optionally be joined to form an R^{1A}-substituted or unsubstituted cycloalkyl (e.g., C₃-C₈, C₃-C₆, C₄-C₆, or C₅-C₆), R^{1A}-substituted or unsubstituted heterocycloalkyl (e.g., 3 to 6 membered, 4 to 6 membered, 4 to 5 membered, or 5 to 6 membered), R^{1A}-substituted or unsubstituted aryl (e.g., C₆-C₁₀ or phenyl), or R^{1A}-substituted or unsubstituted heteroaryl (e.g., 5 to 10 membered, 5 to 9 membered, or 5 to 6 membered). In embodiments, two adjacent R¹ substituents may optionally be joined to form an unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{1A} is independently oxo, halogen, —CX^{1A}₃, —CHX^{1A}₂, —CH₂X^{1A}, —OCX^{1A}₃, —OCH₂X^{1A}—OCHX^{1A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{1B}-substituted or unsubstituted alkyl (e.g., C₁-C₈, C₁-C₆, C₁-C₄, or C₁-C₂), R^{1B}-substituted or unsubstituted heteroalkyl (e.g., 2 to 8 membered, 2 to 6 membered, 4 to 6 membered, or 2 to 3 membered), R^{1B}-substituted or unsubstituted cycloalkyl (e.g., C₃-C₈, C₃-C₆, C₄-C₆, or C₅-C₆), R^{1B}-substituted or unsubstituted heterocycloalkyl (e.g., 3 to 6 membered, 4 to 6 membered, 4 to 5 membered, or 5 to 6 membered), R^{1B}-substituted or unsubstituted aryl (e.g., C₆-C₁₀ or phenyl), or R^{1B}-substituted or unsubstituted heteroaryl (e.g., 5 to 10 membered, 5 to 9 membered, or 5 to 6 membered). X^{1A} is independently —F, —Cl, —Br, or —I. In embodiments, R^{1A} is independently oxo,halogen, —CX^{1A}₃, —CHX^{1A}₂, —CH₂X^{1A}, —OCX^{1A}₃, —OCH₂X^{1A}, —OCHX^{1A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{1B} is independently oxo, halogen, —CX^{1B}₃, —CHX^{1B}₂, —CH₂X^{1B}, —OCX^{1B}₃, —OCH₂X^{1B}, —OCHX^{1B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{1C}-substituted or unsubstituted alkyl (e.g., C₁-C₈, C₁-C₆, C₁-C₄, or C₁-C₂), R^{1C}-substituted or unsubstituted heteroalkyl (e.g., 2 to 8 membered, 2 to 6 membered, 4 to 6 membered, or 2 to 3 membered), R^{1C}-substituted or unsubstituted cycloalkyl (e.g., C₃-C₈, C₃-C₆, C₄-C₆, or C₅-C₆), R^{1C}-substituted or unsubstituted heterocycloalkyl (e.g., 3 to 6 membered, 4 to 6 membered, 4 to 5 membered, or 5 to 6 membered), R^{1C}-substituted or unsubstituted aryl (e.g., C₆-C₁₀ or phenyl), or R^{1C}-substituted or unsubstituted heteroaryl (e.g., 5 to 10 membered, 5 to 9 membered, or 5 to 6 membered). X^{1B} is independently —F, —Cl, —Br, or —I. In embodiments, R^{1B} is independently oxo, halogen, —CX^{1B}₃, —CHX^{1B}₂, —CH₂X^{1B}, —OCX^{1B}₃, —OCH₂X^{1B}, —OCHX^{1B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{1C} is independently oxo, halogen, —CX^{1C}₃, —CHX^{1C}₂, —CH₂X^{1C}, —OCX^{1C}₃, —OCH₂X^{1C}, —OCHX^{1C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{1C} is independently —F, —Cl, —Br, or —I.

In embodiments, R² is independently halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{2A}-substituted or unsubstituted alkyl (e.g., C₁-C₈, C₁-C₆, C₁-C₄, or C₁-C₂), R^{2A}-substituted or unsubstituted heteroalkyl (e.g., 2 to 8 membered, 2 to 6 membered, 4 to 6 membered, or 2 to 3 membered), R^{2A}-substituted or unsubstituted cycloalkyl (e.g., C₃-C₈, C₃-C₆, C₄-C₆, or C₅-C₆), R^{2A}-substituted or unsubstituted heterocycloalkyl (e.g., 3 to 6 membered, 4 to 6 membered, 4 to 5 membered, or 5 to 6 membered), R^{2A}-substituted or unsubstituted aryl (e.g., C₆-C₁₀ or phenyl), or R^{2A}-substituted or unsubstituted heteroaryl (e.g., 5 to 10 membered, 5 to 9 membered, or 5 to 6 membered). X² is independently —F, —Cl, —Br, or —I. In embodiments, R² is independently hydrogen. In embodiments, R² is independently methyl. In embodiments, R² is independently ethyl. In embodiments, R² is independently hydrogen, oxo, halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, two adjacent R² substituents may optionally be joined to form an R^{2A}-substituted or unsubstituted cycloalkyl (e.g., C₃-8, C₃-C₆, C₄-C₆, or C₅-C₆), R^{2A}-substituted or unsubstituted heterocycloalkyl (e.g., 3 to 6 membered, 4 to 6 membered, 4 to 5 membered, or 5 to 6 membered), R^{2A}-substituted or unsubstituted aryl (e.g., C₆-C₁₀ or phenyl), or R^{2A}-substituted or unsubstituted heteroaryl (e.g., 5 to 10 membered, 5 to 9 membered, or 5 to 6 membered). In embodiments, two adjacent R² substituents may optionally be joined to form an unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{2A} is independently oxo, halogen, —CX^{2A}₃, —CHX^{2A}₂, —CH₂X^{2A}, —OCX^{2A}₃, —OCH₂X^{2A}, —OCHX^{2A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{2B}-substituted or unsubstituted alkyl (e.g., C₁-C₈, C₁-C₆, C₁-C₄, or C₁-C₂), R^{2B}-substituted or unsubstituted heteroalkyl (e.g., 2 to 8 membered, 2 to 6 membered, 4 to 6 membered, or 2 to 3 membered), R^{2B}-substituted or unsubstituted cycloalkyl (e.g., C₃-8, C₃-C₆, C₄-C₆, or C₅-C₆), R^{2B}-substituted or unsubstituted heterocycloalkyl (e.g., 3 to 6 membered, 4 to 6 membered, 4 to 5 membered, or 5 to 6 membered), R^{2B}-substituted or unsubstituted aryl (e.g., C₆-C₁₀ or phenyl), or R^{2B}-substituted or unsubstituted heteroaryl (e.g., 5 to 10 membered, 5 to 9 membered, or 5 to 6 membered). X^{2A} is independently —F, —Cl, —Br, or —I. In embodiments, R^{2A} is independently oxo,halogen, —CX^{2A}₃, —CHX^{2A}₂, —CH₂X^{2A}, —OCX^{2A}₃, —OCH₂X^{2A}, —OCHX^{2A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{2B} is independently oxo, halogen, —CX^{2B}₃, —CHX^{2B}₂, —CH₂X^{2B}, —OCX^{2B}₃, —OCH₂X^{2B}, —OCHX^{2B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{2C}-substituted or unsubstituted alkyl (e.g., C₁-C₈, C₁-C₆, C₁-C₄, or C₁-C₂), R^{2C}-substituted or unsubstituted heteroalkyl (e.g., 2 to 8 membered, 2 to 6 membered, 4 to 6 membered, or 2 to 3 membered), R^{2C}-substituted or unsubstituted cycloalkyl (e.g., C₃-C₅, C₃-C₆, C₄-C₆, or C₅-C₆), R^{2C}-substituted or unsubstituted heterocycloalkyl (e.g., 3 to 6 membered, 4 to 6 membered, 4 to 5 membered, or 5 to 6 membered), R^{2C}-substituted or unsubstituted aryl (e.g., C₆-C₁₀ or phenyl), or R^{2C}-substituted or unsubstituted heteroaryl (e.g., 5 to 10 membered, 5 to 9 membered, or 5 to 6 membered). X^{2B} is independently —F, —Cl, —Br, or —I. In embodiments, R^{2B} is independently oxo, halogen, —CX^{2B}₃, —CHX^{2B}₂, —CH₂X^{2B}, —OCX^{2B}₃, —OCH₂X^{2B}, —OCHX^{2B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{2C} is independently oxo, halogen, —CX^{2C}₃, —CHX^{2C}₂, —CH₂X^{2C}, —OCX^{2C}₃, —OCH₂X^{2C}, —OCHX^{2C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{2C} is independently —F, —Cl, —Br, or —I.

In embodiments, R³ is independently halogen, —CX³₃, —CHX³², —CH₂X³, —OCX³₃, —OCH₂X³, —OCHX³², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{3A}-substituted or unsubstituted alkyl (e.g., C₁-C₈, C₁-C₆, C₁-C₄, or C₁-C₂), R^{3A}-substituted or unsubstituted heteroalkyl (e.g., 2 to 8 membered, 2 to 6 membered, 4 to 6 membered, or 2 to 3 membered), R^{3A}-substituted or unsubstituted cycloalkyl (e.g., C₃-C₈, C₃-C₆, C₄-C₆, or C₅-C₆), R^{3A}-substituted or unsubstituted heterocycloalkyl (e.g., 3 to 6 membered, 4 to 6 membered, 4 to 5 membered, or 5 to 6 membered), R^{3A}-substituted or unsubstituted aryl (e.g., C₆-C₁₀ or phenyl), or R^{3A}-substituted or unsubstituted heteroaryl (e.g., 5 to 10 membered, 5 to 9 membered, or 5 to 6 membered). X³ is independently —F, —Cl, —Br, or —I. In embodiments, R³ is independently hydrogen. In embodiments, R³ is independently methyl. In embodiments, R³ is independently ethyl. In embodiments, R³ is independently hydrogen, oxo, halogen, —CX³₃, —CHX³², —CH₂X³, —OCX³₃, —OCH₂X³, —OCHX³², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂, unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₁-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, two adjacent R³ substituents may optionally be joined to form an R^{3A}-substituted or unsubstituted cycloalkyl (e.g., C₃-C₈, C₃-C₆, C₄-C₆, or C₅-C₆), R^{3A}-substituted or unsubstituted heterocycloalkyl (e.g., 3 to 6 membered, 4 to 6 membered, 4 to 5 membered, or 5 to 6 membered), R^{3A}-substituted or unsubstituted aryl (e.g., C₆-C₁₀ or phenyl), or R^{3A}-substituted or unsubstituted heteroaryl (e.g., 5 to 10 membered, 5 to 9 membered, or 5 to 6 membered). In embodiments, two adjacent R³ substituents may optionally be joined to form an unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{3A} is independently oxo, halogen, —CX^{3A}₃, —CHX^{3A}₂, —CH₂X^{3A}, —OCX^{3A}₃, —OCH₂X^{3A}, —OCHX^{3A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{3B}-substituted or unsubstituted alkyl (e.g., C₁-C₈, C₁-C₆, C₁-C₄, or C₁-C₂), R^{3B}-substituted or unsubstituted heteroalkyl (e.g., 2 to 8 membered, 2 to 6 membered, 4 to 6 membered, or 2 to 3 membered), R^{3B}-substituted or unsubstituted cycloalkyl (e.g., C₃-C₈, C₃-C₆, C₄-C₆, or C₅-C₆), R^{3B}-substituted or unsubstituted heterocycloalkyl (e.g., 3 to 6 membered, 4 to 6 membered, 4 to 5 membered, or 5 to 6 membered), R^{3B}-substituted or unsubstituted aryl (e.g., C₆-C₁₀ or phenyl), or R^{3B}-substituted or unsubstituted heteroaryl (e.g., 5 to 10 membered, 5 to 9 membered, or 5 to 6 membered). X^{3A} is independently —F, —Cl, —Br, or —I. In embodiments, R^{3A} is independently oxo, halogen, —CX^{3A}₃, —CHX^{3A}₂, —CH₂X^{3A}, —OCX^{3A}₃, —OCH₂X^{3A}—OCHX^{3A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{3B} is independently oxo, halogen, —CX^{3B}₃, —CHX^{3B}₂, —CH₂X^{3B}, —OCX^{3B}₃, —OCH₂X^{3B}, —OCHX^{3B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{3C}-substituted or unsubstituted alkyl (e.g., C₁-C₈, C₁-C₆, C₁-C₄, or C₁-C₂), R^{3C}-substituted or unsubstituted heteroalkyl (e.g., 2 to 8 membered, 2 to 6 membered, 4 to 6 membered, or 2 to 3 membered), R^{3C}-substituted or unsubstituted cycloalkyl (e.g., C₃-C₈, C₃-C₆, C₄-C₆, or C₅-C₆), R^{3C}-substituted or unsubstituted heterocycloalkyl (e.g., 3 to 6 membered, 4 to 6 membered, 4 to 5 membered, or 5 to 6 membered), R^{3C}-substituted or unsubstituted aryl (e.g., C₆-C₁₀ or phenyl), or R^{3C}-substituted or unsubstituted heteroaryl (e.g., 5 to 10 membered, 5 to 9 membered, or 5 to 6 membered). X^{3B} is independently —F, —Cl, —Br, or —I. In embodiments, R^{3B} is independently oxo, halogen, —CX^{3B}₃, —CHX^{3B}₂, —CH₂X^{3B}, —OCX^{3B}₃, —OCH₂X^{3B}, —OCHX^{3B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{3C} is independently oxo, halogen, —CX^{3C}₃, —CHX^{3C}₂, —CH₂X^{3C}, —OCX³C₃, —OCH₂X^{3C}, —OCHX³C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{3C} is independently —F, —Cl, —Br, or —I.

In embodiments, R⁴ is independently hydrogen, halogen, —CX⁴₃, —CHX⁴₂, —CH₂X⁴, —OCX⁴₃, —OCH₂X⁴, —OCHX⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{4A}-substituted or unsubstituted alkyl (e.g., C₁-C₈, C₁-C₆, C₁-C₄, or C₁-C₂), R^{4A}-substituted or unsubstituted heteroalkyl (e.g., 2 to 8 membered, 2 to 6 membered, 4 to 6 membered, or 2 to 3 membered), R^{4A}-substituted or unsubstituted cycloalkyl (e.g., C₃-C₈, C₃-C₆, C₄-C₆, or C₅-C₆), R^{4A}-substituted or unsubstituted heterocycloalkyl (e.g., 3 to 6 membered, 4 to 6 membered, 4 to 5 membered, or 5 to 6 membered), R^{4A}-substituted or unsubstituted aryl (e.g., C₆-C₁₀ or phenyl), or R^{4A}-substituted or unsubstituted heteroaryl (e.g., 5 to 10 membered, 5 to 9 membered, or 5 to 6 membered). X⁴ is independently —F, —Cl, —Br, or —I. In embodiments, R⁴ is independently hydrogen. In embodiments, R⁴ is independently methyl. In embodiments, R⁴ is independently ethyl. In embodiments, R⁴ is independently hydrogen, oxo, halogen, —CX⁴₃, —CHX⁴₂, —CH₂X⁴, —OCX⁴₃, —OCH₂X⁴, —OCHX⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{4A} is independently oxo, halogen, —CX^{4A}₃, —CHX^{4A}₂, —CH₂X^{4A}, —OCX^{4A}₃, —OCH₂X^{4A}, —OCHX^{4A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{4B}-substituted or unsubstituted alkyl (e.g., C₁-C₈, C₁-C₆, C₁-C₄, or C₁-C₂), R^{4B}-substituted or unsubstituted heteroalkyl (e.g., 2 to 8 membered, 2 to 6 membered, 4 to 6 membered, or 2 to 3 membered), R^{4B}-substituted or unsubstituted cycloalkyl (e.g., C₃-C₈, C₃-C₆, C₄-C₆, or C₅-C₆), R^{4B}-substituted or unsubstituted heterocycloalkyl (e.g., 3 to 6 membered, 4 to 6 membered, 4 to 5 membered, or 5 to 6 membered), R^{4B}-substituted or unsubstituted aryl (e.g., C₆-C₁₀ or phenyl), or R^{4B}-substituted or unsubstituted heteroaryl (e.g., 5 to 10 membered, 5 to 9 membered, or 5 to 6 membered). X^{4A} is independently —F, —Cl, —Br, or —I. In embodiments, R^{4A} is independently oxo,halogen, —CX^{4A}₃, —CHX^{4A}₂, —CH₂X^{4A}, —OCX^{4A}₃, —OCH₂X^{4A}, —OCHX^{4A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₂, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{4B} is independently oxo, halogen, —CX^{4B}₃, —CHX^{4B}₂, —CH₂X^{4B}, —OCX^{4B}₃, —OCH₂X^{4B}—OCHX^{4B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{4C}-substituted or unsubstituted alkyl (e.g., C₁-C₈, C₁-C₂, C₁-C₄, or C₁-C₂), R^{4C}-substituted or unsubstituted heteroalkyl (e.g., 2 to 8 membered, 2 to 6 membered, 4 to 6 membered, or 2 to 3 membered), R^{4C}-substituted or unsubstituted cycloalkyl (e.g., C₃-C₈, C₃-C₆, C₄-C₆, or C₅-C₆), R^{4C}-substituted or unsubstituted heterocycloalkyl (e.g., 3 to 6 membered, 4 to 6 membered, 4 to 5 membered, or 5 to 6 membered), R^{4C}-substituted or unsubstituted aryl (e.g., C₆-C₁₀ or phenyl), or R^{4C}-substituted or unsubstituted heteroaryl (e.g., 5 to 10 membered, 5 to 9 membered, or 5 to 6 membered). X^{4B} is independently —F, —Cl, —Br, or —I. In embodiments, R^{4B} is independently oxo, halogen, —CX^{4B}3, —CHX^{4B}2, —CH₂X^{4B}, —OCX^{4B}3, —OCH₂X^{4B}, —OCHX^{4B}2, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{4C} is independently oxo, halogen, —CX^{4C}₃, —CHX^{4C}₂, —CH₂X^{4C}, —OCX^{4C}, —OCH₂X^{4C}, —OCHX^{4C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4C} is independently —F, —Cl, —Br, or —I.

In embodiments, R⁶ is independently hydrogen, halogen, —CX⁶₃, —CHX⁶₂, —CH₂X⁶, —OCX⁶₃, —OCH₂X⁶, —OCHX⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{6A}-substituted or unsubstituted alkyl (e.g., C₁-C₈, C₁-C₆, C₁-C₄, or C₁-C₂), R^{6A}-substituted or unsubstituted heteroalkyl (e.g., 2 to 8 membered, 2 to 6 membered, 4 to 6 membered, or 2 to 3 membered), R^{6A}-substituted or unsubstituted cycloalkyl (e.g., C₃-C₈, C₃-C₆, C₄-C₆, or C₅-C₆), R^{6A}-substituted or unsubstituted heterocycloalkyl (e.g., 3 to 6 membered, 4 to 6 membered, 4 to 5 membered, or 5 to 6 membered), R^{6A}-substituted or unsubstituted aryl (e.g., C₆-C₁₀ or phenyl), or R^{6A}-substituted or unsubstituted heteroaryl (e.g., 5 to 10 membered, 5 to 9 membered, or 5 to 6 membered). X⁶ is independently —F, —Cl, —Br, or —I. In embodiments, R⁶ is independently hydrogen. In embodiments, R⁶ is independently methyl. In embodiments, R⁶ is independently ethyl. In embodiments, R⁶ is independently hydrogen, oxo, halogen, —CX⁶₃, —CHX⁶₂, —CH₂X⁶, —OCX⁶₃, —OCH₂X⁶, —OCHX⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{6A} is independently oxo, halogen, —CX^{6A}₃, —CHX^{6A}₂, —CH₂X^{6A}, —OCX^{6A}₃, —OCH₂X^{6A}, —OCHX^{6A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{6B}-substituted or unsubstituted alkyl (e.g., C₁-C₈, C₁-C₆, C₁-C₄, or C₁-C₂), R^{6B}-substituted or unsubstituted heteroalkyl (e.g., 2 to 8 membered, 2 to 6 membered, 4 to 6 membered, or 2 to 3 membered), R^{6B}-substituted or unsubstituted cycloalkyl (e.g., C₃-C₈, C₃-C₆, C₄-C₆, or C₅-C₆), R^{6B}-substituted or unsubstituted heterocycloalkyl (e.g., 3 to 6 membered, 4 to 6 membered, 4 to 5 membered, or 5 to 6 membered), R^{6B}-substituted or unsubstituted aryl (e.g., C₆-C₁₀ or phenyl), or R^{6B}-substituted or unsubstituted heteroaryl (e.g., 5 to 10 membered, 5 to 9 membered, or 5 to 6 membered). X^{6A} is independently —F, —Cl, —Br, or —I. In embodiments, R^{6A} is independently oxo,halogen, —CX^{6A}₃, —CHX^{6A}₂, —CH₂X^{6A}, —OCX^{6A}₃, —OCH₂X^{6A}—OCHX^{6A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{6B} is independently oxo, halogen, —CX^{6B}₃, —CHX^{6B}₂, —CH₂X^{6B}, —OCX^{6B}₃, —OCH₂X^{6B}, —OCHX^{6B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R^{6C}-substituted or unsubstituted alkyl (e.g., C₁-C₈, C₁-C₆, C₁-C₄, or C₁-C₂), R^{6C}-substituted or unsubstituted heteroalkyl (e.g., 2 to 8 membered, 2 to 6 membered, 4 to 6 membered, or 2 to 3 membered), R^{6C}-substituted or unsubstituted cycloalkyl (e.g., C₃-C₈, C₃-C₆, C₄-C₆, or C₅-C₆), R^{6C}-substituted or unsubstituted heterocycloalkyl (e.g., 3 to 6 membered, 4 to 6 membered, 4 to 5 membered, or 5 to 6 membered), R^{6C}-substituted or unsubstituted aryl (e.g., C₆-C₁₀ or phenyl), or R^{6C}-substituted or unsubstituted heteroaryl (e.g., 5 to 10 membered, 5 to 9 membered, or 5 to 6 membered). X^{6B} is independently —F, —Cl, —Br, or —I. In embodiments, R^{6B} is independently oxo, halogen, —CX^{6B}₃, —CHX^{6B}₂, —CH₂X^{6B}, —OCX^{6B}₃, —OCH₂X^{6B}, —OCHX^{6B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{6C} is independently oxo, halogen, —CX^{6C}₃, —CHX^{6C}₂, —CH₂X^{6C}, —OCX^{6C}₃, —OCH₂X⁶c, —OCHX^{6C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{6C} is independently —F, —Cl, —Br, or —I.

In embodiments, R⁷ is independently hydrogen, —CX⁷³, —CN, —COOH, —CONH₂, —CHX⁷₂, —CH₂X⁷, R^{7A}-substituted or unsubstituted alkyl (e.g., C₁-C₈, C₁-C₆, C₁-C₄, or C₁-C₂), R^{7A}-substituted or unsubstituted heteroalkyl (e.g., 2 to 8 membered, 2 to 6 membered, 4 to 6 membered, or 2 to 3 membered), R^{7A}-substituted or unsubstituted cycloalkyl (e.g., C₃-C₈, C₃-C₆, C₄-C₆, or C₅-C₆), R^{7A}-substituted or unsubstituted heterocycloalkyl (e.g., 3 to 6 membered, 4 to 6 membered, 4 to 5 membered, or 5 to 6 membered), R^{7A}-substituted or unsubstituted aryl (e.g., C₆-C₁₀ or phenyl), or R^{7A}-substituted or unsubstituted heteroaryl (e.g., 5 to 10 membered, 5 to 9 membered, or 5 to 6 membered). X⁷ is independently —F, —Cl, —Br, or —I. In embodiments, R⁷ is independently hydrogen. In embodiments, R⁷ is independently methyl. In embodiments, R⁷ is independently ethyl. In embodiments, R⁷ is independently hydrogen, CX⁷³, —CN, —COOH, —CONH₂, —CHX⁷₂, —CH₂X⁷, unsubstituted alkyl (e.g., C₁-C₈, C₁-C₆, C₁-C₄, or C₁-C₂, unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁷ and R⁸ substituents bonded to the same nitrogen atom may optionally be joined to form a R^{7A}-substituted or unsubstituted heterocycloalkyl or R^{7A}-substituted or unsubstituted heteroaryl. In embodiments, R⁷ and R⁸ substituents bonded to the same nitrogen atom may optionally be joined to form a R^{7A}-substituted or unsubstituted 3 to 6 membered heterocycloalkyl or R^{7A}-substituted or unsubstituted 5 to 6 membered heteroaryl. In embodiments, R⁷ and R⁸ substituents bonded to the same nitrogen atom may optionally be joined to form an unsubstituted 3 to 6 membered heterocycloalkyl or unsubstituted 5 to 6 membered heteroaryl. In embodiments, R⁷ and R⁸ substituents bonded to the same nitrogen atom may optionally be joined to form a R^{7A}-substituted or unsubstituted piperazinyl. In embodiments, R⁷ and R⁸ substituents bonded to the same nitrogen atom may optionally be joined to form a R^{7A}-substituted or unsubstituted piperidinyl. In embodiments, R⁷ and R⁸ substituents bonded to the same nitrogen atom may optionally be joined to form a R^{7A}-substituted or unsubstituted pyrrolidinyl. In embodiments, R⁷ and R⁸ substituents bonded to the same nitrogen atom may optionally be joined to form a R^{7A}-substituted or unsubstituted azetidinyl. In embodiments, R⁷ and R⁸ substituents bonded to the same nitrogen atom may optionally be joined to form a R^{7A}-substituted or unsubstituted morpholinyl. In embodiments, R⁷ and R⁸ substituents bonded to the same nitrogen atom may optionally be joined to form a R^{7A}-substituted or unsubstituted azeridinyl.

R^{7A} is independently oxo, halogen, —CX^{7A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{7A}₂, —CH₂X^{7A}, —OCX^{7A}₃, —OCH₂X^{7A}, —OCHX^{7A}₂, R^{7B}-substituted or unsubstituted alkyl (e.g., C₁-C₈, C₁-C₆, C₁-C₄, or C₁-C₂), R^{7B}-substituted or unsubstituted heteroalkyl (e.g., 2 to 8 membered, 2 to 6 membered, 4 to 6 membered, or 2 to 3 membered), R^{7B}-substituted or unsubstituted cycloalkyl (e.g., C₃-C₈, C₃-C₆, C₄-C₆, or C₅-C₆), R^{7B}-substituted or unsubstituted heterocycloalkyl (e.g., 3 to 6 membered, 4 to 6 membered, 4 to 5 membered, or 5 to 6 membered), R^{7B}-substituted or unsubstituted aryl (e.g., C₆-C₁₀ or phenyl), or R^{7B}-substituted or unsubstituted heteroaryl (e.g., 5 to 10 membered, 5 to 9 membered, or 5 to 6 membered). X^{7A} is independently —F, —Cl, —Br, or —I. In embodiments, R^{7A} is independently oxo, halogen, —CX^{7A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{7A}₂, —CH₂X^{7A}, —OCX^{7A}₃, —OCH₂X^{7A}, —OCHX^{7A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{7B} is independently oxo, halogen, —CX^{7B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{7B}₂, —CH₂X^{7B}, —OCX^{7B}₃, —OCH₂X^{7B}, —OCHX^{7B}₂, R^{7C}-substituted or unsubstituted alkyl (e.g., C₁-C₈, C₁-C₆, C₁-C₄, or C₁-C₂), R^{7C}-substituted or unsubstituted heteroalkyl (e.g., 2 to 8 membered, 2 to 6 membered, 4 to 6 membered, or 2 to 3 membered), R^{7C}-substituted or unsubstituted cycloalkyl (e.g., C₃-C₅, C₃-C₆, C₄-C₆, or C₅-C₆), R^{7C}-substituted or unsubstituted heterocycloalkyl (e.g., 3 to 6 membered, 4 to 6 membered, 4 to 5 membered, or 5 to 6 membered), R^{7C}-substituted or unsubstituted aryl (e.g., C₆-C₁₀ or phenyl), or R^{7C}-substituted or unsubstituted heteroaryl (e.g., 5 to 10 membered, 5 to 9 membered, or 5 to 6 membered). X^{7B} is independently —F, —Cl, —Br, or —I. In embodiments, R^{7B} is independently oxo, halogen, —CX^{7B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{7B}₂, —CH₂X^{7B}, —OCX^{7B}₃, —OCH₂X^{7B}, —OCHX^{7B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{7C} is independently oxo, halogen, —CX^{7C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{7C}₂, —CH₂X^{7C}, —OCX^{7C}₃, —OCH₂X^{7C}, —OCHX^{7C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{7C} is independently —F, —Cl, —Br, or —I.

In embodiments, R⁸ is independently hydrogen, —CX⁸₃, —CN, —COOH, —CONH₂, —CHX⁸₂, —CH₂X⁸, R^{8A}-substituted or unsubstituted alkyl (e.g., C₁-C₈, C₁-C₆, C₁-C₄, or C₁-C₂), R^{8A}-substituted or unsubstituted heteroalkyl (e.g., 2 to 8 membered, 2 to 6 membered, 4 to 6 membered, or 2 to 3 membered), R^{8A}-substituted or unsubstituted cycloalkyl (e.g., C₃-C₈, C₃-C₆, C₄-C₆, or C₅-C₆), R^{8A}-substituted or unsubstituted heterocycloalkyl (e.g., 3 to 6 membered, 4 to 6 membered, 4 to 5 membered, or 5 to 6 membered), R^{8A}-substituted or unsubstituted aryl (e.g., C₆-C₁₀ or phenyl), or R^{8A}-substituted or unsubstituted heteroaryl (e.g., 5 to 10 membered, 5 to 9 membered, or 5 to 6 membered). X⁸ is independently —F, —Cl, —Br, or —I. In embodiments, R⁸ is independently hydrogen. In embodiments, R⁸ is independently methyl. In embodiments, R⁸ is independently ethyl. In embodiments, R⁸ is independently hydrogen, —CX⁸₃, —CN, —COOH, —CONH₂, —CHX⁸₂, —CH₂X⁸, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁸ and R⁷ substituents bonded to the same nitrogen atom may optionally be joined to form a R^{8A}-substituted or unsubstituted heterocycloalkyl or R^{8A}-substituted or unsubstituted heteroaryl. In embodiments, R⁸ and R⁷ substituents bonded to the same nitrogen atom may optionally be joined to form a R^{8A}-substituted or unsubstituted 3 to 6 membered heterocycloalkyl or R^{8A}-substituted or unsubstituted 5 to 6 membered heteroaryl. In embodiments, R⁸ and R⁷ substituents bonded to the same nitrogen atom may optionally be joined to form an unsubstituted 3 to 6 membered heterocycloalkyl or unsubstituted 5 to 6 membered heteroaryl. In embodiments, R⁸ and R⁷ substituents bonded to the same nitrogen atom may optionally be joined to form a R^{8A}-substituted or unsubstituted piperazinyl. In embodiments, R⁸ and R⁷ substituents bonded to the same nitrogen atom may optionally be joined to form a R^{8A}-substituted or unsubstituted piperidinyl. In embodiments, R⁸ and R⁷ substituents bonded to the same nitrogen atom may optionally be joined to form a R^{8A}-substituted or unsubstituted pyrrolidinyl. In embodiments, R⁸ and R⁷ substituents bonded to the same nitrogen atom may optionally be joined to form a R^{8A}-substituted or unsubstituted azetidinyl. In embodiments, R⁸ and R⁷ substituents bonded to the same nitrogen atom may optionally be joined to form a R^{8A}-substituted or unsubstituted morpholinyl. In embodiments, R⁸ and R⁷ substituents bonded to the same nitrogen atom may optionally be joined to form a R^{8A}-substituted or unsubstituted azeridinyl.

R^{8A} is independently oxo, halogen, —CX^{8A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{8A}₂, —CH₂X^{8A}, —OCX^{8A}₃, —OCH₂X^{8A}, —OCHX^{8A}₂, R^{8B}-substituted or unsubstituted alkyl (e.g., C₁-C₈, C₁-C₆, C₁-C₄, or C₁-C₂), R^{8B}-substituted or unsubstituted heteroalkyl (e.g., 2 to 8 membered, 2 to 6 membered, 4 to 6 membered, or 2 to 3 membered), R^{8B}-substituted or unsubstituted cycloalkyl (e.g., C₃-C₈, C₃-C₆, C₄-C₆, or C₅-C₆), R^{8B}-substituted or unsubstituted heterocycloalkyl (e.g., 3 to 6 membered, 4 to 6 membered, 4 to 5 membered, or 5 to 6 membered), R^{8B}-substituted or unsubstituted aryl (e.g., C₆-C₁₀ or phenyl), or R^{8B}-substituted or unsubstituted heteroaryl (e.g., 5 to 10 membered, 5 to 9 membered, or 5 to 6 membered). X^{8A} is independently —F, —Cl, —Br, or —I. In embodiments, R^{8A} is independently oxo, halogen, —CX^{8A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{8A}₂, —CH₂X^{8A}, —OCX^{8A}₃, —OCH₂X^{8A}, —OCHX^{8A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{8B} is independently oxo, halogen, —CX^{8B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{8B}₂, —CH₂X^{8B}, —OCX^{8B}₃, —OCH₂X^{8B}, —OCHX^{8B}₂, R^{8C}-substituted or unsubstituted alkyl (e.g., C₁-C₈, C₁-C₆, C₁-C₄, or C₁-C₂), R^{8C}-substituted or unsubstituted heteroalkyl (e.g., 2 to 8 membered, 2 to 6 membered, 4 to 6 membered, or 2 to 3 membered), R^{8C}-substituted or unsubstituted cycloalkyl (e.g., C₃-C₈, C₃-C₆, C₄-C₆, or C₅-C₆), R^{8C}-substituted or unsubstituted heterocycloalkyl (e.g., 3 to 6 membered, 4 to 6 membered, 4 to 5 membered, or 5 to 6 membered), R^{8C}-substituted or unsubstituted aryl (e.g., C₆-C₁₀ or phenyl), or R^{8C}-substituted or unsubstituted heteroaryl (e.g., 5 to 10 membered, 5 to 9 membered, or 5 to 6 membered). X^{8B} is independently —F, —Cl, —Br, or —I. In embodiments, R^{8B} is independently oxo, halogen, —CX^{8B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{8B}₂, —CH₂X^{8B}, —OCX^{8B}₃, —OCH₂X^{8B}, —OCHX^{8B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{8C} is independently oxo, halogen, —CX^{8C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{8C}₂, —CH₂X^{8C}, —OCX^{8C}₃, —OCH₂X^{8C}, —OCHX^{8C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{8C} is independently —F, —Cl, —Br, or —I.

In embodiments, R⁹ is independently hydrogen, —CX⁹³, —CN, —COOH, —CONH₂, —CHX⁹₂, —CH₂X⁹, R^{9A}-substituted or unsubstituted alkyl (e.g., C₁-C₈, C₁-C₆, C₁-C₄, or C₁-C₂), R^{9A}-substituted or unsubstituted heteroalkyl (e.g., 2 to 8 membered, 2 to 6 membered, 4 to 6 membered, or 2 to 3 membered), R^{9A}-substituted or unsubstituted cycloalkyl (e.g., C₃-C₈, C₃-C₆, C₄-C₆, or C₅-C₆), R^{9A}-substituted or unsubstituted heterocycloalkyl (e.g., 3 to 6 membered, 4 to 6 membered, 4 to 5 membered, or 5 to 6 membered), R^{9A}-substituted or unsubstituted aryl (e.g., C₆-C₁₀ or phenyl), or R^{9A}-substituted or unsubstituted heteroaryl (e.g., 5 to 10 membered, 5 to 9 membered, or 5 to 6 membered). X⁹ is independently —F, —Cl, —Br, or —I. In embodiments, R⁹ is independently hydrogen. In embodiments, R⁹ is independently methyl. In embodiments, R⁹ is independently ethyl. In embodiments, R⁹ is independently hydrogen, —CX⁹³, —CN, —COOH, —CONH₂, —CHX⁹₂, —CH₂X⁹, unsubstituted alkyl (e.g., C₁-C₈, C₁-C₆, C₁-C₄, or C₁-C₂, unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{9A} is independently oxo, halogen, —CX^{9A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{9A}₂, —CH₂X^{9A}, —OCX^{9A}₃, —OCH₂X^{9A}, —OCHX^{9A}₂, R^{9B}-substituted or unsubstituted alkyl (e.g., C₁-C₈, C₁-C₆, C₁-C₄, or C₁-C₂), R^{9B}-substituted or unsubstituted heteroalkyl (e.g., 2 to 8 membered, 2 to 6 membered, 4 to 6 membered, or 2 to 3 membered), R^{9B}-substituted or unsubstituted cycloalkyl (e.g., C₃-C₅, C₃-C₆, C₄-C₆, or C₅-C₆), R^{9B}-substituted or unsubstituted heterocycloalkyl (e.g., 3 to 6 membered, 4 to 6 membered, 4 to 5 membered, or 5 to 6 membered), R^{9B}-substituted or unsubstituted aryl (e.g., C₆-C₁₀ or phenyl), or R^{9B}-substituted or unsubstituted heteroaryl (e.g., 5 to 10 membered, 5 to 9 membered, or 5 to 6 membered). X^{9A} is independently —F, —Cl, —Br, or —I. In embodiments, R^{9A} is independently oxo, halogen, —CX^{9A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{9A}₂, —CH₂X^{9A}, —OCX^{9A}₃, —OCH₂X^{9A}, —OCHX^{9A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{9B} is independently oxo, halogen, —CX^{9B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{9B}₂, —CH₂X^{9B}, —OCX^{9B}₃, —OCH₂X^{9B}, —OCHX^{9B}₂, R^{9C}-substituted or unsubstituted alkyl (e.g., C₁-C₈, C₁-C₆, C₁-C₄, or C₁-C₂), R^{9C}-substituted or unsubstituted heteroalkyl (e.g., 2 to 8 membered, 2 to 6 membered, 4 to 6 membered, or 2 to 3 membered), R^{9C}-substituted or unsubstituted cycloalkyl (e.g., C₃-C₈, C₃-C₆, C₄-C₆, or C₅-C₆), R^{9C}-substituted or unsubstituted heterocycloalkyl (e.g., 3 to 6 membered, 4 to 6 membered, 4 to 5 membered, or 5 to 6 membered), R^{9C}-substituted or unsubstituted aryl (e.g., C₆-C₁₀ or phenyl), or R^{9C}-substituted or unsubstituted heteroaryl (e.g., 5 to 10 membered, 5 to 9 membered, or 5 to 6 membered). X^{9B} is independently —F, —Cl, —Br, or —I. In embodiments, R^{9B} is independently oxo, halogen, —CX^{9B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{9B}₂, —CH₂X^{9B}, —OCX^{9B}₃, —OCH₂X^{9B}, —OCHX^{9B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{9C} is independently oxo, halogen, —CX^{9C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{9C}₂, —CH₂X^{9C}, —OCX⁹C₃, —OCH₂X^{9C}, —OCHX^{9C}₂, unsubstituted alkyl (e.g., C₁-C₆, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{9C} is independently —F, —Cl, —Br, or —I.

In embodiments, R¹⁰ is independently hydrogen, —CX¹⁰₃, —CN, —COOH, —CONH₂, —CHX¹⁰₂, —CH₂X¹⁰, R^{10A}-substituted or unsubstituted alkyl (e.g., C₁-C₈, C₁-C₆, C₁-C₄, or C₁-C₂), R^{10A}-substituted or unsubstituted heteroalkyl (e.g., 2 to 8 membered, 2 to 6 membered, 4 to 6 membered, or 2 to 3 membered), R^{10A}-substituted or unsubstituted cycloalkyl (e.g., C₃-C₈, C₃-C₆, C₄-C₆, or C₅-C₆), R^{10A}-substituted or unsubstituted heterocycloalkyl (e.g., 3 to 6 membered, 4 to 6 membered, 4 to 5 membered, or 5 to 6 membered), R^{10A}-substituted or unsubstituted aryl (e.g., C₆-C₁₀ or phenyl), or R^{10A}-substituted or unsubstituted heteroaryl (e.g., 5 to 10 membered, 5 to 9 membered, or 5 to 6 membered). X¹⁰ is independently —F, —Cl, —Br, or —I. In embodiments, R¹⁰ is independently hydrogen. In embodiments, R¹⁰ is independently methyl. In embodiments, R¹⁰ is independently ethyl. In embodiments, R¹⁰ is independently hydrogen, —CX¹⁰³, —CN, —COOH, —CONH₂, —CHX¹⁰₂, —CH₂X¹⁰, unsubstituted alkyl (e.g., C₁-C₈, C₁-C₆, C₁-C₄, or C₁-C₂, unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{10A} is independently oxo, halogen, —CX^{10A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{10A}₂, —CH₂X^{10A}, —OCX^{10A}₃, —OCH₂X^{10A}, —OCHX^{10A}₂, R^{10B}-substituted or unsubstituted alkyl (e.g., C₁-C₈, C₁-C₆, C₁-C₄, or C₁-C₂), R^{10B}-substituted or unsubstituted heteroalkyl (e.g., 2 to 8 membered, 2 to 6 membered, 4 to 6 membered, or 2 to 3 membered), R^{10B} substituted or unsubstituted cycloalkyl (e.g., C₃-C₅, C₃-C₆, C₄-C₆, or C₅-C₆), R^{10B}-substituted or unsubstituted heterocycloalkyl (e.g., 3 to 6 membered, 4 to 6 membered, 4 to 5 membered, or 5 to 6 membered), R^{10B}-substituted or unsubstituted aryl (e.g., C₆-C₁₀ or phenyl), or R^{10B} substituted or unsubstituted heteroaryl (e.g., 5 to 10 membered, 5 to 9 membered, or 5 to 6 membered). X^{10A} is independently —F, —Cl, —Br, or —I. In embodiments, R^{10A} is independently oxo, halogen, —CX^{10A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{10A}₂, —CH₂X^{10A}, —OCX^{10A}₃, —OCH₂X^{10A}, —OCHX^{10A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{10B} is independently oxo, halogen, —CX^{10B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{10B}₂, —CH₂X^{10B}, —OCX^{10B}₃, —OCH₂X^{10B}, —OCHX^{10B}₂, R^{10C}-substituted or unsubstituted alkyl (e.g., C₁-C₈, C₁-C₆, C₁-C₄, or C₁-C₂), R^{10C}-substituted or unsubstituted heteroalkyl (e.g., 2 to 8 membered, 2 to 6 membered, 4 to 6 membered, or 2 to 3 membered), R^{10C}-substituted or unsubstituted cycloalkyl (e.g., C₃-C₈, C₃-C₆, C₄-C₆, or C₅-C₆), R^{10C}-substituted or unsubstituted heterocycloalkyl (e.g., 3 to 6 membered, 4 to 6 membered, 4 to 5 membered, or 5 to 6 membered), R^{10C}-substituted or unsubstituted aryl (e.g., C₆-C₁₀ or phenyl), or R^{10C}-substituted or unsubstituted heteroaryl (e.g., 5 to 10 membered, 5 to 9 membered, or 5 to 6 membered). X^{10B} is independently —F, —Cl, —Br, or —I. In embodiments, R^{10B} is independently oxo, halogen, —CX^{10B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{10B}₂, —CH₂X^{10B}, —OCX^{10B}₃, —OCH₂X^{10B}, —OCHX^{10B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{10C} is independently oxo, halogen, —CX^{10C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{10C}₂, —CH₂X^{10C}, —OCX^{10C}₃, —OCH₂X^{10C}, —OCHX^{10C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{10C} is independently —F, —Cl, —Br, or —I.

In embodiments, R¹¹ is independently hydrogen, —CX¹¹₃, —CN, —COOH, —CONH₂, —CHX¹¹₂, —CH₂X¹¹, R^{11A}-substituted or unsubstituted alkyl (e.g., C₁-C₈, C₁-C₆, C₁-C₄, or C₁-C₂), R^{llA}-substituted or unsubstituted heteroalkyl (e.g., 2 to 8 membered, 2 to 6 membered, 4 to 6 membered, or 2 to 3 membered), R^{11A}-substituted or unsubstituted cycloalkyl (e.g., C₃-C₈, C₃-C₆, C₄-C₆, or C₅-C₆), R^{11A}-substituted or unsubstituted heterocycloalkyl (e.g., 3 to 6 membered, 4 to 6 membered, 4 to 5 membered, or 5 to 6 membered), R^{11A}-substituted or unsubstituted aryl (e.g., C₆-C₁₀ or phenyl), or R^{11A}-substituted or unsubstituted heteroaryl (e.g., 5 to 10 membered, 5 to 9 membered, or 5 to 6 membered). X¹¹ is independently —F, —Cl, —Br, or —I. In embodiments, R¹¹ is independently hydrogen. In embodiments, R¹¹ is independently methyl. In embodiments, R¹¹ is independently ethyl. In embodiments, R¹¹ is independently hydrogen, —CX¹¹₃, —CN, —COOH, —CONH₂, —CHX¹¹₂, —CH₂X¹¹, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹¹ and R¹² substituents bonded to the same nitrogen atom may optionally be joined to form a R^{11A}-substituted or unsubstituted heterocycloalkyl or R^{11A}-substituted or unsubstituted heteroaryl. In embodiments, R¹¹ and R¹² substituents bonded to the same nitrogen atom may optionally be joined to form a R^{11A}-substituted or unsubstituted 3 to 6 membered heterocycloalkyl or R^{11A}-substituted or unsubstituted 5 to 6 membered heteroaryl. In embodiments, R¹¹ and R¹² substituents bonded to the same nitrogen atom may optionally be joined to form an unsubstituted 3 to 6 membered heterocycloalkyl or unsubstituted 5 to 6 membered heteroaryl. In embodiments, R¹¹ and R¹² substituents bonded to the same nitrogen atom may optionally be joined to form a R^{11A}-substituted or unsubstituted piperazinyl. In embodiments, R¹¹ and R¹² substituents bonded to the same nitrogen atom may optionally be joined to form a R^{11A}-substituted or unsubstituted piperidinyl. In embodiments, R¹¹ and R¹² substituents bonded to the same nitrogen atom may optionally be joined to form a R^{11A}-substituted or unsubstituted pyrrolidinyl. In embodiments, R¹¹ and R¹² substituents bonded to the same nitrogen atom may optionally be joined to form a R^{11A}-substituted or unsubstituted azetidinyl. In embodiments, R¹¹ and R¹² substituents bonded to the same nitrogen atom may optionally be joined to form a R^{11A}-substituted or unsubstituted morpholinyl. In embodiments, R¹¹ and R¹² substituents bonded to the same nitrogen atom may optionally be joined to form a R^{llA}-substituted or unsubstituted azeridinyl.

R^{11A} is independently oxo, halogen, —CX^{llA}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{11A}₂, —CH₂X^{11A}, —OCX^{11A}₃, —OCH₂X^{11A}, —OCHX^{11A}₂, R^{11B}-substituted or unsubstituted alkyl (e.g., C₁-C₈, C₁-C₆, C₁-C₄, or C₁-C₂), R^{11B}-substituted or unsubstituted heteroalkyl (e.g., 2 to 8 membered, 2 to 6 membered, 4 to 6 membered, or 2 to 3 membered), R^{11B}-substituted or unsubstituted cycloalkyl (e.g., C₃-C₈, C₃-C₆, C₄-C₆, or C₅-C₆), R^{11B}-substituted or unsubstituted heterocycloalkyl (e.g., 3 to 6 membered, 4 to 6 membered, 4 to 5 membered, or 5 to 6 membered), R^{11B}-substituted or unsubstituted aryl (e.g., C₆-C₁₀ or phenyl), or R^{11B}-substituted or unsubstituted heteroaryl (e.g., 5 to 10 membered, 5 to 9 membered, or 5 to 6 membered). X^{11A} is independently —F, —Cl, —Br, or —I. In embodiments, R^{11A} is independently oxo, halogen, —CX^{llA}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{11A}₂, —CH₂X^{11A}, —OCX^{11A}₃, —OCH₂X^{11A}, —OCHX^{11A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{11B} is independently oxo, halogen, —CX^{11B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{11B}₂, —CH₂X^{11B}, —OCX^{11B}₃, —OCH₂X^{1lB}, —OCHX^{11B}₂, R^{11C}-substituted or unsubstituted alkyl (e.g., C₁-C₈, C₁-C₆, C₁-C₄, or C₁-C₂), R^{11C}-substituted or unsubstituted heteroalkyl (e.g., 2 to 8 membered, 2 to 6 membered, 4 to 6 membered, or 2 to 3 membered), R^{11C}-substituted or unsubstituted cycloalkyl (e.g., C₃-C₈, C₃-C₆, C₄-C₆, or C₅-C₆), R^{11C}-substituted or unsubstituted heterocycloalkyl (e.g., 3 to 6 membered, 4 to 6 membered, 4 to 5 membered, or 5 to 6 membered), R^{11C}-substituted or unsubstituted aryl (e.g., C₆-C₁₀ or phenyl), or R^{11C}-substituted or unsubstituted heteroaryl (e.g., 5 to 10 membered, 5 to 9 membered, or 5 to 6 membered). X^{11B} is independently —F, —Cl, —Br, or —I. In embodiments, R^{11B} is independently oxo, halogen, —CX^{11B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{11B}₂, —CH₂X^{11B}, —OCX^{11B}₃, —OCH₂X^{11B}, —OCHX^{11B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{11C} is independently oxo, halogen, —CX^{11C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{11C}₂, —CH₂X^{11C}, —OCX^{11C}₃, —OCH₂X^{11C}, —OCHX^{11C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{11C} is independently —F, —Cl, —Br, or —I.

In embodiments, R¹² is independently hydrogen, —CX¹²₃, —CN, —COOH, —CONH₂, —CHX¹²₂, —CH₂X¹², R^{12A}-substituted or unsubstituted alkyl (e.g., C₁-C₈, C₁-C₆, C₁-C₄, or C₁-C₂), R^{12A}-substituted or unsubstituted heteroalkyl (e.g., 2 to 8 membered, 2 to 6 membered, 4 to 6 membered, or 2 to 3 membered), R^{12A}-substituted or unsubstituted cycloalkyl (e.g., C₃-C₈, C₃-C₆, C₄-C₆, or C₅-C₆), R^{12A}-substituted or unsubstituted heterocycloalkyl (e.g., 3 to 6 membered, 4 to 6 membered, 4 to 5 membered, or 5 to 6 membered), R^{12A}-substituted or unsubstituted aryl (e.g., C₆-C₁₀ or phenyl), or R^{12A}-substituted or unsubstituted heteroaryl (e.g., 5 to 10 membered, 5 to 9 membered, or 5 to 6 membered). X¹² is independently —F, —Cl, —Br, or —I. In embodiments, R¹² is independently hydrogen. In embodiments, R¹² is independently methyl. In embodiments, R¹² is independently ethyl. In embodiments, R¹² is independently hydrogen, —CX¹²₃, —CN, —COOH, —CONH₂, —CHX¹²₂, —CH₂X¹², unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹² and R¹¹ substituents bonded to the same nitrogen atom may optionally be joined to form a R^{12A}-substituted or unsubstituted heterocycloalkyl or R^{12A}-substituted or unsubstituted heteroaryl. In embodiments, R¹² and R¹¹ substituents bonded to the same nitrogen atom may optionally be joined to form a R^{12A}-substituted or unsubstituted 3 to 6 membered heterocycloalkyl or R^{12A}-substituted or unsubstituted 5 to 6 membered heteroaryl. In embodiments, R¹² and R¹¹ substituents bonded to the same nitrogen atom may optionally be joined to form an unsubstituted 3 to 6 membered heterocycloalkyl or unsubstituted 5 to 6 membered heteroaryl. In embodiments, R¹² and R¹¹ substituents bonded to the same nitrogen atom may optionally be joined to form a R^{12A}-substituted or unsubstituted piperazinyl. In embodiments, R¹² and R¹¹ substituents bonded to the same nitrogen atom may optionally be joined to form a R^{12A}-substituted or unsubstituted piperidinyl. In embodiments, R¹² and R¹¹ substituents bonded to the same nitrogen atom may optionally be joined to form a R^{12A}-substituted or unsubstituted pyrrolidinyl. In embodiments, R¹² and R¹¹ substituents bonded to the same nitrogen atom may optionally be joined to form a R^{12A}-substituted or unsubstituted azetidinyl. In embodiments, R¹² and R¹¹ substituents bonded to the same nitrogen atom may optionally be joined to form a R^{12A}-substituted or unsubstituted morpholinyl. In embodiments, R¹² and R¹¹ substituents bonded to the same nitrogen atom may optionally be joined to form a R^{12A}-substituted or unsubstituted azeridinyl.

R^{12A} is independently oxo, halogen, —CX^{12A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{12A}₂, —CH₂X^{12A}, —OCX^{12A}₃, —OCH₂X^{12A}, —OCHX^{12A}₂, R^{12B}-substituted or unsubstituted alkyl (e.g., C₁-C₈, C₁-C₆, C₁-C₄, or C₁-C₂), R^{12B}-substituted or unsubstituted heteroalkyl (e.g., 2 to 8 membered, 2 to 6 membered, 4 to 6 membered, or 2 to 3 membered), R^{12B}-substituted or unsubstituted cycloalkyl (e.g., C₃-C₈, C₃-C₆, C₄-C₆, or C₅-C₆), R^{12B}-substituted or unsubstituted heterocycloalkyl (e.g., 3 to 6 membered, 4 to 6 membered, 4 to 5 membered, or 5 to 6 membered), R^{12B}-substituted or unsubstituted aryl (e.g., C₆-C₁₀ or phenyl), or R^{12B}-substituted or unsubstituted heteroaryl (e.g., 5 to 10 membered, 5 to 9 membered, or 5 to 6 membered). X^{12A} is independently —F, —Cl, —Br, or —I. In embodiments, R^{12A} is independently oxo, halogen, —CX^{12A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{12A}₂, —CH₂X^{12A}, —OCX^{12A}₃, —OCH₂X^{12A}, —OCHX^{12A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{12B} is independently oxo, halogen, —CX^{12B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{12B}₂, —CH₂X^{12B}, —OCX^{12B}₃, —OCH₂X^{12B}, —OCHX^{12B}₂, R^{12C}-substituted or unsubstituted alkyl (e.g., C₁-C₈, C₁-C₆, C₁-C₄, or C₁-C₂), R^{12C}-substituted or unsubstituted heteroalkyl (e.g., 2 to 8 membered, 2 to 6 membered, 4 to 6 membered, or 2 to 3 membered), R^{12C}-substituted or unsubstituted cycloalkyl (e.g., C₃-C₈, C₃-C₆, C₄-C₆, or C₅-C₆), R^{12C}-substituted or unsubstituted heterocycloalkyl (e.g., 3 to 6 membered, 4 to 6 membered, 4 to 5 membered, or 5 to 6 membered), R^{12C}-substituted or unsubstituted aryl (e.g., C₆-C₁₀ or phenyl), or R^{12C}-substituted or unsubstituted heteroaryl (e.g., 5 to 10 membered, 5 to 9 membered, or 5 to 6 membered). X^{12B} is independently —F, —Cl, —Br, or —I. In embodiments, R^{12B} is independently oxo, halogen, —CX^{12B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{12B}₂, —CH₂X^{12B}, —OCX^{12B}₃, —OCH₂X^{12B}, —OCHX^{12B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{12C} is independently oxo, halogen, —CX^{12C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{12C}₂, —CH₂X^{12C}, —OCX¹²C₃, —OCH₂X^{12C}, —OCHX^{12C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{12C} is independently —F, —Cl, —Br, or —I.

In embodiments, R¹³ is independently hydrogen, —CX¹³₃, —CN, —COOH, —CONH₂, —CHX¹³₂, —CH₂X¹, R^{13A}-substituted or unsubstituted alkyl (e.g., C₁-C₈, C₁-C₆, C₁-C₄, or C₁-C₂), R^{13A}-substituted or unsubstituted heteroalkyl (e.g., 2 to 8 membered, 2 to 6 membered, 4 to 6 membered, or 2 to 3 membered), R^{13A}-substituted or unsubstituted cycloalkyl (e.g., C₃-C₈, C₃-C₆, C₄-C₆, or C₅-C₆), R^{13A}-substituted or unsubstituted heterocycloalkyl (e.g., 3 to 6 membered, 4 to 6 membered, 4 to 5 membered, or 5 to 6 membered), R^{13A}-substituted or unsubstituted aryl (e.g., C₆-C₁₀ or phenyl), or R^{13A}-substituted or unsubstituted heteroaryl (e.g., 5 to 10 membered, 5 to 9 membered, or 5 to 6 membered). X¹³ is independently —F, —Cl, —Br, or —I. In embodiments, R¹³ is independently hydrogen. In embodiments, R¹³ is independently methyl. In embodiments, R¹³ is independently ethyl. In embodiments, R¹³ is independently hydrogen, —CX¹³₃, —CN, —COOH, —CONH₂, —CHX¹³₂, —CH₂X¹³, unsubstituted alkyl (e.g., C₁-C₈, C₁-C₆, C₁-C₄, or C₁-C₂, unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{13A} is independently oxo, halogen, —CX^{13A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{13A}₂, —CH₂X^{13A}, —OCX^{13A}₃, —OCH₂X^{13A}, —OCHX^{13A}₂, R^{13B}-substituted or unsubstituted alkyl (e.g., C₁-C₈, C₁-C₆, C₁-C₄, or C₁-C₂), R^{13B}-substituted or unsubstituted heteroalkyl (e.g., 2 to 8 membered, 2 to 6 membered, 4 to 6 membered, or 2 to 3 membered), R^{13B}-substituted or unsubstituted cycloalkyl (e.g., C₃-C₈, C₃-C₆, C₄-C₆, or C₅-C₆), R^{13B}-substituted or unsubstituted heterocycloalkyl (e.g., 3 to 6 membered, 4 to 6 membered, 4 to 5 membered, or 5 to 6 membered), R^{13B}-substituted or unsubstituted aryl (e.g., C₆-C₁₀ or phenyl), or R^{13B}-substituted or unsubstituted heteroaryl (e.g., 5 to 10 membered, 5 to 9 membered, or 5 to 6 membered). X^{13A} is independently —F, —Cl, —Br, or —I. In embodiments, R^{13A} is independently oxo, halogen, —CX^{13A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{13A}₂, —CH₂X^{13A}—OCX^{13A}₃, —OCH₂X^{13A}, —OCHX^{13A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{13B} is independently oxo, halogen, —CX^{13B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{13B}₂, —CH₂X^{13B}, —OCX^{13B}₃, —OCH₂X^{13B}, —OCHX^{13B}₂, R^{13C}-substituted or unsubstituted alkyl (e.g., C₁-C₈, C₁-C₆, C₁-C₄, or C₁-C₂), R^{13C}-substituted or unsubstituted heteroalkyl (e.g., 2 to 8 membered, 2 to 6 membered, 4 to 6 membered, or 2 to 3 membered), R^{13C}-substituted or unsubstituted cycloalkyl (e.g., C₃-C₈, C₃-C₆, C₄-C₆, or C₅-C₆), R^{13C}-substituted or unsubstituted heterocycloalkyl (e.g., 3 to 6 membered, 4 to 6 membered, 4 to 5 membered, or 5 to 6 membered), R^{13C}-substituted or unsubstituted aryl (e.g., C₆-C₁₀ or phenyl), or R^{13C}-substituted or unsubstituted heteroaryl (e.g., 5 to 10 membered, 5 to 9 membered, or 5 to 6 membered). X^{13B} is independently —F, —Cl, —Br, or —I. In embodiments, R^{13B} is independently oxo, halogen, —CX^{13B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{13B}₂, —CH₂X^{13B}, —OCX^{13B}₃, —OCH₂X^{13B}, —OCHX^{13B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{13C} is independently oxo, halogen, —CX^{13C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{13C}₂, —CH₂X^{13C}, —OCX^{13C}₃, —OCH₂X^{13C}, —OCHX^{13C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{13C} is independently —F, —Cl, —Br, or —I.

In embodiments, R¹⁴ is independently hydrogen, —CX¹⁴₃, —CN, —COOH, —CONH₂, —CHX¹⁴₂, —CH₂X¹⁴, R^{14A}-substituted or unsubstituted alkyl (e.g., C₁-C₈, C₁-C₆, C₁-C₄, or C₁-C₂), R^{14A}-substituted or unsubstituted heteroalkyl (e.g., 2 to 8 membered, 2 to 6 membered, 4 to 6 membered, or 2 to 3 membered), R^{14A}-substituted or unsubstituted cycloalkyl (e.g., C₃-C₈, C₃-C₆, C₄-C₆, or C₅-C₆), R^{14A}-substituted or unsubstituted heterocycloalkyl (e.g., 3 to 6 membered, 4 to 6 membered, 4 to 5 membered, or 5 to 6 membered), R^{14A}-substituted or unsubstituted aryl (e.g., C₆-C₁₀ or phenyl), or R^{14A}-substituted or unsubstituted heteroaryl (e.g., 5 to 10 membered, 5 to 9 membered, or 5 to 6 membered). X¹⁴ is independently —F, —Cl, —Br, or —I. In embodiments, R¹⁴ is independently hydrogen. In embodiments, R¹⁴ is independently methyl. In embodiments, R¹⁴ is independently ethyl. In embodiments, R¹⁴ is independently hydrogen, —CX¹⁴₃, —CN, —COOH, —CONH₂, —CHX¹⁴₂, —CH₂X¹⁴, unsubstituted alkyl (e.g., C₁-C₈, C₁-C₆, C₁-C₄, or C₁-C₂, unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{14A} is independently oxo, halogen, —CX^{14A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{14A}₂, —CH₂X^{14A}, —OCX^{14A}₃, —OCH₂X^{14A}, —OCHX^{14A}₂, R^{14B}-substituted or unsubstituted alkyl (e.g., C₁-C₈, C₁-C₆, C₁-C₄, or C₁-C₂), R^{14B}-substituted or unsubstituted heteroalkyl (e.g., 2 to 8 membered, 2 to 6 membered, 4 to 6 membered, or 2 to 3 membered), R^{14B}-substituted or unsubstituted cycloalkyl (e.g., C₃-C₅, C₃-C₆, C₄-C₆, or C₅-C₆), R^{14B}-substituted or unsubstituted heterocycloalkyl (e.g., 3 to 6 membered, 4 to 6 membered, 4 to 5 membered, or 5 to 6 membered), R^{14B}-substituted or unsubstituted aryl (e.g., C₆-C₁₀ or phenyl), or R^{14B}-substituted or unsubstituted heteroaryl (e.g., 5 to 10 membered, 5 to 9 membered, or 5 to 6 membered). X^{14A} is independently —F, —Cl, —Br, or —I. In embodiments, R^{14A} is independently oxo, halogen, —CX^{14A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{14A}₂, —CH₂X^{14A}, —OCX^{14A}₃, —OCH₂X^{14A}, —OCHX^{14A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{14B} is independently oxo, halogen, —CX^{14B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{14B}₂, —CH₂X^{14B}, —OCX^{14B}₃, —OCH₂X^{14B}, —OCHX^{14B}₂, R^{14C}-substituted or unsubstituted alkyl (e.g., C₁-C₈, C₁-C₆, C₁-C₄, or C₁-C₂), R^{14C}-substituted or unsubstituted heteroalkyl (e.g., 2 to 8 membered, 2 to 6 membered, 4 to 6 membered, or 2 to 3 membered), R^{14C}-substituted or unsubstituted cycloalkyl (e.g., C₃-C₈, C₃-C₆, C₄-C₆, or C₅-C₆), R^{14C}-substituted or unsubstituted heterocycloalkyl (e.g., 3 to 6 membered, 4 to 6 membered, 4 to 5 membered, or 5 to 6 membered), R^{14C}-substituted or unsubstituted aryl (e.g., C₆-C₁₀ or phenyl), or R^{14C}-substituted or unsubstituted heteroaryl (e.g., 5 to 10 membered, 5 to 9 membered, or 5 to 6 membered). X^{14B} is independently —F, —Cl, —Br, or —I. In embodiments, R^{14B} is independently oxo, halogen, —CX^{14B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{14B}₂, —CH₂X^{14B}, —OCX^{14B}₃, —OCH₂X^{14B}, —OCHX^{14B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{14C} is independently oxo, halogen, —CX^{14C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{14C}₂, —CH₂X^{14C}, —OCX^{14C}₃, —OCH₂X^{14C}, —OCHX^{14C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{14C} is independently —F, —Cl, —Br, or —I.

In embodiments, R¹⁵ is independently hydrogen, —CX¹⁵³, —CN, —COOH, —CONH₂, —CHX¹⁵₂, —CH₂X¹⁵, R^{15A}-substituted or unsubstituted alkyl (e.g., C₁-C₈, C₁-C₆, C₁-C₄, or C₁-C₂), R^{15A}-substituted or unsubstituted heteroalkyl (e.g., 2 to 8 membered, 2 to 6 membered, 4 to 6 membered, or 2 to 3 membered), R^{15A}-substituted or unsubstituted cycloalkyl (e.g., C₃-C₈, C₃-C₆, C₄-C₆, or C₅-C₆), R^{15A}-substituted or unsubstituted heterocycloalkyl (e.g., 3 to 6 membered, 4 to 6 membered, 4 to 5 membered, or 5 to 6 membered), R^{15A}-substituted or unsubstituted aryl (e.g., C₆-C₁₀ or phenyl), or R^{15A}-substituted or unsubstituted heteroaryl (e.g., 5 to 10 membered, 5 to 9 membered, or 5 to 6 membered). X¹⁵ is independently —F, —Cl, —Br, or —I. In embodiments, R¹⁵ is independently hydrogen. In embodiments, R¹⁵ is independently methyl. In embodiments, R¹⁵ is independently ethyl. In embodiments, R¹⁵ is independently hydrogen, —CX¹⁵₃, —CN, —COOH, —CONH₂, —CHX¹⁵₂, —CH₂X¹⁵, unsubstituted alkyl (e.g., C₁-C₈, C₁-C₆, C₁-C₄, or C₁-C₂, unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁵ and R¹⁶ substituents bonded to the same nitrogen atom may optionally be joined to form a R^{15A}-substituted or unsubstituted heterocycloalkyl or R^{15A}-substituted or unsubstituted heteroaryl. In embodiments, R¹⁵ and R¹⁶ substituents bonded to the same nitrogen atom may optionally be joined to form a R^{15A}-substituted or unsubstituted 3 to 6 membered heterocycloalkyl or R^{15A}-substituted or unsubstituted 5 to 6 membered heteroaryl. In embodiments, R¹⁵ and R¹⁶ substituents bonded to the same nitrogen atom may optionally be joined to form an unsubstituted 3 to 6 membered heterocycloalkyl or unsubstituted 5 to 6 membered heteroaryl. In embodiments, R¹⁵ and R¹⁶ substituents bonded to the same nitrogen atom may optionally be joined to form a R^{15A}-substituted or unsubstituted piperazinyl. In embodiments, R¹⁵ and R¹⁶ substituents bonded to the same nitrogen atom may optionally be joined to form a R^{15A}-substituted or unsubstituted piperidinyl. In embodiments, R¹⁵ and R¹⁶ substituents bonded to the same nitrogen atom may optionally be joined to form a R^{15A}-substituted or unsubstituted pyrrolidinyl. In embodiments, R¹⁵ and R¹⁶ substituents bonded to the same nitrogen atom may optionally be joined to form a R^{15A}-substituted or unsubstituted azetidinyl. In embodiments, R¹⁵ and R¹⁶ substituents bonded to the same nitrogen atom may optionally be joined to form a R^{15A}-substituted or unsubstituted morpholinyl. In embodiments, R¹⁵ and R¹⁶ substituents bonded to the same nitrogen atom may optionally be joined to form a R^{15A}-substituted or unsubstituted azeridinyl.

R^{15A} is independently oxo, halogen, —CX^{15A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{15A}₂, —CH₂X^{15A}, —OCX^{15A}₃, —OCH₂X^{15A}, —OCHX^{15A}₂, R^{15B}-substituted or unsubstituted alkyl (e.g., C₁-C₈, C₁-C₆, C₁-C₄, or C₁-C₂), R^{15B}-substituted or unsubstituted heteroalkyl (e.g., 2 to 8 membered, 2 to 6 membered, 4 to 6 membered, or 2 to 3 membered), R^{15B}-substituted or unsubstituted cycloalkyl (e.g., C₃-C₈, C₃-C₆, C₄-C₆, or C₅-C₆), R^{15B}-substituted or unsubstituted heterocycloalkyl (e.g., 3 to 6 membered, 4 to 6 membered, 4 to 5 membered, or 5 to 6 membered), R^{15B}-substituted or unsubstituted aryl (e.g., C₆-C₁₀ or phenyl), or R^{15B}-substituted or unsubstituted heteroaryl (e.g., 5 to 10 membered, 5 to 9 membered, or 5 to 6 membered). X^{15A} is independently —F, —Cl, —Br, or —I. In embodiments, R^{15A} is independently oxo, halogen, —CX^{15A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{15A}₂, —CH₂X^{15A}—OCX^{15A}₃, —OCH₂X^{15A}, —OCHX^{15A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{15B} is independently oxo, halogen, —CX^{15B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{15B}₂, —CH₂X^{15B}, —OCX^{15B}₃, —OCH₂X^{15B}, —OCHX^{15B}₂, R^{15C}-substituted or unsubstituted alkyl (e.g., C₁-C₈, C₁-C₆, C₁-C₄, or C₁-C₂), R^{15C}-substituted or unsubstituted heteroalkyl (e.g., 2 to 8 membered, 2 to 6 membered, 4 to 6 membered, or 2 to 3 membered), R^{15C}-substituted or unsubstituted cycloalkyl (e.g., C₃-C₅, C₃-C₆, C₄-C₆, or C₅-C₆), R^{15C}-substituted or unsubstituted heterocycloalkyl (e.g., 3 to 6 membered, 4 to 6 membered, 4 to 5 membered, or 5 to 6 membered), R^{15C}-substituted or unsubstituted aryl (e.g., C₆-C₁₀ or phenyl), or R^{15C}-substituted or unsubstituted heteroaryl (e.g., 5 to 10 membered, 5 to 9 membered, or 5 to 6 membered). X^{15B} is independently —F, —Cl, —Br, or —I. In embodiments, R^{15B} is independently oxo, halogen, —CX^{15B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{15B}₂, —CH₂X^{15B}, —OCX^{15B}₃, —OCH₂X^{15B}, —OCHX^{15B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{15C} is independently oxo, halogen, —CX^{15C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{15C}₂, —CH₂X^{15C}, —OCX^{15C}₃, —OCH₂X^{15C}, —OCHX^{15C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{15C} is independently —F, —Cl, —Br, or —I.

In embodiments, R¹⁶ is independently hydrogen, —CX¹⁶₃, —CN, —COOH, —CONH₂, —CHX¹⁶₂, —CH₂X¹⁶, R^{16A}-substituted or unsubstituted alkyl (e.g., C₁-C₈, C₁-C₆, C₁-C₄, or C₁-C₂), R^{16A}-substituted or unsubstituted heteroalkyl (e.g., 2 to 8 membered, 2 to 6 membered, 4 to 6 membered, or 2 to 3 membered), R^{16A}-substituted or unsubstituted cycloalkyl (e.g., C₃-C₈, C₃-C₆, C₄-C₆, or C₅-C₆), R^{16A}-substituted or unsubstituted heterocycloalkyl (e.g., 3 to 6 membered, 4 to 6 membered, 4 to 5 membered, or 5 to 6 membered), R^{16A}-substituted or unsubstituted aryl (e.g., C₆-C₁₀ or phenyl), or R^{16A}-substituted or unsubstituted heteroaryl (e.g., 5 to 10 membered, 5 to 9 membered, or 5 to 6 membered). X¹⁶ is independently —F, —Cl, —Br, or —I. In embodiments, R¹⁶ is independently hydrogen. In embodiments, R¹⁶ is independently methyl. In embodiments, R¹⁶ is independently ethyl. In embodiments, R¹⁶ is independently hydrogen, —CX¹⁶₃, —CN, —COOH, —CONH₂, —CHX¹⁶₂, —CH₂X¹⁶, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹⁶ and R¹⁵ substituents bonded to the same nitrogen atom may optionally be joined to form a R^{16A}-substituted or unsubstituted heterocycloalkyl or R^{16A}-substituted or unsubstituted heteroaryl. In embodiments, R¹⁶ and R¹⁵ substituents bonded to the same nitrogen atom may optionally be joined to form a R^{16A}-substituted or unsubstituted 3 to 6 membered heterocycloalkyl or R^{16A}-substituted or unsubstituted 5 to 6 membered heteroaryl. In embodiments, R¹⁶ and R¹⁵ substituents bonded to the same nitrogen atom may optionally be joined to form an unsubstituted 3 to 6 membered heterocycloalkyl or unsubstituted 5 to 6 membered heteroaryl. In embodiments, R¹⁶ and R¹⁵ substituents bonded to the same nitrogen atom may optionally be joined to form a R^{16A}-substituted or unsubstituted piperazinyl. In embodiments, R¹⁶ and R¹⁵ substituents bonded to the same nitrogen atom may optionally be joined to form a R^{16A}-substituted or unsubstituted piperidinyl. In embodiments, R¹⁶ and R¹⁵ substituents bonded to the same nitrogen atom may optionally be joined to form a R^{16A}-substituted or unsubstituted pyrrolidinyl. In embodiments, R¹⁶ and R¹⁵ substituents bonded to the same nitrogen atom may optionally be joined to form a R^{16A}-substituted or unsubstituted azetidinyl. In embodiments, R¹⁶ and R¹⁵ substituents bonded to the same nitrogen atom may optionally be joined to form a R^{16A}-substituted or unsubstituted morpholinyl. In embodiments, R¹⁶ and R¹⁵ substituents bonded to the same nitrogen atom may optionally be joined to form a R^{16A}-substituted or unsubstituted azeridinyl.

R^{16A} is independently oxo, halogen, —CX^{16A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{16A}₂, —CH₂X^{16A}, —OCX^{16A}₃, —OCH₂X^{16A}, —OCHX^{16A}₂, R^{16B}-substituted or unsubstituted alkyl (e.g., C₁-C₈, C₁-C₆, C₁-C₄, or C₁-C₂), R^{16B}-substituted or unsubstituted heteroalkyl (e.g., 2 to 8 membered, 2 to 6 membered, 4 to 6 membered, or 2 to 3 membered), R^{16B}-substituted or unsubstituted cycloalkyl (e.g., C₃-C₈, C₃-C₆, C₄-C₆, or C₅-C₆), R^{16B}-substituted or unsubstituted heterocycloalkyl (e.g., 3 to 6 membered, 4 to 6 membered, 4 to 5 membered, or 5 to 6 membered), R^{16B}-substituted or unsubstituted aryl (e.g., C₆-C₁₀ or phenyl), or R^{16B}-substituted or unsubstituted heteroaryl (e.g., 5 to 10 membered, 5 to 9 membered, or 5 to 6 membered). X^{16A} is independently —F, —Cl, —Br, or —I. In embodiments, R^{16A} is independently oxo, halogen, —CX^{16A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{16A}₂, —CH₂X^{16A}, —OCX^{16A}₃, —OCH₂X^{16A}, —OCHX^{16A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{16B} is independently oxo, halogen, —CX^{16B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{16B}₂, —CH₂X^{16B}, —OCX^{16B}₃, —OCH₂X^{16B}, —OCHX^{16B}₂, R^{16C}-substituted or unsubstituted alkyl (e.g., C₁-C₈, C₁-C₆, C₁-C₄, or C₁-C₂), R^{16C}-substituted or unsubstituted heteroalkyl (e.g., 2 to 8 membered, 2 to 6 membered, 4 to 6 membered, or 2 to 3 membered), R^{16C}-substituted or unsubstituted cycloalkyl (e.g., C₃-C₅, C₃-C₆, C₄-C₆, or C₅-C₆), R^{16C}-substituted or unsubstituted heterocycloalkyl (e.g., 3 to 6 membered, 4 to 6 membered, 4 to 5 membered, or 5 to 6 membered), R^{16C}-substituted or unsubstituted aryl (e.g., C₆-C₁₀ or phenyl), or R^{16C}-substituted or unsubstituted heteroaryl (e.g., 5 to 10 membered, 5 to 9 membered, or 5 to 6 membered). X^{16B} is independently —F, —Cl, —Br, or —I. In embodiments, R^{16B} is independently oxo, halogen, —CX^{16B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{16B}₂, —CH₂X^{16B}, —OCX^{16B}₃, —OCH₂X^{16B}, —OCHX^{16B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{16C} is independently oxo, halogen, —CX^{16C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{16C}₂, —CH₂X^{16C}, —OCX^{16C}₃, —OCH₂X^{16C}, —OCHX^{16C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{16C} is independently —F, —Cl, —Br, or —I.

In embodiments, R¹⁷ is independently hydrogen, —CX¹⁷₃, —CN, —COOH, —CONH₂, —CHX¹⁷₂, —CH₂X¹⁷, R^{17A}-substituted or unsubstituted alkyl (e.g., C₁-C₈, C₁-C₆, C₁-C₄, or C₁-C₂), R^{17A}-substituted or unsubstituted heteroalkyl (e.g., 2 to 8 membered, 2 to 6 membered, 4 to 6 membered, or 2 to 3 membered), R^{17A}-substituted or unsubstituted cycloalkyl (e.g., C₃-C₈, C₃-C₆, C₄-C₆, or C₅-C₆), R^{17A}-substituted or unsubstituted heterocycloalkyl (e.g., 3 to 6 membered, 4 to 6 membered, 4 to 5 membered, or 5 to 6 membered), R^{17A}-substituted or unsubstituted aryl (e.g., C₆-C₁₀ or phenyl), or R^{17A}-substituted or unsubstituted heteroaryl (e.g., 5 to 10 membered, 5 to 9 membered, or 5 to 6 membered). X¹⁷ is independently —F, —Cl, —Br, or —I. In embodiments, R¹⁷ is independently hydrogen. In embodiments, R¹⁷ is independently methyl. In embodiments, R¹⁷ is independently ethyl. In embodiments, R¹⁷ is independently hydrogen, —CX¹⁷₃, —CN, —COOH, —CONH₂, —CHX¹⁷₂, —CH₂X¹⁷, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{17A} is independently oxo, halogen, —CX^{17A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{17A}₂, —CH₂X^{17A}, —OCX^{17A}₃, —OCH₂X^{17A}, —OCHX^{17A}₂, R^{17B}-substituted or unsubstituted alkyl (e.g., C₁-C₈, C₁-C₆, C₁-C₄, or C₁-C₂), R^{17B}-substituted or unsubstituted heteroalkyl (e.g., 2 to 8 membered, 2 to 6 membered, 4 to 6 membered, or 2 to 3 membered), R^{17B}-substituted or unsubstituted cycloalkyl (e.g., C₃-C₈, C₃-C₆, C₄-C₆, or C₅-C₆), R^{17B}-substituted or unsubstituted heterocycloalkyl (e.g., 3 to 6 membered, 4 to 6 membered, 4 to 5 membered, or 5 to 6 membered), R^{17B}-substituted or unsubstituted aryl (e.g., C₆-C₁₀ or phenyl), or R^{17B}-substituted or unsubstituted heteroaryl (e.g., 5 to 10 membered, 5 to 9 membered, or 5 to 6 membered). X^{17A} is independently —F, —Cl, —Br, or —I. In embodiments, R^{17A} is independently oxo, halogen, —CX^{17A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{17A}₂, —CH₂X^{17A}, —OCX^{17A}₃, —OCH₂X^{17A}, —OCHX^{17A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{17B} is independently oxo, halogen, —CX^{17B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{17B}₂, —CH₂X^{17B}, —OCX^{17B}₃, —OCH₂X^{17B}, —OCHX^{17B}₂, R^{17C}-substituted or unsubstituted alkyl (e.g., C₁-C₈, C₁-C₆, C₁-C₄, or C₁-C₂), R^{17C}-substituted or unsubstituted heteroalkyl (e.g., 2 to 8 membered, 2 to 6 membered, 4 to 6 membered, or 2 to 3 membered), R^{17C}-substituted or unsubstituted cycloalkyl (e.g., C₃-C₈, C₃-C₆, C₄-C₆, or C₅-C₆), R^{17C}-substituted or unsubstituted heterocycloalkyl (e.g., 3 to 6 membered, 4 to 6 membered, 4 to 5 membered, or 5 to 6 membered), R^{17C}-substituted or unsubstituted aryl (e.g., C₆-C₁₀ or phenyl), or R^{17C}-substituted or unsubstituted heteroaryl (e.g., 5 to 10 membered, 5 to 9 membered, or 5 to 6 membered). X^{17B} is independently —F, —Cl, —Br, or —I. In embodiments, R^{17B} is independently oxo, halogen, —CX^{17B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{17B}₂, —CH₂X^{17B}—OCX^{17B}₃, —OCH₂X^{17B}, —OCHX^{17B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{17C} is independently oxo, halogen, —CX^{17C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{17C}₂, —CH₂X^{17C}, —OCX¹⁷C₃, —OCH₂X^{17C}, —OCHX^{17C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{17C} is independently —F, —Cl, —Br, or —I.

In embodiments, R¹⁸ is independently hydrogen, —CX¹⁸₃, —CN, —COOH, —CONH₂, —CHX¹⁸₂, —CH₂X¹⁸, R^{18A}-substituted or unsubstituted alkyl (e.g., C₁-C₈, C₁-C₆, C₁-C₄, or C₁-C₂, R^{18A}-substituted or unsubstituted heteroalkyl (e.g., 2 to 8 membered, 2 to 6 membered, 4 to 6 membered, or 2 to 3 membered), R^{18A}-substituted or unsubstituted cycloalkyl (e.g., C₃-C₈, C₃-C₆, C₄-C₆, or C₅-C₆), R^{18A}-substituted or unsubstituted heterocycloalkyl (e.g., 3 to 6 membered, 4 to 6 membered, 4 to 5 membered, or 5 to 6 membered), R^{18A}-substituted or unsubstituted aryl (e.g., C₆-C₁₀ or phenyl), or R^{18A}-substituted or unsubstituted heteroaryl (e.g., 5 to 10 membered, 5 to 9 membered, or 5 to 6 membered). X¹⁸ is independently —F, —Cl, —Br, or —I. In embodiments, R¹⁸ is independently hydrogen. In embodiments, R¹⁸ is independently methyl. In embodiments, R¹⁸ is independently ethyl. In embodiments, R¹⁸ is independently hydrogen, —CX¹⁸₃, —CN, —COOH, —CONH₂, —CHX¹⁸₂, —CH₂X¹⁸, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{18A} is independently oxo, halogen, —CX^{18A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{18A}₂, —CH₂X^{18A}, —OCX^{18A}₃, —OCH₂X^{18A}, —OCHX^{18A}₂, R^{18B}-substituted or unsubstituted alkyl (e.g., C₁-C₈, C₁-C₆, C₁-C₄, or C₁-C₂), R^{18B}-substituted or unsubstituted heteroalkyl (e.g., 2 to 8 membered, 2 to 6 membered, 4 to 6 membered, or 2 to 3 membered), R^{18B}-substituted or unsubstituted cycloalkyl (e.g., C₃-C₈, C₃-C₆, C₄-C₆, or C₅-C₆), R^{18B}-substituted or unsubstituted heterocycloalkyl (e.g., 3 to 6 membered, 4 to 6 membered, 4 to 5 membered, or 5 to 6 membered), R^{18B}-substituted or unsubstituted aryl (e.g., C₆-C₁₀ or phenyl), or R^{18B}-substituted or unsubstituted heteroaryl (e.g., 5 to 10 membered, 5 to 9 membered, or 5 to 6 membered). X^{18A} is independently —F, —Cl, —Br, or —I. In embodiments, R^{18A} is independently oxo, halogen, —CX^{18A}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{18A}₂, —CH₂X^{18A}, —OCX^{18A}₃, —OCH₂X^{18A}, —OCHX^{18A}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{18B} is independently oxo, halogen, —CX^{18B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{18B}₂, —CH₂X^{18B}, —OCX^{18B}₃, —OCH₂X^{18B}, —OCHX^{18B}₂R^{18C}-substituted or unsubstituted alkyl (e.g., C₁-C₈, C₁-C₆, C₁-C₄, or C₁-C₂), R^{18C}-substituted or unsubstituted heteroalkyl (e.g., 2 to 8 membered, 2 to 6 membered, 4 to 6 membered, or 2 to 3 membered), R^{18C}-substituted or unsubstituted cycloalkyl (e.g., C₃-C₈, C₃-C₆, C₄-C₆, or C₅-C₆), R^{18C}-substituted or unsubstituted heterocycloalkyl (e.g., 3 to 6 membered, 4 to 6 membered, 4 to 5 membered, or 5 to 6 membered), R^{18C}-substituted or unsubstituted aryl (e.g., C₆-C₁₀ or phenyl), or R^{18C}-substituted or unsubstituted heteroaryl (e.g., 5 to 10 membered, 5 to 9 membered, or 5 to 6 membered). X^{18B} is independently —F, —Cl, —Br, or —I. In embodiments, R^{18B} is independently oxo, halogen, —CX^{18B}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{18B}₂, —CH₂X^{18B}, —OCX^{18B}₃, —OCH₂X^{18B}, —OCHX^{18B}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R^{18C} is independently oxo, halogen, —CX^{18C}₃, —CN, —OH, —NH₂, —COOH, —CONH₂, —SH, —NHNH₂, —ONH₂, —NHC(O)NHNH₂, —NHC(O)NH₂, —NHC(O)H, —NHC(O)OH, —NHOH, —CHX^{18C}₂, —CH₂X^{18C}, —OCX^{18C}₃, —OCH₂X^{18C}, —OCHX^{18C}₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{18C} is independently —F, —Cl, —Br, or —I.

In embodiments, L¹ is independently a bond, —O—, —NH—, —C(O)—, —C(O)NH—, —NHC(O)—, —C(O)O—, —OC(O)—, —NHC(O)NH—, —S—, R²³-substituted or unsubstituted alkylene (e.g., C₁-C₈, C₁-C₆, C₁-C₄, or C₁-C₂) or R²³-substituted or unsubstituted heteroalkylene (e.g., 2 to 8 membered, 2 to 6 membered, 4 to 6 membered, or 2 to 3 membered). In embodiments, L¹ is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂) or unsubstituted heteroalkylene (e.g., 2 to 8 membered, 2 to 6 membered, 4 to 6 membered, or 2 to 3 membered).

R²³ is independently oxo, halogen, —CX²³₃, —CHX²³₂, —CH₂X²³, —OCX²³₃, —OCH₂X²³, —OCHX²³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁴-substituted or unsubstituted alkyl (e.g., C₁-C₈, C₁-C₆, C₁-C₄, or C₁-C₂), R²⁴-substituted or unsubstituted heteroalkyl (e.g., 2 to 8 membered, 2 to 6 membered, 4 to 6 membered, or 2 to 3 membered), R²⁴-substituted or unsubstituted cycloalkyl (e.g., C₃-C₈, C₃-C₆, C₄-C₆, or C₅-C₆), R²⁴-substituted or unsubstituted heterocycloalkyl (e.g., 3 to 6 membered, 4 to 6 membered, 4 to 5 membered, or 5 to 6 membered), R²⁴-substituted or unsubstituted aryl (e.g., C₆-C₁₀ or phenyl), or R²⁴-substituted or unsubstituted heteroaryl (e.g., 5 to 10 membered, 5 to 9 membered, or 5 to 6 membered). X²³ is independently —F, —Cl, —Br, or —I. In embodiments, R²³ is independently oxo, halogen, —CX²³₃, —CHX²³₂, —CH₂X²³, —OCX²³₃, —OCH₂X²³, —OCHX²³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R²⁴ is independently oxo, halogen, —CX²⁴₃, —CHX²⁴₂, —CH₂X²⁴, —OCX²⁴₃, —OCH₂X²⁴, —OCHX²⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁵-substituted or unsubstituted alkyl (e.g., C₁-C₈, C₁-C₆, C₁-C₄, or C₁-C₂), R²⁵-substituted or unsubstituted heteroalkyl (e.g., 2 to 8 membered, 2 to 6 membered, 4 to 6 membered, or 2 to 3 membered), R²⁵-substituted or unsubstituted cycloalkyl (e.g., C₃-C₅, C₃-C₆, C₄-C₆, or C₅-C₆), R²⁵-substituted or unsubstituted heterocycloalkyl (e.g., 3 to 6 membered, 4 to 6 membered, 4 to 5 membered, or 5 to 6 membered), R²⁵-substituted or unsubstituted aryl (e.g., C₆-C₁₀ or phenyl), or R²⁵-substituted or unsubstituted heteroaryl (e.g., 5 to 10 membered, 5 to 9 membered, or 5 to 6 membered). X²⁴ is independently —F, —Cl, —Br, or —I. In embodiments, R²⁴ is independently oxo, halogen, —CX²⁴₃, —CHX²⁴₂, —CH₂X²⁴, —OCX²⁴₃, —OCH₂X²⁴, —OCHX²⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R²⁵ is independently oxo, halogen, —CX²⁵³, —CHX²⁵₂, —CH₂X²⁵, —OCX²⁵³, —OCH₂X²⁵, —OCHX²⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁵ is independently —F, —Cl, —Br, or —I.

In embodiments, L² is independently a bond, —O—, —NH—, —C(O)—, —C(O)NH—, —NHC(O)—, —C(O)O—, —OC(O)—, —NHC(O)NH—, —S—, R²⁶-substituted or unsubstituted alkylene (e.g., C₁-C₈, C₁-C₆, C₁-C₄, or C₁-C₂) or R²⁶-substituted or unsubstituted heteroalkylene (e.g., 2 to 8 membered, 2 to 6 membered, 4 to 6 membered, or 2 to 3 membered). In embodiments, L² is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂) or unsubstituted heteroalkylene (e.g., 2 to 8 membered, 2 to 6 membered, 4 to 6 membered, or 2 to 3 membered).

R²⁶ is independently oxo, halogen, —CX²⁶₃, —CHX²⁶₂, —CH₂X²⁶, —OCX²⁶₃, —OCH₂X²⁶, —OCHX²⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁷-substituted or unsubstituted alkyl (e.g., C₁-C₈, C₁-C₆, C₁-C₄, or C₁-C₂), R²⁷-substituted or unsubstituted heteroalkyl (e.g., 2 to 8 membered, 2 to 6 membered, 4 to 6 membered, or 2 to 3 membered), R²⁷-substituted or unsubstituted cycloalkyl (e.g., C₃-C₈, C₃-C₆, C₄-C₆, or C₅-C₆), R²⁷-substituted or unsubstituted heterocycloalkyl (e.g., 3 to 6 membered, 4 to 6 membered, 4 to 5 membered, or 5 to 6 membered), R²⁷-substituted or unsubstituted aryl (e.g., C₆-C₁₀ or phenyl), or R²⁷-substituted or unsubstituted heteroaryl (e.g., 5 to 10 membered, 5 to 9 membered, or 5 to 6 membered). X²⁶ is independently —F, —Cl, —Br, or —I. In embodiments, R²⁶ is independently oxo, halogen, —CX²⁶₃, —CHX²⁶₂, —CH₂X²⁶, —OCX²⁶₃, —OCH₂X²⁶, —OCHX²⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R²⁷ is independently oxo, halogen, —CX²⁷³, —CHX²⁷², —CH₂X²⁷, —OCX²⁷³, —OCH₂X²⁷, —OCHX²⁷², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁸-substituted or unsubstituted alkyl (e.g., C₁-C₈, C₁-C₆, C₁-C₄, or C₁-C₂), R²⁸-substituted or unsubstituted heteroalkyl (e.g., 2 to 8 membered, 2 to 6 membered, 4 to 6 membered, or 2 to 3 membered), R²⁸-substituted or unsubstituted cycloalkyl (e.g., C₃-C₈, C₃-C₆, C₄-C₆, or C₅-C₆), R²⁸-substituted or unsubstituted heterocycloalkyl (e.g., 3 to 6 membered, 4 to 6 membered, 4 to 5 membered, or 5 to 6 membered), R²⁸-substituted or unsubstituted aryl (e.g., C₆-C₁₀ or phenyl), or R²⁸-substituted or unsubstituted heteroaryl (e.g., 5 to 10 membered, 5 to 9 membered, or 5 to 6 membered). X²⁷ is independently —F, —Cl, —Br, or —I. In embodiments, R²⁷ is independently oxo, halogen, —CX²⁷³, —CHX²⁷², —CH₂X²⁷, —OCX²⁷³, —OCH₂X²⁷, —OCHX²⁷², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R²⁸ is independently oxo, halogen, —CX²⁸³, —CHX²⁸₂, —CH₂X²⁸, —OCX²⁸³, —OCH₂X²⁸, —OCHX²⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁸ is independently —F, —Cl, —Br, or —I.

In embodiments, L^{4A} is independently a bond, —O—, —NH—, —C(O)—, —C(O)NH—, —NHC(O)—, —C(O)O—, —OC(O)—, —NHC(O)NH—, —S—, R²⁹-substituted or unsubstituted alkylene (e.g., C₁-C₈, C₁-C₆, C₁-C₄, or C₁-C₂), R²⁹-substituted or unsubstituted heteroalkylene (e.g., 2 to 8 membered, 2 to 6 membered, 4 to 6 membered, or 2 to 3 membered), R²⁹-substituted or unsubstituted cycloalkylene (e.g., C₃-C₈, C₃-C₆, C₄-C₆, or C₅-C₆), R²⁹-substituted or unsubstituted heterocycloalkylene (e.g., 3 to 6 membered, 4 to 6 membered, 4 to 5 membered, or 5 to 6 membered), R²⁹-substituted or unsubstituted arylene (e.g., C₆-C₁₀ or phenyl), or R²⁹-substituted or unsubstituted heteroarylene (e.g., 5 to 10 membered, 5 to 9 membered, or 5 to 6 membered). In embodiments, L^{4A} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered).

R²⁹ is independently oxo, halogen, —CX²⁹₃, —CHX²⁹₂, —CH₂X²⁹, —OCX²⁹₃, —OCH₂X²⁹, —OCHX²⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁰-substituted or unsubstituted alkyl (e.g., C₁-C₈, C₁-C₆, C₁-C₄, or C₁-C₂), R³⁰-substituted or unsubstituted heteroalkyl (e.g., 2 to 8 membered, 2 to 6 membered, 4 to 6 membered, or 2 to 3 membered), R³⁰-substituted or unsubstituted cycloalkyl (e.g., C₃-C₈, C₃-C₆, C₄-C₆, or C₅-C₆), R³⁰-substituted or unsubstituted heterocycloalkyl (e.g., 3 to 6 membered, 4 to 6 membered, 4 to 5 membered, or 5 to 6 membered), R³⁰-substituted or unsubstituted aryl (e.g., C₆-C₁₀ or phenyl), or R³⁰-substituted or unsubstituted heteroaryl (e.g., 5 to 10 membered, 5 to 9 membered, or 5 to 6 membered). X²⁹ is independently —F, —Cl, —Br, or —I. In embodiments, R²⁹ is independently oxo, halogen, —CX²⁹₃, —CHX²⁹₂, —CH₂X²⁹, —OCX²⁹₃, —OCH₂X²⁹, —OCHX²⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³⁰ is independently oxo, halogen, —CX³⁰₃, —CHX³⁰₂, —CH₂X³⁰, —OCX³⁰₃, —OCH₂X³⁰, —OCHX³⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, or 2 to 3 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered). X³⁰ is independently —F, —Cl, —Br, or —I. In embodiments, R³⁰ is independently oxo, halogen, —CX³⁰₃, —CHX³⁰₂, —CH₂X³⁰, —OCX³⁰₃, —OCH₂X³⁰, —OCHX³⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³¹ is independently oxo, halogen, —CX³¹₃, —CHX³¹₂, —CH₂X³¹, —OCX³¹₃, —OCH₂X³¹, —OCHX³¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³¹ is independently —F, —Cl, —Br, or —I.

In embodiments, L^{4B} is independently a bond, —O—, —NH—, —C(O)—, —C(O)NH—, —NHC(O)—, —C(O)O—, —OC(O)—, —NHC(O)NH—, —S—, R³₂-substituted or unsubstituted alkylene (e.g., C₁-C₈, C₁-C₆, C₁-C₄, or C₁-C₂), R³₂-substituted or unsubstituted heteroalkylene (e.g., 2 to 8 membered, 2 to 6 membered, 4 to 6 membered, or 2 to 3 membered), R³₂-substituted or unsubstituted cycloalkylene (e.g., C₃-C₈, C₃-C₆, C₄-C₆, or C₅-C₆), R³₂-substituted or unsubstituted heterocycloalkylene (e.g., 3 to 6 membered, 4 to 6 membered, 4 to 5 membered, or 5 to 6 membered), R³₂-substituted or unsubstituted arylene (e.g., C₆-C₁₀ or phenyl), or R³₂-substituted or unsubstituted heteroarylene (e.g., 5 to 10 membered, 5 to 9 membered, or 5 to 6 membered). In embodiments, L^{4B} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered).

R³₂ is independently oxo, halogen, —CX³³₃, —CHX³²₂, —CH₂X³², —OCX³³₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³³-substituted or unsubstituted alkyl (e.g., C₁-C₈, C₁-C₆, C₁-C₄, or C₁-C₂), R³³-substituted or unsubstituted heteroalkyl (e.g., 2 to 8 membered, 2 to 6 membered, 4 to 6 membered, or 2 to 3 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered). X³² is independently —F, —Cl, —Br, or —I. In embodiments, R³₂ is independently oxo, halogen, —CX³³₃, —CHX³²₂, —CH₂X³², —OCX³³₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³³ is independently oxo, halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁴-substituted or unsubstituted alkyl (e.g., C₁-C₈, C₁-C₆, C₁-C₄, or C₁-C₂), R³⁴-substituted or unsubstituted heteroalkyl (e.g., 2 to 8 membered, 2 to 6 membered, 4 to 6 membered, or 2 to 3 membered), R³⁴-substituted or unsubstituted cycloalkyl (e.g., C₃-C₈, C₃-C₆, C₄-C₆, or C₅-C₆), R³⁴-substituted or unsubstituted heterocycloalkyl (e.g., 3 to 6 membered, 4 to 6 membered, 4 to 5 membered, or 5 to 6 membered), R³⁴-substituted or unsubstituted aryl (e.g., C₆-C₁₀ or phenyl), or R³⁴-substituted or unsubstituted heteroaryl (e.g., 5 to 10 membered, 5 to 9 membered, or 5 to 6 membered). X³₃ is independently —F, —Cl, —Br, or —I. In embodiments, R³³ is independently oxo, halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³⁴ is independently oxo, halogen, —CX³⁴₃, —CHX³⁴₂, —CH₂X³⁴, —OCX³⁴₃, —OCH₂X³⁴, —OCHX³⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁴ is independently —F, —Cl, —Br, or —I.

In embodiments, L^{4C} is independently a bond, —O—, —NH—, —C(O)—, —C(O)NH—, —NHC(O)—, —C(O)O—, —OC(O)—, —NHC(O)NH—, —S—, R³⁵-substituted or unsubstituted alkylene (e.g., C₁-C₈, C₁-C₆, C₁-C₄, or C₁-C₂), R³⁵-substituted or unsubstituted heteroalkylene (e.g., 2 to 8 membered, 2 to 6 membered, 4 to 6 membered, or 2 to 3 membered), R³⁵-substituted or unsubstituted cycloalkylene (e.g., C₃-C₈, C₃-C₆, C₄-C₆, or C₅-C₆), R³⁵-substituted or unsubstituted heterocycloalkylene (e.g., 3 to 6 membered, 4 to 6 membered, 4 to 5 membered, or 5 to 6 membered), R³⁵-substituted or unsubstituted arylene (e.g., C₆-C₁₀ or phenyl), or R³⁵-substituted or unsubstituted heteroarylene (e.g., 5 to 10 membered, 5 to 9 membered, or 5 to 6 membered). In embodiments, L^{4C} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered).

R³⁵ is independently oxo, halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁶-substituted or unsubstituted alkyl (e.g., C₁-C₈, C₁-C₆, C₁-C₄, or C₁-C₂), R³⁶-substituted or unsubstituted heteroalkyl (e.g., 2 to 8 membered, 2 to 6 membered, 4 to 6 membered, or 2 to 3 membered), R³⁶-substituted or unsubstituted cycloalkyl (e.g., C₃-C₈, C₃-C₆, C₄-C₆, or C₅-C₆), R³⁶-substituted or unsubstituted heterocycloalkyl (e.g., 3 to 6 membered, 4 to 6 membered, 4 to 5 membered, or 5 to 6 membered), R³⁶-substituted or unsubstituted aryl (e.g., C₆-C₁₀ or phenyl), or R³⁶-substituted or unsubstituted heteroaryl (e.g., 5 to 10 membered, 5 to 9 membered, or 5 to 6 membered). X³⁵ is independently —F, —Cl, —Br, or —I. In embodiments, R³⁵ is independently oxo, halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³⁶ is independently oxo, halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, or 2 to 3 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered). X³⁶ is independently —F, —Cl, —Br, or —I. In embodiments, R³⁶ is independently oxo, halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³⁷ is independently oxo, halogen, —CX³⁷₃, —CHX³⁷₂, —CH₂X³⁷, —OCX³⁷₃, —OCH₂X³⁷, —OCHX³⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁷ is independently —F, —Cl, —Br, or —I.

In embodiments, L^{5A} is independently a bond, —O—, —NH—, —C(O)—, —C(O)NH—, —NHC(O)—, —C(O)O—, —OC(O)—, —NHC(O)NH—, —S—, R³⁸-substituted or unsubstituted alkylene (e.g., C₁-C₈, C₁-C₆, C₁-C₄, or C₁-C₂), R³⁸-substituted or unsubstituted heteroalkylene (e.g., 2 to 8 membered, 2 to 6 membered, 4 to 6 membered, or 2 to 3 membered), R³⁸-substituted or unsubstituted cycloalkylene (e.g., C₃-C₈, C₃-C₆, C₄-C₆, or C₅-C₆), R³⁸-substituted or unsubstituted heterocycloalkylene (e.g., 3 to 6 membered, 4 to 6 membered, 4 to 5 membered, or 5 to 6 membered), R³⁸-substituted or unsubstituted arylene (e.g., C₆-C₁₀ or phenyl), or R³⁸-substituted or unsubstituted heteroarylene (e.g., 5 to 10 membered, 5 to 9 membered, or 5 to 6 membered). In embodiments, L^{5A} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered). In embodiments, L^{5A} is substituted or unsubstituted piperazinylene.

R³⁸ is independently oxo, halogen, —CX³⁸₃, —CHX³⁸₂, —CH₂X³⁸, —OCX³⁸₃, —OCH₂X³⁸, —OCHX³⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³⁹-substituted or unsubstituted alkyl (e.g., C₁-C₈, C₁-C₆, C₁-C₄, or C₁-C₂), R³⁹-substituted or unsubstituted heteroalkyl (e.g., 2 to 8 membered, 2 to 6 membered, 4 to 6 membered, or 2 to 3 membered), R³⁹-substituted or unsubstituted cycloalkyl (e.g., C₃-C₈, C₃-C₆, C₄-C₆, or C₅-C₆), R³⁹-substituted or unsubstituted heterocycloalkyl (e.g., 3 to 6 membered, 4 to 6 membered, 4 to 5 membered, or 5 to 6 membered), R³⁹-substituted or unsubstituted aryl (e.g., C₆-C₁₀ or phenyl), or R³⁹-substituted or unsubstituted heteroaryl (e.g., 5 to 10 membered, 5 to 9 membered, or 5 to 6 membered). X³⁸ is independently —F, —Cl, —Br, or —I. In embodiments, R³⁸ is independently oxo, halogen, —CX³⁸₃, —CHX³⁸₂, —CH₂X³⁸, —OCX³⁸₃, —OCH₂X³⁸, —OCHX³⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R³⁹ is independently oxo, halogen, —CX³⁹₃, —CHX³⁹₂, —CH₂X³⁹, —OCX³⁹₃, —OCH₂X³⁹, —OCHX³⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴⁰-substituted or unsubstituted alkyl (e.g., C₁-C₈, C₁-C₆, C₁-C₄, or C₁-C₂), R⁴⁰-substituted or unsubstituted heteroalkyl (e.g., 2 to 8 membered, 2 to 6 membered, 4 to 6 membered, or 2 to 3 membered), R⁴⁰-substituted or unsubstituted cycloalkyl (e.g., C₃-C₈, C₃-C₆, C₄-C₆, or C₅-C₆), R⁴⁰-substituted or unsubstituted heterocycloalkyl (e.g., 3 to 6 membered, 4 to 6 membered, 4 to 5 membered, or 5 to 6 membered), R⁴⁰-substituted or unsubstituted aryl (e.g., C₆-C₁₀ or phenyl), or R⁴⁰-substituted or unsubstituted heteroaryl (e.g., 5 to 10 membered, 5 to 9 membered, or 5 to 6 membered). X³⁹ is independently —F, —Cl, —Br, or —I. In embodiments, R³⁹ is independently oxo, halogen, —CX³⁹₃, —CHX³⁹₂, —CH₂X³⁹, —OCX³⁹₃, —OCH₂X³⁹, —OCHX³⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁴⁰ is independently oxo, halogen, —CX⁴⁰₃, —CHX⁴⁰₂, —CH₂X⁴⁰, —OCX⁴⁰₃, —OCH₂X⁴⁰, —OCHX⁴⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴⁰ is independently —F, —Cl, —Br, or —I.

In embodiments, L^{5B} is independently a bond, —O—, —NH—, —C(O)—, —C(O)NH—, —NHC(O)—, —C(O)O—, —OC(O)—, —NHC(O)NH—, —S—, R⁴¹-substituted or unsubstituted alkylene (e.g., C₁-C₈, C₁-C₆, C₁-C₄, or C₁-C₂), R⁴¹-substituted or unsubstituted heteroalkylene (e.g., 2 to 8 membered, 2 to 6 membered, 4 to 6 membered, or 2 to 3 membered), R⁴¹-substituted or unsubstituted cycloalkylene (e.g., C₃-C₈, C₃-C₆, C₄-C₆, or C₅-C₆), R⁴¹-substituted or unsubstituted heterocycloalkylene (e.g., 3 to 6 membered, 4 to 6 membered, 4 to 5 membered, or 5 to 6 membered), R⁴¹-substituted or unsubstituted arylene (e.g., C₆-C₁₀ or phenyl), or R⁴¹-substituted or unsubstituted heteroarylene (e.g., 5 to 10 membered, 5 to 9 membered, or 5 to 6 membered). In embodiments, L^{5B} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered). In embodiments, L^{5B} is substituted or unsubstituted piperazinylene.

R⁴¹ is independently oxo, halogen, —CX⁴¹₃, —CHX⁴¹₂, —CH₂X⁴¹, —OCX⁴¹₃, —OCH₂X⁴¹, —OCHX⁴¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴₂-substituted or unsubstituted alkyl (e.g., C₁-C₈, C₁-C₆, C₁-C₄, or C₁-C₂), R⁴₂-substituted or unsubstituted heteroalkyl (e.g., 2 to 8 membered, 2 to 6 membered, 4 to 6 membered, or 2 to 3 membered), R⁴₂-substituted or unsubstituted cycloalkyl (e.g., C₃-C₈, C₃-C₆, C₄-C₆, or C₅-C₆), R⁴₂-substituted or unsubstituted heterocycloalkyl (e.g., 3 to 6 membered, 4 to 6 membered, 4 to 5 membered, or 5 to 6 membered), R⁴₂-substituted or unsubstituted aryl (e.g., C₆-C₁₀ or phenyl), or R⁴₂-substituted or unsubstituted heteroaryl (e.g., 5 to 10 membered, 5 to 9 membered, or 5 to 6 membered). X⁴¹ is independently —F, —Cl, —Br, or —I. In embodiments, R⁴¹ is independently oxo, halogen, —CX⁴¹₃, —CHX⁴¹₂, —CH₂X⁴¹, —OCX⁴¹₃, —OCH₂X⁴¹, —OCHX⁴¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, DNHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁴² is independently oxo, halogen, —CX⁴²₃, —CHX⁴²₂, —CH₂X⁴², —OCX⁴²₃, —OCH₂X⁴², —OCHX⁴²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴₃-substituted or unsubstituted alkyl (e.g., C₁-C₈, C₁-C₆, C₁-C₄, or C₁-C₂), R⁴₃-substituted or unsubstituted heteroalkyl (e.g., 2 to 8 membered, 2 to 6 membered, 4 to 6 membered, or 2 to 3 membered), R⁴₃-substituted or unsubstituted cycloalkyl (e.g., C₃-C₈, C₃-C₆, C₄-C₆, or C₅-C₆), R⁴₃-substituted or unsubstituted heterocycloalkyl (e.g., 3 to 6 membered, 4 to 6 membered, 4 to 5 membered, or 5 to 6 membered), R⁴₃-substituted or unsubstituted aryl (e.g., C₆-C₁₀ or phenyl), or R⁴³-substituted or unsubstituted heteroaryl (e.g., 5 to 10 membered, 5 to 9 membered, or 5 to 6 membered). X⁴² is independently —F, —Cl, —Br, or —I. In embodiments, R⁴₂ is independently oxo, halogen, —CX⁴²₃, —CHX⁴²₂, —CH₂X⁴², —OCX⁴²₃, —OCH₂X⁴², —OCHX⁴²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁴³ is independently oxo, halogen, —CX⁴³₃, —CHX⁴³₂, —CH₂X⁴³, —OCX⁴³₃, —OCH₂X⁴³, —OCHX⁴³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴₃ is independently —F, —Cl, —Br, or —I.

In embodiments, L^{5C} is independently a bond, —O—, —NH—, —C(O)—, —C(O)NH—, —NHC(O)—, —C(O)O—, —OC(O)—, —NHC(O)NH—, —S—, R⁴⁴-substituted or unsubstituted alkylene (e.g., C₁-C₈, C₁-C₆, C₁-C₄, or C₁-C₂), R⁴⁴-substituted or unsubstituted heteroalkylene (e.g., 2 to 8 membered, 2 to 6 membered, 4 to 6 membered, or 2 to 3 membered), R⁴⁴-substituted or unsubstituted cycloalkylene (e.g., C₃-C₈, C₃-C₆, C₄-C₆, or C₅-C₆), R⁴⁴-substituted or unsubstituted heterocycloalkylene (e.g., 3 to 6 membered, 4 to 6 membered, 4 to 5 membered, or 5 to 6 membered), R⁴⁴-substituted or unsubstituted arylene (e.g., C₆-C₁₀ or phenyl), or R⁴⁴-substituted or unsubstituted heteroarylene (e.g., 5 to 10 membered, 5 to 9 membered, or 5 to 6 membered). In embodiments, L^{5C} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered). In embodiments, L^{5C} is substituted or unsubstituted piperazinylene.

R⁴⁴ is independently oxo, halogen, —CX⁴⁴₃, —CHX⁴⁴₂, —CH₂X⁴⁴, —OCX⁴⁴₃, —OCH₂X⁴⁴, —OCHX⁴⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴⁵-substituted or unsubstituted alkyl (e.g., C₁-C₈, C₁-C₆, C₁-C₄, or C₁-C₂), R⁴⁵-substituted or unsubstituted heteroalkyl (e.g., 2 to 8 membered, 2 to 6 membered, 4 to 6 membered, or 2 to 3 membered), R⁴⁵-substituted or unsubstituted cycloalkyl (e.g., C₃-C₈, C₃-C₆, C₄-C₆, or C₅-C₆), R⁴⁵-substituted or unsubstituted heterocycloalkyl (e.g., 3 to 6 membered, 4 to 6 membered, 4 to 5 membered, or 5 to 6 membered), R⁴⁵-substituted or unsubstituted aryl (e.g., C₆-C₁₀ or phenyl), or R⁴⁵-substituted or unsubstituted heteroaryl (e.g., 5 to 10 membered, 5 to 9 membered, or 5 to 6 membered). X⁴⁴ is independently —F, —Cl, —Br, or —I. In embodiments, R⁴⁴ is independently oxo, halogen, —CX⁴⁴₃, —CHX⁴⁴₂, —CH₂X⁴⁴, —OCX⁴⁴₃, —OCH₂X⁴⁴, —OCHX⁴⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁴⁵ is independently oxo, halogen, —CX⁴⁵₃, —CHX⁴⁵₂, —CH₂X⁴⁵, —OCX⁴⁵₃, —OCH₂X⁴⁵, —OCHX⁴⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴⁶-substituted or unsubstituted alkyl (e.g., C₁-C₈, C₁-C₆, C₁-C₄, or C₁-C₂), R⁴⁶-substituted or unsubstituted heteroalkyl (e.g., 2 to 8 membered, 2 to 6 membered, 4 to 6 membered, or 2 to 3 membered), R⁴⁶-substituted or unsubstituted cycloalkyl (e.g., C₃-C₈, C₃-C₆, C₄-C₆, or C₅-C₆), R⁴⁶-substituted or unsubstituted heterocycloalkyl (e.g., 3 to 6 membered, 4 to 6 membered, 4 to 5 membered, or 5 to 6 membered), R⁴⁶-substituted or unsubstituted aryl (e.g., C₆-C₁₀ or phenyl), or R⁴⁶-substituted or unsubstituted heteroaryl (e.g., 5 to 10 membered, 5 to 9 membered, or 5 to 6 membered). X⁴⁵ is independently —F, —Cl, —Br, or —I. In embodiments, R⁴⁵ is independently oxo, halogen, —CX⁴⁵₃, —CHX⁴⁵₂, —CH₂X⁴⁵, —OCX⁴⁵₃, —OCH₂X⁴⁵, —OCHX⁴⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁴⁶ is independently oxo, halogen, —CX⁴⁶₃, —CHX⁴⁶₂, —CH₂X⁴⁶, —OCX⁴⁶₃, —OCH₂X⁴⁶, —OCHX⁴⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴⁶ is independently —F, —Cl, —Br, or —I.

In embodiments, L^{5D} is independently a bond, —O—, —NH—, —C(O)—, —C(O)NH—, —NHC(O)—, —C(O)O—, —OC(O)—, —NHC(O)NH—, —S—, R⁴⁷-substituted or unsubstituted alkylene (e.g., C₁-C₈, C₁-C₆, C₁-C₄, or C₁-C₂), R⁴⁷-substituted or unsubstituted heteroalkylene (e.g., 2 to 8 membered, 2 to 6 membered, 4 to 6 membered, or 2 to 3 membered), R⁴⁷-substituted or unsubstituted cycloalkylene (e.g., C₃-C₈, C₃-C₆, C₄-C₆, or C₅-C₆), R⁴⁷-substituted or unsubstituted heterocycloalkylene (e.g., 3 to 6 membered, 4 to 6 membered, 4 to 5 membered, or 5 to 6 membered), R⁴⁷-substituted or unsubstituted arylene (e.g., C₆-C₁₀ or phenyl), or R⁴⁷-substituted or unsubstituted heteroarylene (e.g., 5 to 10 membered, 5 to 9 membered, or 5 to 6 membered). In embodiments, L^{5D} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered). In embodiments, L^{5D} is substituted or unsubstituted piperazinylene.

R⁴⁷ is independently oxo, halogen, —CX⁴⁷₃, —CHX⁴⁷₂, —CH₂X⁴⁷, —OCX⁴⁷₃, —OCH₂X⁴⁷, —OCHX⁴⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴⁸-substituted or unsubstituted alkyl (e.g., C₁-C₈, C₁-C₆, C₁-C₄, or C₁-C₂), R⁴⁸-substituted or unsubstituted heteroalkyl (e.g., 2 to 8 membered, 2 to 6 membered, 4 to 6 membered, or 2 to 3 membered), R⁴⁸-substituted or unsubstituted cycloalkyl (e.g., C₃-C₈, C₃-C₆, C₄-C₆, or C₅-C₆), R⁴⁸-substituted or unsubstituted heterocycloalkyl (e.g., 3 to 6 membered, 4 to 6 membered, 4 to 5 membered, or 5 to 6 membered), R⁴⁸-substituted or unsubstituted aryl (e.g., C₆-C₁₀ or phenyl), or R⁴⁸-substituted or unsubstituted heteroaryl (e.g., 5 to 10 membered, 5 to 9 membered, or 5 to 6 membered). X⁴⁷ is independently —F, —Cl, —Br, or —I. In embodiments, R⁴⁷ is independently oxo, halogen, —CX⁴⁷₃, —CHX⁴⁷₂, —CH₂X⁴⁷, —OCX⁴⁷₃, —OCH₂X⁴⁷, —OCHX⁴⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁴⁸ is independently oxo, halogen, —CX⁴⁸₃, —CHX⁴⁸₂, —CH₂X⁴⁸, —OCX⁴⁸₃, —OCH₂X⁴⁸, —OCHX⁴⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁴⁹-substituted or unsubstituted alkyl (e.g., C₁-C₈, C₁-C₆, C₁-C₄, or C₁-C₂), R⁴⁹-substituted or unsubstituted heteroalkyl (e.g., 2 to 8 membered, 2 to 6 membered, 4 to 6 membered, or 2 to 3 membered), R⁴⁹-substituted or unsubstituted cycloalkyl (e.g., C₃-C₈, C₃-C₆, C₄-C₆, or C₅-C₆), R⁴⁹-substituted or unsubstituted heterocycloalkyl (e.g., 3 to 6 membered, 4 to 6 membered, 4 to 5 membered, or 5 to 6 membered), R⁴⁹-substituted or unsubstituted aryl (e.g., C₆-C₁₀ or phenyl), or R⁴⁹-substituted or unsubstituted heteroaryl (e.g., 5 to 10 membered, 5 to 9 membered, or 5 to 6 membered). X⁴⁸ is independently —F, —Cl, —Br, or —I. In embodiments, R⁴⁸ is independently oxo, halogen, —CX⁴⁸₃, —CHX⁴⁸₂, —CH₂X⁴⁸, —OCX⁴⁸₃, —OCH₂X⁴⁸, —OCHX⁴⁸₂, —CN, —OH, —NH₂, —COOH, —CONH₂, —NO₂, —SH, —SO₃H, —SO₄H, —SO₂NH₂, DNHNH₂, DONH₂, DNHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁴⁹ is independently oxo, halogen, —CX⁴⁹₃, —CHX⁴⁹₂, —CH₂X⁴⁹, —OCX⁴⁹₃, —OCH₂X⁴⁹, —OCHX⁴⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₅, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴⁹ is independently —F, —Cl, —Br, or —I.

In embodiments, L^{5E} is independently a bond, —O—, —NH—, —C(O)—, —C(O)NH—, —NHC(O)—, —C(O)O—, —OC(O)—, —NHC(O)NH—, —S—, R⁵⁰-substituted or unsubstituted alkylene (e.g., C₁-C₈, C₁-C₆, C₁-C₄, or C₁-C₂), R⁵⁰-substituted or unsubstituted heteroalkylene (e.g., 2 to 8 membered, 2 to 6 membered, 4 to 6 membered, or 2 to 3 membered), R⁵⁰-substituted or unsubstituted cycloalkylene (e.g., C₃-C₈, C₃-C₆, C₄-C₆, or C₅-C₆), R⁵⁰-substituted or unsubstituted heterocycloalkylene (e.g., 3 to 6 membered, 4 to 6 membered, 4 to 5 membered, or 5 to 6 membered), R⁵⁰-substituted or unsubstituted arylene (e.g., C₆-C₁₀ or phenyl), or R⁵⁰-substituted or unsubstituted heteroarylene (e.g., 5 to 10 membered, 5 to 9 membered, or 5 to 6 membered). In embodiments, L^{5E} is independently a bond, —O—, —NH—, —C(O)—, —C(O)NH—, —NHC(O)—, —C(O)O—, —OC(O)—, —NHC(O)NH—, —S—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, or 2 to 3 membered), unsubstituted cycloalkylene (e.g., C₃-C₈, C₃-C₆, C₄-C₆, or C₅-C₆), unsubstituted heterocycloalkylene (e.g., 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered). In embodiments, L^{5E} is substituted or unsubstituted piperazinylene.

R⁵⁰ is independently oxo, halogen, —CX⁵⁰₃, —CHX⁵⁰₂, —CH₂X⁵⁰, —OCX⁵⁰₃, —OCH₂X⁵⁰, —OCHX⁵⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁵¹-substituted or unsubstituted alkyl (e.g., C₁-C₈, C₁-C₆, C₁-C₄, or C₁-C₂), R⁵¹-substituted or unsubstituted heteroalkyl (e.g., 2 to 8 membered, 2 to 6 membered, 4 to 6 membered, or 2 to 3 membered), R⁵¹-substituted or unsubstituted cycloalkyl (e.g., C₃-C₈, C₃-C₆, C₄-C₆, or C₅-C₆), R⁵¹-substituted or unsubstituted heterocycloalkyl (e.g., 3 to 6 membered, 4 to 6 membered, 4 to 5 membered, or 5 to 6 membered), R⁵¹-substituted or unsubstituted aryl (e.g., C₆-C₁₀ or phenyl), or R⁵¹-substituted or unsubstituted heteroaryl (e.g., 5 to 10 membered, 5 to 9 membered, or 5 to 6 membered). X⁵⁰ is independently —F, —Cl, —Br, or —I. In embodiments, R⁵⁰ is independently oxo, halogen, —CX⁵⁰₃, —CHX⁵⁰₂, —CH₂X⁵⁰, —OCX⁵⁰₃, —OCH₂X⁵⁰, —OCHX⁵⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁵¹ is independently oxo, halogen, —CX⁵¹₃, —CHX⁵¹₂, —CH₂X⁵¹, —OCX⁵¹₃, —OCH₂X⁵¹, —OCHX⁵¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R⁵²-substituted or unsubstituted alkyl (e.g., C₁-C₈, C₁-C₆, C₁-C₄, or C₁-C₂), R⁵²-substituted or unsubstituted heteroalkyl (e.g., 2 to 8 membered, 2 to 6 membered, 4 to 6 membered, or 2 to 3 membered), R⁵²-substituted or unsubstituted cycloalkyl (e.g., C₃-C₈, C₃-C₆, C₄-C₆, or C₅-C₆), R⁵²-substituted or unsubstituted heterocycloalkyl (e.g., 3 to 6 membered, 4 to 6 membered, 4 to 5 membered, or 5 to 6 membered), R⁵²-substituted or unsubstituted aryl (e.g., C₆-C₁₀ or phenyl), or R⁵²-substituted or unsubstituted heteroaryl (e.g., 5 to 10 membered, 5 to 9 membered, or 5 to 6 membered). X⁵¹ is independently —F, —Cl, —Br, or —I. In embodiments, R⁵¹ is independently oxo, halogen, —CX⁵¹₃, —CHX⁵¹₂, —CH₂X⁵¹, —OCX⁵¹₃, —OCH₂X⁵¹, —OCHX⁵¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

R⁵² is independently oxo, halogen, —CX⁵²₃, —CHX⁵²₂, —CH₂X⁵², —OCX⁵²₃, —OCH₂X⁵², —OCHX⁵²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, or 2 to 3 membered), unsubstituted cycloalkyl (e.g., C₃-C₈, C₃-C₆, C₄-C₆, or C₅-C₆), unsubstituted heterocycloalkyl (e.g., 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴⁹ is independently —F, —Cl, —Br, or —I.

In embodiments, a compound is:

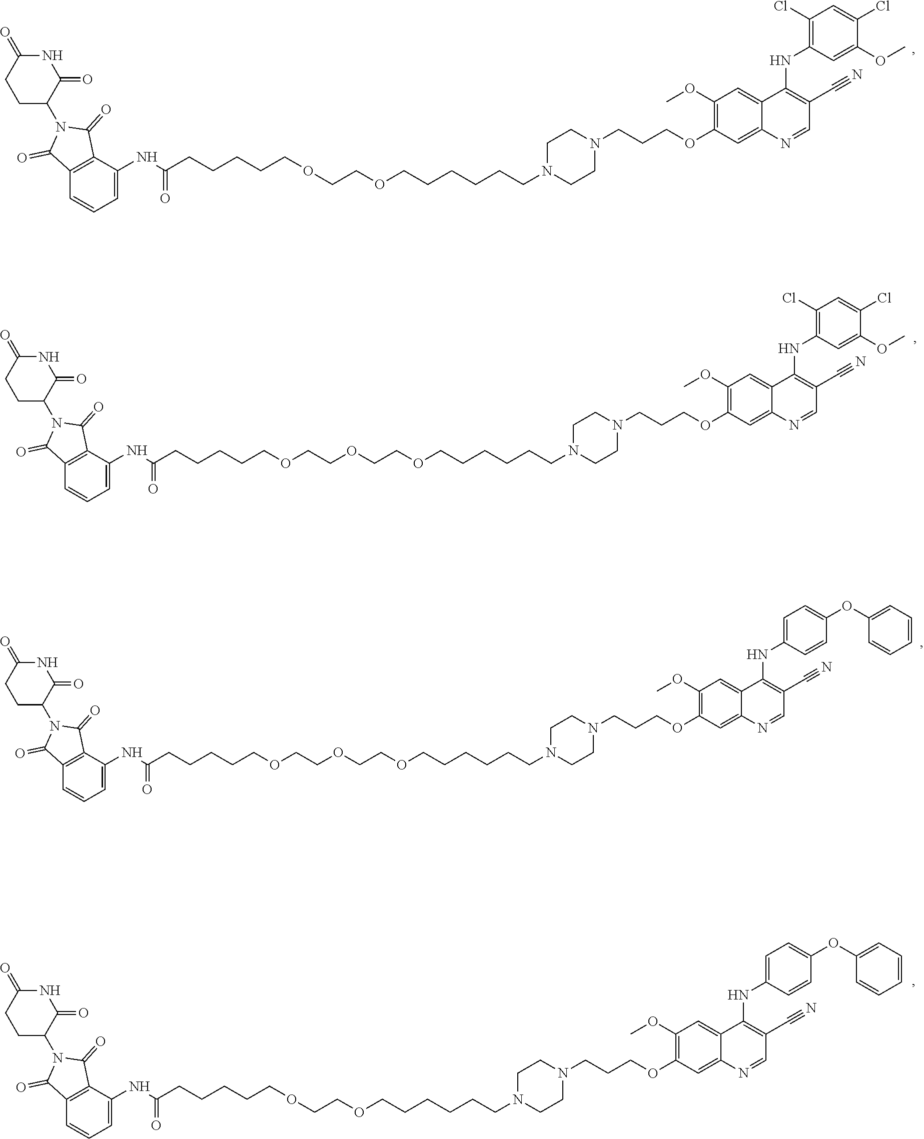 [SMILES: COc1cc(Nc2c(C#N)cnc3cc(OCCCN4CCN(CCCCCCOCCOCCCCCC(=O)Nc5cccc6c5C(=O)N(C5CCC(=O)NC5=O)C6=O)CC4)c(OC)cc23)c(Cl)cc1Cl ; COc1cc(Nc2c(C#N)cnc3cc(OCCCN4CCN(CCCCCCOCCOCCOCCCCCC(=O)Nc5cccc6c5C(=O)N(C5CCC(=O)NC5=O)C6=O)CC4)c(OC)cc23)c(Cl)cc1Cl ; COc1cc2c(Nc3ccc(Oc4ccccc4)cc3)c(C#N)cnc2cc1OCCCN1CCN(CCCCCCOCCOCCOCCCCCC(=O)Nc2cccc3c2C(=O)N(C2CCC(=O)NC2=O)C3=O)CC1 ; COc1cc2c(Nc3ccc(Oc4ccccc4)cc3)c(C#N)cnc2cc1OCCCN1CCN(CCCCCCOCCOCCCCCC(=O)Nc2cccc3c2C(=O)N(C2CCC(=O)NC2=O)C3=O)CC1]

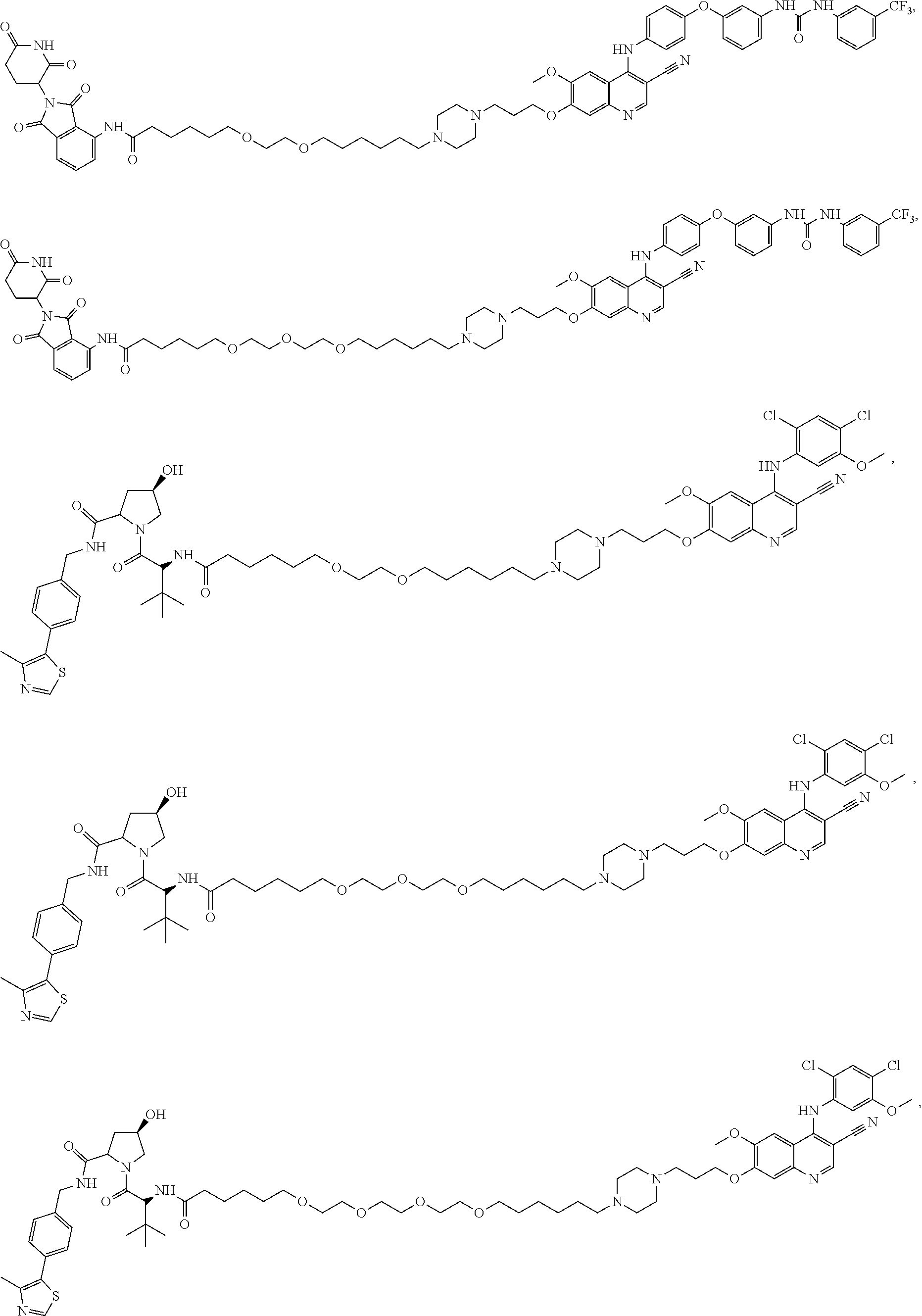

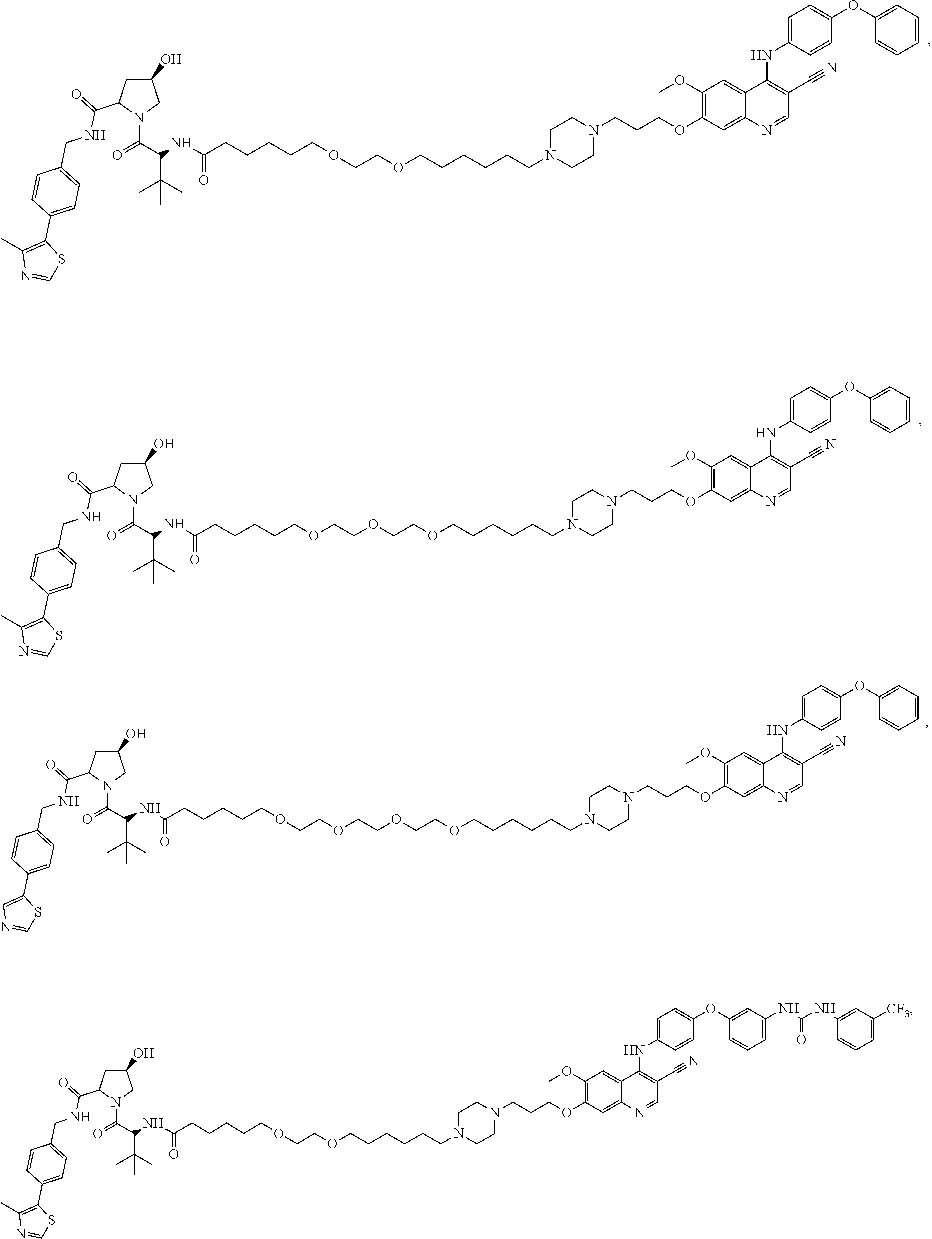

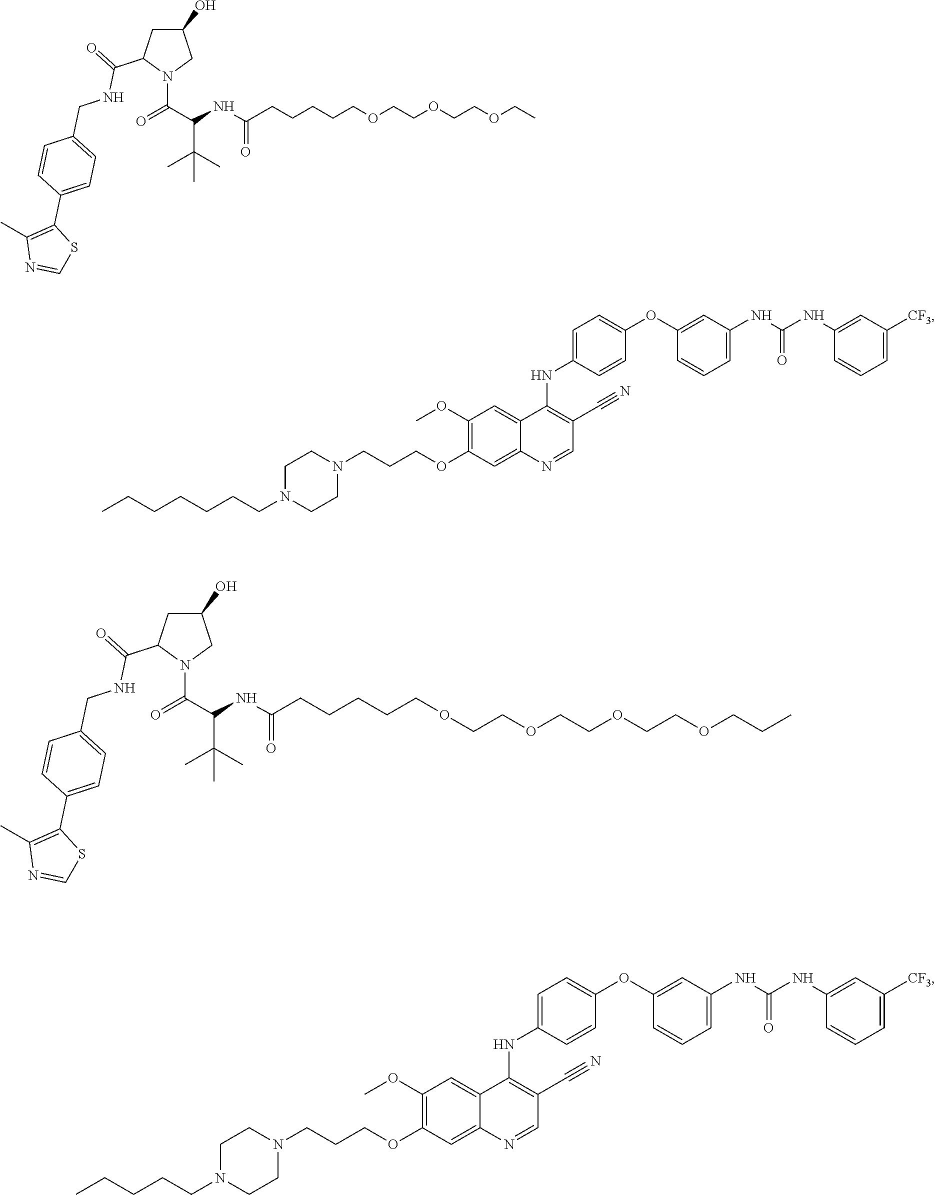

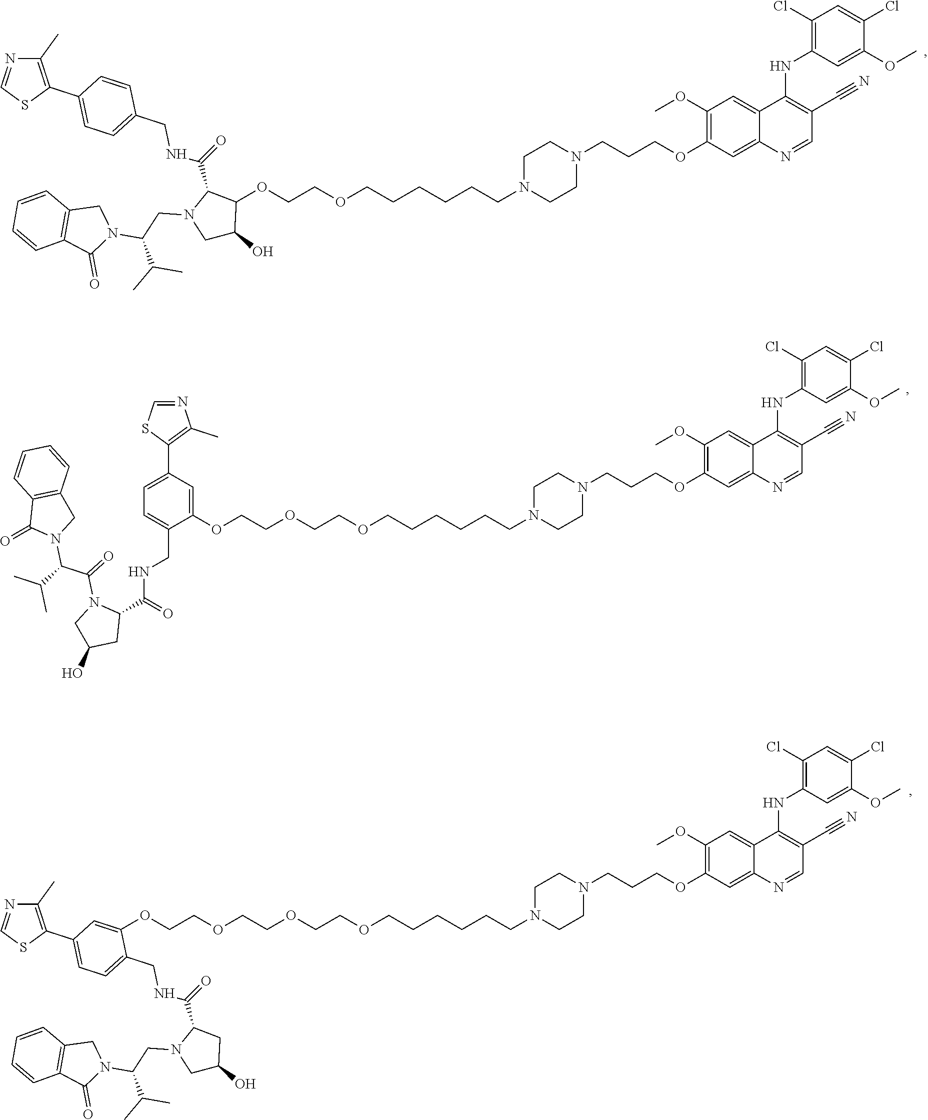 [SMILES: COc1cc(Nc2c(C#N)cnc3cc(OCCCN4CCN(CCCCCCOCCOC5[C@@H](C(=O)NCc6ccc(-c7scnc7C)cc6)N(C[C@H](C(C)C)N6Cc7ccccc7C6=O)C[C@@H]5O)CC4)c(OC)cc23)c(Cl)cc1Cl ; COc1cc(Nc2c(C#N)cnc3cc(OCCCN4CCN(CCCCCCOCCOCCOc5cc(-c6scnc6C)ccc5CNC(=O)[C@@H]5C[C@@H](O)CN5C(=O)[C@H](C(C)C)N5Cc6ccccc6C5=O)CC4)c(OC)cc23)c(Cl)cc1Cl ; COc1cc(Nc2c(C#N)cnc3cc(OCCCN4CCN(CCCCCCOCCOCCOCCOc5cc(-c6scnc6C)ccc5CNC(=O)[C@@H]5C[C@@H](O)CN5C[C@H](C(C)C)N5Cc6ccccc6C5=O)CC4)c(OC)cc23)c(Cl)cc1Cl]

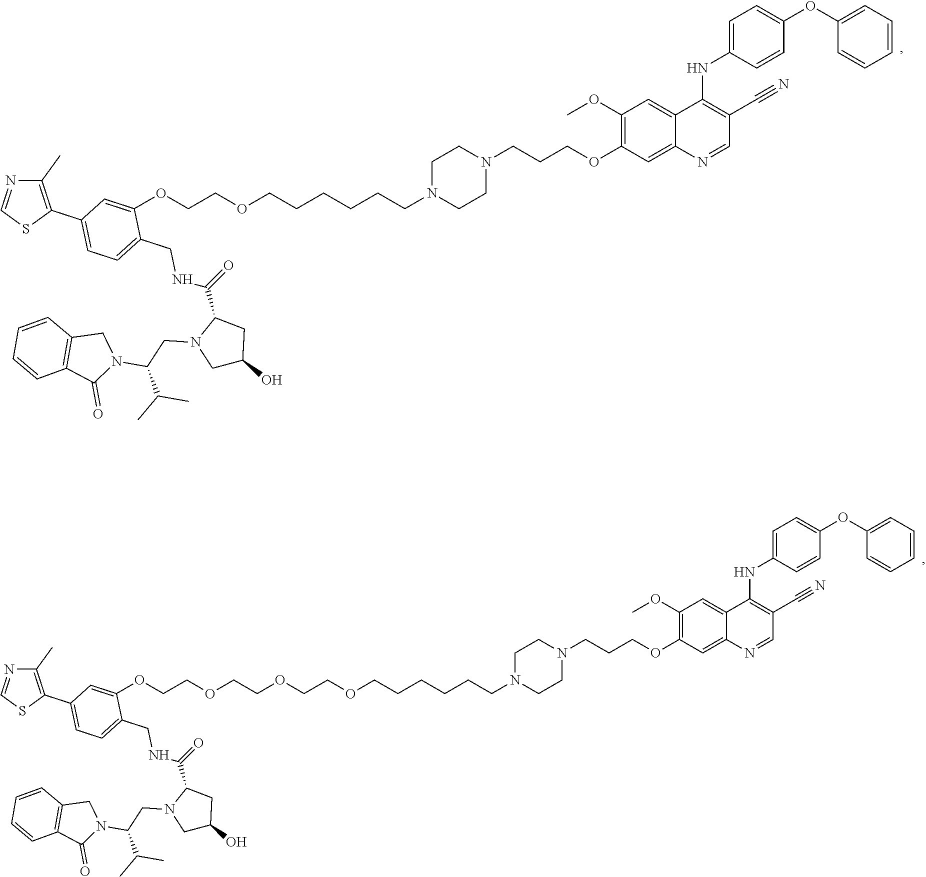 [SMILES: COc1cc2c(Nc3ccc(Oc4ccccc4)cc3)c(C#N)cnc2cc1OCCCN1CCN(CCCCCCOCCOc2cc(-c3scnc3C)ccc2CNC(=O)[C@@H]2C[C@@H](O)CN2C[C@H](C(C)C)N2Cc3ccccc3C2=O)CC1 ; COc1cc2c(Nc3ccc(Oc4ccccc4)cc3)c(C#N)cnc2cc1OCCCN1CCN(CCCCCCOCCOCCOCCOc2cc(-c3scnc3C)ccc2CNC(=O)[C@@H]2C[C@@H](O)CN2C[C@H](C(C)C)N2Cc3ccccc3C2=O)CC1]

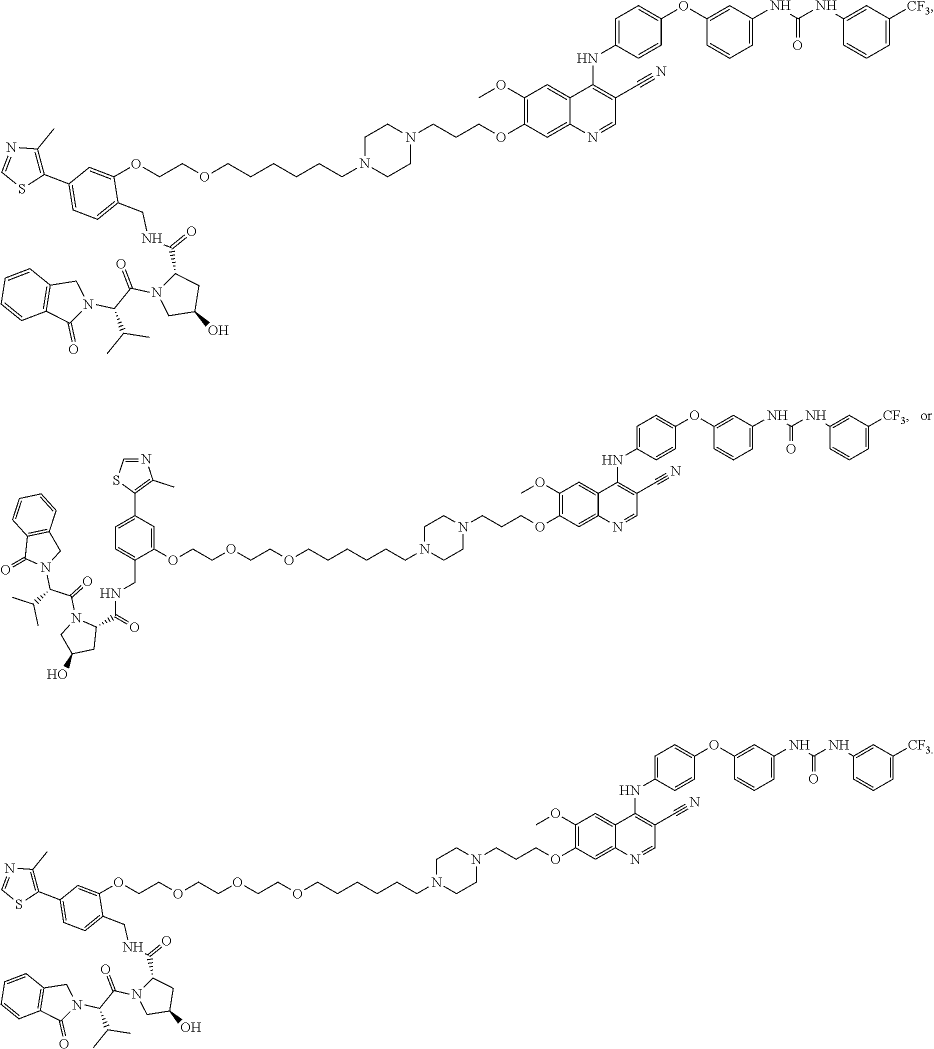

In embodiments, a compound is a compound described herein, including in an aspect, embodiment, table, figure, example, scheme, or claim.

In embodiments, a compound described herein, including a degradation-increasing moiety (e.g., R⁵) increases the degradation of a protein in contact with (e.g., bound to) the compound. In embodiments, the protein contacting the compound is HER protein. In embodiments, the protein contacting the compound is EGFR. In embodiments, the protein contacting the compound is HER2. In embodiments, the protein contacting the compound is HER3. In embodiments, the protein contacting the compound is HER4. In embodiments, a compound described herein, including a degradation-increasing moiety (e.g., R⁴ or R⁵) increases the degradation of a complex including a protein in contact with (e.g., bound to) the compound (e.g., EGFR, HER2, HER3, or HER4). In embodiments, the complex includes a second protein selected from EGFR, HER2, HER3, or HER4.

### C. Pharmaceutical Compositions

In another aspect is provided a pharmaceutical composition including a pharmaceutically acceptable excipient and a compound, or pharmaceutically acceptable salt thereof, as described herein, including embodiments.

In embodiments, the pharmaceutical compositions include the active ingredient (e.g., compound described herein or pharmaceutically acceptable salt thereof) in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., inhibiting cell proliferation. In embodiments, the pharmaceutical composition includes an anti-cancer agent. In embodiments, the anti-cancer agent is an EGFR modulator, HER2 modulator, HER4 modulator, c-MET modulator, PI3K modulator, MEK modulator, MAPK modulator, RAF modulator, BRAF modulator, AKT modulator, RAS modulator, KRAS modulator, heregulin modulator, neuregulin modulator, or mTOR modulator. In embodiments, the anti-cancer agent is lapatinib, vemurafenib, or selumetinib.

### D. Methods of Treatment

The compounds described herein are useful, inter alia, in methods of treating cancer. Such methods include administering to a subject in need thereof an effective amount of a compound described herein, including embodiments and pharmaceutically acceptable salts thereof. In embodiments, the cancer is lung cancer, non-small cell lung cancer, ovarian cancer, breast cancer, triple negative breast cancer, melanoma, head and neck cancer, colon cancer, gatric cancer, glioma, or glioblastoma.

In an aspect is provided, a method of treating cancer, wherein the method includes administering a compound described herein, or a pharmaceutically acceptable salt thereof, to a subject in need thereof. In embodiments, the cancer is lung cancer, non-small cell lung cancer, ovarian cancer, breast cancer, triple negative breast cancer, melanoma, head and neck cancer, colon cancer, gatric cancer, glioma, or glioblastoma.

In another aspect a compound described herein is provided for use as a medicament.

In another aspect is provided, a method of treating a disease associated with HER3 activity, wherein the method includes administering a compound described herein, or a pharmaceutically acceptable salt thereof, to a subject in need thereof.

In embodiments, the method includes administering a therapeutically effective amount of the compound.

In embodiments, the method includes administering a second agent (e.g., therapeutic agent). In embodiments, the second agent is an anti-cancer agent. In embodiments, the anti-cancer agent is an EGFR modulator, HER2 modulator, HER4 modulator, c-MET modulator, PI3K modulator, MEK modulator, MAPK modulator, RAF modulator, BRAF modulator, AKT modulator, RAS modulator, KRAS modulator, heregulin modulator, neuregulin modulator, or mTOR modulator. In embodiments, the anti-cancer agent is lapatinib, vemurafenib, or selumetinib. In embodiments, the proteins described above are human proteins.

In an aspect is provided a method of treating a disease associated with EGFR activity, HER2 activity, HER4 activity, c-MET activity, PI3K activity, MEK activity, MAPK activity, RAF activity, BRAF activity, AKT activity, RAS activity, KRAS activity, heregulin activity, or neuregulin activity in a patient in need of such treatment, the method including administering a therapeutically effective amount of a compound described herein, or a pharmaceutically acceptable salt thereof. In embodiments, the proteins described above are human proteins. In embodiments, the method includes contacting HER3 with a compound described herein (e.g., including a degradation-increasing moiety, for example as described herein). In embodiments, the method includes contacting HER3 with a compound described herein (e.g., including a degradation-increasing moiety, for example as described herein) and decreasing the level of EGFR activity, HER2 activity, HER4 activity, c-MET activity, PI3K activity, MEK activity, MAPK activity, RAF activity, BRAF activity, AKT activity, RAS activity, KRAS activity, heregulin activity, or neuregulin activity by inhibiting the activity (e.g., binding to a second protein) of HER3 or increasing the degradation of HER3.

### E. Methods of Inhibiting HER3

In an aspect is provided a method of inhibiting HER3 activity, the method including contacting HER3 with an effective amount of a compound described herein, or a pharmaceutically acceptable salt thereof.

In embodiments, the HER3 is a human HER3.

### F. Numbered Embodiments

Embodiment 1. A compound having the formula:

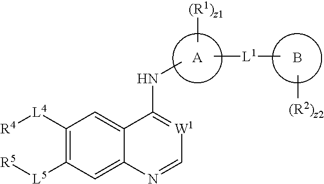

wherein:
Ring A is aryl or heteroaryl;
Ring B is aryl or heteroaryl;
W¹ is C(R⁶);
R¹ is independently a halogen, —CX¹₃, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is independently a halogen, —CX²₃, —CHX²₂, —CH₂X², —CN, —SOₙ₂R¹⁴, —SOᵥ₂NR¹¹R¹², —NHNH₂, —ONR¹¹R¹², —NHC═(O)NHNR¹¹R¹², —NHC═(O)NR¹¹R¹², —N(O)ₘ₂, —NR¹¹R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR¹¹OR¹³, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴₃, —OCHX⁴₂, —OCH₂X⁴₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is a degradation-increasing moiety;
R⁶ is hydrogen, halogen, —CX⁶₃, —CHX⁶₂, —CH₂X⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁶₃, —OCHX⁶₂, —OCH₂X⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷, R⁸, R⁹, R¹⁰, R¹¹, R¹², R¹³, and R¹⁴ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R¹¹ and R¹² substituents bonded to the same nitrogen atom may optionally be joined to form a substituted or unsubstituted heterocycloalkyl or substituted or unsubstituted heteroaryl;
L¹ is a bond, —O—, —NH—, —C(O)—, —C(O)NH—, —NHC(O)—, —C(O)O—, —OC(O)—, —NHC(O)NH—, —S—, substituted or unsubstituted alkylene or substituted or unsubstituted heteroalkylene;
L⁴ is a bond or a divalent linker;
L⁵ is a divalent linker;
z1 and z2 are independently an integer from 0 to 7;
m1, m2, v1, and v2 are independently 1 or 2;
n1 and n2 are independently an integer from 0 to 4; and
X¹, X², X⁴, X⁶, and X^{A} are independently —Cl, —Br, —I, or —F.

Embodiment 2. The compound of embodiment 1 having the formula:

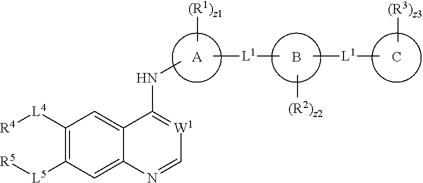

wherein
Ring A is phenyl or 5 or 6 membered heteroaryl;
Ring B is phenyl or 5 or 6 membered heteroaryl;
Ring C is C₃-C₆ cycloalkyl, 3 to 6 membered heterocycloalkyl, phenyl, or 5 to 6 membered heteroaryl;
R¹ is independently a halogen, —CX¹₃, —CHX¹₂, —CH₂X¹, —CN, —OH, —NH₂, —COOH, —CONH₂, —NO₂, —SH, substituted or unsubstituted C₁-C₄ alkyl, or 2 to 4 membered substituted or unsubstituted heteroalkyl;
R² is independently a halogen, —CX²₃, —CHX²₂, —CH₂X², —CN, —OH, —NH₂, —COOH, —CONH₂, —NO₂, —SH, substituted or unsubstituted C₁-C₄ alkyl, or 2 to 4 membered substituted or unsubstituted heteroalkyl;
R³ is independently a halogen, —CX³₃, —CHX³², —CH₂X³, —CN, —SOₙ₃R¹⁸, —SOᵥ₃NR¹⁵R¹⁶, —NHNH₂, —ONR¹⁵R¹⁶, —NHC═(O)NHNR¹⁵R¹⁶, —NHC═(O)NR¹⁵R¹⁶, —N(O)ₘ₃, —NR¹⁵R¹⁶, —C(O)R¹⁷, —C(O)—OR¹⁷, —C(O)NR¹⁵R¹⁶, —OR¹⁸, —NR¹⁵SO₂R¹⁷, —NR¹⁵C═(O)R¹⁷, —NR¹⁵C(O)—OR¹⁷, —NR¹⁵OR¹⁷, —OCX³₃, —OCHX³², —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl;
R⁶ is hydrogen, halogen, —CN, —OH, —NH₂, —COOH, —CONH₂, —NO₂, —SH, substituted or unsubstituted C₁-C₄ alkyl, or 2 to 4 membered substituted or unsubstituted heteroalkyl;
R¹⁵, R¹⁶, R¹⁷, and R¹⁸ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁵ and R¹⁶ substituents bonded to the same nitrogen atom may optionally be joined to form a substituted or unsubstituted heterocycloalkyl or substituted or unsubstituted heteroaryl;
L¹ is —O—, substituted or unsubstituted C₁-C₃ alkylene or substituted or unsubstituted 2 to 3 membered heteroalkylene;
L² is a bond, —O—, —NH—, —C(O)—, —C(O)NH—, —NHC(O)—, —C(O)O—, —OC(O)—, —NHC(O)NH—, —S—, substituted or unsubstituted C₁-C₃ alkylene or substituted or unsubstituted 2 to 3 membered heteroalkylene;
z1 and z2 are independently an integer from 0 to 4;
z3 is an integer from 0 to 5;
m3 and v3 are independently 1 or 2;
n3 is an integer from 0 to 4; and
X³ is —Cl, —Br, —I, or —F.

Embodiment 3. The compound of embodiment 2 having the formula:

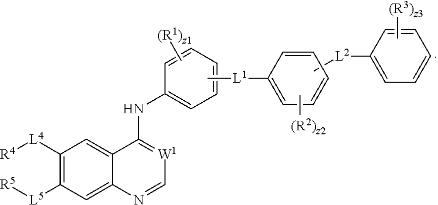

Embodiment 4. The compound of embodiment 3, having the formula:

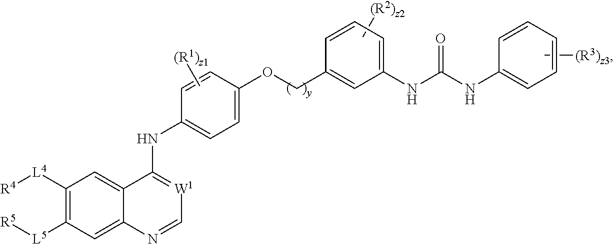

wherein Y is 0 or 1.

Embodiment 5. The compound of embodiment 3, having the formula:

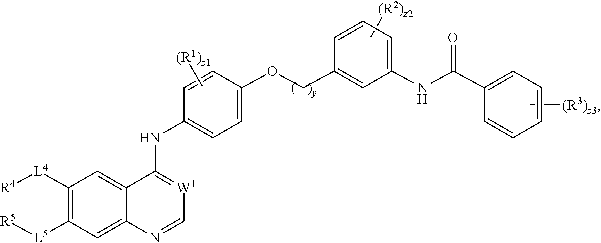

wherein Y is 0 or 1.

Embodiment 6. The compound of any one of embodiments 1 to 5, wherein z1 and z2 are 0, and z3 is 1.

Embodiment 7. The compound of any one of embodiments 2 to 5, wherein R³ is —CF₃ or halogen.

Embodiment 8. The compound of any one of embodiments 1 to 5, wherein R⁶ is —CN.

Embodiment 9. The compound of any one of embodiments 1 to 5, wherein -L⁴-R⁴ is unsubstituted methoxy.

Embodiment 10. The compound of any one of embodiments 1 to 9, wherein L⁵ is L^{5A}-L^{5B}-L^{5C}-L^{5D}-L^{5E}, and wherein L^{5A} L^{5B} L^{5C}, L^{5D}, and L^{5E} are each independently a bond, —O—, —NH—,a —C(O)—, —C(O)NH—, —NHC(O)—, —C(O)O—, —OC(O)—, —NHC(O)NH—, —S—, substituted or unsubstituted alkylene, substituted or unsubstituted heteroalkylene, substituted or unsubstituted, substituted or unsubstituted cycloalkylene, substituted or unsubstituted heterocycloalkylene, substituted or unsubstituted arylene or substituted or unsubstituted heteroarylene.

Embodiment 11. The compound of embodiment 10, wherein L^{5A} L^{5B} L^{5C}, L^{5D} and L^{5E} are each independently a bond, —O—, —NH—, —C(O)—, —C(O)NH—, —NHC(O)—, —C(O)O—, —OC(O)—, —NHC(O)NH—, —S—, substituted or unsubstituted C₁-C₃ alkylene, substituted or unsubstituted 2 to 4 membered heteroalkylene, substituted or unsubstituted C₃-C₆ cycloalkylene, substituted or unsubstituted 3 to 6 membered heterocycloalkylene, substituted or unsubstituted C₆ arylene, or substituted or unsubstituted 5 to 6 membered heteroarylene.

Embodiment 12. The compound of embodiment 11, wherein at least one of L^{5A} L^{5B}L^{5C}, L^{5D}, and L^{5E} is substituted or unsubstituted piperazinylene.

Embodiment 13. The compound of embodiment 12, wherein L^{5D} is substituted or unsubstituted piperazinylene.

Embodiment 14. The compound of any one of embodiments 10 to 13, wherein L^{5A} L^{5B} L^{5C}, and L^{5E} are each independently a bond, O, substituted or unsubstituted C₁-C₃ alkylene or substituted or unsubstituted 2 to 4 membered heteroalkylene.

Embodiment 15. The compound of any one of embodiments 10 to 14, wherein L^{5A} is a bond.

Embodiment 16. The compound of any one of embodiments 10 to 14, wherein L^{5B} is a bond or O.

Embodiment 17. The compound of any one of embodiments 10 to 14, wherein L^{5C} is a bond, O or substituted or unsubstituted 2 to 4 membered heteroalkylene.

Embodiment 18. The compound of any one of embodiments 10 to 17, wherein L^{5A} is a bond, L^{5B} is O, and L^{5C} is substituted or unsubstituted C₁-C₃ alkylene.

Embodiment 19. The compound of any one of embodiments 10 to 18, wherein L^{5E} is a bond, or substituted or unsubstituted C₁-C₃ alkylene.

Embodiment 20. The compound of embodiment 1, wherein R⁵ is substituted or unsubstituted heteroalkylene.

Embodiment 21. The compound of embodiment 20, wherein R⁵ is substituted or unsubstituted 2 to 22 membered heteroalkylene having a

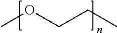

group, wherein n is 2 to 4.

Embodiment 22. The compound of embodiment 21, wherein R⁵ is substituted or unsubstituted 10 to 22 membered heteroalkylene.

Embodiment 23. The compound of embodiment 22, wherein R⁵ is substituted or unsubstituted 15 to 22 membered heteroalkylene.

Embodiment 24. The compound of any one of embodiments 1 to 23, wherein the compound is:

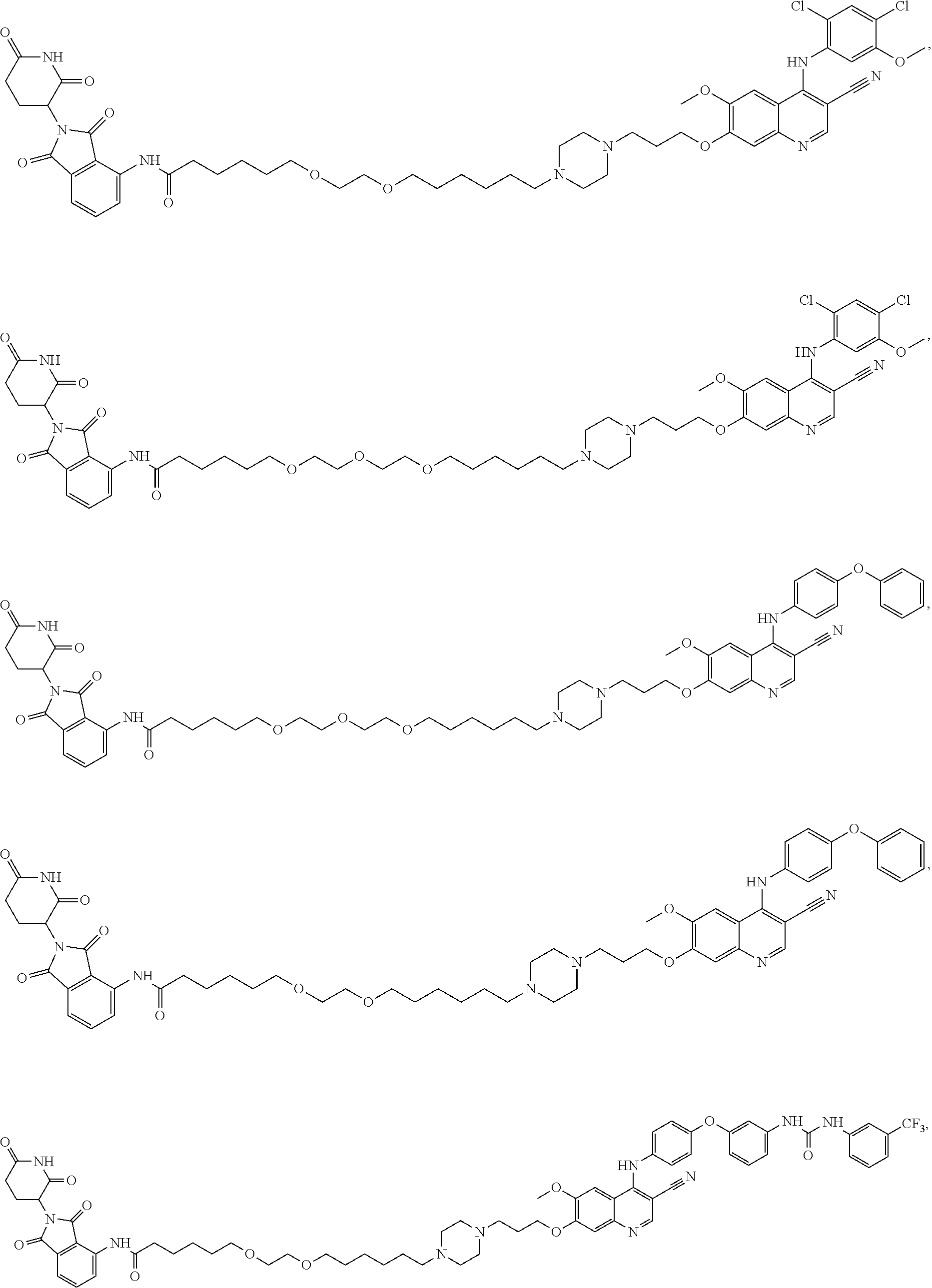

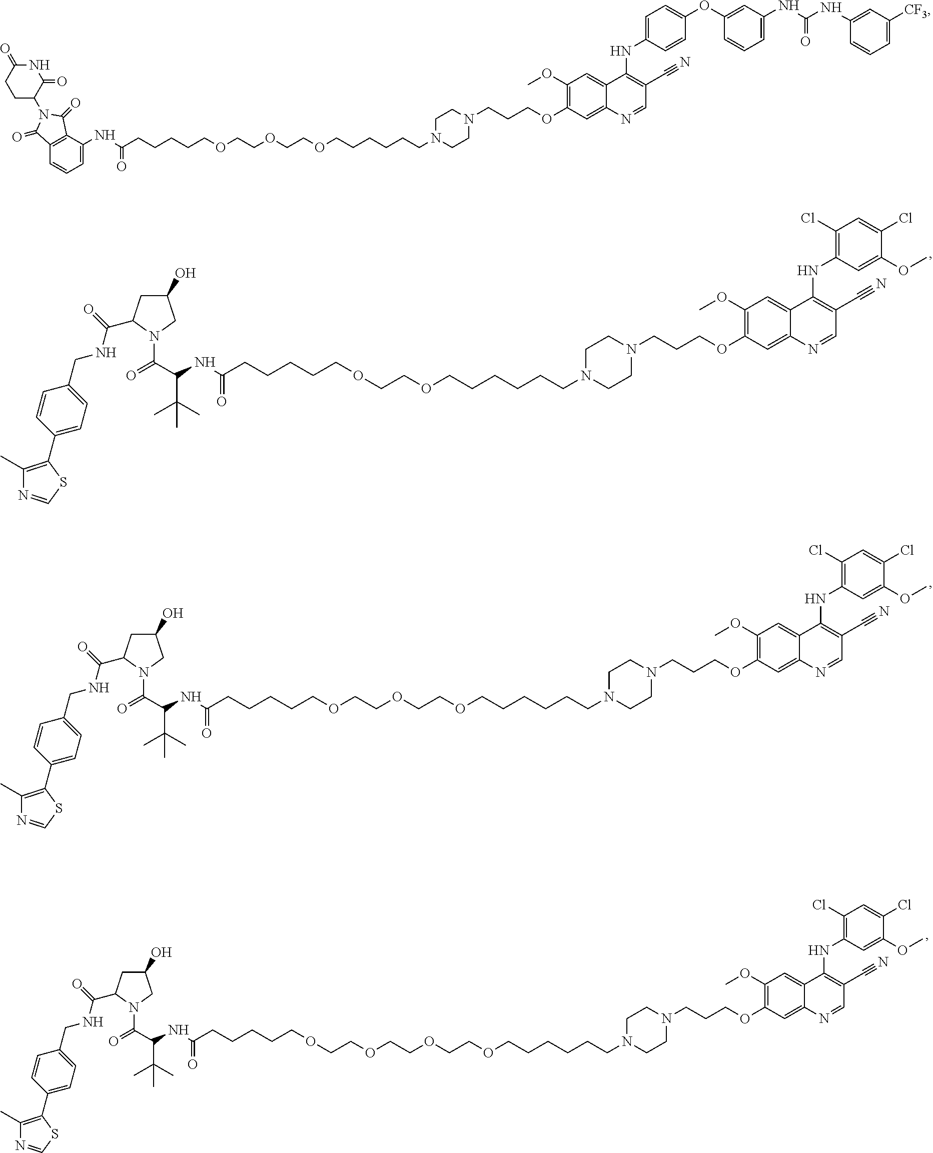

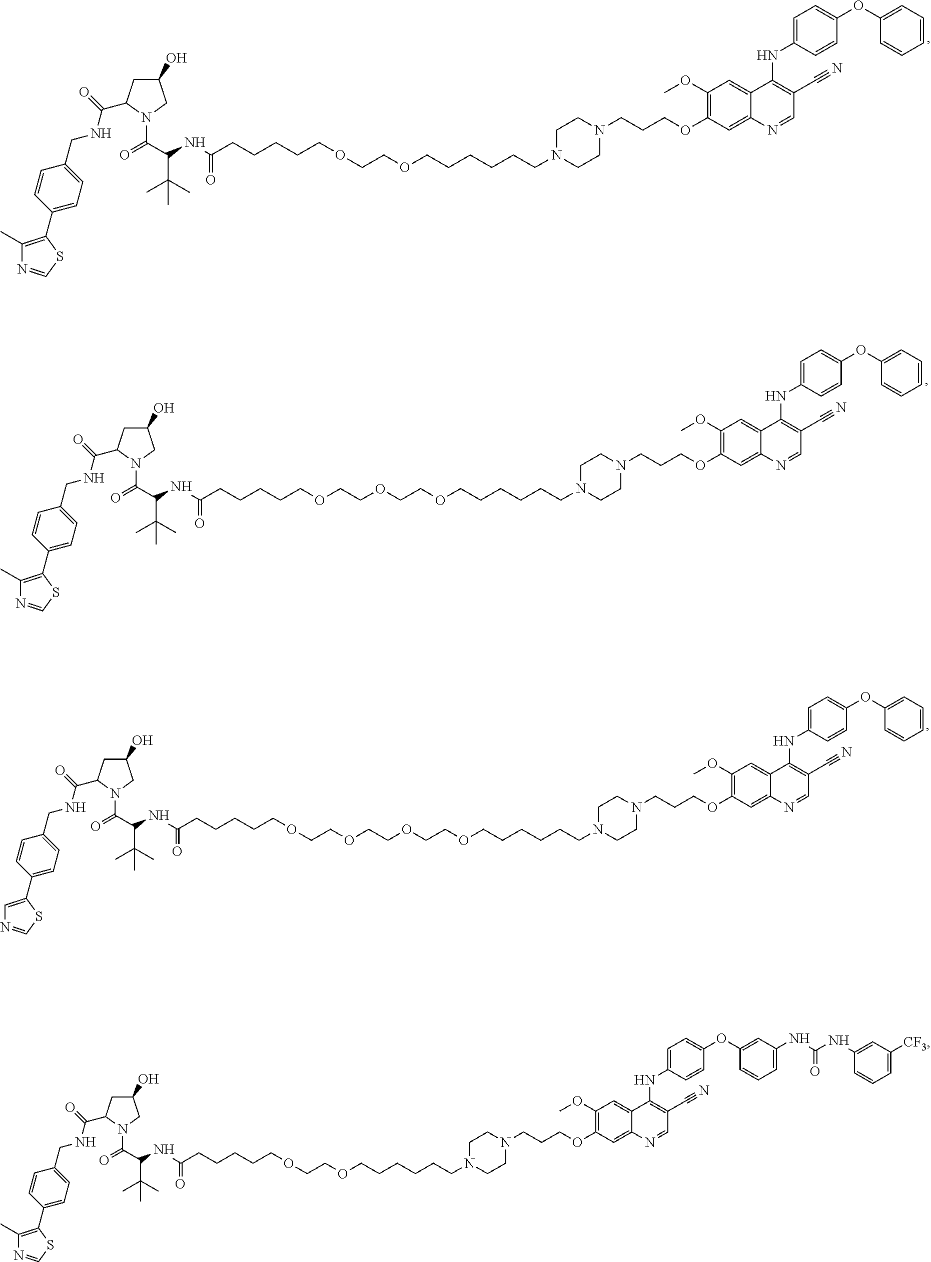

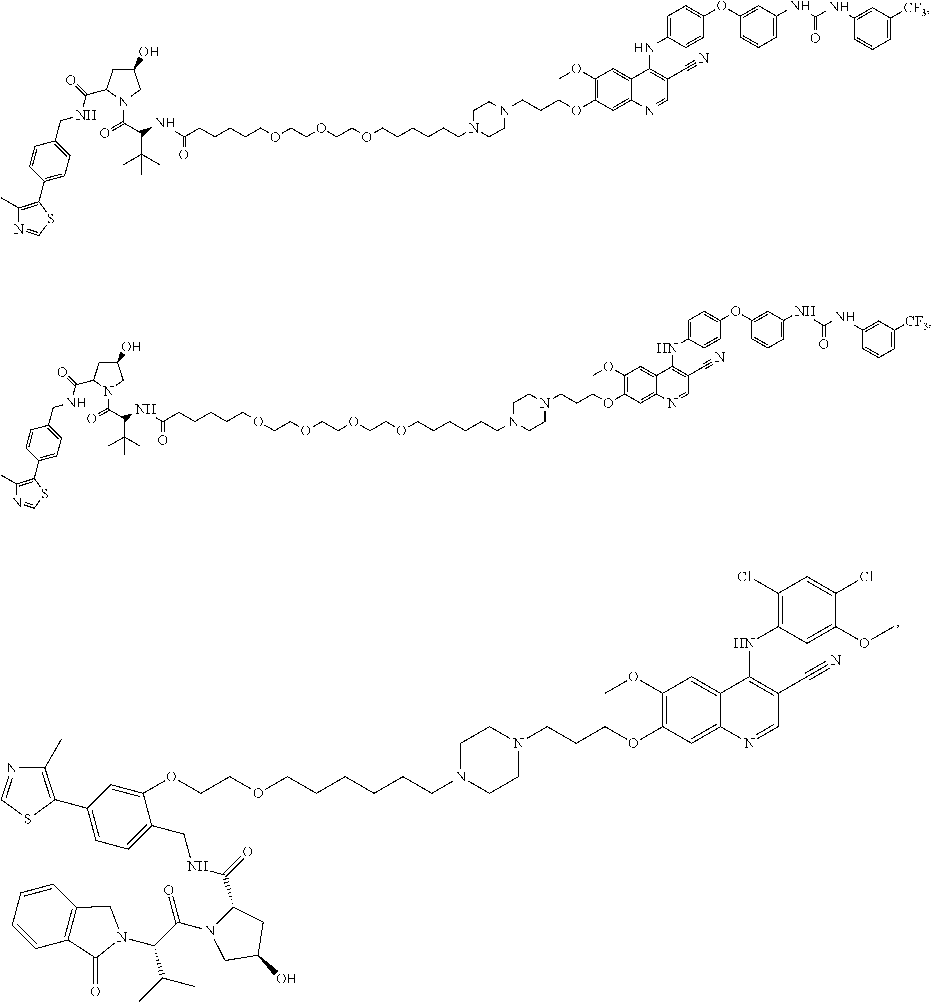

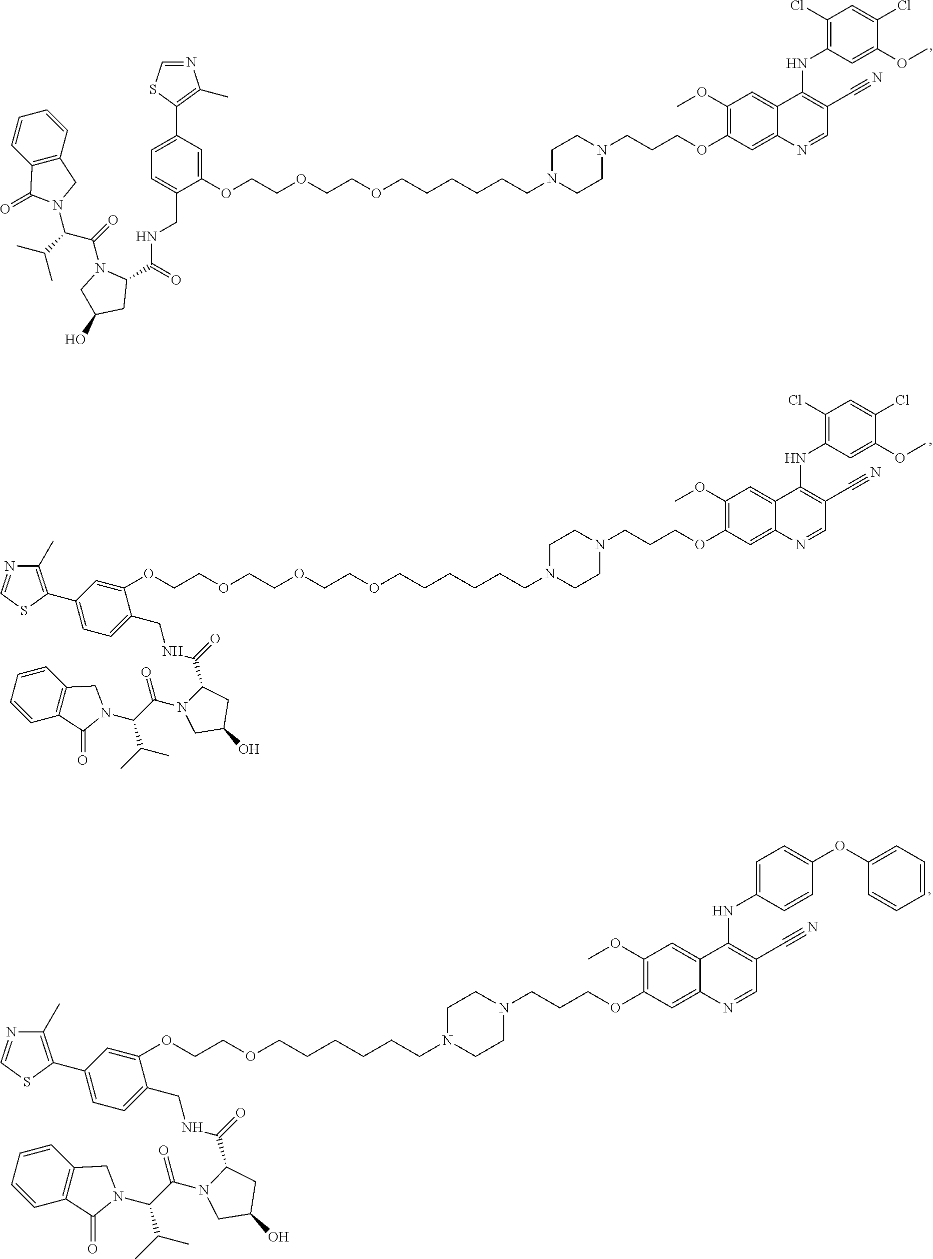 [SMILES: COc1cc(Nc2c(C#N)cnc3cc(OCCCN4CCN(CCCCCCOCCOCCOc5cc(-c6scnc6C)ccc5CNC(=O)[C@@H]5C[C@@H](O)CN5C(=O)[C@H](C(C)C)N5Cc6ccccc6C5=O)CC4)c(OC)cc23)c(Cl)cc1Cl ; COc1cc(Nc2c(C#N)cnc3cc(OCCCN4CCN(CCCCCCOCCOCCOCCOc5cc(-c6scnc6C)ccc5CNC(=O)[C@@H]5C[C@@H](O)CN5C(=O)[C@H](C(C)C)N5Cc6ccccc6C5=O)CC4)c(OC)cc23)c(Cl)cc1Cl ; COc1cc2c(Nc3ccc(Oc4ccccc4)cc3)c(C#N)cnc2cc1OCCCN1CCN(CCCCCCOCCOc2cc(-c3scnc3C)ccc2CNC(=O)[C@@H]2C[C@@H](O)CN2C(=O)[C@H](C(C)C)N2Cc3ccccc3C2=O)CC1]

Embodiment 25. A pharmaceutical composition comprising a compound of any one of embodiments 1 to 24, or a pharmaceutically acceptable salt thereof, and a pharmaceutically acceptable excipient.

Embodiment 26. A method of treating a disease associated with HER3 activity in a patient in need of such treatment, said method comprising administering a therapeutically effective amount of the compound of any one of embodiments 1 to 24, or a pharmaceutically acceptable salt thereof.

Embodiment 27. A method of treating a disease associated with EGFR activity, HER2 activity, HER4 activity, c-MET activity, PI3K activity, MEK activity, MAPK activity, RAF activity, BRAF activity, AKT activity, RAS activity, KRAS activity, heregulin activity, or neuregulin activity in a patient in need of such treatment, said method comprising administering a therapeutically effective amount of the compound of any one of embodiments 1 to 24, or a pharmaceutically acceptable salt thereof.

Embodiment 28. A method of treating cancer in a patient in need of such treatment, said method comprising administering a therapeutically effective amount of the compound of any one of embodiments 1 to 24, or a pharmaceutically acceptable salt thereof.

Embodiment 29. A method of inhibiting HER3 activity, said method comprising contacting HER3 with an effective amount of the compound of any one of embodiments 1 to 24, or a pharmaceutically acceptable salt thereof.

### G. EXAMPLES

HER3 Thermofluor Assay. Wt HER3 TKD was purified according to the previously published protocol¹. Thermofluor reactions were performed in duplicate and set up as follows. 1 μL of an inhibitor or DMSO dilution in 40% DMSO:water was added to 19 μL of the HER3 kinase domain in reaction buffer. The final reaction solution contained 100 mM MOPS, 200 mM NaCl, 5% glycerol, 5 mM MgCl2, 0.1 mM DTT, 5× SYPRO Orange, 2 μM kinase, 2% DMSO and 20 μM inhibitor in the wells of a 96-well, low profile, white, PCR plate (USA scientific). The solution was pipetted up and down to mix, sealed with TempAssure clear PCR flat caps (USA Scientific), centrifuged at 500×g for 30 s, and heated in a Stratagene Mx3005P RT-PCR machine from 25° C. to 95° C. in 0.5° C. increments every 30 s after an initial incubation at 25° C. for 10 min. Fluorescence was measured at the end of each 30 s period with an excitation wavelength of 492 nm and an emission wavelength of 610 nm. To obtain the melting temperature, fluorescent signals were normalized to the maximum fluorescent signal for that well. Values after the well had reached a maximum signal were discarded and the signals were fit to the Boltzmann equation in Graphpad Prism 6. ΔTₘ was calculated as the difference in melting temperature between the compound-treated kinase compared to the DMSO control.

In vitro Kinase Assays. In vitro kinase assays with the HER2 kinase domain (SignalChem) were performed in triplicate as follows. To 9 μL of a 2.5× solution of kinase and substrate in reaction buffer was added 3 μL of a 5× DMSO or inhibitor dilution in 10% DMSO:water. The inhibitor/kinase solution was incubated at room temperature for 10 minutes. The kinase assay was initiated by the addition of 3 μL of a 5× solution of ATP, and ran for 15 minutes. The final reaction conditions were 50 mM Tris (pH7.4), 5 mM MnCl₂, 0.01% Tween-20, 2 mM DTT, 100 μM E₄Y substrate (SignalChem), 15 nM HER2, 2% DMSO, 50 μM ATP, and 1 μCi γ³²P-ATP. After 15 minutes, 3 μL of each reaction was pipetted onto phosphocellulose sheets (P81, Whatman) and allowed to dry. The sheets were then washed 4×5 min with a solution of 0.5% phosphoric acid, dried, and exposed to a phosphor screen overnight. Src Kinase assays were conducted as previously described². Phosphorimaging was conducted on a Typhoon 9500, image intensities were quantified in ImageQuant 5.2, normalized to the DMSO control and plotted in GraphPad Prism 6.

Immunoblotting. CHLL-1 cells were grown in 6-well plates and treated according to the indicated conditions at which point the media was aspirated, cells were washed with 1 mL of cold PBS, which was then aspirated and the plates were frozen at −80° C. The frozen cells were thawed on the plates in a buffer containing 50 mM Tris pH 7.5, 150 mM NaCl, 1 mM EDTA, and 1% Triton X-100 supplemented with 1× phosphatase (PhoSTOP, Roche) and 1× protease (complete-mini tablets, Roche) inhibitors. Lysates were scraped, transferred to Eppendorf tubes, and cleared by centrifugation at 20,000×g for 20 min at 4° C. The clarified lysates were transferred to chilled, clean tubes, and normalized for protein concentration by Bradford (Bio-Rad). The normalized lysates were diluted with Laemmli loading buffer, and 10 μg of total protein was run on a 4-12% gradient gel (Invitrogen), which was then transferred to 0.45 μM nitrocellulose (Bio-Rad) and analyzed using the indicated primary antibodies according to the manufacturer's recommendations (1:1000 antibody dilution). Primary antibodies were detected using IRDye secondary antibodies (Li-Cor) according to the manufacturer's recommendations and scanned on an Odyssey imager (Li-Cor). Scanned images were cropped and assembled in Adobe Illustrator 6.

### Chemical Synthesis

General Methods: Reactions were performed in sealed vials with magnetic stirring. All commercial reagents were used without further purification. Silica gel chromatography was performed on a Combiflash Rf+ using column cartridges pre-packed with 40-60 micron silica (Teledyne Isco). All RP-HPLC purifications were performed with a Waters 2545 binary gradient module equipped with an XBridge prep C₁₈ column using H2O+0.1% formic acid and CH3CN+0.1% formic acid (5-95% gradient) while monitoring at 254 nm. Low resolution mass spectra (LC/ESI-MS) were recorded in positive and negative mode on a Waters TQ detector with an Acquity UPLC equipped with a BEH C18 column.

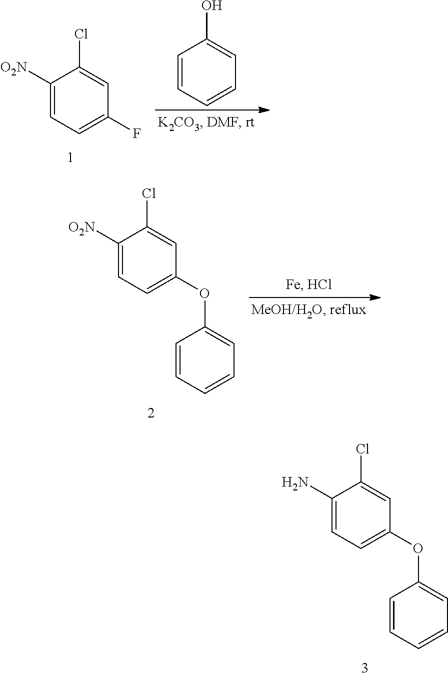 [SMILES: O=[N+]([O-])c1ccc(F)cc1Cl ; Oc1ccccc1 ; O=[N+]([O-])c1ccc(Oc2ccccc2)cc1Cl ; Nc1ccc(Oc2ccccc2)cc1Cl]

2-chloro-4-phenoxyaniline (3). 5.25 g of 1 was added to a vial with a stir bar. Potassium carbonate and phenol were added in 1.5 and 1.2 eq respectively to the reaction. The vial was capped, purged with argon, and 10 mL of dry dimethylformamide was added. The solution was allowed to stir at room temperature overnight. Compound 2 was obtained in 76% yield after silica column purification and used in the next step. Compound 2 was reduced using diluted hydrochloride chloride acid with iron in a MeOH/H2O solvent mixture to obtain 3 in 65% yield after silica column purification.

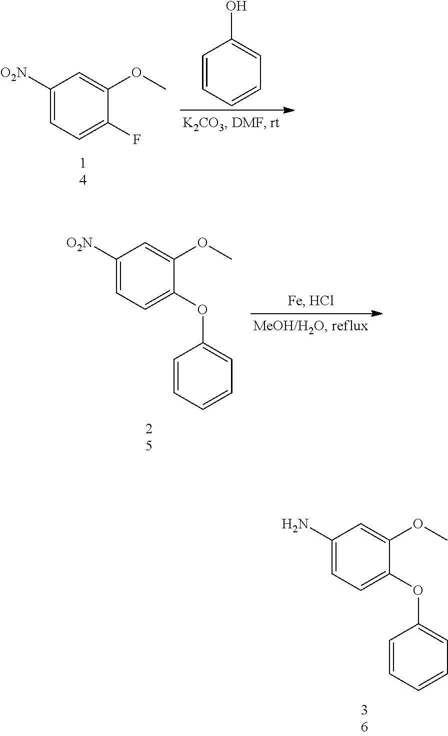

3-methoxy-4-phenoxyaniline (6). 2.50 g of 4 was added to a vial with a stir bar. Potassium carbonate and phenol were added in 1.5 and 1.2 eq respectively to the reaction. The vial was capped, purged with argon, and 5 mL of dry dimethylformamide was added. The solution was allowed to stir at room temperature overnight. Compound 2 was obtained in 65% yield after silica column purification and used in the next step. Compound 5 was reduced using diluted hydrochloride chloride acid with iron in a MeOH/H2O solvent mixture to obtain 6 in 63% yield after silica column purification.

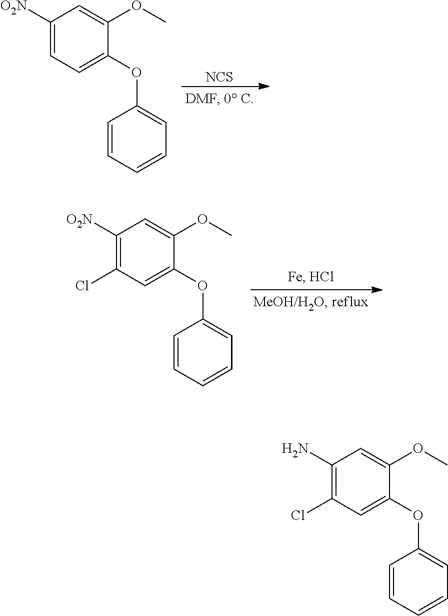 [SMILES: COc1cc([N+](=O)[O-])ccc1Oc1ccccc1 ; "NCS" ; COc1cc([N+](=O)[O-])c(Cl)cc1Oc1ccccc1 ; COc1cc(N)c(Cl)cc1Oc1ccccc1]

2-chloro-3-methoxy-4-phenoxyaniline (8). 1.01 g of 5 was added to a vial with a stir bar. The vial was capped, purged with argon, and 1.5 mL of dry dimethylformamide was added. The reaction was N-chlorosuccinimide was added in 1.1 eq to the reaction. The reaction was stirred at 0° C. for 5 min and N-chlorosuccinimide was added in 1.1 eq. Compound 7 was obtained in 65% yield after silica column purification and used in the next step. Compound 7 was reduced using diluted hydrochloride chloride acid with iron in a MeOH/H2O solvent mixture to obtain 8 in 67% yield after silica column purification.

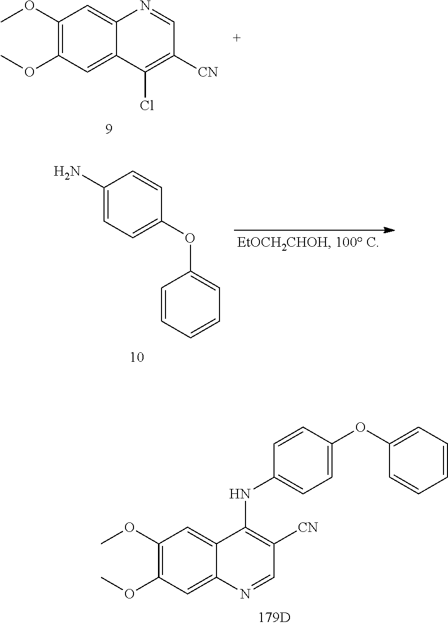 [SMILES: COc1cc2ncc(C#N)c(Cl)c2cc1OC ; Nc1ccc(Oc2ccccc2)cc1 ; COc1cc2ncc(C#N)c(Nc3ccc(Oc4ccccc4)cc3)c2cc1OC]

6,7-dimethoxy-4-((4-phenoxyphenyl) amino) quinoline-3-carbonitrile (179D). 10 mg of 9 and 7.46 mg of 4-phenoxyaniline (10) was added to a vial with a magnetic stir bar. The vial was capped, purged with argon, and 1 mL of 2-ethoxyethanol was added. The vial was heated at 150° C. for 3 h. The reaction was then cooled and purified by RP-HPLC. Product containing fractions were pooled and dried under reduced pressure to yield 6.4 mg (40%) of 179D as a yellow solid. MS (ES+) m/z 398.3 (M+H)⁺[0348]

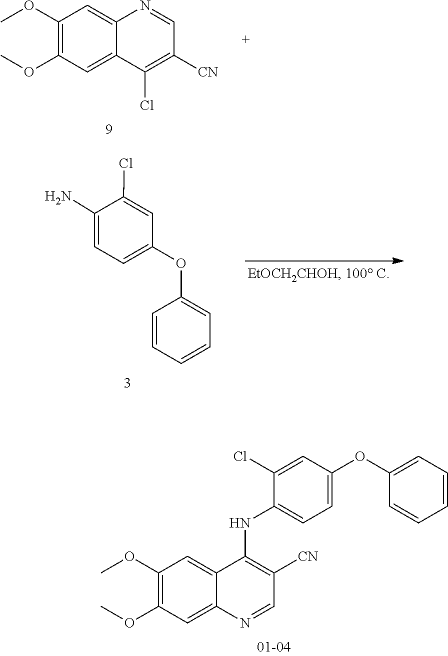 [SMILES: COc1cc2ncc(C#N)c(Cl)c2cc1OC ; Nc1ccc(Oc2ccccc2)cc1Cl ; COc1cc2ncc(C#N)c(Nc3ccc(Oc4ccccc4)cc3Cl)c2cc1OC]

4-(2-chloro-4-phenoxyanilino)-6,7-dimethoxyquinoline-3-carbonitrile (01-04). 10 mg of 9 and 7.46 mg of 4-phenoxyaniline (3) was added to a vial with a magnetic stir bar. The vial was capped, purged with argon, and 1 mL of 2-ethoxyethanol was added. The vial was heated at 150° C. for 3 h. The reaction was then cooled and purified by RP-HPLC. Product containing fractions were pooled and dried under reduced pressure to yield 6.4 mg (69%) of 01-04 as a yellow solid. MS (ES+) m/z 432.7 (M+H)⁺

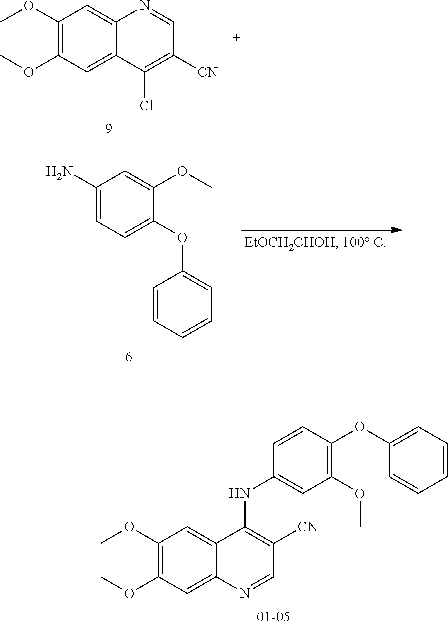 [SMILES: COc1cc2ncc(C#N)c(Cl)c2cc1OC ; COc1cc(N)ccc1Oc1ccccc1 ; COc1cc2ncc(C#N)c(Nc3ccc(Oc4ccccc4)c(OC)c3)c2cc1OC]

4-(3-methoxy-4-phenoxyanilino)-6,7-dimethoxyquinoline-3-carbonitrile (01-05). 10 mg of 9 and 7.46 mg of 4-phenoxyaniline (6) was added to a vial with a magnetic stir bar. The vial was capped, purged with argon, and 1 mL of 2-ethoxyethanol was added. The vial was heated at 150° C. for 3 h. The reaction was then cooled and purified by RP-HPLC. Product containing fractions were pooled and dried under reduced pressure to yield 6.4 mg (69%) of 01-05 as a yellow solid. MS (ES+) m/z 432.7 (M+H)⁺

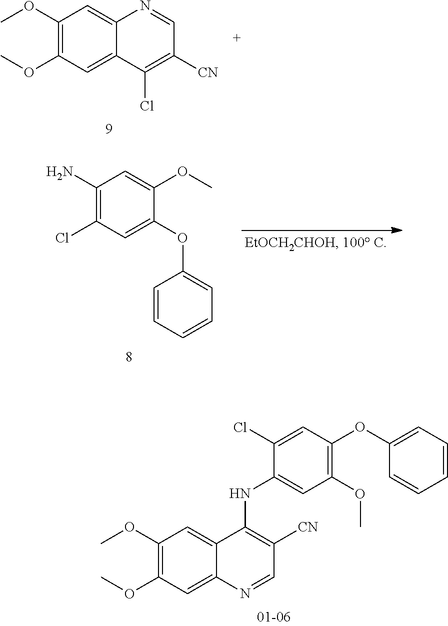 [SMILES: COc1cc2ncc(C#N)c(Cl)c2cc1OC ; COc1cc(N)c(Cl)cc1Oc1ccccc1 ; COc1cc2ncc(C#N)c(Nc3cc(OC)c(Oc4ccccc4)cc3Cl)c2cc1OC]

4-(2-chloro-3-methoxy-4-phenoxyanilino)-6,7-dimethoxyquinoline-3-carbonitrile (01-06). 125 mg of 9 and 1.1 eq of 2-chloro-3-methoxy-4-phenoxyaniline (8) was added to a vial with a magnetic stir bar. The vial was capped, purged with argon, and 10 mL of 2-ethoxyethanol was added. The vial was heated at 150° C. for 3 h. The reaction was then cooled and purified by RP-HPLC. Product containing fractions were pooled and dried under reduced pressure to yield 6.4 mg (48.5%) of 01-06 as a yellow solid. MS (ES+) m/z 462.7 (M+H)⁺

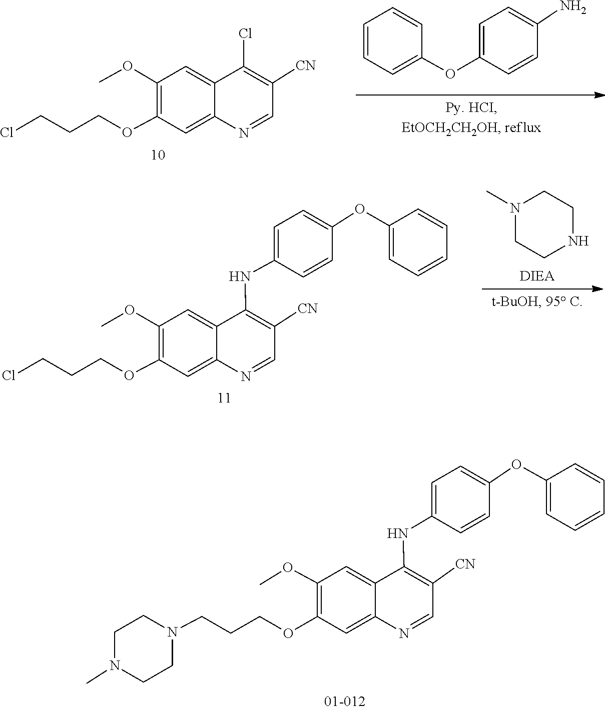

4-(4-phenoxyanilino)-4-chloro-6-methoxy-7-[3-(4-methylpiperazin-1-yl) propoxy]quinoline-3-carbonitrile (01-012): 250 mg of 10 and 1.1 eq of 4-phenoxyaniline and pyridine hydrochloride was added to a vial with a magnetic stir bar. The vial was capped, purged with argon, and 10 mL of 2-ethoxyethanol added. The vial was heated at 150° C. for 3 h. The reaction was then cooled and purified by RP-HPLC. Product containing fractions were pooled and dried under reduced pressure to yield 6.4 mg (48.5%) of 11 and used in the next step. Compound 01-012 was obtained and purified by RP-HPLC, after reaction of compound 11 with methyl piperazine in presence of N,N-Diisopropylethylamine (DIEA) in tert-Butyl alcohol at 95° C. in 67% yield. MS (ES+) m/z 524.5 (M+H)⁺

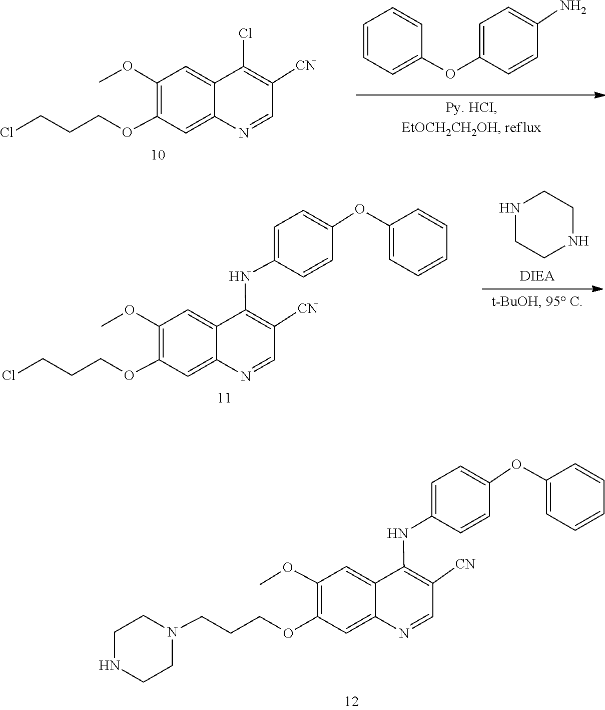 [SMILES: COc1cc2c(Cl)c(C#N)cnc2cc1OCCCCl ; Nc1ccc(Oc2ccccc2)cc1 ; COc1cc2c(Nc3ccc(Oc4ccccc4)cc3)c(C#N)cnc2cc1OCCCCl ; C1CNCCN1 ; COc1cc2c(Nc3ccc(Oc4ccccc4)cc3)c(C#N)cnc2cc1OCCCN1CCNCC1]

4-(4-phenoxyanilino)-4-chloro-6-methoxy-7-[3-(piperazin-1-yl) propoxy]quinoline-3-carbonitrile (12): 250 mg of 10 and 1.1 eq of 4-phenoxyaniline and pyridine hydrochloride was added to a vial with a magnetic stir bar. The vial was capped, purged with argon, and 10 mL of 2-ethoxyethanol added. The vial was heated at 150° C. for 3 h. The reaction was then cooled and purified by RP-HPLC. Product containing fractions were pooled and dried under reduced pressure to yield 48.5% of 11 and used in the next step. Compound 01-012 was obtained and purified by RP-HPLC, after reaction of compound 11 with piperazine in presence of N,N-Diisopropylethylamine (DIEA) in tert-Butyl alcohol at 95° C. in 67% yield. MS (ES+) m/z 524.5 (M+H)⁺

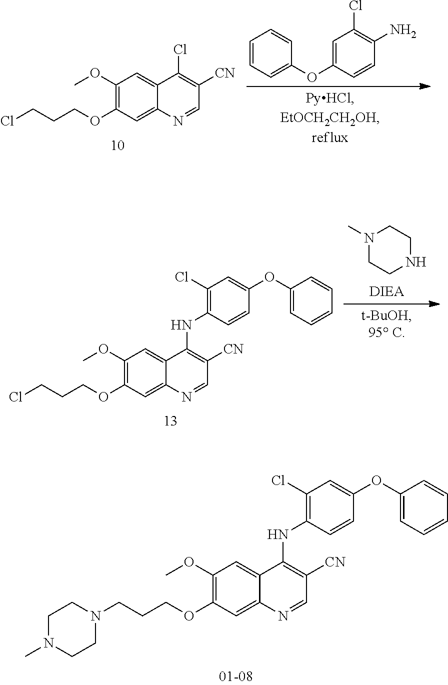

4-(2-chloro-4-phenoxyanilino)-6-methoxy-7-[3-(4-methylpiperazin-1-yl) propoxy]quinoline-3-carbonitrile (01-08): 250 mg of 10 and 1.1 eq of 2-chloro-4-phenoxyaniline and pyridine hydrochloride was added to a vial with a magnetic stir bar. The vial was capped, purged with argon, and 10 mL of 2-ethoxyethanol added. The vial was heated at 150° C. for 3 h. The reaction was then cooled and purified by RP-HPLC. Product containing fractions were pooled and dried under reduced pressure to yield 49% of 13 and used in the next step. Compound 01-08 was obtained and purified by RP-HPLC, after reaction of compound 13 with methyl piperazine in presence of N,N-Diisopropylethylamine (DIEA) in tert-Butyl alcohol at 95° C. in 68% yield. MS (ES+) m/z 559.0 (M+H)⁺

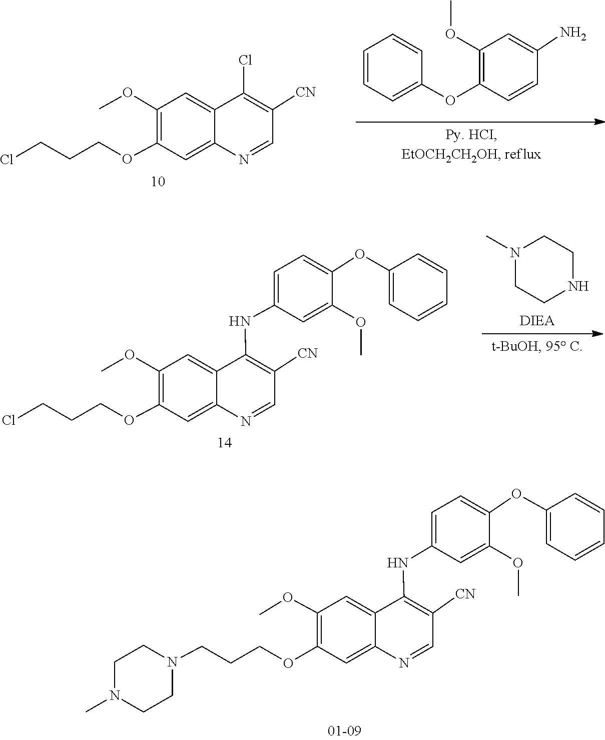

4-(3-methoxy-4-phenoxyanilino)-6-methoxy-7-[3-(4-methylpiperazin-1-yl) propoxy]quinoline-3-carbonitrile (01-09): 250 mg of 10 and 1.1 eq of 3-methoxy-4-phenoxyaniline and pyridine hydrochloride was added to a vial with a magnetic stir bar. The vial was capped, purged with argon, and 10 mL of 2-ethoxyethanol added. The vial was heated at 150° C. for 3 h. The reaction was then cooled and purified by RP-HPLC. Product containing fractions were pooled and dried under reduced pressure to yield 510% of 14 and used in the next step. Compound 01-09 was obtained and purified by RP-HPLC, after reaction of compound 14 with methyl piperazine in presence of N,N-Diisopropylethylamine (DIEA) in tert-Butyl alcohol at 95° C. in 71% yield. MS (ES+) m/z 554.5 (M+H)⁺

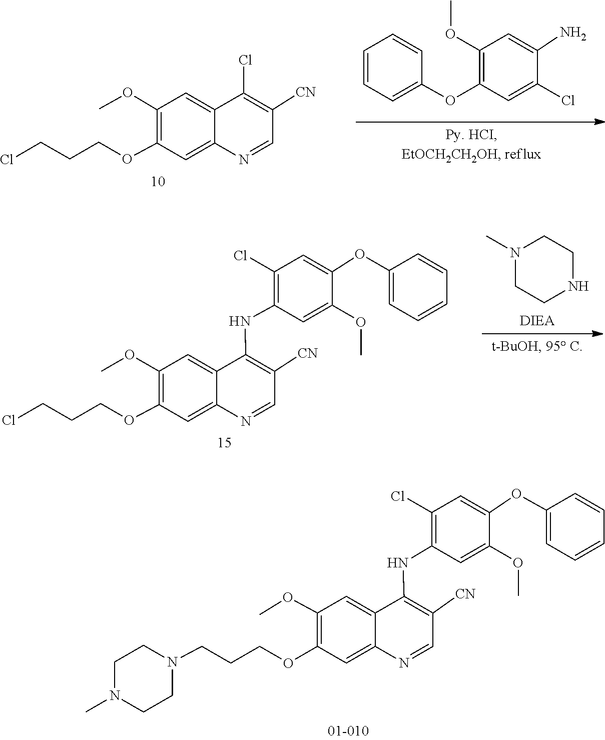

4-(2-chloro-3-methoxy-4-phenoxyanilino)-6-methoxy-7-[3-(4-methylpiperazin-1-yl) propoxy]quinoline-3-carbonitrile (01-010): 250 mg of 10 and 1.1 eq of 2-chloro-3-methoxy-4-phenoxyaniline and pyridine hydrochloride was added to a vial with a magnetic stir bar. The vial was capped, purged with argon, and 10 mL of 2-ethoxyethanol added. The vial was heated at 150° C. for 3 h. The reaction was then cooled and purified by RP-HPLC. Product containing fractions were pooled and dried under reduced pressure to yield 49% of 15 and used in the next step. Compound 01-010 was obtained and purified by RP-HPLC, after reaction of compound 15 with methyl piperazine in presence of N,N-Diisopropylethylamine (DIEA) in tert-Butyl alcohol at 95° C. in 71% yield. MS (ES+) m/z 589 (M+H)⁺

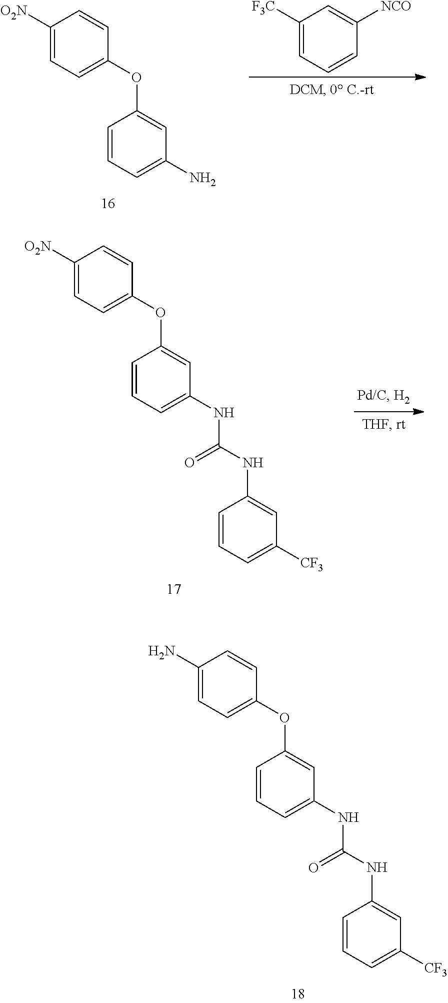

N-[3-(4-aminophenoxy)phenyl]-N′-[3-(trifluoromethyl)phenyl]urea (18): 50 mg of 16 was added to a vial with a stir bar. The vial was capped, purged with argon, and 1 mL of dry dichloromethane was added. The solution was allowed to stir at 0° C. for 15 minutes after which 31 μL of 3-(Trifluoromethyl)phenyl isocyanate was added. The reaction was stirred at 0° C. for 5 min and was then allowed to warm to 25° C. over the course of an hour. The solution was then cooled to −20° C. and filtered to yield 69.3 mg of 17 as a crude solid, which was used in the next step without further purification. 44.8 mg of the crude 17 was added to a vial containing 1 mL of THF and a stir bar. 6.0 mg of 10% palladium on carbon was added, the vial was capped and stirred at 25° C. under a hydrogen atmosphere for 14 h. The reaction was filtered, concentrated in vacuo and purified by silica gel chromatography (eluent, 20% EtOAc/Hex to 100% EtOAc) to give 34.4 mg (63% over 2 steps) of 18 as a white solid. MS (ES+) m/z 388.3 (M+H)⁺¹

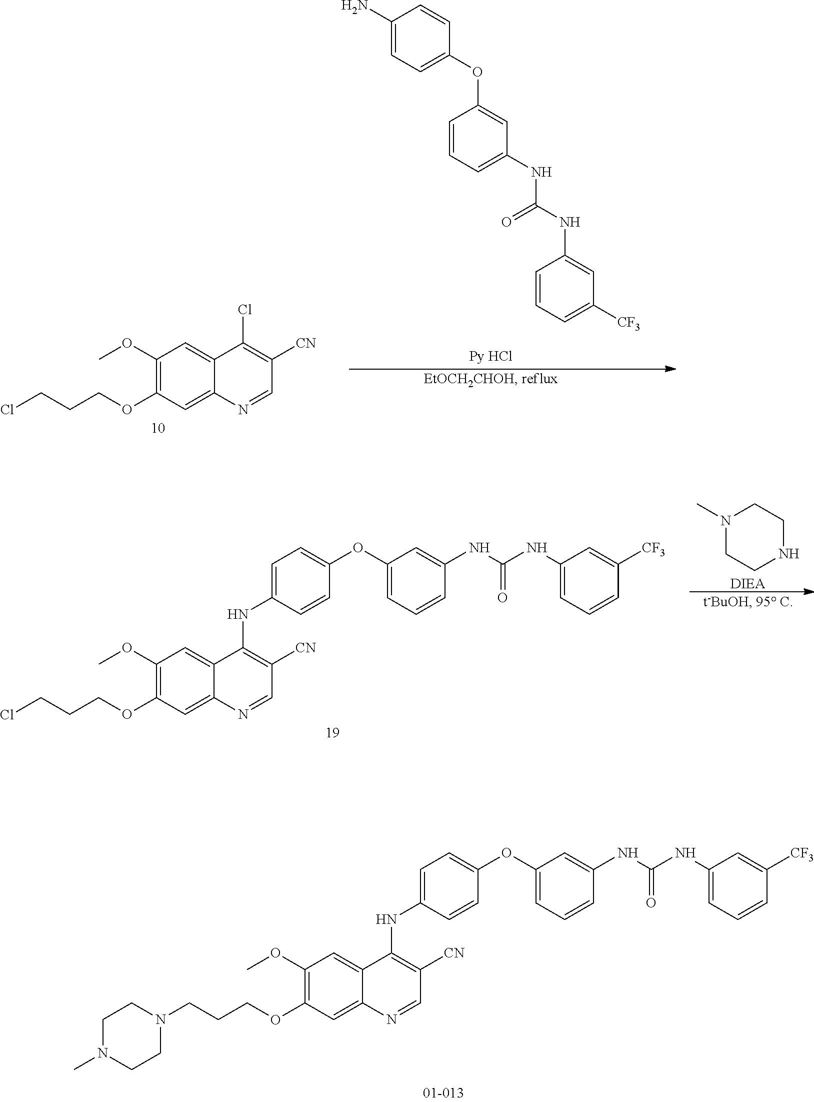

4-[N-(4-aminophenoxy)phenyl]-N′-[3-(trifluoromethyl)phenyl]urea-6-methoxy-7-[3-(4-methylpiperazin-1-yl) propoxy]quinoline-3-carbonitrile (01-013): 250 mg of 10 and 1.1 eq of N-[3-(4-aminophenoxy)phenyl]-N′-[3-(trifluoromethyl)phenyl]urea (18) and pyridine hydrochloride was added to a vial with a magnetic stir bar. The vial was capped, purged with argon, and 10 mL of 2-ethoxyethanol added. The vial was heated at 150° C. for 3 h. The reaction was then cooled and purified by RP-HPLC. Product containing fractions were pooled and dried under reduced pressure to yield 49% of 19 and used in the next step. Compound 01-013 was obtained and purified by RP-HPLC, after reaction of compound 19 with methyl piperazine in presence of N,N-Diisopropylethylamine (DIEA) in tert-Butyl alcohol at 95° C. in 71 % yield. MS (ES+) m/z 726.7 (M+H)⁺

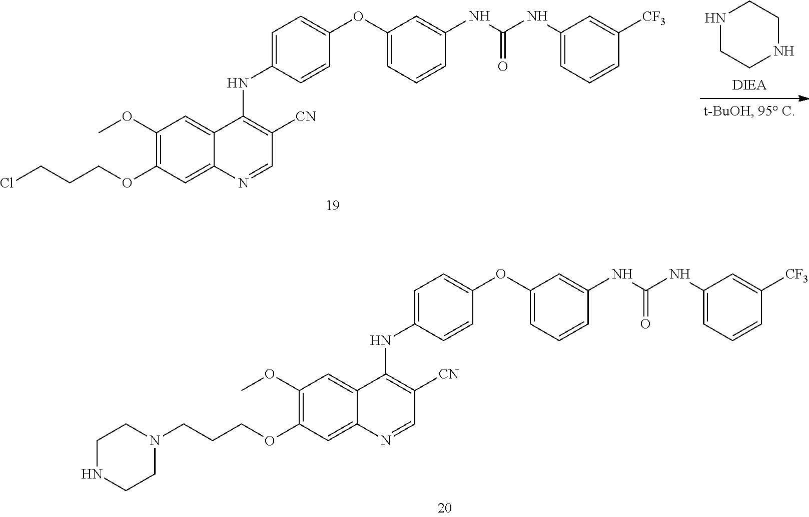 [SMILES: COc1cc2c(Nc3ccc(Oc4cccc(NC(=O)Nc5cccc(C(F)(F)F)c5)c4)cc3)c(C#N)cnc2cc1OCCCCl ; C1CNCCN1 ; COc1cc2c(Nc3ccc(Oc4cccc(NC(=O)Nc5cccc(C(F)(F)F)c5)c4)cc3)c(C#N)cnc2cc1OCCCN1CCNCC1]

4-[N-(4-aminophenoxy)phenyl]-N′-[3-(trifluoromethyl)phenyl]urea-6-methoxy-7-[3-(piperazin-1-yl) propoxy]quinoline-3-carbonitrile (20): 250 mg of 19 and 1.1 eq of piperazine in presence of N,N-Diisopropylethylamine (DIEA). The vial was capped, purged with argon, and 10 mL of tert-Butyl alcohol added. The vial was heated at 95° C. for 5 h. The reaction was then cooled and purified by RP-HPLC. Product containing fractions were pooled and dried under reduced pressure to yield 92% of 20. MS (ES+) m/z 712.7 (M+H)⁺

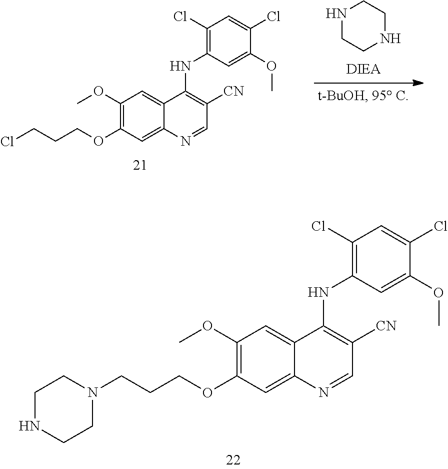 [SMILES: COc1cc(Nc2c(C#N)cnc3cc(OCCCCl)c(OC)cc23)c(Cl)cc1Cl ; C1CNCCN1 ; COc1cc(Nc2c(C#N)cnc3cc(OCCCN4CCNCC4)c(OC)cc23)c(Cl)cc1Cl]

7-[3-(piperazin-1-yl)propoxy]-4-(2,4-dichloro-5-methoxyanilino)-6-methoxyquinoline-3-carbonitrile (22): 250 mg of 21 and 1.1 eq of piperazine in presence of N,N-Diisopropylethylamine (DIEA). The vial was capped, purged with argon, and 10 mL of tert-Butyl alcohol added. The vial was heated at 95° C. for 5 h. The reaction was then cooled and purified by RP-HPLC. Product containing fractions were pooled and dried under reduced pressure to yield 90% of 22. MS (ES+) m/z 517.4 (M+H)⁺

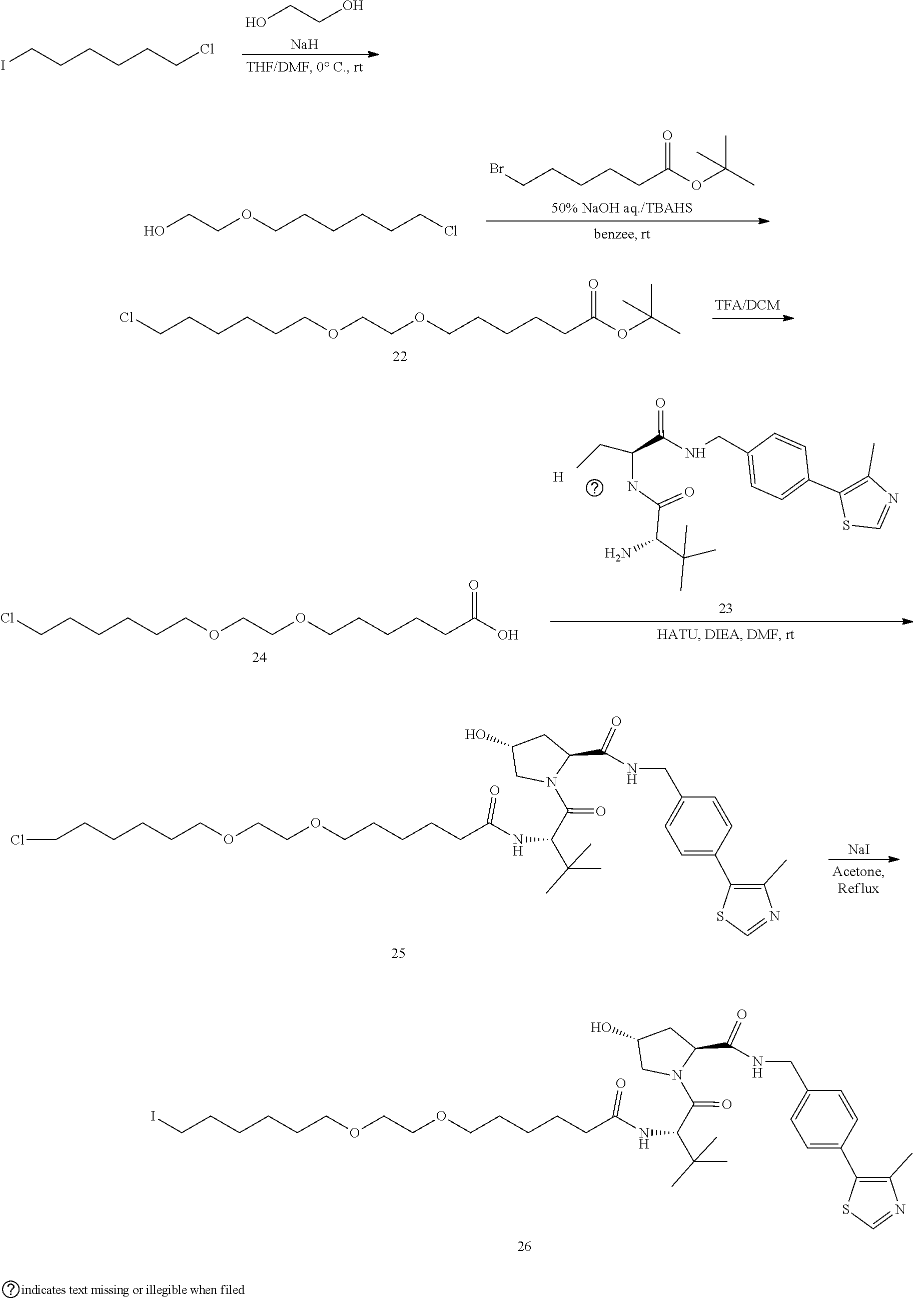

Intermediated 27: 10 g of 1-chloro-6-iodohexane (22) and 1 eq of NaH were added to a vial with a stir bar. The vial was capped, purged with argon, and 200 mL of a mix of dry tetrahydrofuran and dimethylformamide was added. The solution was allowed to stir at 0° C. for 15 minutes after which 1.1 eq. of ethylene glycol was added. The reaction was stirred at 0° C. for 5 min and was then allowed to warm to 25° C. over the course of an hour. The solution was then cooled to −20° C. and filtered to yield 11 g of 23 as a crude solid, which was used in the next step without further purification. 11 g of the crude 23 was added to a vial containing tert-butyl 6-bromohexanoate and 100 mL of benzene and 50% aqueous solution of NaOH and a stir bar. Tetra butyl ammonium hydrogen sulfate (TBAHS) was added, and the vial was capped and stirred at 25° C. under an inert atmosphere for 14 h. The reaction was filtered, concentrated in vacuo to give 5 g of the crude 24. 5 g of the crude 24 were mixed in dry dichloromethane with a 1.1 eq of trifluoroacetic acid to obtain 5 g of the crude 25. 1 g of compound 25 was conjugated with von Hippel-Lindau (VHL) recruiting ligand ((S, R, S)-AHPC) using 1-[Bis(dimethylamino)methylene]-1H-1,2,3-triazolo[4,5-b]pyridinium 3-oxid hexafluorophosphate (HATU), and diisopropylethylamine (DIEA) in dry dimethylformamide (DMF) to obtain 780 mg of compound 26. 780 mg of compound 26 and 1.1 eq of sodium iodide were added to a vial with a stir bar. The vial was capped, purged with argon, and 15 mL of dry acetone was added. The reaction was stirred at room temperature for 5 min and was then refluxed by 5 hours to give 850 mg of 27.

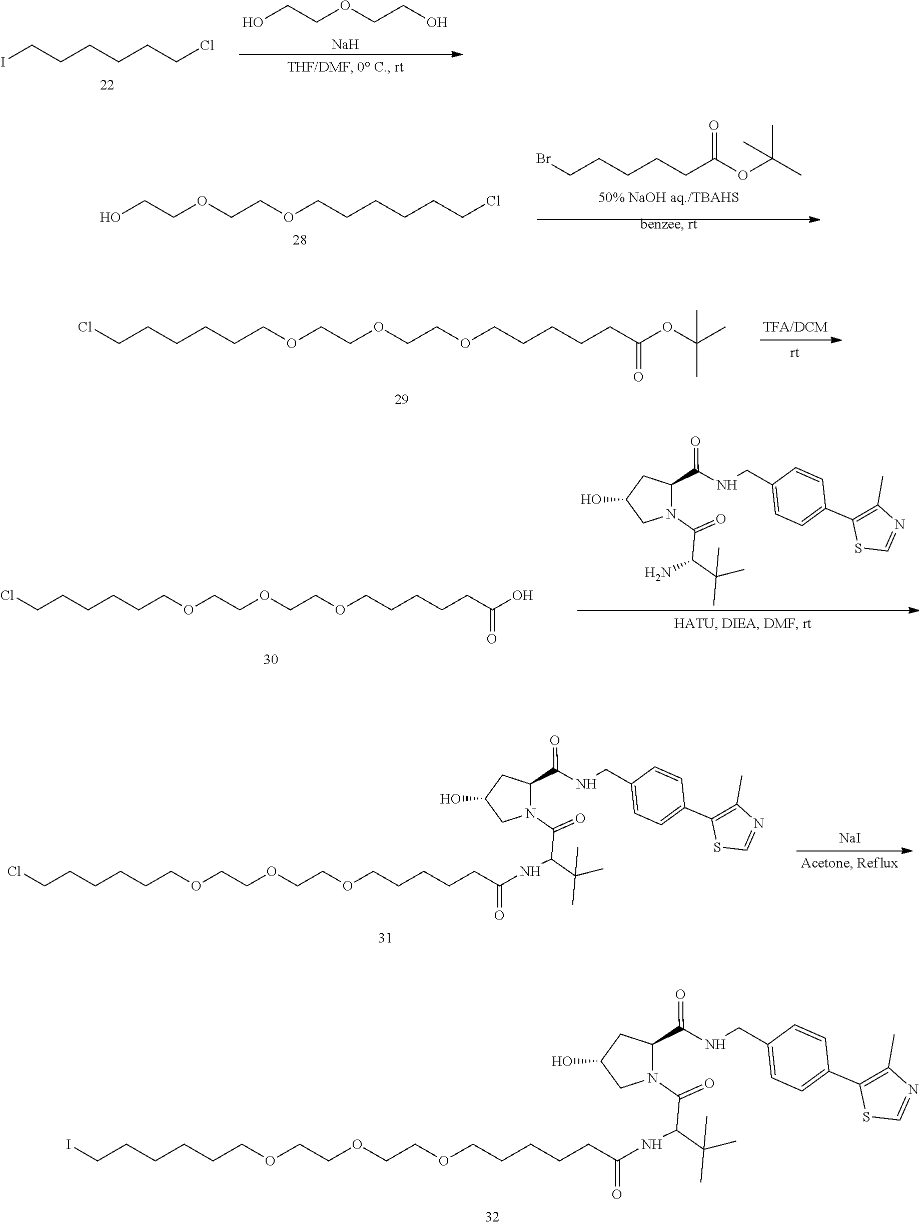

Intermediate 32: 320 mg of 32 was obtained by a method similar to the one described for 27 using diethylene glycol instead ethylene glycol in the first synthetic step.[0376]

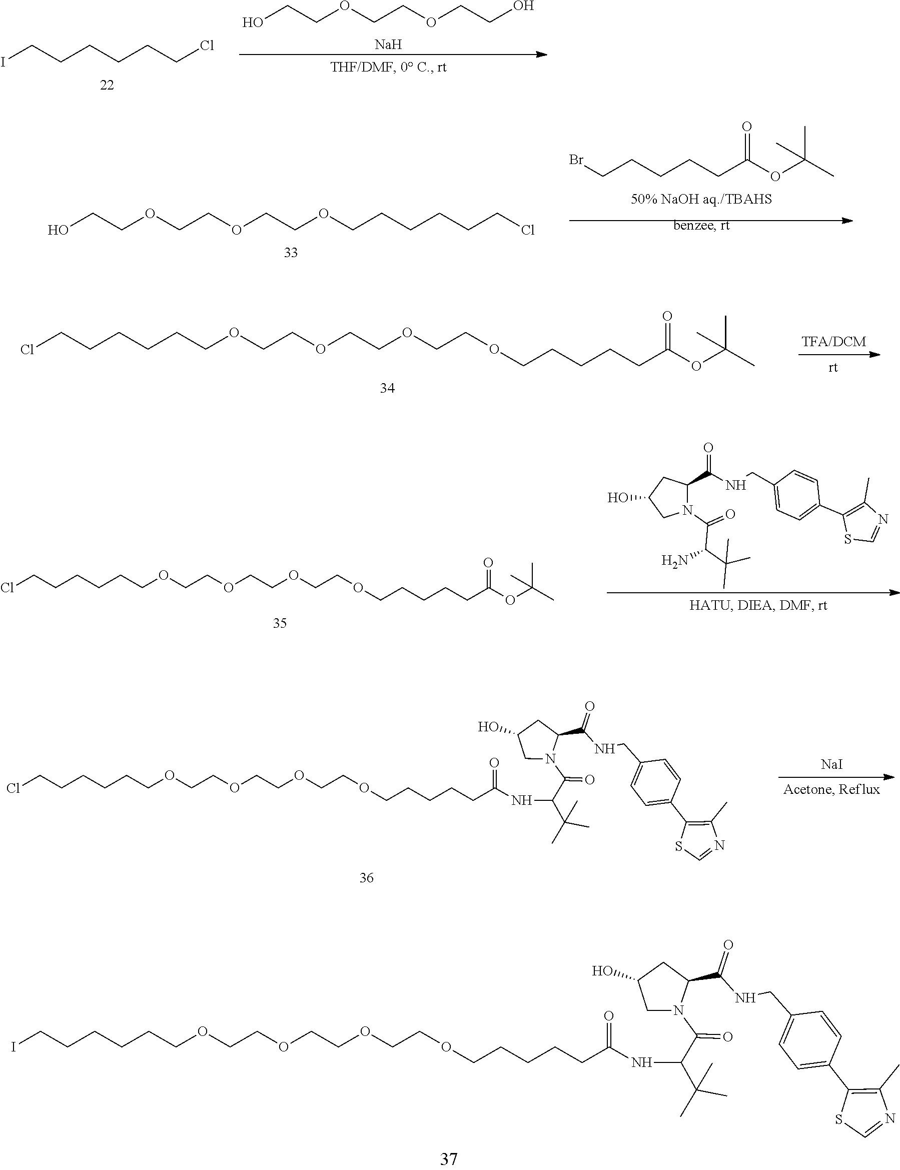

Intermediate 37: 800 mg of 33 was obtained by a method similar to the one described for 27 using triethylene glycol instead ethylene glycol.

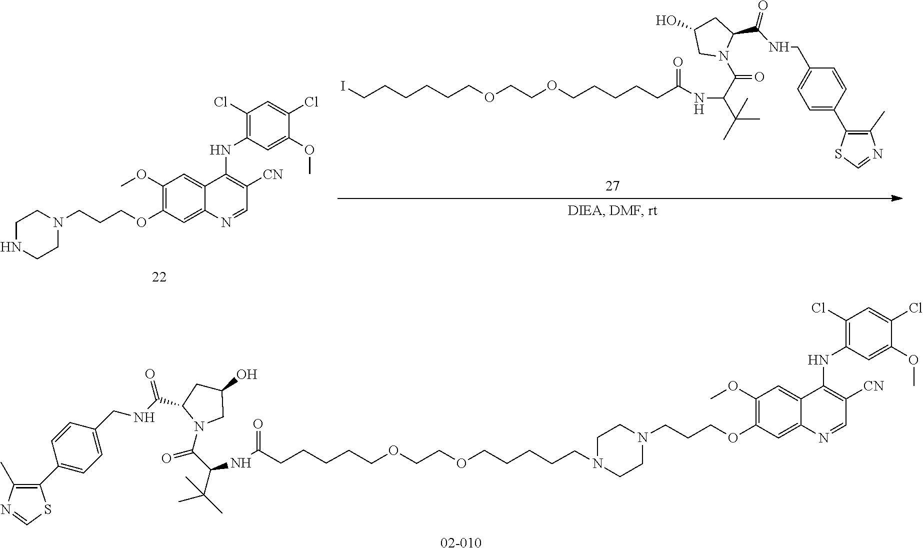

Compound 02-010: 60 mg of 22 and 1.1 eq of intermediate 27 were added to a vial with a magnetic stir bar. The vial was capped, purged with argon, and 1 [Bis(dimethylamino)methylene]-1H-1,2,3-triazolo[4,5-b]pyridinium 3-oxid hexafluorophosphate (HATU), and diisopropylethylamine (DIEA) in dry dimethylformamide (DMF) were added. The reaction was leave at constantly stirring overn [symbol] action was then cooled and purified by RP-HPLC. Product containing fractions were [symbol] dried under reduced pressure to yield 20% of 02-010.

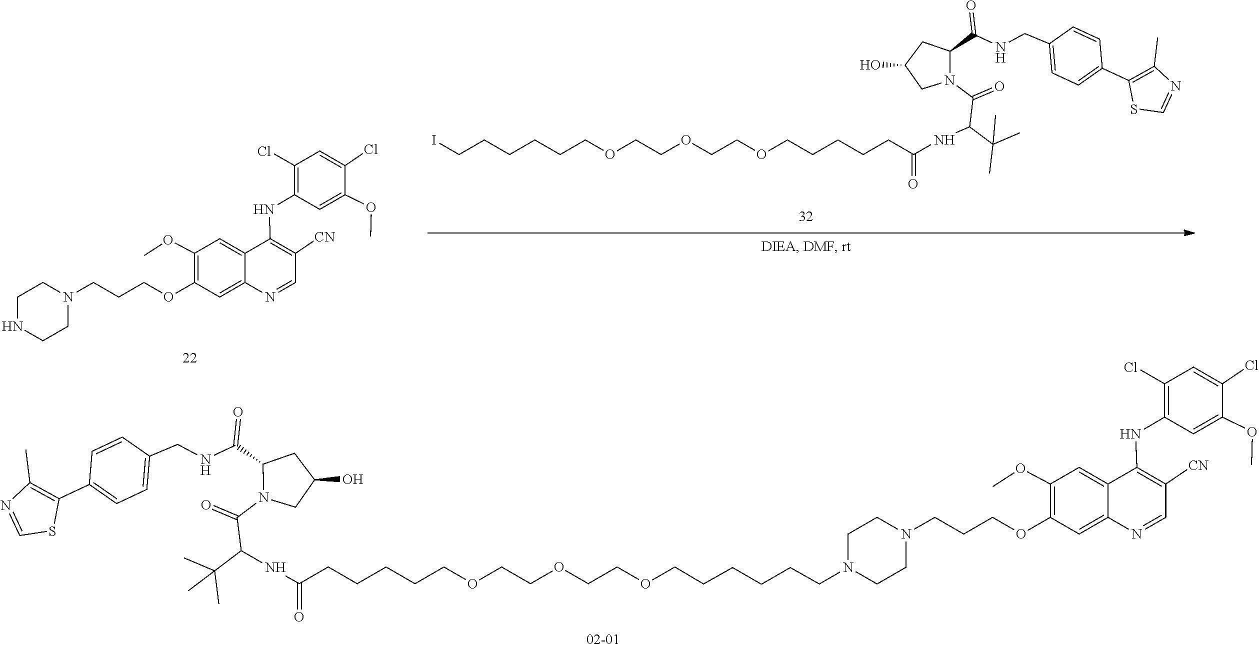 [SMILES: COc1cc(Nc2c(C#N)cnc3cc(OCCCN4CCNCC4)c(OC)cc23)c(Cl)cc1Cl ; Cc1ncsc1-c1ccc(CNC(=O)[C@@H]2C[C@@H](O)CN2C(=O)C(NC(=O)CCCCCOCCOCCOCCCCCCI)C(C)(C)C)cc1 ; COc1cc(Nc2c(C#N)cnc3cc(OCCCN4CCN(CCCCCCOCCOCCOCCCCCC(=O)NC(C(=O)N5C[C@H](O)C[C@H]5C(=O)NCc5ccc(-c6scnc6C)cc5)C(C)(C)C)CC4)c(OC)cc23)c(Cl)cc1Cl]

Compound 02-01: 26 mg of 02-01 was obtained by a method similar to the one described for compound 02-010 using intermediate 32 instead of intermediate 27.

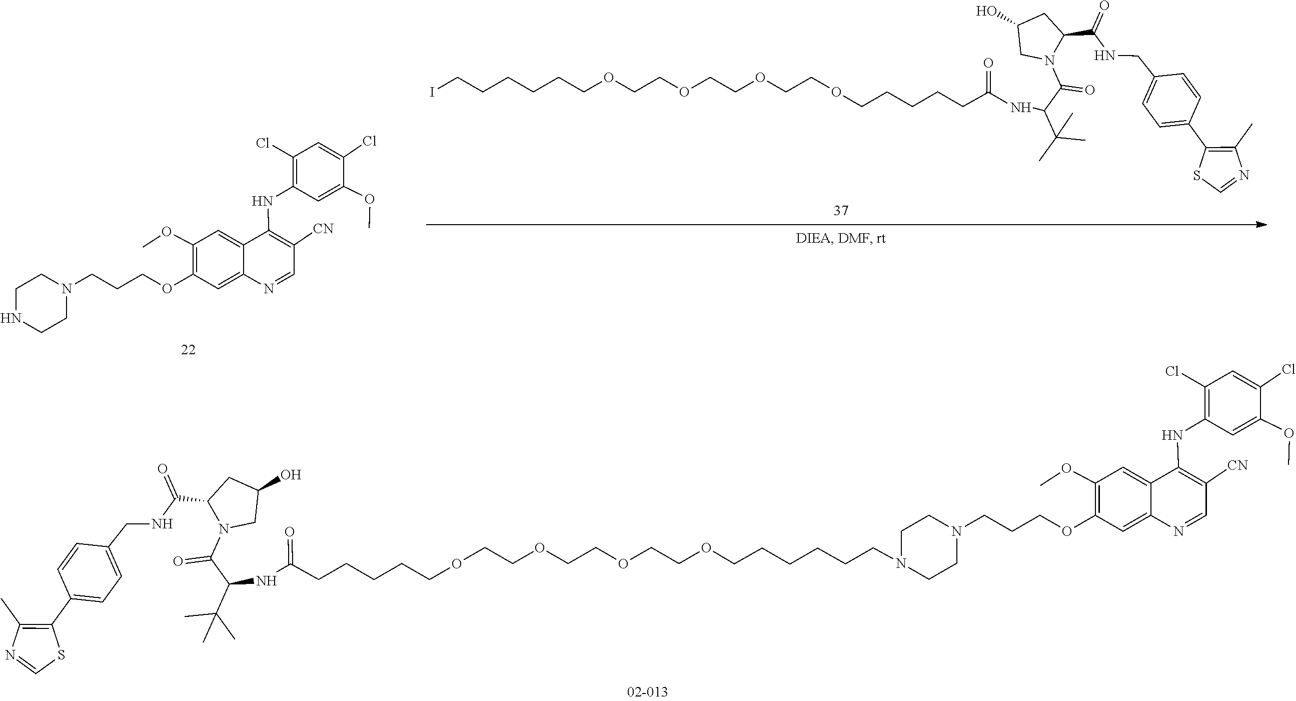

Compound 02-013: 26 mg of 02-013 was obtained by a method similar to the one described for compound 02-010 using intermediate 37 instead of intermediate 27.

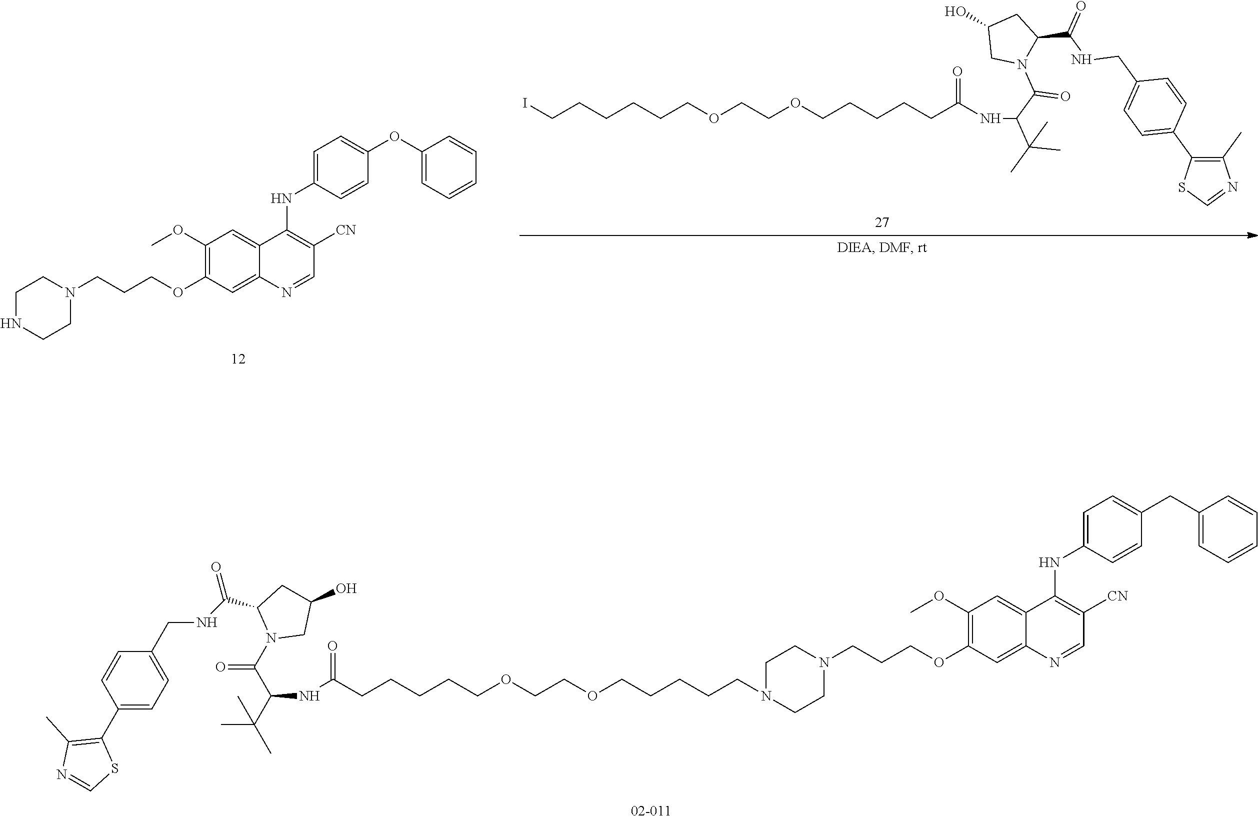 [SMILES: COc1cc2c(Nc3ccc(Oc4ccccc4)cc3)c(C#N)cnc2cc1OCCCN1CCNCC1 ; Cc1ncsc1-c1ccc(CNC(=O)[C@@H]2C[C@@H](O)CN2C(=O)C(NC(=O)CCCCCOCCOCCCCCCI)C(C)(C)C)cc1 ; COc1cc2c(Nc3ccc(Cc4ccccc4)cc3)c(C#N)cnc2cc1OCCCN1CCN(CCCCCOCCOCCCCCC(=O)N[C@H](C(=O)N2C[C@H](O)C[C@H]2C(=O)NCc2ccc(-c3scnc3C)cc2)C(C)(C)C)CC1]

Compound 02-011: 26 mg of 02-011 was obtained by a method similar to the one described for compound 02-010 using 12 as a starting material instead of 22.

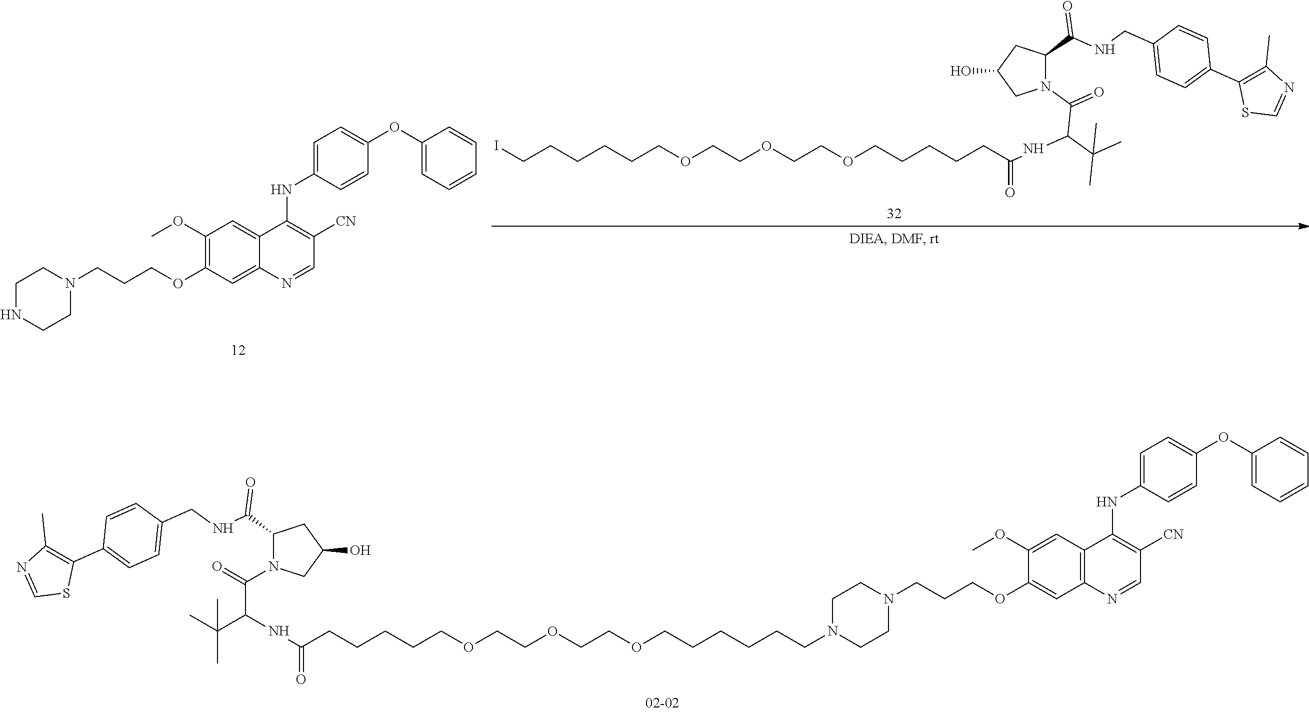

Compound 02-012: 26 mg of 02-012 was obtained by a method similar to the one described for compound 02-01 using 12 as a starting material instead of 22.

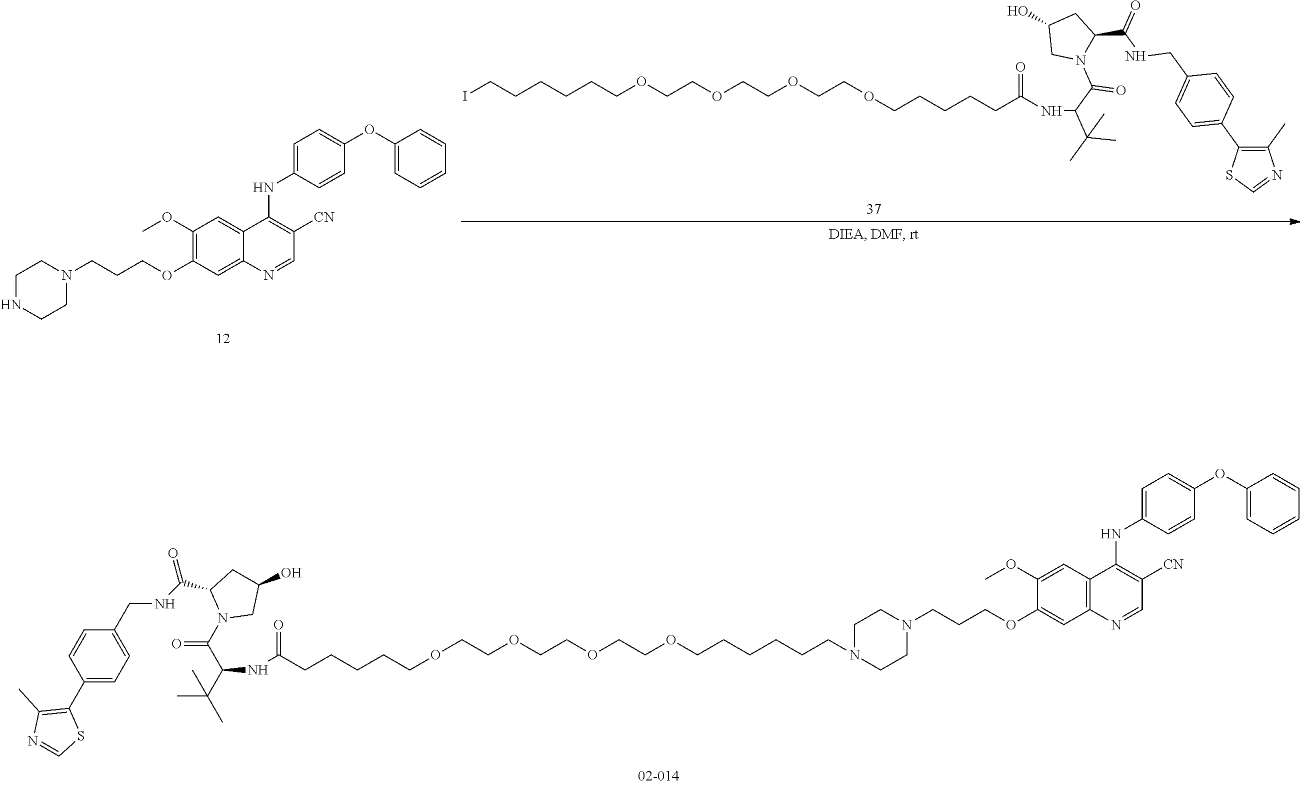

Compound 02-014: 26 mg of 02-014 was obtained by a method similar to the one described for compound 02-013 using 12 as a starting material instead of 22.

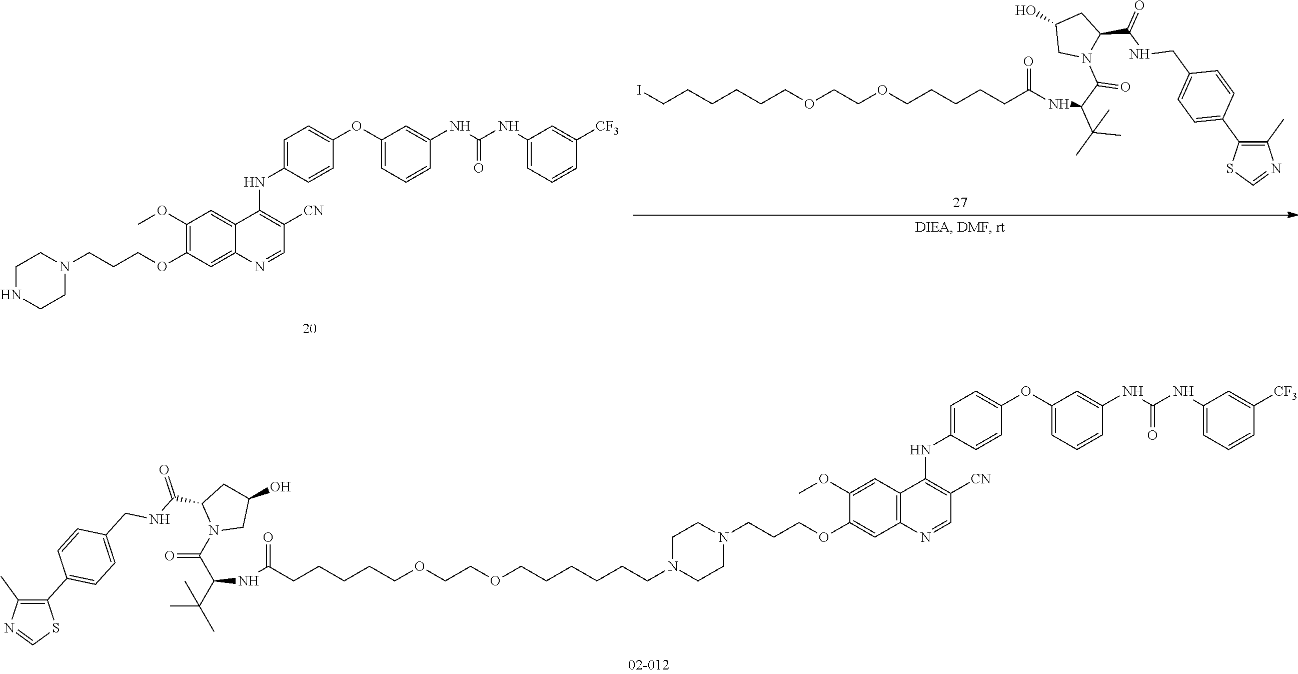 [SMILES: COc1cc2c(Nc3ccc(Oc4cccc(NC(=O)Nc5cccc(C(F)(F)F)c5)c4)cc3)c(C#N)cnc2cc1OCCCN1CCNCC1 ; Cc1ncsc1-c1ccc(CNC(=O)[C@@H]2C[C@@H](O)CN2C(=O)[C@H](NC(=O)CCCCCOCCOCCCCCCI)C(C)(C)C)cc1 ; COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOCCCCCC(=O)N[C@H](C(=O)N2C[C@H](O)C[C@H]2C(=O)NCc2ccc(-c3scnc3C)cc2)C(C)(C)C)CC1]

Compound 02-012: 26 mg of 02-012 was obtained by a method similar to the one described for compound 02-010 using 12 as a starting material instead of 22. MS (ES+) m/z 1382.6 (M)+

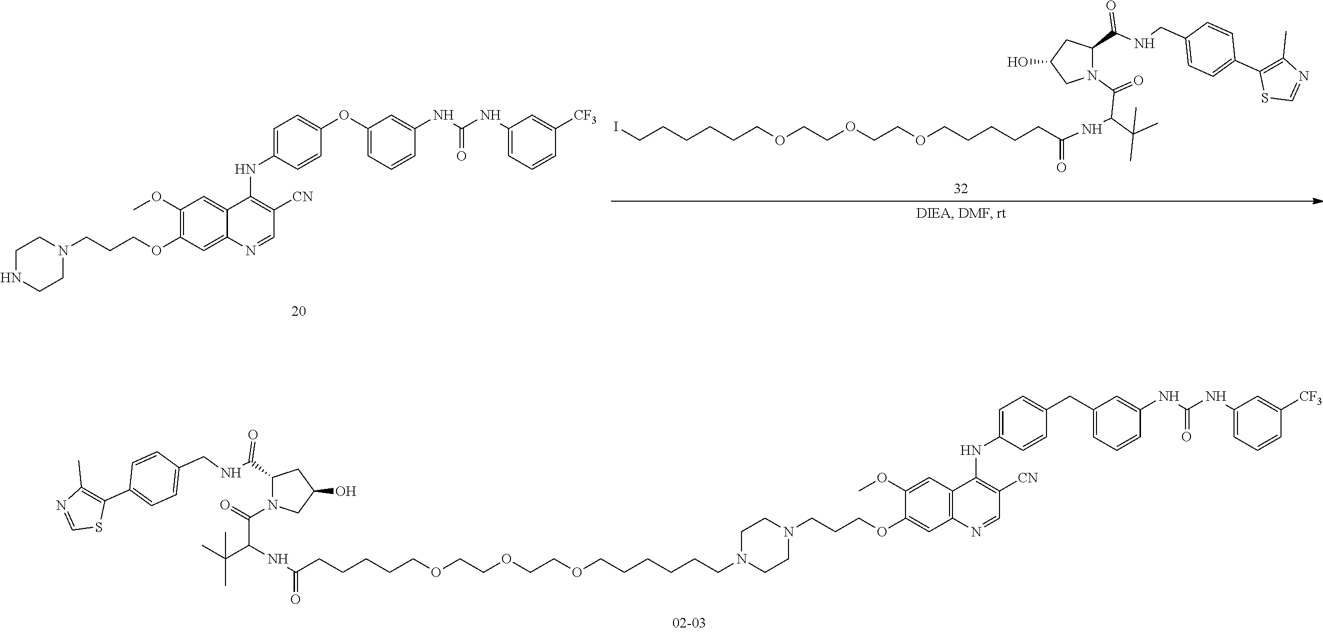 [SMILES: COc1cc2c(Nc3ccc(Oc4cccc(NC(=O)Nc5cccc(C(F)(F)F)c5)c4)cc3)c(C#N)cnc2cc1OCCCN1CCNCC1 ; Cc1ncsc1-c1ccc(CNC(=O)[C@@H]2C[C@@H](O)CN2C(=O)C(NC(=O)CCCCCOCCOCCOCCCCCCI)C(C)(C)C)cc1 ; COc1cc2c(Nc3ccc(Cc4cccc(NC(=O)Nc5cccc(C(F)(F)F)c5)c4)cc3)c(C#N)cnc2cc1OCCCN1CCN(CCCCCCOCCOCCOCCCCCC(=O)NC(C(=O)N2C[C@H](O)C[C@H]2C(=O)NCc2ccc(-c3scnc3C)cc2)C(C)(C)C)CC1]

Compound 02-03: 26 mg of 02-03 was obtained by a method similar to the one described for compound 02-02 using 20 as a starting material instead of 12.

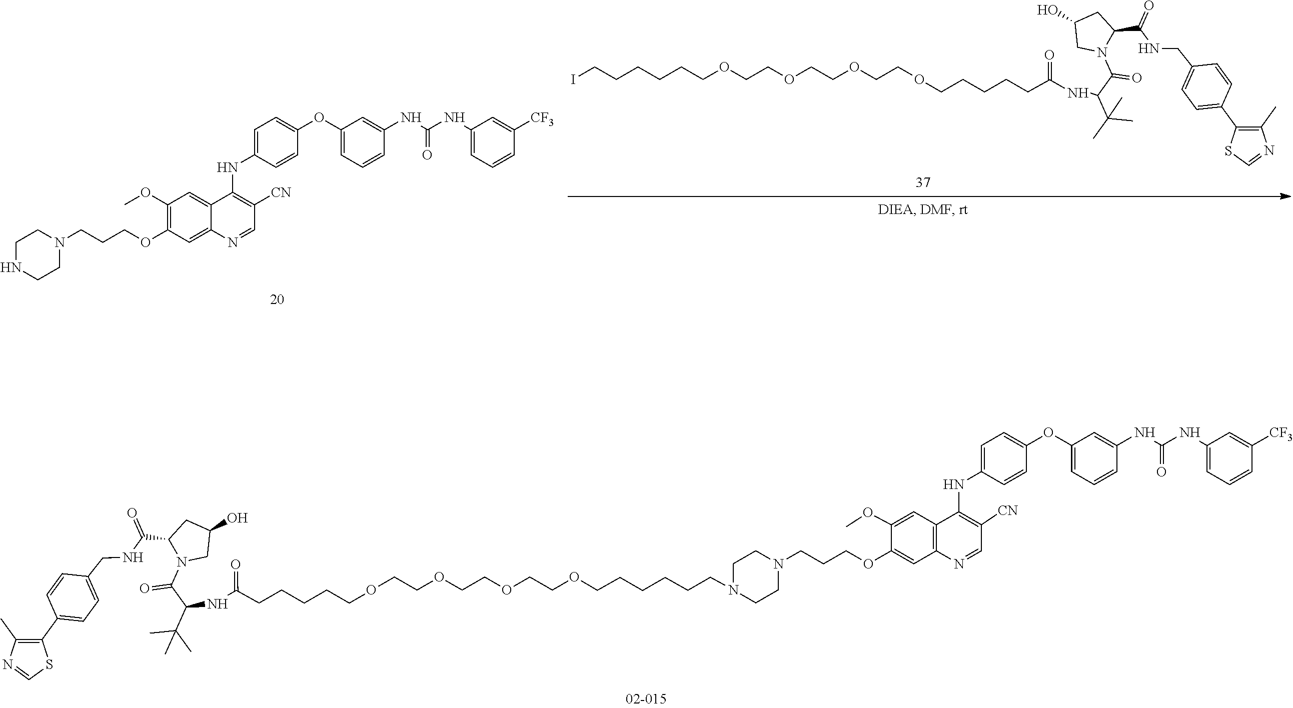 [SMILES: COc1cc2c(Nc3ccc(Oc4cccc(NC(=O)Nc5cccc(C(F)(F)F)c5)c4)cc3)c(C#N)cnc2cc1OCCCN1CCNCC1 ; Cc1ncsc1-c1ccc(CNC(=O)[C@@H]2C[C@@H](O)CN2C(=O)C(NC(=O)CCCCCOCCOCCOCCOCCCCCCI)C(C)(C)C)cc1 ; COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOCCOCCOCCCCCC(=O)N[C@H](C(=O)N2C[C@H](O)C[C@H]2C(=O)NCc2ccc(-c3scnc3C)cc2)C(C)(C)C)CC1]

Compound 02-015: 26 mg of 02-015 was obtained by a method similar to the one described for compound 02-014 using 20 as a starting material instead of 12. MS (ES+) m/z 1470.6 (M)

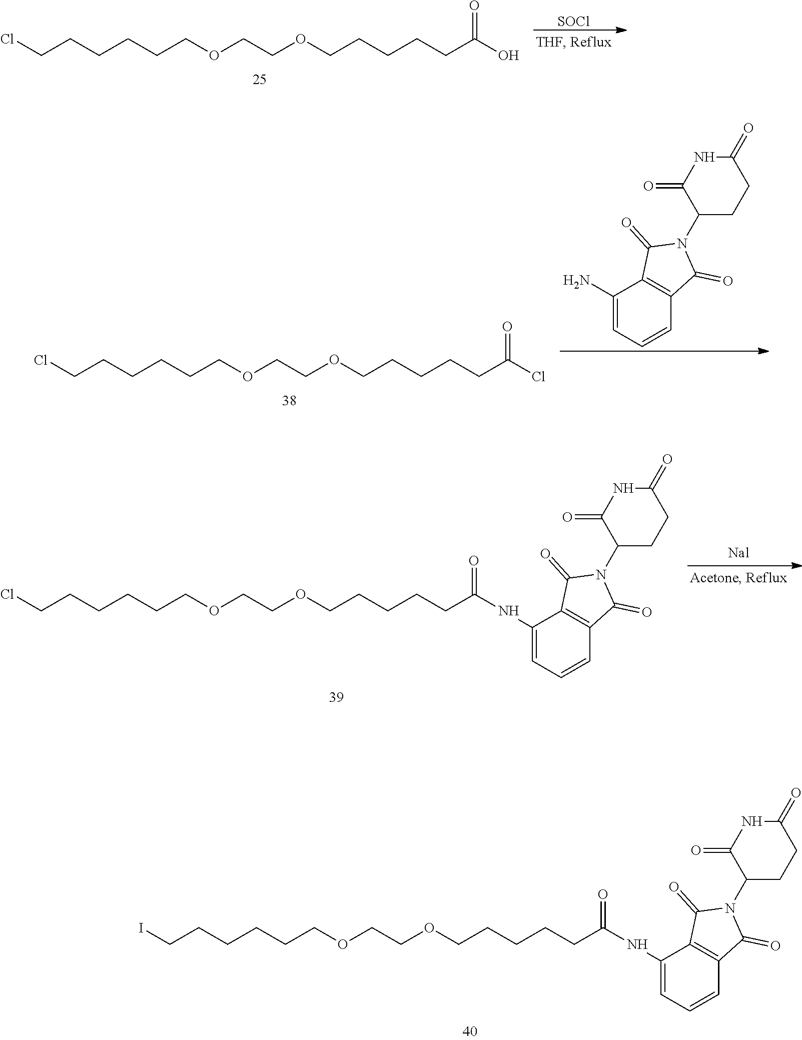 [SMILES: O=C(O)CCCCCOCCOCCCCCCCl ; "SOCl" ; O=C(Cl)CCCCCOCCOCCCCCCCl ; Nc1cccc2c1C(=O)N(C1CCC(=O)NC1=O)C2=O ; O=C1CCC(N2C(=O)c3cccc(NC(=O)CCCCCOCCOCCCCCCCl)c3C2=O)C(=O)N1 ; O=C1CCC(N2C(=O)c3cccc(NC(=O)CCCCCOCCOCCCCCCI)c3C2=O)C(=O)N1]

Intermediate 40: 1.5 g of 25 were added to a vial with a magnetic stir bar. The vial was capped, purged with argon, and 1.1 eq of Thionyl chloride in dry tetrahydrofuran were added. The reaction was leave at constantly stirring and reflux for about 5 h. The reaction was then cooled. After workup 1.5 g of crude 25 were obtained. Compound 25 was used without further purification. 1.5 g of compound 25 were mixed with 4-amino-2-(2,6-dioxopiperidin-3-yl)-1H-isoindole-1,3(2H)-dione (0.3 eq.) were added to a vial with a magnetic stir bar. The vial was capped, purged with argon, and dry tetrahydrofuran were added. 600 mg of compound 39 was obtained as a crude and used without further purification. 600 mg of compound 39 and 1.1 eq of sodium iodide were added to a vial with a stir bar. The vial was capped, purged with argon, and 15 mL of dry acetone was added. The reaction was stirred at room temperature for 5 min and was then refluxed by 5 hours to give 620 mg of intermediate 40.

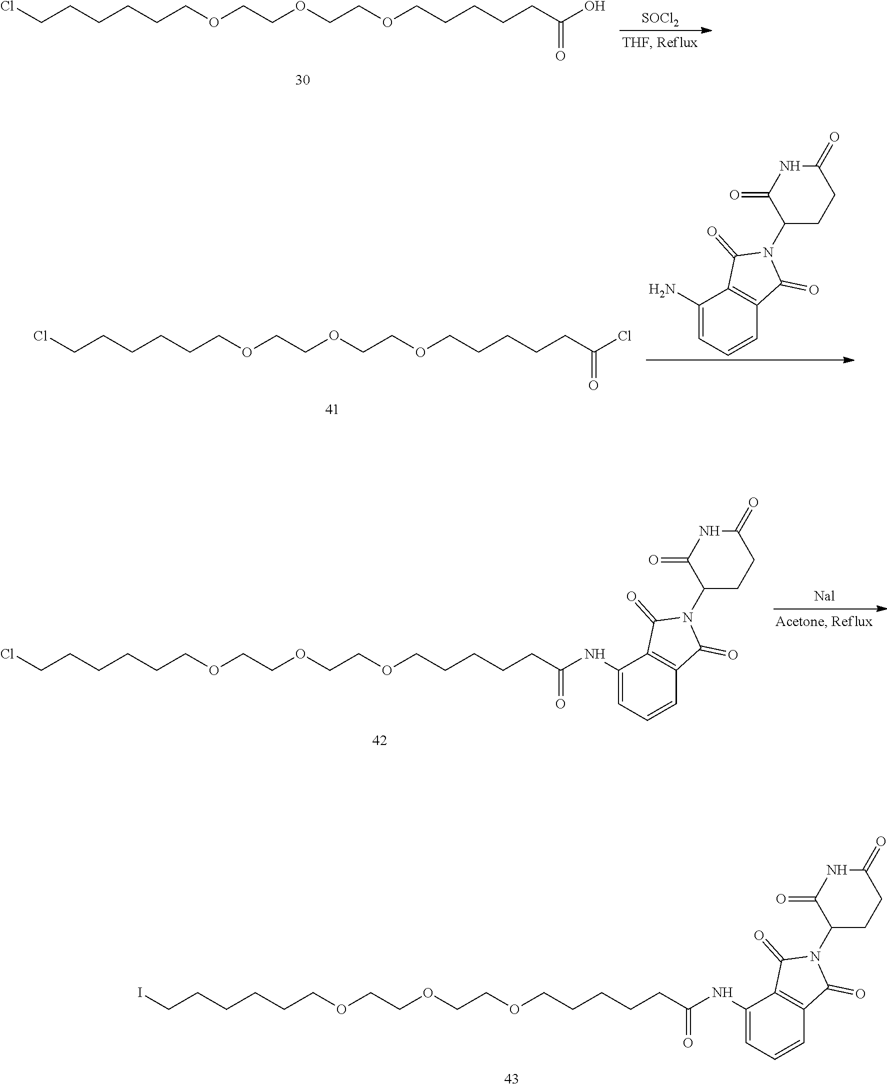

Intermediate 43: 600 mg mg of 43 was obtained by a method similar to the one described for intermediate 40 using 30 as a starting material instead of 25.

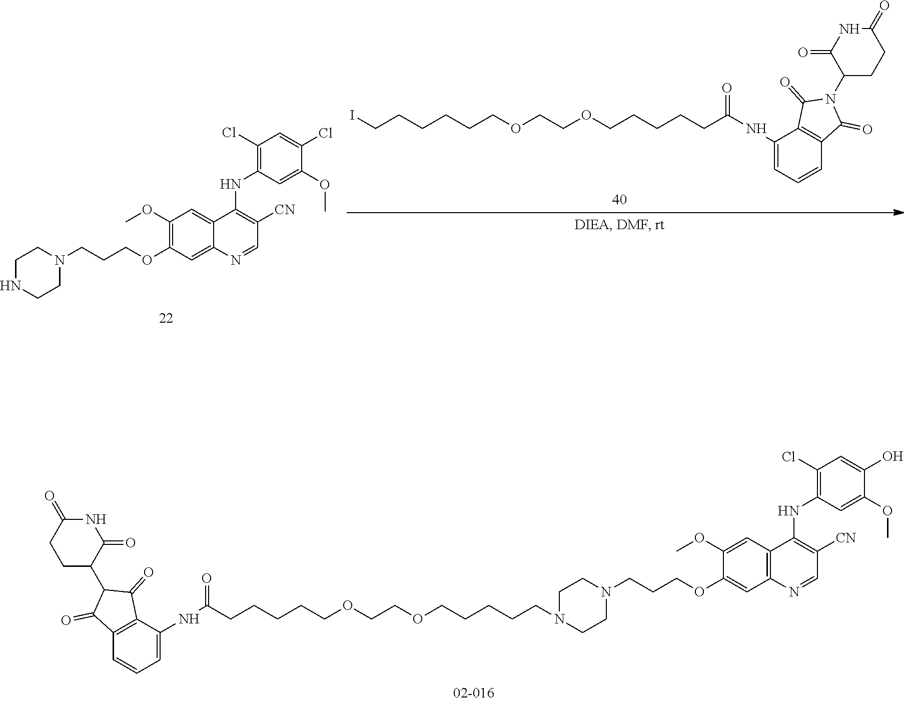

Compound 02-016: 26 mg of 02-016 was obtained by a method similar to the one described for compound 02-010 using intermediate 40 instead of intermediate 27.

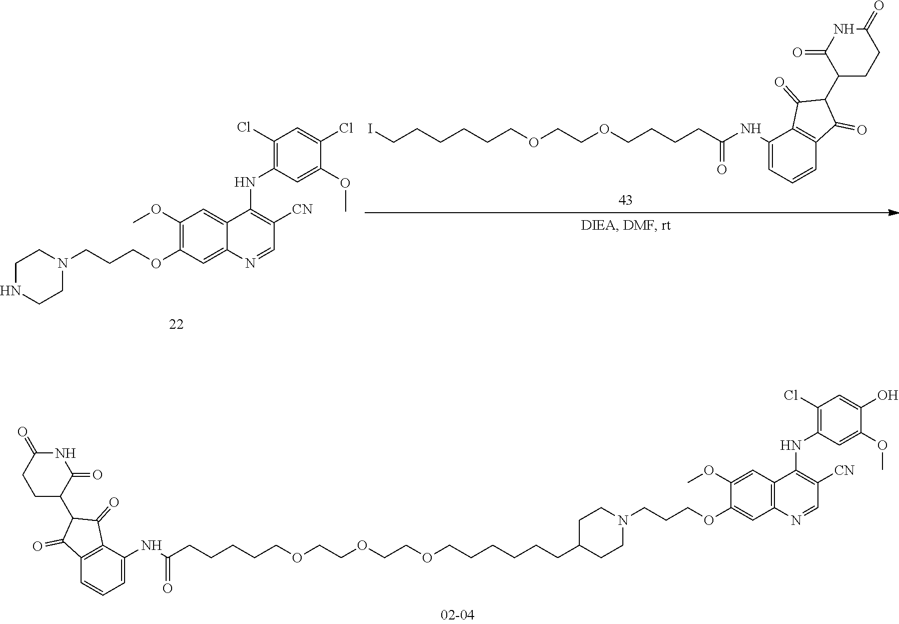

Compound 02-04: 26 mg of 02-04 was obtained by a method similar to the one described for compound 02-010 using intermediate 43 instead of intermediate 27.

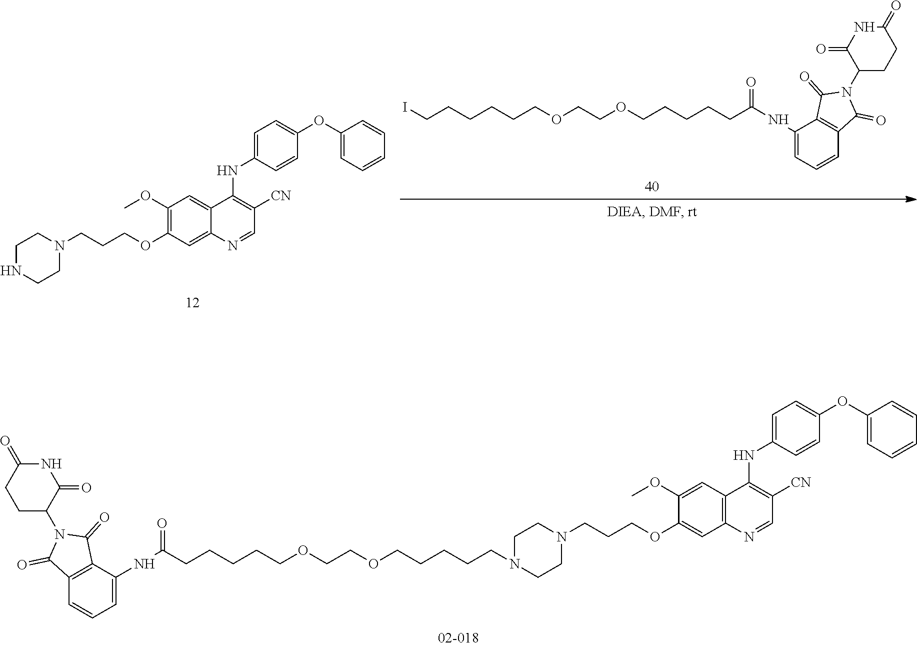

Compound 02-018: 26 mg of 02-018 was obtained by a method similar to the one described for compound 02-010 using intermediate 40 instead of intermediate 27.

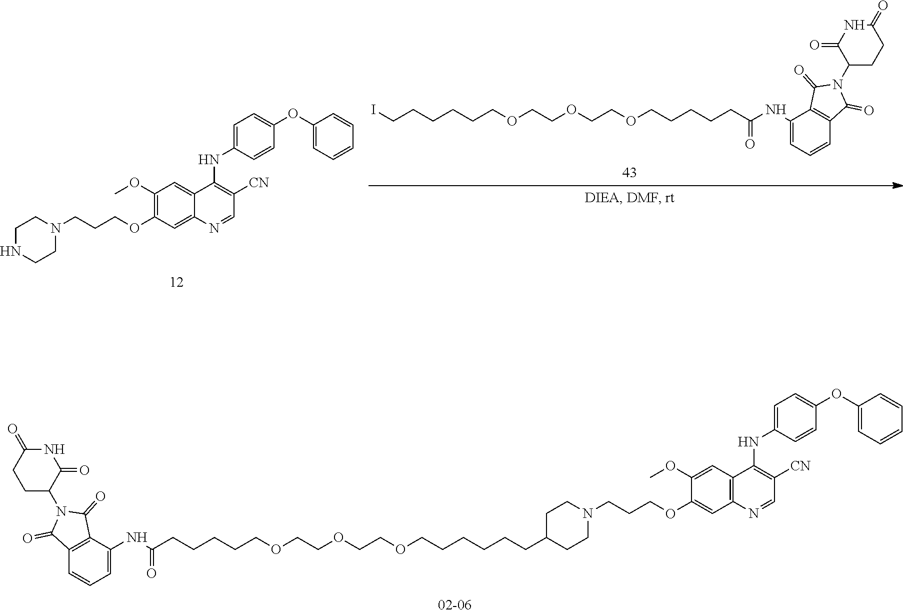

Compound 02-06: 26 mg of 02-06 was obtained by a method similar to the one described for compound 02-010 using intermediate 43 instead of intermediate 27.

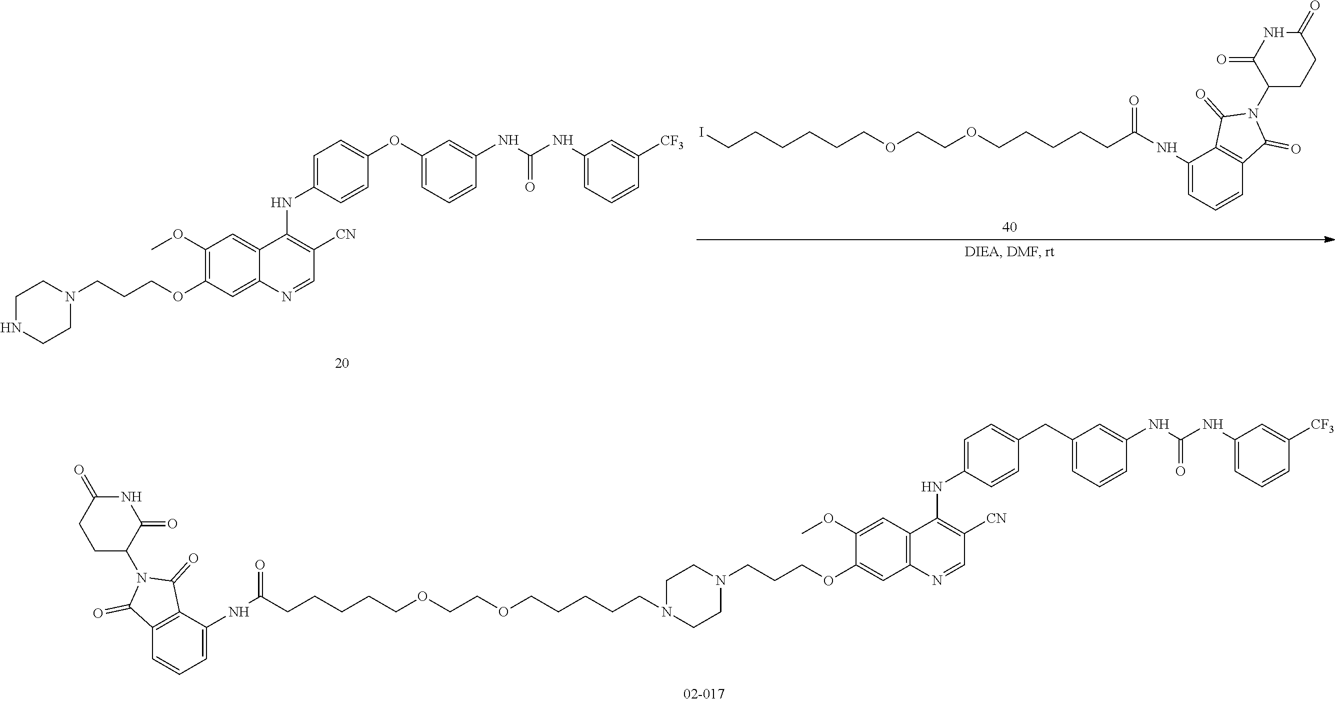

Compound 02-017: 26 mg of 02-017 was obtained by a method similar to the one described for compound 02-010 using intermediate 40 instead of intermediate 27.

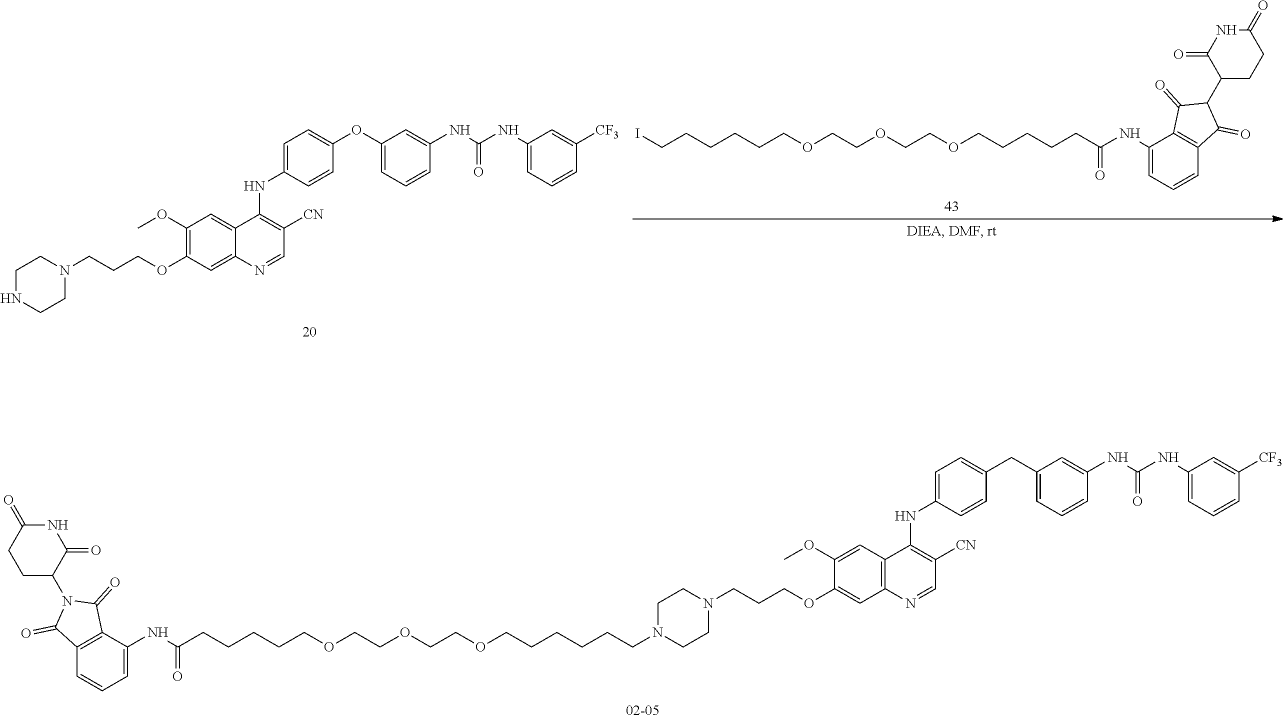

Compound 02-05: 26 mg of 02-05 was obtained by a method similar to the one described for compound 02-010 using intermediate 43 instead of intermediate 27.

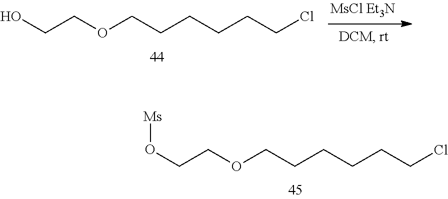 [SMILES: OCCOCCCCCCCl ; CS(=O)(=O)OCCOCCCCCCCl]

2-[(6-chlorohexyl)oxy]ethyl methanesulfonate (45): 3 g of 2-[(6-chlorohexyl)oxy]ethan-1-ol (44) were added to a vial with a magnetic stir bar. The vial was capped, purged with argon, and 1.1 eq of Methanesulfonyl chloride in dry dichlorometane were added. The reaction was leave at constantly stirring at room temperature for about 5 h. The reaction was then cooled. After workup 3 g of crude 45 were obtained, and used without further purification.

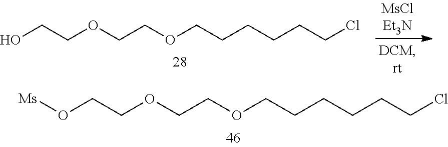 [SMILES: OCCOCCOCCCCCCCl ; CS(=O)(=O)OCCOCCOCCCCCCCl]

2-{2-[(6-chlorohexyl)oxy]ethoxy}ethyl methanesulfonate (46): 5 mg of 46 was obtained by a method similar to the one described for compound 45 using 28 as starting material instead of 44.

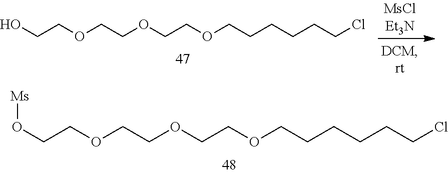 [SMILES: OCCOCCOCCOCCCCCCCl ; CS(=O)(=O)OCCOCCOCCOCCCCCCCl]

2-(2-{2-[(6-chlorohexyl)oxy]ethoxy}ethoxy)ethyl methanesulfonate (48): 5 mg of 48 was obtained by a method similar to the one described for compound 45 using 47 as starting material instead of 44.

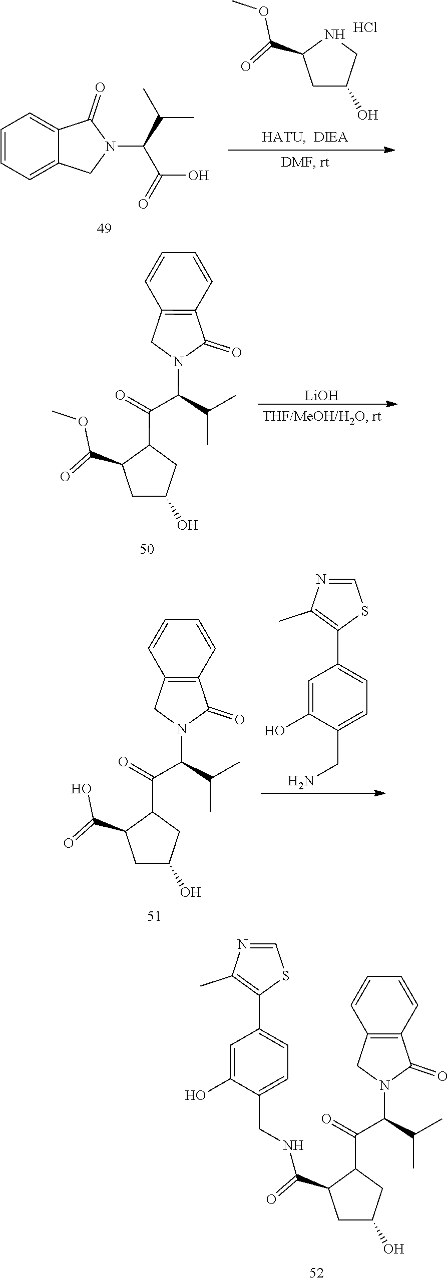

Intermediate 52: 3 g of (2S)-3-methyl-2-(1-oxo-1,3-dihydro-2H-isoindol-2-yl)butanoic acid (49) and 1.2 eq. of 1-[Bis(dimethylamino)methylene]-1H-1,2,3-triazolo[4,5-b]pyridinium 3-oxid hexafluorophosphate (HATU), were added to a vial with a stir bar. The vial was capped, purged with argon, and 200 mL of dry dimethylformamide and 1.2 eq. diisopropylethylamine (DIEA) was added. The reaction was stirred at 0° C. for 5 min and was then allowed to warm to 25° C. over the course of an hour. The solution was then cooled to −20° C. and filtered to yield 3.3 mg (71% yield) of 50 as a crude solid, which was used in the next step without further purification. 3.3 g of the crude 50 was added to a vial containing lithium oxide and 100 mL of a mixture of tetrahydrofuran and methanol and a stir bar. The reaction was filtered, concentrated in vacuo to give 2.8 g of the crude 51. 2.5 g of compound 51 was and 1.2 eq. of 1-Ethyl-3-(3-dimethylaminopropyl) carbodiimide, were added to a vial with a stir bar. The vial was capped, purged with argon, and 200 mL of dry dichloromethane and 1.2 eq. diisopropylethylamine (DIEA) were added. The solution was then cooled to −20° C. and filtered to obtain intermediate 52 as a crude solid in 75% yield, which was used without further purification.

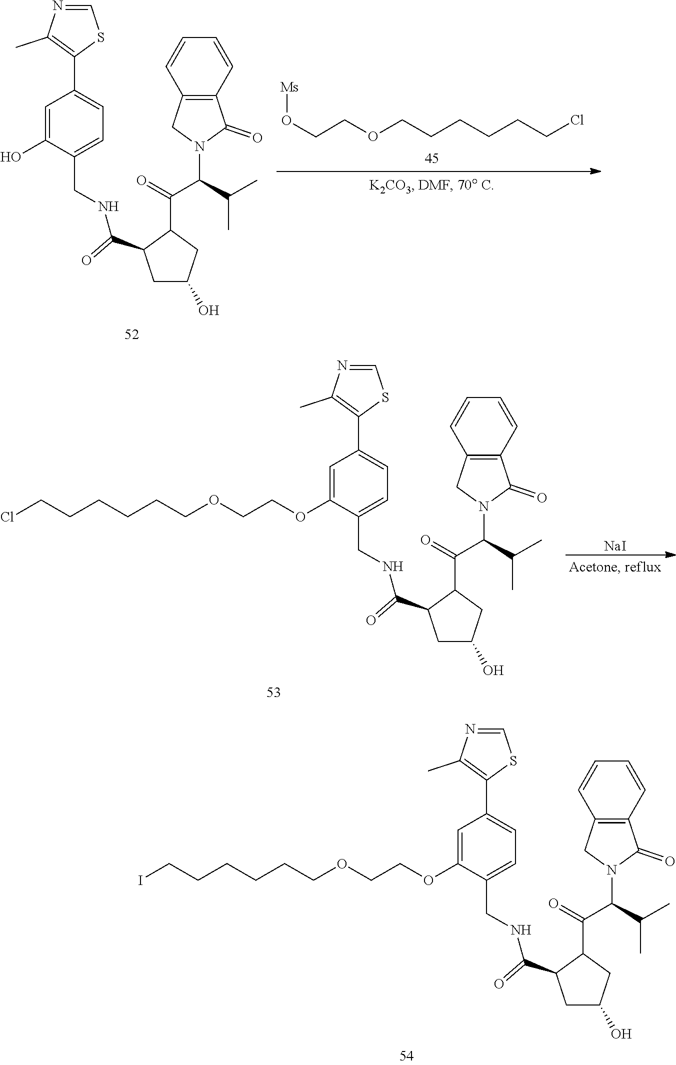

Intermediate 54: 100 mg of 52 and excess of potassium carbonate were added to a vial with a stir bar. The vial was capped, purged with argon, and 20 mL of dry dimethylformamide and 1.2 eq. of 45 were added. The reaction was stirred at 70° overnight and was then allowed to warm to 25° C. over the course of an hour. The solution was then cooled to −20° C. and filtered to yield 101 mg of 53 as a crude solid, which was used in the next step without further purification. 60 mg of compound 54 and 1.1 eq of sodium iodide were added to a vial with a stir bar. The vial was capped, purged with argon, and 15 mL of dry acetone was added. The reaction was stirred at room temperature for 5 min and was then refluxed by 5 hours to give 45 mg of intermediate 54.

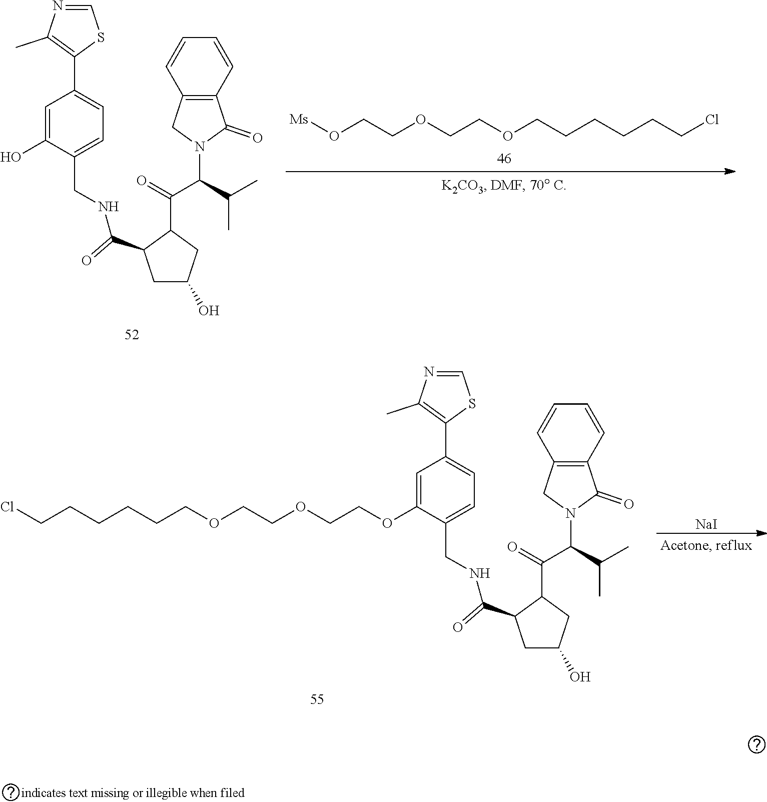 [SMILES: Cc1ncsc1-c1ccc(CNC(=O)[C@@H]2C[C@@H](O)CC2C(=O)[C@H](C(C)C)N2Cc3ccccc3C2=O)c(O)c1 ; CS(=O)(=O)OCCOCCOCCCCCCCl ; Cc1ncsc1-c1ccc(CNC(=O)[C@@H]2C[C@@H](O)CC2C(=O)[C@H](C(C)C)N2Cc3ccccc3C2=O)c(OCCOCCOCCCCCCCl)c1 ; "NaI"]

Intermediate 56: 50 mg of 56 was obtained by a method similar to the one described for compound 54 using 46 as starting material instead of 45.

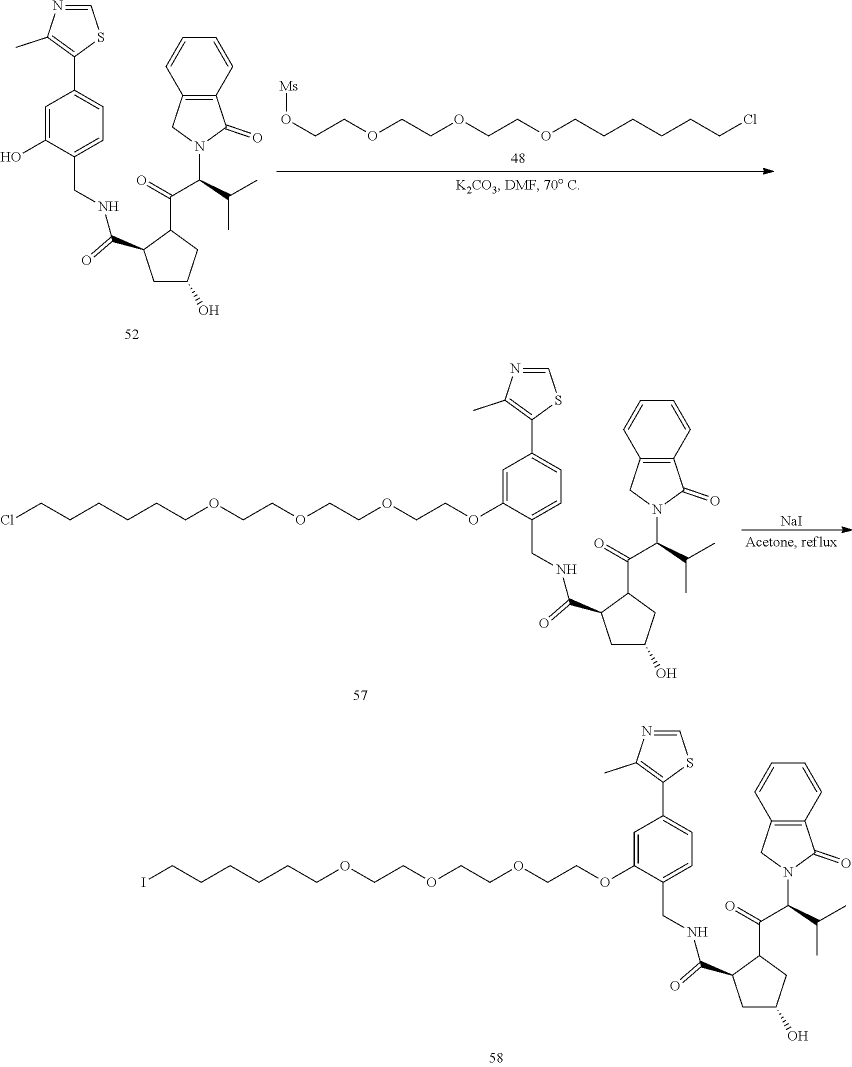

Intermediate 58: 50 mg of 58 was obtained by a method similar to the one described for compound 54 using 48 as starting material instead of 45

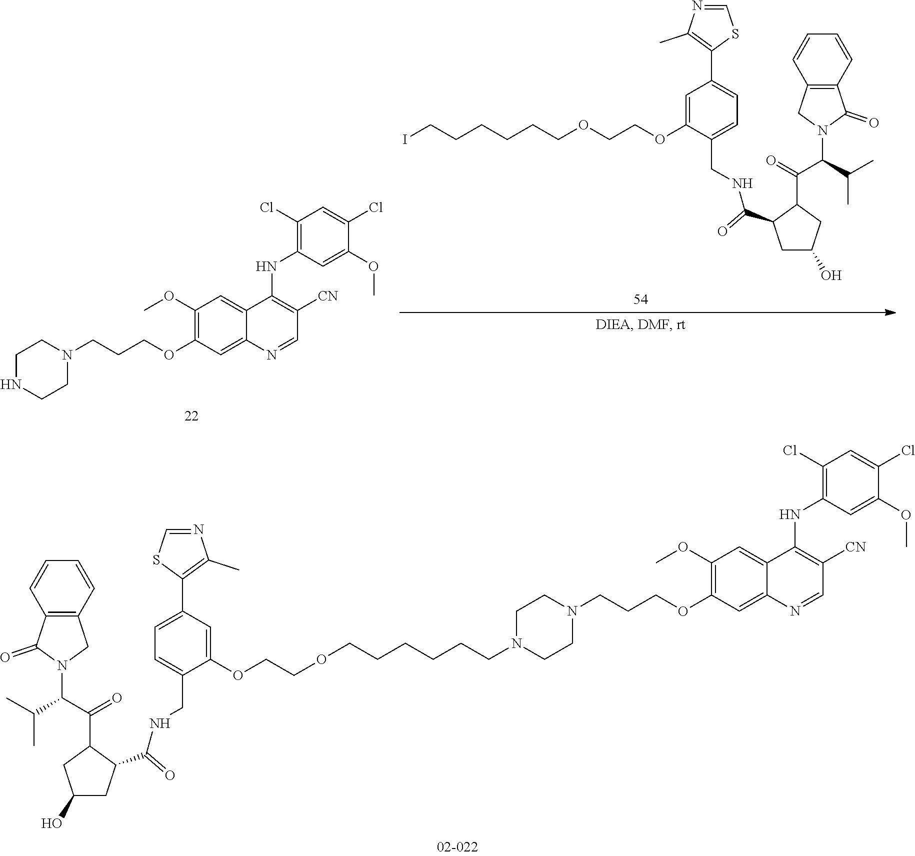 [SMILES: COc1cc(Nc2c(C#N)cnc3cc(OCCCN4CCNCC4)c(OC)cc23)c(Cl)cc1Cl ; Cc1ncsc1-c1ccc(CNC(=O)[C@@H]2C[C@@H](O)CC2C(=O)[C@H](C(C)C)N2Cc3ccccc3C2=O)c(OCCOCCCCCCI)c1 ; COc1cc(Nc2c(C#N)cnc3cc(OCCCN4CCN(CCCCCCOCCOc5cc(-c6scnc6C)ccc5CNC(=O)[C@@H]5C[C@@H](O)CC5C(=O)[C@H](C(C)C)N5Cc6ccccc6C5=O)CC4)c(OC)cc23)c(Cl)cc1Cl]

Compound 02-022: 60 mg of 22 and 1.1 eq of intermediate 54 were added to a vial with a magnetic stir bar. The vial was capped, purged with argon, and 1.2 eq. of diisopropylethylamine (DIEA) in dry dimethylformamide (DMF) were added. The reaction was leave at constantly stirring overnight. The reaction was then cooled and purified by RP-HPLC. Product containing fractions were pooled and dried under reduced pressure to yield 20% of 02-022.

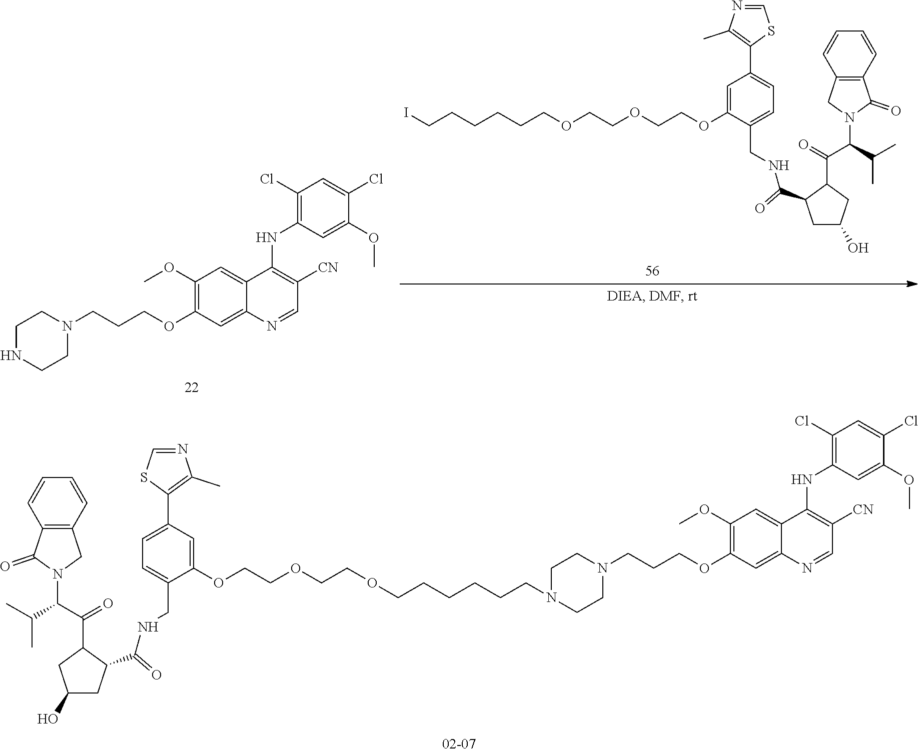 [SMILES: COc1cc(Nc2c(C#N)cnc3cc(OCCCN4CCNCC4)c(OC)cc23)c(Cl)cc1Cl ; Cc1ncsc1-c1ccc(CNC(=O)[C@@H]2C[C@@H](O)CC2C(=O)[C@H](C(C)C)N2Cc3ccccc3C2=O)c(OCCOCCOCCCCCCI)c1 ; COc1cc(Nc2c(C#N)cnc3cc(OCCCN4CCN(CCCCCCOCCOCCOc5cc(-c6scnc6C)ccc5CNC(=O)[C@@H]5C[C@@H](O)CC5C(=O)[C@H](C(C)C)N5Cc6ccccc6C5=O)CC4)c(OC)cc23)c(Cl)cc1Cl]

Compound 02-07: 20 mg of 02-07 was obtained by a method similar to the one described for compound 02-022 using 56 as starting material instead of 54.

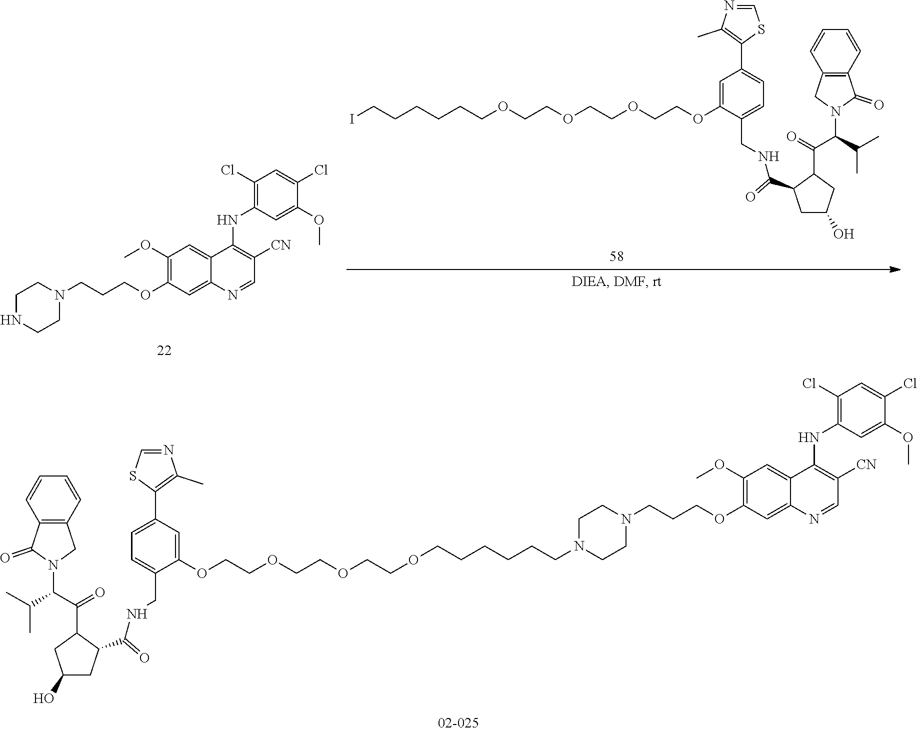 [SMILES: COc1cc(Nc2c(C#N)cnc3cc(OCCCN4CCNCC4)c(OC)cc23)c(Cl)cc1Cl ; Cc1ncsc1-c1ccc(CNC(=O)[C@@H]2C[C@@H](O)CC2C(=O)[C@H](C(C)C)N2Cc3ccccc3C2=O)c(OCCOCCOCCOCCCCCCI)c1 ; COc1cc(Nc2c(C#N)cnc3cc(OCCCN4CCN(CCCCCCOCCOCCOCCOc5cc(-c6scnc6C)ccc5CNC(=O)[C@@H]5C[C@@H](O)CC5C(=O)[C@H](C(C)C)N5Cc6ccccc6C5=O)CC4)c(OC)cc23)c(Cl)cc1Cl]

Compound 02-025: 20 mg of 02-025 was obtained by a method similar to the one described for compound 02-022 using 58 as starting material instead of 54.

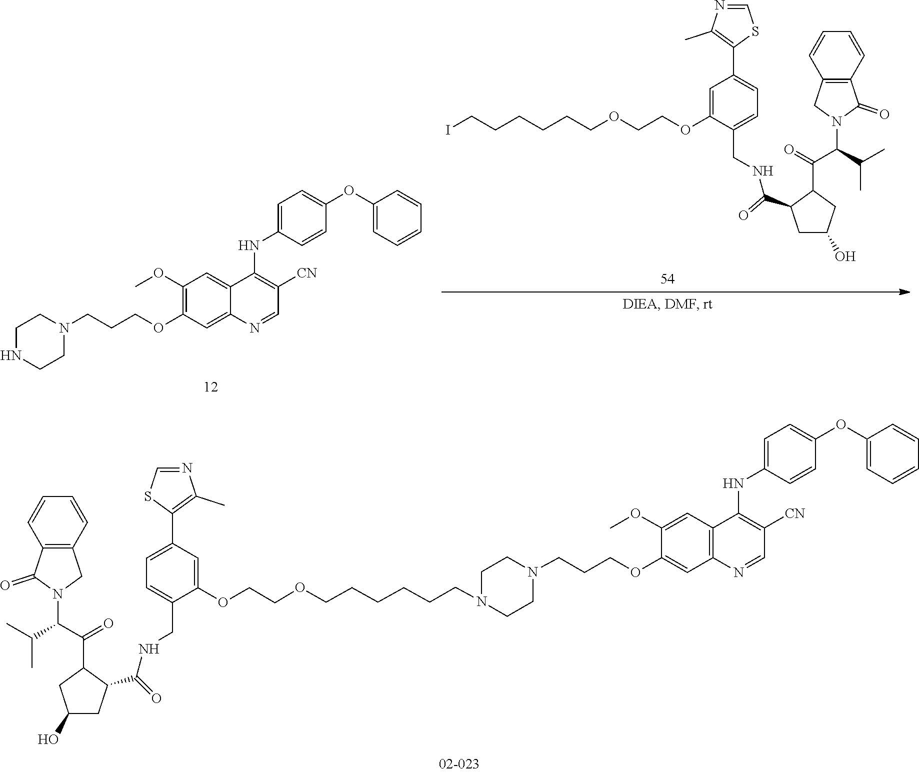 [SMILES: COc1cc2c(Nc3ccc(Oc4ccccc4)cc3)c(C#N)cnc2cc1OCCCN1CCNCC1 ; Cc1ncsc1-c1ccc(CNC(=O)[C@@H]2C[C@@H](O)CC2C(=O)[C@H](C(C)C)N2Cc3ccccc3C2=O)c(OCCOCCCCCCI)c1 ; COc1cc2c(Nc3ccc(Oc4ccccc4)cc3)c(C#N)cnc2cc1OCCCN1CCN(CCCCCCOCCOc2cc(-c3scnc3C)ccc2CNC(=O)[C@@H]2C[C@@H](O)CC2C(=O)[C@H](C(C)C)N2Cc3ccccc3C2=O)CC1]

Compound 02-022: 60 mg of 12 and 1.1 eq of intermediate 54 were added to a vial with a magnetic stir bar. The vial was capped, purged with argon, and 1.2 eq. diisopropylethylamine (DIEA) in dry dimethylformamide (DMF) were added. The reaction was leave at constantly stirring overnight. The reaction was then cooled and purified by RP-HPLC. Product containing fractions were pooled and dried under reduced pressure to yield 20% of 02-023.

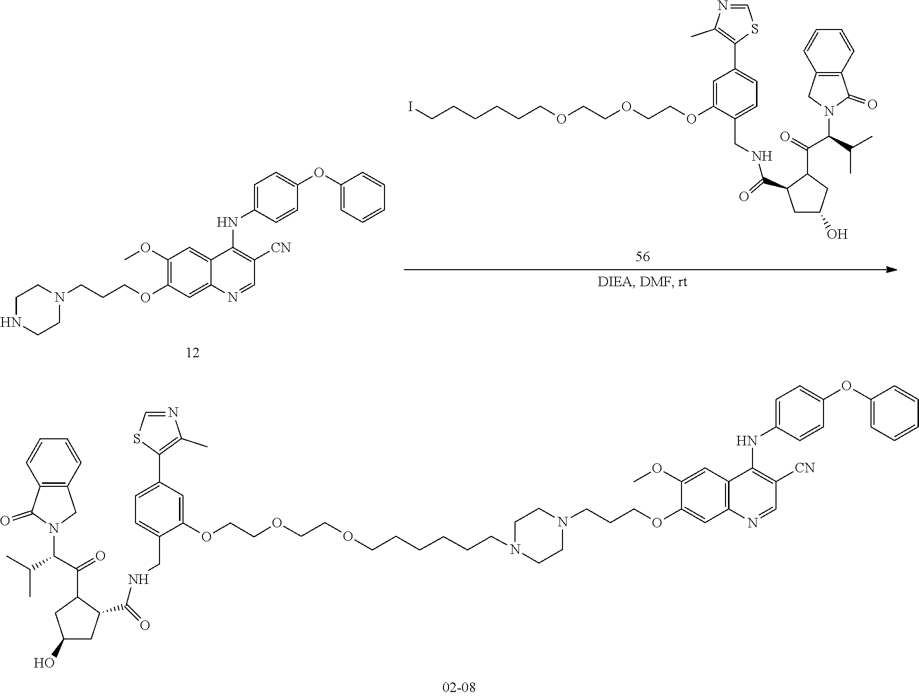 [SMILES: COc1cc2c(Nc3ccc(Oc4ccccc4)cc3)c(C#N)cnc2cc1OCCCN1CCNCC1 ; Cc1ncsc1-c1ccc(CNC(=O)[C@@H]2C[C@@H](O)CC2C(=O)[C@H](C(C)C)N2Cc3ccccc3C2=O)c(OCCOCCOCCCCCCI)c1 ; COc1cc2c(Nc3ccc(Oc4ccccc4)cc3)c(C#N)cnc2cc1OCCCN1CCN(CCCCCCOCCOCCOc2cc(-c3scnc3C)ccc2CNC(=O)[C@@H]2C[C@@H](O)CC2C(=O)[C@H](C(C)C)N2Cc3ccccc3C2=O)CC1]

Compound 02-08: 25 mg of 02-028 was obtained by a method similar to the one described for compound 02-022 using 56 as starting material instead of 54.

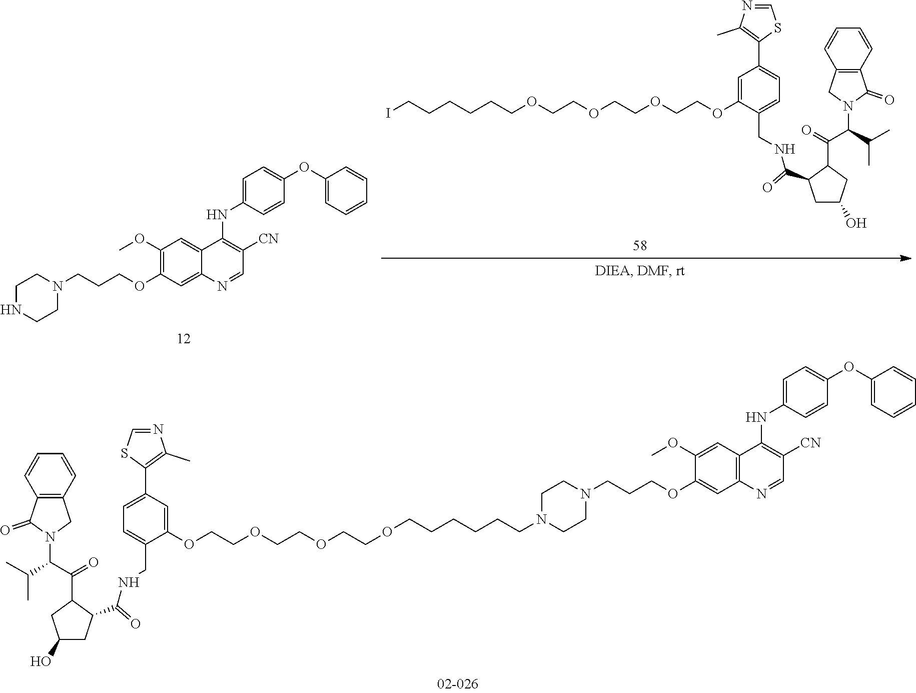 [SMILES: COc1cc2c(Nc3ccc(Oc4ccccc4)cc3)c(C#N)cnc2cc1OCCCN1CCNCC1 ; Cc1ncsc1-c1ccc(CNC(=O)[C@@H]2C[C@@H](O)CC2C(=O)[C@H](C(C)C)N2Cc3ccccc3C2=O)c(OCCOCCOCCOCCCCCCI)c1 ; COc1cc2c(Nc3ccc(Oc4ccccc4)cc3)c(C#N)cnc2cc1OCCCN1CCN(CCCCCCOCCOCCOCCOc2cc(-c3scnc3C)ccc2CNC(=O)[C@@H]2C[C@@H](O)CC2C(=O)[C@H](C(C)C)N2Cc3ccccc3C2=O)CC1]

Compound 02-026: 20 mg of 02-026 was obtained by a method similar to the one described for compound 02-022 using 58 as starting material instead of 54.

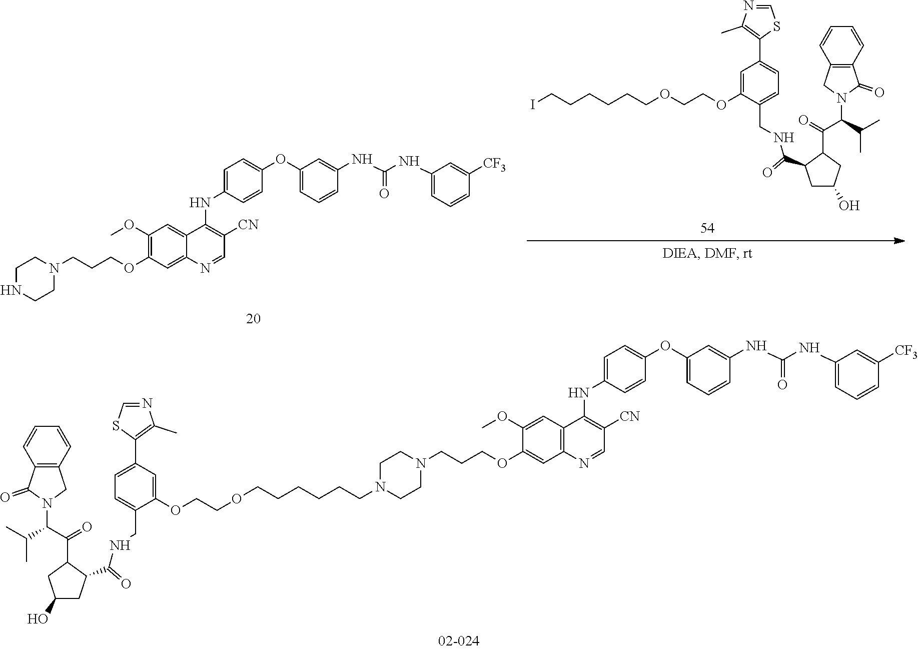 [SMILES: COc1cc2c(Nc3ccc(Oc4cccc(NC(=O)Nc5cccc(C(F)(F)F)c5)c4)cc3)c(C#N)cnc2cc1OCCCN1CCNCC1 ; Cc1ncsc1-c1ccc(CNC(=O)[C@@H]2C[C@@H](O)CC2C(=O)[C@H](C(C)C)N2Cc3ccccc3C2=O)c(OCCOCCCCCCI)c1 ; COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOc2cc(-c3scnc3C)ccc2CNC(=O)[C@@H]2C[C@@H](O)CC2C(=O)[C@H](C(C)C)N2Cc3ccccc3C2=O)CC1]

Compound 02-024: 60 mg of 12 and 1.1 eq of intermediate 54 were added to a vial with a magnetic stir bar. The vial was capped, purged with argon, and 1.2 eq. diisopropylethylamine (DIEA) in dry dimethylformamide (DMF) were added. The reaction was leave at constantly stirring overnight. The reaction was then cooled and purified by RP-HPLC. Product containing fractions were pooled and dried under reduced pressure to yield 20% of 02-024.

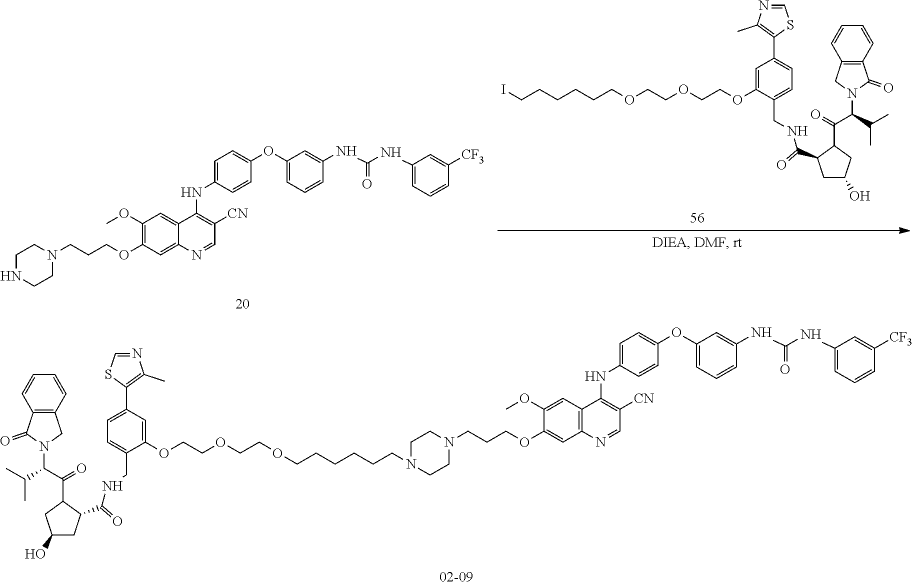 [SMILES: COc1cc2c(Nc3ccc(Oc4cccc(NC(=O)Nc5cccc(C(F)(F)F)c5)c4)cc3)c(C#N)cnc2cc1OCCCN1CCNCC1 ; Cc1ncsc1-c1ccc(CNC(=O)[C@@H]2C[C@@H](O)CC2C(=O)[C@H](C(C)C)N2Cc3ccccc3C2=O)c(OCCOCCOCCCCCCI)c1 ; COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOCCOc2cc(-c3scnc3C)ccc2CNC(=O)[C@@H]2C[C@@H](O)CC2C(=O)[C@H](C(C)C)N2Cc3ccccc3C2=O)CC1]

Compound 02-09: 25 mg of 02-09 was obtained by a method similar to the one described for compound 02-024 using 56 as starting material instead of 54.

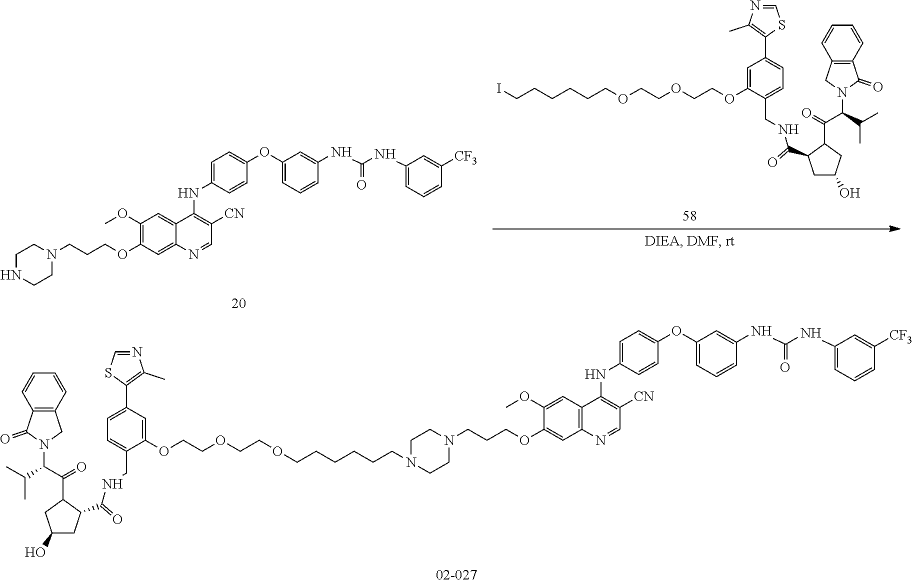 [SMILES: COc1cc2c(Nc3ccc(Oc4cccc(NC(=O)Nc5cccc(C(F)(F)F)c5)c4)cc3)c(C#N)cnc2cc1OCCCN1CCNCC1 ; Cc1ncsc1-c1ccc(CNC(=O)[C@@H]2C[C@@H](O)CC2C(=O)[C@H](C(C)C)N2Cc3ccccc3C2=O)c(OCCOCCOCCCCCCI)c1 ; COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOCCOc2cc(-c3scnc3C)ccc2CNC(=O)[C@@H]2C[C@@H](O)CC2C(=O)[C@H](C(C)C)N2Cc3ccccc3C2=O)CC1]

Compound 02-027: 25 mg of 02-027 was obtained by a method similar to the one described for compound 02-024 using 58 as starting material instead of 54.

References: 1.Shi, F., Telesco, S. E., Liu, Y., Radhakrishnan, R. & Lemmon, M. A. ErbB3/HER3 intracellular domain is competent to bind ATP and catalyze autophosphorylation. Proceedings of the National Academy of Sciences 107, 7692 (2010). 2.Garske, A. L., Peters, U., Cortesi, A. T., Perez, J. L. & Shokat, K. M. Chemical genetic strategy for targeting protein kinases based on covalent complementarity. Proceedings of the National Academy of Sciences 108, 15046-15052 (2011). 3.Wissner, A. et al. 4-Anilino-6,7-dialkoxyquinoline-3-carbonitrile inhibitors of epidermal growth factor receptor kinase and their bioisosteric relationship to the 4-anilino-6,7-dialkoxyquinazoline inhibitors. Journal of Medicinal Chemistry 43, 3244-3256 (2000). 4.Okaniwa, M. et al. Design and synthesis of novel DFG-out RAF/vascular endothelial growth factor receptor 2 (VEGFR2) inhibitors. 1. Exploration of [5,6]-fused bicyclic scaffolds. Journal of Medicinal Chemistry 55, 3452-3478 (2012). 5.Wissner, A. et al. Synthesis and structure-activity relationships of 6,7-disubstituted 4-anilinoquinoline-3-carbonitriles. The design of an orally active, irreversible inhibitor of the tyrosine kinase activity of the epidermal growth factor receptor (EGFR) and the human epidermal growth factor receptor-2 (HER-2). Journal of Medicinal Chemistry 46, 49-63 (2002).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having the formula: 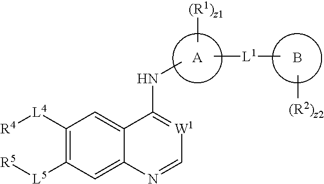
wherein:
Ring A is aryl or heteroaryl;
Ring B is aryl or heteroaryl;
W¹ is C(R⁶);
R¹ is independently halogen, —CX¹₃, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —CN, —SOₙ₂R¹⁴, —SOᵥ₂NR¹¹R¹², —NHNH₂, —ONR¹¹R¹², —NHC═(O)NHNR¹¹R¹², —NHC═(O)NR¹¹R¹², —N(O)ₘ₂, —NR¹¹R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR¹¹OR¹³, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R² substituents may optionally be joined to form a substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴₃, —OCHX⁴₂, —OCH₂X⁴₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is a degradation-increasing moiety;
R⁶ is hydrogen, halogen, —CX⁶₃, —CHX⁶₂, —CH₂X⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX⁶₃, —OCHX⁶₂, —OCH₂X⁶, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷, R⁸, R⁹, R¹⁰, R¹¹, R¹², R¹³, and R¹⁴ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R¹¹ and R¹² substituents bonded to the same nitrogen atom may optionally be joined to form a substituted or unsubstituted heterocycloalkyl or substituted or unsubstituted heteroaryl;
L¹ is a bond, —O—, —NH—, —C(O)—, —C(O)NH—, —NHC(O)—, —C(O)O—, —OC(O)—, —NHC(O)NH—, —S—, substituted or unsubstituted alkylene or substituted or unsubstituted heteroalkylene;
L⁴ is a bond or a divalent linker;
L⁵ is a divalent linker;
z1 and z2 are independently an integer from 0 to 7;
m1, m2, v1, and v2 are independently 1 or 2;
n1 and n2 are independently an integer from 0 to 4; and
X¹, X², X⁴, X⁶, and X^{A} are independently —Cl, —Br, —I, or —F.

2. The compound of claim 1 having the formula: 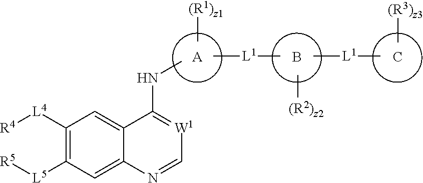
wherein
Ring A is phenyl or 5 or 6 membered heteroaryl;
Ring B is phenyl or 5 or 6 membered heteroaryl;
Ring C is C₃-C₆ cycloalkyl, 3 to 6 membered heterocycloalkyl, phenyl, or 5 to 6 membered heteroaryl;
R¹ is independently a halogen, —CX¹₃, —CHX¹₂, —CH₂X¹, —CN, —OH, —NH₂, —COOH, —CONH₂, —NO₂, —SH, substituted or unsubstituted C₁-C₄ alkyl, or 2 to 4 membered substituted or unsubstituted heteroalkyl;
R² is independently a halogen, —CX²₃, —CHX²₂, —CH₂X², —CN, —OH, —NH₂, —COOH, —CONH₂, —NO₂, —SH, substituted or unsubstituted C₁-C₄ alkyl, or 2 to 4 membered substituted or unsubstituted heteroalkyl;
R³ is independently a halogen, —CX³₃, —CHX³², —CH₂X³, —CN, —SOₙ₃R¹⁸, —SOᵥ₃NR¹⁵R¹⁶, —NHNH₂, —ONR¹⁵R¹⁶, —NHC═(O)NHNR¹⁵R¹⁶, —NHC═(O)NR¹⁵R¹⁶, —N(O)ₘ₃, —NR¹⁵R¹⁶, —C(O)R¹⁷, —C(O)—OR¹⁷, —C(O)NR¹⁵R¹⁶, —OR¹⁸, —NR¹⁵SO₂R¹⁷, —NR¹⁵C═(O)R¹⁷, —NR¹⁵C(O)—OR¹⁷, —NR¹⁵OR¹⁷, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl;
R⁶ is hydrogen, halogen, —CN, —OH, —NH₂, —COOH, —CONH₂, —NO₂, —SH, substituted or unsubstituted C₁-C₄ alkyl, or 2 to 4 membered substituted or unsubstituted heteroalkyl;
R¹⁵, R¹⁶, R¹⁷, and R¹⁸ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁵ and R¹⁶ substituents bonded to the same nitrogen atom may optionally be joined to form a substituted or unsubstituted heterocycloalkyl or substituted or unsubstituted heteroaryl;
L¹ is —O—, substituted or unsubstituted C₁-C₃ alkylene or substituted or unsubstituted 2 to 3 membered heteroalkylene;
L² is a bond, —O—, —NH—, —C(O)—, —C(O)NH—, —NHC(O)—, —C(O)O—, —OC(O)—, —NHC(O)NH—, —S—, substituted or unsubstituted C₁-C₃ alkylene or substituted or unsubstituted 2 to 3 membered heteroalkylene;
z1 and z2 are independently an integer from 0 to 4;
z3 is an integer from 0 to 5;
m3 and v3 are independently 1 or 2;
n3 is an integer from 0 to 4; and
X³ is —Cl, —Br, —I, or —F.

3. The compound of claim 2 having the formula:

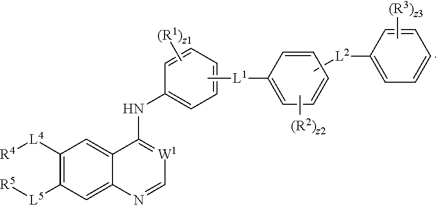

4. The compound of claim 3, having the formula: 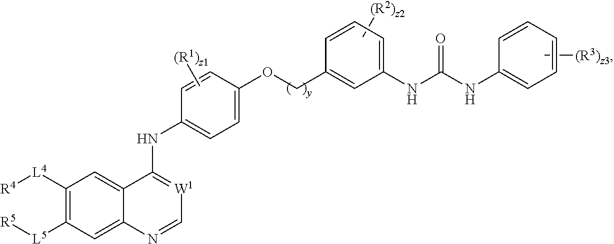
wherein Y is 0 or 1.

5. The compound of claim 3, having the formula: 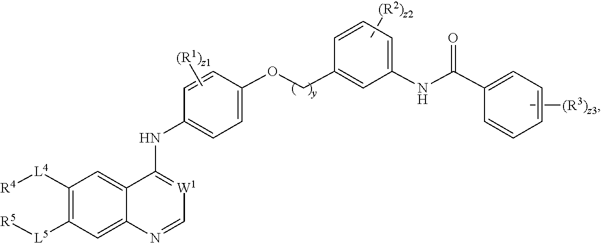
wherein Y is 0 or 1.

6. The compound of claim 1, wherein z1 and z2 are 0, and z3 is 1.

7. The compound of claim 2, wherein R³ is —CF₃ or halogen.

8. The compound of claim 1, wherein R⁶ is —CN.

9. The compound of claim 1, wherein -L⁴-R⁴ is unsubstituted methoxy.

10. The compound of claim 1, wherein L⁵ is L^{5A}-L^{5B}-L^{5C}-L^{5D}-L^{5E} and wherein L^{5A} L^{5B} L^{5C}, L^{5D}, and L^{5E} are each independently a bond, —O—, —NH—,a —C(O)—, —C(O)NH—, —NHC(O)—, —C(O)O—, —OC(O)—, —NHC(O)NH—, —S—, substituted or unsubstituted alkylene, substituted or unsubstituted heteroalkylene, substituted or unsubstituted, substituted or unsubstituted cycloalkylene, substituted or unsubstituted heterocycloalkylene, substituted or unsubstituted arylene or substituted or unsubstituted heteroarylene.

11. The compound of claim 10, wherein L^{5A} L^{5B} L^{5C}, L^{5D}, and L^{5E} are each independently a bond, —O—, —NH—, —C(O)—, —C(O)NH—, —NHC(O)—, —C(O)O—, —OC(O)—, —NHC(O)NH—, —S—, substituted or unsubstituted C₁-C₃ alkylene, substituted or unsubstituted 2 to 4 membered heteroalkylene, substituted or unsubstituted C₃-C₆ cycloalkylene, substituted or unsubstituted 3 to 6 membered heterocycloalkylene, substituted or unsubstituted C₆ arylene, or substituted or unsubstituted 5 to 6 membered heteroarylene.

12. The compound of claim 11, wherein at least one of L^{5A} L^{5B} L^{5C}, L^{5D} and L^{5E} is substituted or unsubstituted piperazinylene.

13. The compound of claim 12, wherein L^{5D} is substituted or unsubstituted piperazinylene.

14. The compound of claim 10, wherein L^{5A} L^{5B} L^{5C}, and L^{5E} are each independently a bond, O, substituted or unsubstituted C₁-C₃ alkylene or substituted or unsubstituted 2 to 4 membered heteroalkylene.

15. The compound of claim 10, wherein L^{5A} is a bond.

16. The compound of claim 10, wherein L^{5B} is a bond or 0.

17. The compound of claim 10, wherein L^{5C} is a bond, 0 or substituted or unsubstituted 2 to 4 membered heteroalkylene.

18. The compound of claim 10, wherein L^{5A} is a bond, L^{5B} is O, and L^{5C} is substituted or unsubstituted C₁-C₃ alkylene.

19. The compound of claim 10, wherein L^{5E} is a bond, or substituted or unsubstituted C₁-C₃ alkylene.

20. The compound of claim 1, wherein R⁵ is substituted or unsubstituted heteroalkylene.

21. The compound of claim 20, wherein R⁵ is substituted or unsubstituted 2 to 22 membered heteroalkylene having a 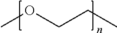
group, wherein n is 2 to 4.

22. The compound of claim 21, wherein R⁵ is substituted or unsubstituted 10 to 22 membered heteroalkylene.

23. The compound of claim 22, wherein R⁵ is substituted or unsubstituted 15 to 22 membered heteroalkylene.

24. The compound of claim 1, wherein the compound is:

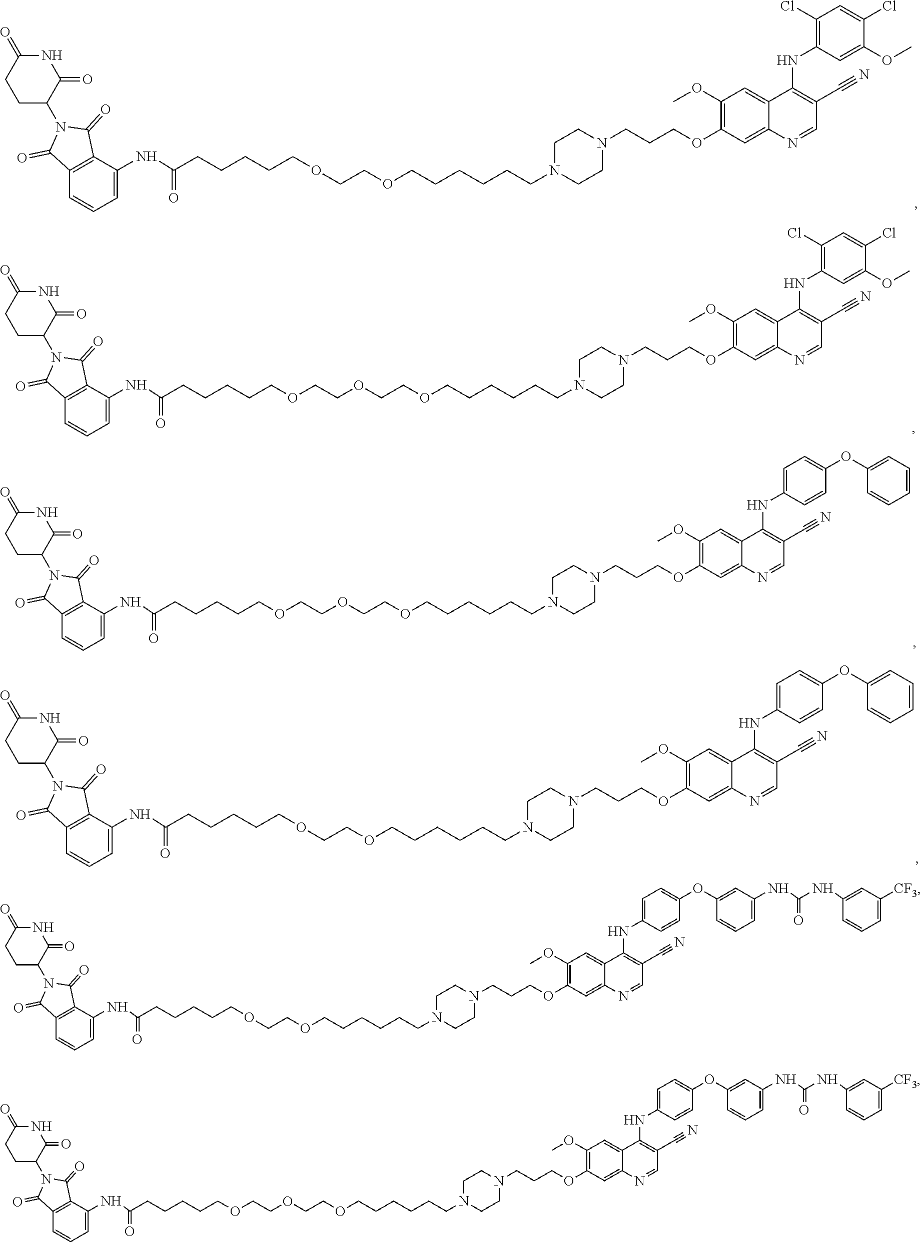

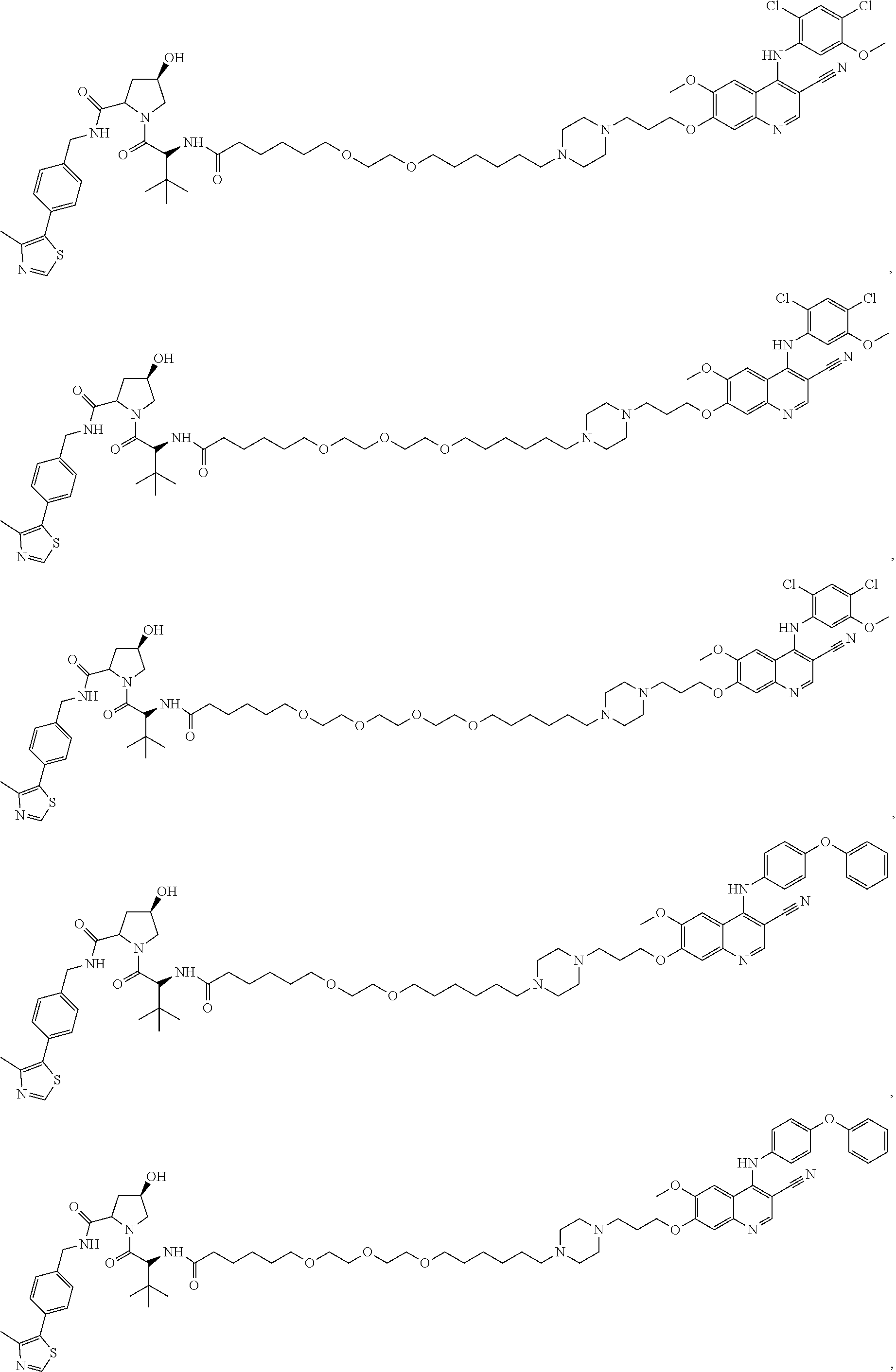

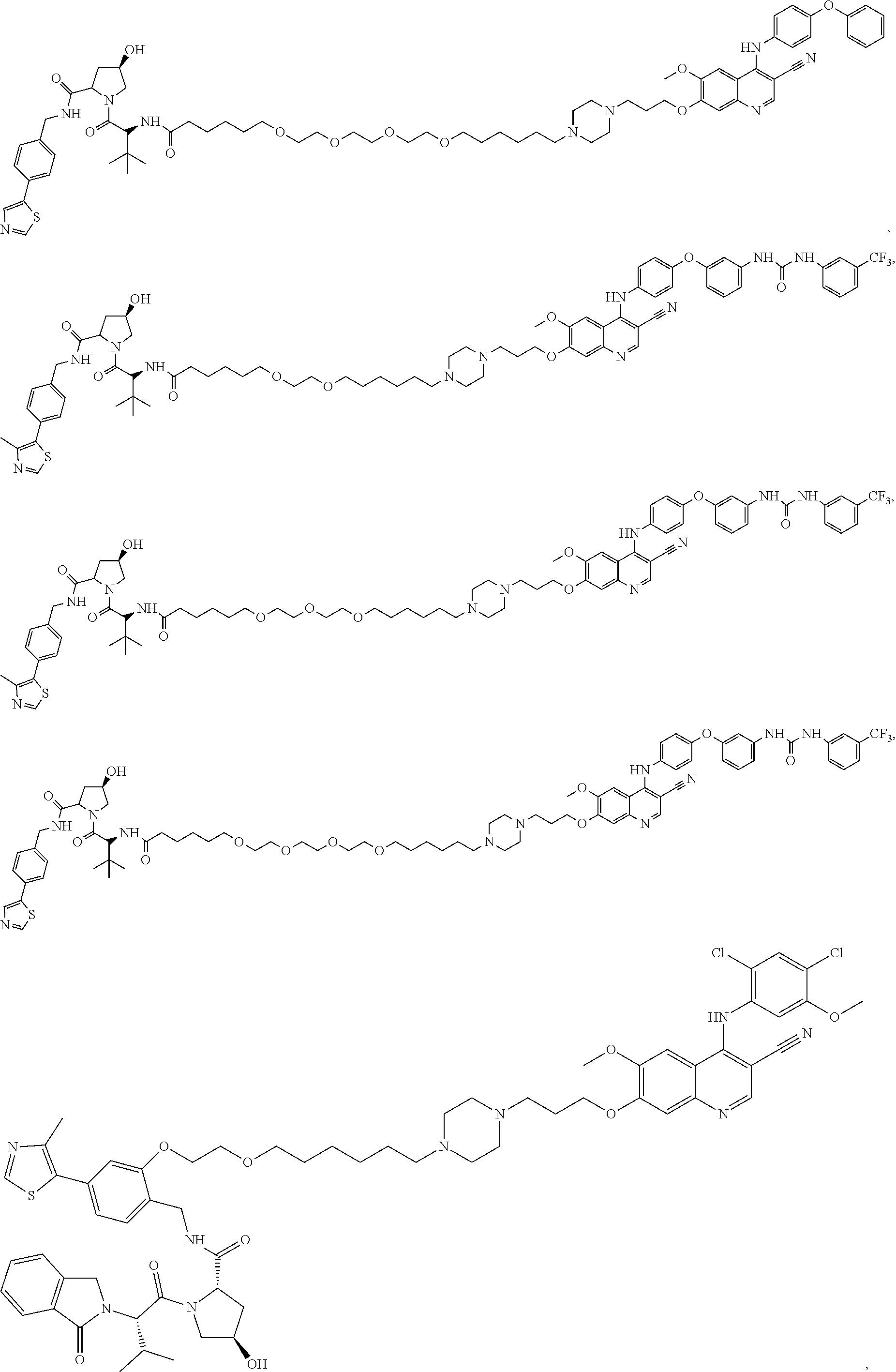

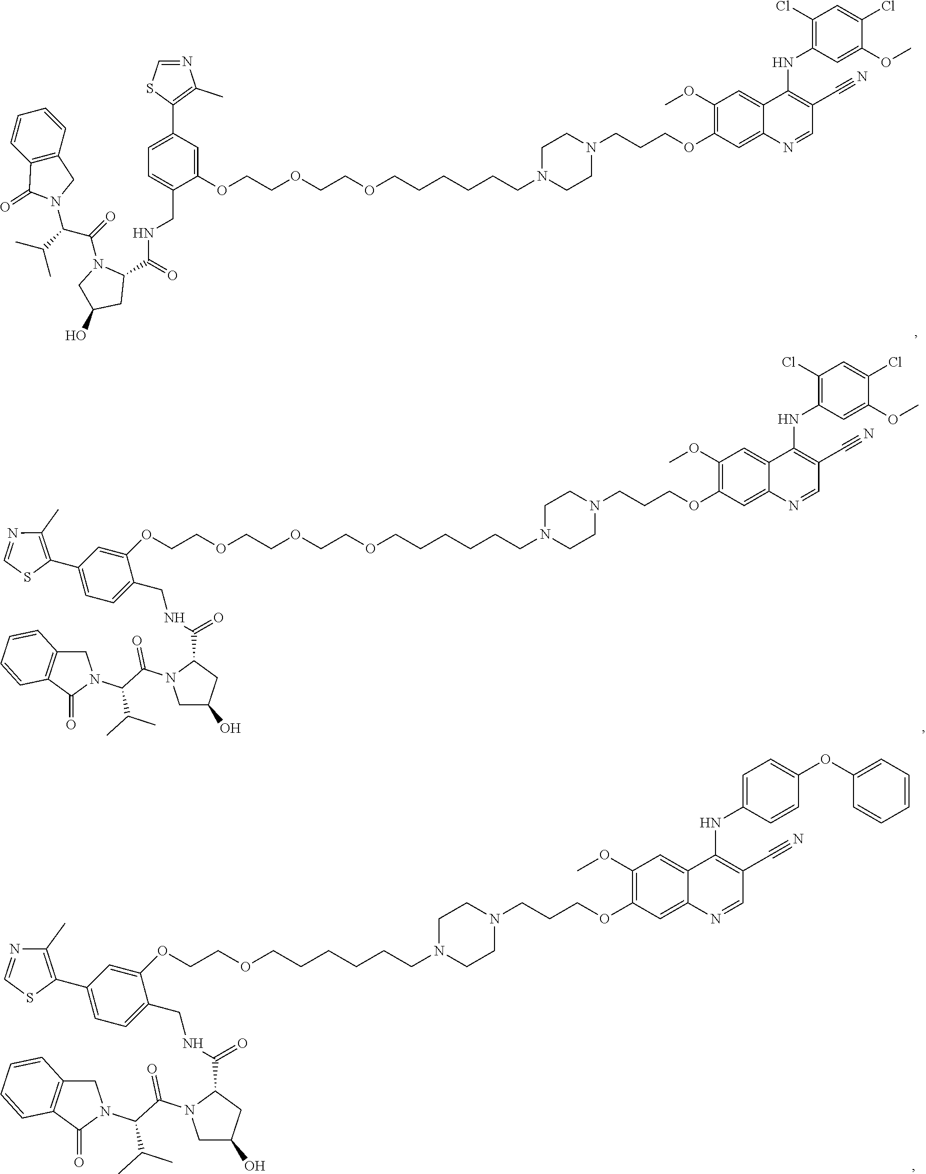 [SMILES: COc1cc(Nc2c(C#N)cnc3cc(OCCCN4CCN(CCCCCCOCCOCCOc5cc(-c6scnc6C)ccc5CNC(=O)[C@@H]5C[C@@H](O)CN5C(=O)[C@H](C(C)C)N5Cc6ccccc6C5=O)CC4)c(OC)cc23)c(Cl)cc1Cl ; COc1cc(Nc2c(C#N)cnc3cc(OCCCN4CCN(CCCCCCOCCOCCOCCOc5cc(-c6scnc6C)ccc5CNC(=O)[C@@H]5C[C@@H](O)CN5C(=O)[C@H](C(C)C)N5Cc6ccccc6C5=O)CC4)c(OC)cc23)c(Cl)cc1Cl ; COc1cc2c(Nc3ccc(Oc4ccccc4)cc3)c(C#N)cnc2cc1OCCCN1CCN(CCCCCCOCCOc2cc(-c3scnc3C)ccc2CNC(=O)[C@@H]2C[C@@H](O)CN2C(=O)[C@H](C(C)C)N2Cc3ccccc3C2=O)CC1]

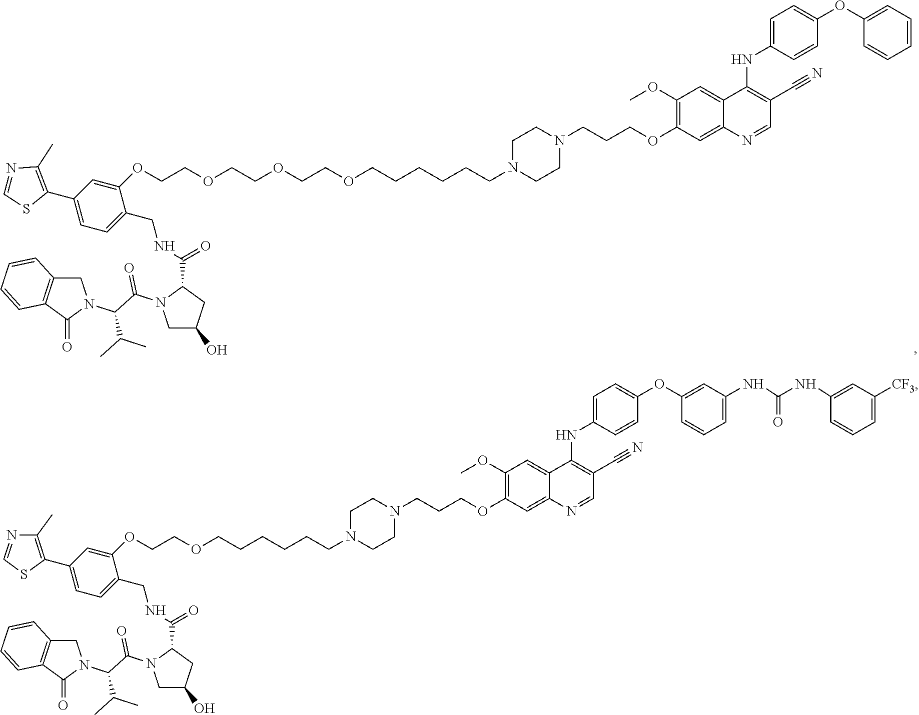 [SMILES: COc1cc2c(Nc3ccc(Oc4ccccc4)cc3)c(C#N)cnc2cc1OCCCN1CCN(CCCCCCOCCOCCOCCOc2cc(-c3scnc3C)ccc2CNC(=O)[C@@H]2C[C@@H](O)CN2C(=O)[C@H](C(C)C)N2Cc3ccccc3C2=O)CC1 ; COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOc2cc(-c3scnc3C)ccc2CNC(=O)[C@@H]2C[C@@H](O)CN2C(=O)[C@H](C(C)C)N2Cc3ccccc3C2=O)CC1]

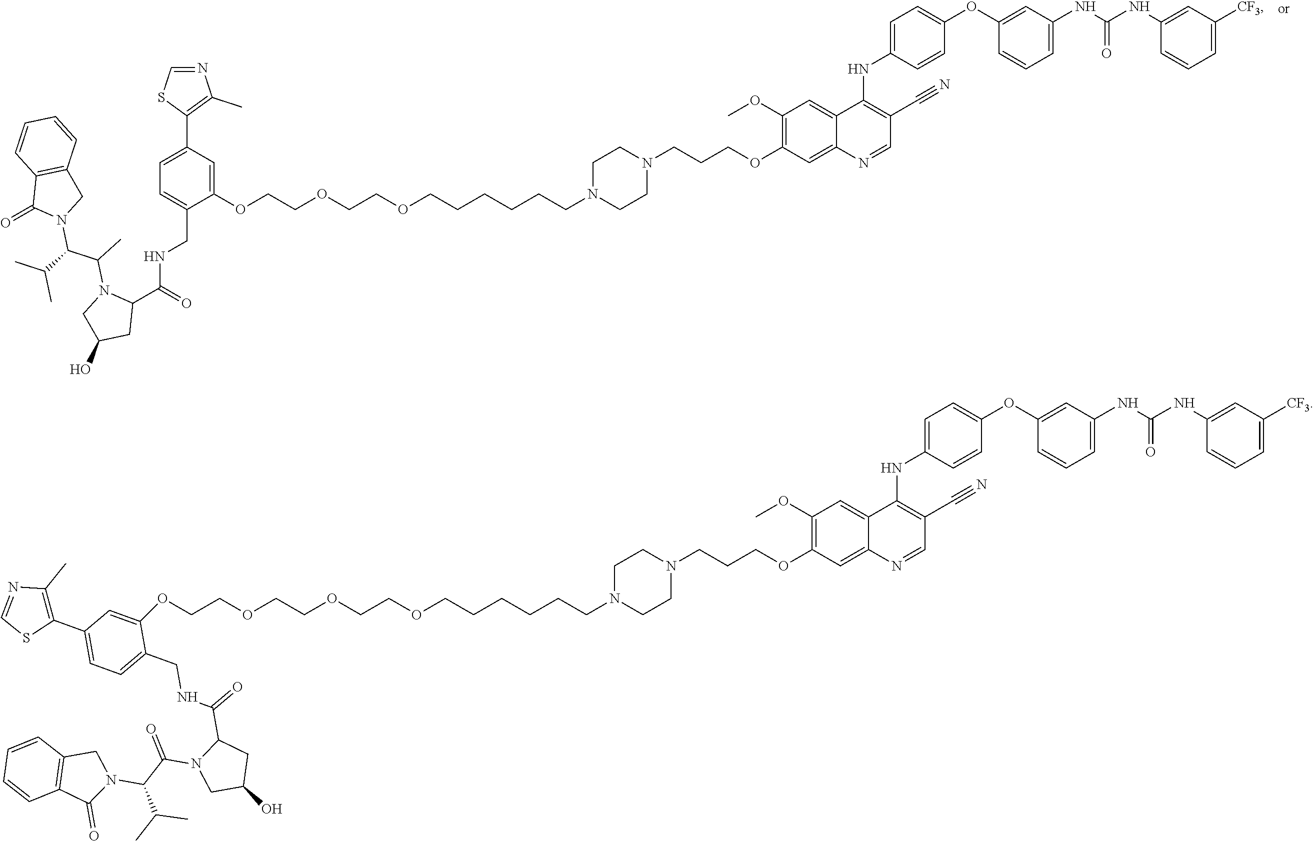 [SMILES: COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOCCOc2cc(-c3scnc3C)ccc2CNC(=O)C2C[C@@H](O)CN2C(C)[C@H](C(C)C)N2Cc3ccccc3C2=O)CC1 ; COc1cc2c(Nc3ccc(Oc4cccc(NC(=O)Nc5cccc(C(F)(F)F)c5)c4)cc3)c(C#N)cnc2cc1OCCCN1CCN(CCCCCCOCCOCCOCCOc2cc(-c3scnc3C)ccc2CNC(=O)C2C[C@@H](O)CN2C(=O)[C@H](C(C)C)N2Cc3ccccc3C2=O)CC1]

25. A pharmaceutical composition comprising a compound of claim 1, or a pharmaceutically acceptable salt thereof, and a pharmaceutically acceptable excipient.

26. A method of treating a disease associated with HER3 activity in a patient in need of such treatment, said method comprising administering a therapeutically effective amount of the compound of claim 1, or a pharmaceutically acceptable salt thereof.

27. A method of treating a disease associated with EGFR activity, HER2 activity, HER4 activity, c-MET activity, PI3K activity, MEK activity, MAPK activity, RAF activity, BRAF activity, AKT activity, RAS activity, KRAS activity, heregulin activity, or neuregulin activity in a patient in need of such treatment, said method comprising administering a therapeutically effective amount of the compound of claim 1, or a pharmaceutically acceptable salt thereof.

28. A method of treating cancer in a patient in need of such treatment, said method comprising administering a therapeutically effective amount of the compound of claim 1, or a pharmaceutically acceptable salt thereof.

29. A method of inhibiting HER3 activity, said method comprising contacting HER3 with an effective amount of the compound of claim 1, or a pharmaceutically acceptable salt thereof.
